(19) United States
(12) Patent Application Publication
(10) Pub. No.: US 2024/0123087 A1
(43) Pub. Date: Apr. 18, 2024

(54) POLYNUCLEOTIDE COMPOSITIONS, RELATED FORMULATIONS, AND METHODS OF USE THEREOF

(71) Applicant: ReCode Therapeutics, Inc., Menlo Park, CA (US)

(72) Inventors: Daniella Ishimaru, Menlo Park, CA (US); Brandon Wustman, Menlo Park, CA (US); Mirko Hennig, Menlo Park, CA (US); Dave Liston, Menlo Park, CA (US); Rumpa Bhattacharjee, Menlo Park, CA (US)

(21) Appl. No.: 18/282,181

(22) PCT Filed: Mar. 18, 2022
(86) PCT No.: PCT/US2022/021032

§ 371 Date: Sep. 14, 2023

Related U.S. Application Data
(60) Provisional application No. 63/163,484, filed on Mar. 19, 2021.

(51) Int. Cl. A61K 48/00; A61K 9/127; A61K 9/51; A61P 11/00; C07K 14/47

(52) U.S. Cl. CPC A61K 48/005; A61K 9/1271; A61K 9/5123; A61K 9/5146; A61P 11/00; C07K 14/47

(57) ABSTRACT

Compositions of polynucleotide(s) are disclosed. A polynucleotide may encode for a polypeptide, protein, or functional fragment thereof associated with primary ciliary dyskinesia (PCD). Pharmaceutical compositions, kits, and methods for treating a disease or condition associated with cilia maintenance and function, and impaired function of the axoneme are also disclosed. The polynucleotide may be combined with a lipid composition.

### CROSS-REFERENCE

This application claims the benefit of U.S. Provisional Application Ser. No. 63/163,484 filed on Mar. 19, 2021, the entirety of which is hereby incorporated by reference herein.

### BACKGROUND

Nucleic acids, such as messenger ribonucleic acid (mRNA) may be used by cells to express proteins and polypeptides. Some cells may be deficient in a certain protein or nucleic acid and result in disease states. A cell can also take up and translate an exogenous RNA, but many factors influence efficient uptake and translation. For instance, the immune system recognizes many exogenous RNAs as foreign and triggers a response that is aimed at inactivating the RNAs.

### SUMMARY

Provided here are composition comprising polynucleotides encoding a primary ciliary dyskinesia (PCD)-associated protein. The polynucleotides may be used a therapeutic. In particular, a polynucleotide may be mRNA to be delivered to a cell of a subject. Upon delivery of a nucleic acid to a cell, the polynucleotides may be used to synthesize a polypeptide. In the case of cell or subject with a disease or disorder, the polynucleotides may be effective at acting as a therapeutic by increasing the expression of a polypeptide. In cases, where a disorder or disease is caused or correlated to aberrant expression or activity of polypeptide, the increase in expression of the polypeptide may be beneficial.

Additionally, the compositions may comprise additional components such to improve treatment of a condition such as PCD. Many different types of compounds can be coupled or conjugated or allowed to encapsulate the polynucleotides such that delivery of the polynucleotide may be performed

In some aspects, present disclosure provides A synthetic polynucleotide encoding a primary ciliary dyskinesia (PCD)-associated protein, wherein the synthetic polynucleotide comprises a nucleic acid sequence (e.g., an open reading frame (ORF) sequence) having at least about 70% sequence identity over at least 500 or 1000 bases to a sequence selected from SEQ ID NOs: 1-32, 61, or 62. In some embodiments, the nucleic acid sequence has at least about 75% (e.g., at least about 80%, 81%, 82%, 83%, 84%, 85%, 86%, 87%, 88%, 89%, 90%, 91%, 92%, 93%, 94%, 95%, 96%, 97%, 98%, or 99%) sequence identity over at least 500 or 1000 bases to a sequence selected from SEQ ID NOs: 1-32, 61, or 62. In some embodiments, the nucleic acid sequence has 100% sequence identity over at least 500 or 1000 bases to a sequence selected from SEQ ID NOs: 1-32, 61, or 62. In some embodiments, the nucleic acid sequence has at least about 70% sequence identity to a sequence selected from SEQ ID NOs: 1-32, 61, or 62. In some embodiments, the nucleic acid sequence has at least about 75% (e.g., at least about 80%, 81%, 82%, 83%, 84%, 85%, 86%, 87%, 88%, 89%, 90%, 91%, 92%, 93%, 94%, 95%, 96%, 97%, 98%, or 99%) sequence identity to a sequence selected from SEQ ID NOs: 1-32, 61, or 62. In some embodiments, the nucleic acid sequence comprises a reduced number or frequency of at least one codon selected from the group consisting of GCG, GCA, GCT, TGT, GAT, GAG, TTT, GGG, GGT, CAT, ATA, ATT, AAG, TTG, TTA, CTA, CTT, CTC, AAT, CCG, CCA, CCT, CAG, AGG, CGG, CGA, CGT, CGC, TCG, TCA, TCT, TCC, ACG, ACT, GTA, GTT, GTC, and TAT, as compared to a corresponding wild-type sequence selected from SEQ ID NOs: 33-39. In some embodiments, the nucleic acid sequence comprises an increased number or frequency of at least one codon comprising one or more codons selected from: GCC, TGC, GAC, GAA, TTC, GGA, GGC, CAC, ATC, AAA, CTG, AAC, CCT, CCC, CAA, AGA, AGC, ACA, ACC, GTG, and TAC, as compared to a corresponding wild-type sequence selected from SEQ ID NOs: 33-39. In some embodiments, the nucleic acid sequence comprises fewer codon types encoding an amino acid as compared to a corresponding wild-type sequence selected from SEQ ID NOs: 33-39. In some embodiments, at least one type of an isoleucine-encoding codon in the corresponding wild-type sequence is substituted with a synonymous codon type in the nucleic acid sequence. In some embodiments, at least one type of a valine-encoding codon in the corresponding wild-type sequence is substituted with a synonymous codon type in the nucleic acid sequence. In some embodiments, at least one type of an alanine-encoding codon in the corresponding wild-type sequence is substituted with a synonymous codon type in the nucleic acid sequence. In some embodiments, at least one type of a glycine-encoding codon in the corresponding wild-type sequence is substituted with a synonymous codon type in the nucleic acid sequence. In some embodiments, at least one type of a proline-encoding codon in the corresponding wild-type sequence is substituted with a synonymous codon type in the nucleic acid sequence. In some embodiments, at least one type of a threonine-encoding codon in the corresponding wild-type sequence is substituted with a synonymous codon type in the nucleic acid sequence. In some embodiments, at least one type of a leucine-encoding codon in the corresponding wild-type sequence is substituted with a synonymous codon type in the nucleic acid sequence. In some embodiments, at least one type of an arginine-encoding codon in the corresponding wild-type sequence is substituted with a synonymous codon type in the nucleic acid sequence. In some embodiments, at least one type of a serine-encoding codon in the corresponding wild-type sequence is substituted with a synonymous codon type in the nucleic acid sequence.

In some embodiments, at least about 90% phenylalanine-encoding codons of the synthetic polynucleotide are TTC (as opposed to TTT). In some embodiments, at least about 60% cysteine-encoding codons of the synthetic polynucleotide are TGC (as opposed to TGT). In some embodiments, the at least about 70% aspartic acid-encoding codons of the synthetic polynucleotide are GAC (as opposed to GAT). In some embodiments, at least about 50% glutamic acid-encoding codons of the synthetic polynucleotide are GAG (as opposed to GAA). In some embodiments, at least about 60% histidine-encoding codons of the synthetic polynucleotide are CAC (as opposed to CAT). In some embodiments, at least about 60% lysine-encoding codons of the synthetic polynucleotide are AAG (as opposed to AAA). In some embodiments, at least about 60% asparagine-encoding codons of the synthetic polynucleotide are AAC (as opposed to AAT). In some embodiments, at least about 70% glutamine-encoding codons of the synthetic polynucleotide are CAG (as opposed to CAA). In some embodiments, at least about 80% tyrosine-encoding codons of the synthetic polynucleotide are TAC (as opposed to TAT). In some embodiments, at least about 90% isoleucine-encoding codons of the synthetic polynucleotide are ATC. In some embodiments, the synthetic polynucleotide comprises no more than 2 types of isoleucine-encoding codons. In some embodiments, the synthetic polynucleotide comprises no more than 3 types of alanine (Ala)-encoding codons. In some embodiments, the synthetic polynucleotide comprises no more than 3 types of glycine (Gly)-encoding codons. In some embodiments, the synthetic polynucleotide comprises no more than 3 types of proline (Pro)-encoding codons. In some embodiments, the synthetic polynucleotide comprises no more than 3 types of threonine (Thr)-encoding codons. In some embodiments, the synthetic polynucleotide comprises no more than 5 or 4 type(s) of arginine (Arg)-encoding codons. In some embodiments, the synthetic polynucleotide comprises no more than 5 or 4 type(s) of serine (Ser)-encoding codons. In some embodiments, a frequency of GCC codon is higher than a frequency of GCA codon. In some embodiments, a frequency of GCC codon is higher than a frequency of GCT codon. In some embodiments, a frequency of GCT codon is lower than a frequency of GCA codon. In some embodiments, a frequency of GCT codon is higher than a frequency of GCA codon. In some embodiments, a frequency of GCG codon is no more than about 10% or 5%. In some embodiments, a frequency of GCA codon is no more than about 20%. In some embodiments, a frequency of GCT codon is at least about 1%, 5%, 10%, 15%, 20%, or 25%. In some embodiments, a frequency of GCT codon is no more than about 30%, 25%, 20%, 15%, 10%, or 5%. In some embodiments, a frequency of GCC codon is at least about 60%, 70%, 80%, or 90%. In some embodiments, a frequency of GCC codon is no more than about 95%, 90%, 85%, 80%, or 75%. In some embodiments, a frequency of GGC codon is lower than a frequency of GGA codon. In some embodiments, a frequency of GGC codon is higher than a frequency of GGA codon. In some embodiments, a frequency of GGG codon is no more than about 10% or 5%. In some embodiments, a frequency of GGG codon is at least about 1%. In some embodiments, a frequency of GGA codon is no more than about 30% or 20%. In some embodiments, a frequency of GGA codon is at least about 10% or 20%. In some embodiments, a frequency of GGT codon is no more than about 10% or 5%. In some embodiments, a frequency of GGC codon is no more than about 90%, 80%, or 70%. In some embodiments, a frequency of GGC codon is at least about 60%, 70%, or 80%. In some embodiments, a frequency of CCC codon is lower than a frequency of CCT codon. In some embodiments, a frequency of CCC codon is higher than a frequency of CCT codon. In some embodiments, a frequency of CCC codon is lower than a frequency of CCA codon. In some embodiments, a frequency of CCC codon is higher than a frequency of CCA codon. In some embodiments, a frequency of CCT codon is lower than a frequency of CCA codon. In some embodiments, a frequency of CCT codon is higher than a frequency of CCA codon. In some embodiments, a frequency of CCG codon is no more than about 10% or 5%. In some embodiments, a frequency of CCA codon is no more than about 30%, 20%, or 10%. In some embodiments, a frequency of CCA codon is at least about 5%, 10%, 15%, 20%, or 25%. In some embodiments, a frequency of CCT codon is no more than about 60%, 50%, 40%, or 30%. In some embodiments, a frequency of CCT codon is at least about 20%, 30%, 40%, or 50%. In some embodiments, a frequency of CCC codon is no more than about 60%, 50%, or 40%. In some embodiments, a frequency of CCC codon is at least about 30%, 40%, 50%, 60%, or 70%. In some embodiments, a frequency of ACA codon is higher than a frequency of ACT codon. In some embodiments, a frequency of ACC codon is higher than a frequency of ACT codon. In some embodiments, a frequency of ACC codon is lower than a frequency of ACA codon. In some embodiments, a frequency of ACC codon is higher than a frequency of ACA codon. In some embodiments, a frequency of ACG codon is no more than about 10% or 5%. In some embodiments, a frequency of ACA codon is no more than about 60%, 50%, 40%, or 30%. In some embodiments, a frequency of ACA codon is at least about 10%, 20%, 30%, 40%, or 50%. In some embodiments, a frequency of ACT codon is no more than about 10% or 5%. In some embodiments, a frequency of ACC codon is no more than about 90%, 80%, 70%, 60%, or 50%. In some embodiments, a frequency of ACC codon is at least about 40%, 50%, 60%, 70%, or 80%. In some embodiments, a frequency of AGA codon is lower than a frequency of AGG codon. In some embodiments, a frequency of AGA codon is higher than a frequency of AGG codon. In some embodiments, a frequency of AGA codon is lower than a frequency of CGG codon. In some embodiments, a frequency of AGA codon is higher than a frequency of CGG codon. In some embodiments, a frequency of CGG codon is higher than a frequency of CGA codon. In some embodiments, a frequency of CGG codon is higher than a frequency of CGC codon. In some embodiments, a frequency of AGG codon is no more than about 10%. In some embodiments, a frequency of AGG codon is less than about 10%. In some embodiments, a frequency of AGA codon is no more than about 70%, 60%, or 50%. In some embodiments, a frequency of AGA codon is at least about 40%, 50%, 60%, or 70%. In some embodiments, a frequency of CGG codon is no more than about 50%, 40%, or 30%. In some embodiments, a frequency of CGG codon is at least about 20%, 30%, or 40%. In some embodiments, a frequency of CGA codon is at least about 1%. In some embodiments, a frequency of CGA codon is no more than about 10% or 5%. In some embodiments, a frequency of CGT codon is no more about 10% or 5%. In some embodiments, a frequency of CGC codon is no more than about 20%, 10%, or 5%. In some embodiments, a frequency of CGC codon is at least about 1%, 2%, 3%, 4%, or 5%. In some embodiments, a frequency of AGC codon is higher than a frequency of TCT codon. In some embodiments, a frequency of TCT codon is higher than a frequency of TCG codon. In some embodiments, a frequency of TCT codon is higher than a frequency of TCA codon. In some embodiments, a frequency of TCT codon is higher than a frequency of TCC codon. In some embodiments, a frequency of AGT codon is no more than about 10%. In some embodiments, a frequency of AGT codon is at least about 1%. In some embodiments, a frequency of AGC codon is no more about 95%, 90%, 85%, or 80%. In some embodiments, a frequency of AGC codon is at least about 70%, 80%, or 90%. In some embodiments, a frequency of TCG codon is no more than about 10% or 5%. In some embodiments, a frequency of TCA codon is no more than about 10% or 5%. In some embodiments, a frequency of TCT codon is no more than about 30%, 20%, or 10%. In some embodiments, a frequency of TCT codon is at least about 10%, or 20%. In some embodiments, a frequency of TCC codon is no more than about 10% or 5%.

In some embodiments, the polynucleotide further comprises a 3′ or 5′ noncoding region. In some embodiments, the 3′ or 5′ noncoding region enhances an expression of the PCD-associated polypeptide encoded by the synthetic polynucleotide within cells. In some embodiments, the polynucleotide further comprises a 5′ cap structure. In some embodiments, the 5′ cap structure improves a pharmacokinetic characteristic (e.g., a prolonged half-life) of the polynucleotide in a subject. In some embodiments, the 5′cap structure is a Cap-1 structure. In some embodiments, the 3′ noncoding region comprises a poly adenosine tail. In some embodiments, the poly adenosine tail comprises at most 200 adenosines. In some embodiments, the poly adenosine tail improves a pharmacokinetic characteristic (e.g., a prolonged half-life) of the polynucleotide in a subject. In some embodiments, the synthetic polynucleotide encodes a cytoplasmic dynein assembly factor. In some embodiments, the synthetic polynucleotide encodes a cytoplasmic or axonemal dynein component protein. In some embodiments, the synthetic polynucleotide is a messenger ribonucleotide (mRNA) of a gene set forth in Table 1. In some embodiments, the synthetic polynucleotide is an mRNA of a gene selected from the group consisting of DNAHS, ARMC4, ZMYND10, DNAAF4, CCDC40, CCDC39, DNAAF1, DNAI2, and DAAF2. In some embodiments, the synthetic polynucleotide is not a messenger ribonucleotide (mRNA) of DNAIl. In some embodiments, the synthetic polynucleotide comprises one or more nucleoside analogue(s) (e.g., one or more uridine analogue(s), such as 1-methylpseudouridine). In some embodiments, no more than 50% of nucleosides within the synthetic polynucleotide are nucleoside analogue(s) (e.g., uridine analogue(s), such as 1-methylpseudouridine). In some embodiments, no more than 20% of nucleosides within the synthetic polynucleotide are nucleoside analogue(s). In some embodiments, substantially all (e.g., at least about 80%, 90%, 95%, 97%, or 99%) nucleosides replacing uridine within the synthetic polynucleotide are nucleoside analogues.

In another aspect, the present disclosure provides a pharmaceutical composition comprising a synthetic polynucleotide as described elsewhere herein combined with a lipid composition.

In another aspect, the present disclosure provides a polynucleotide combined with a lipid composition, which polynucleotide (1) encodes a primary ciliary dyskinesia (PCD)-associated protein and (2) comprises a nucleic acid sequence having at least about 70% sequence identity over at least 500 or 1000 bases to a sequence selected from SEQ ID NOs: 1-32, 61, or 62. In some embodiments, the pharmaceutical composition comprises a cationic lipid or a cationic polymer. In some embodiments, the pharmaceutical composition further comprises a phospholipid. In some embodiments, pharmaceutical composition further comprises a polymer-conjugated lipid (e.g., poly(ethylene glycol) (PEG)-conjugated lipid). In some embodiments, pharmaceutical composition further comprises a steroid or steroid derivative. In some embodiments, the pharmaceutical formulation is formulated in a nanoparticle or a nanocapsule. In some embodiments, the pharmaceutical formulation is formulated for local or systemic administration.

In another aspect, the present disclosure provides a method for treating a subject having or suspected of having primary ciliary dyskinesia (PCD), comprising administering to the subject in need thereof a composition comprising a synthetic polynucleotide that encodes a PCD-associated protein, which synthetic polynucleotide comprises a nucleic acid sequence having at least about 70% sequence identity over at least 500 or 1,000 bases to a sequence selected from SEQ ID NOs: 1-32, 61, or 62, thereby resulting in a heterologous expression of the PCD-associated protein within cells of the subject.

In another aspect, the present disclosure provides a method for treating a subject having or suspected of having primary ciliary dyskinesia (PCD), comprising administering to the subject in need thereof a pharmaceutical composition as disclosed elsewhere herein, thereby resulting in a heterologous expression of the PCD-associated protein within cells of the subject.

In another aspect, the present disclosure provides a method for treating a subject having or suspected of having primary ciliary dyskinesia (PCD), comprising administering to the subject in need thereof a pharmaceutical composition comprising a polynucleotide combined with a lipid composition, which polynucleotide (1) encodes a primary ciliary dyskinesia (PCD)-associated protein and (2) comprises a nucleic acid sequence having at least about 70% sequence identity over at least 500 or 1,000 bases to a sequence selected from SEQ ID NOs: 1-32, 61, or 62, thereby resulting in a heterologous expression of the PCD-associated protein within cells of the subject.

In some embodiments, the subject is a human. In some embodiments, the subject is determined to have an aberrant expression or activity of a PCD-associated gene or protein. In some embodiments, the cells are ciliated cells. In some embodiments, the cells are differentiated cells. In some embodiments, the cells are undifferentiated cells. In some embodiments, the ciliated cells are ciliated epithelial cells (e.g., ciliated airway epithelial cells). In some embodiments, the ciliated epithelial cells are undifferentiated. In some embodiments, the ciliated epithelial cells are differentiated. In some embodiments, the cells are in a lung of the subject.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### Incorporation by Reference

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIGS. 1A-1D show western blots of cells expressing proteins. FIG. 1A shows an anti-FLAG blot of DNAH5 expression. FIG. 1B shows an anti-HA blot of DNAAF1, DNAAF2, and DNAAF4 expression. FIG. 1C shows anti-HA blot of ARMC4 expression. FIG. 1D shows an anti-ZMYND10 blot of ZMYND 10 expression.

FIG. 2A shows an anti-DNAI1 and anti-DNAI2 blot of and DNAI2 expression. FIG. 2B shows a western blot of a co-immunoprecipitation of DNAI1 and DNAI2 co-transfections.

FIG. 3A illustrates immunofluorescent staining of the fixed cells with cell type-specific antibodies: ciliated cell (acetylated tubulin antibody); basal cell (cytokeratin 5 antibody); club cells (SCGB1a1/CC10 antibody), and nuclei (Hoechst).

FIG. 3B illustrates axoneme incorporation of CCDC39-HA in the CCDC39 negative PCD patient cell (HNEC) after single dose or two doe treatment.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “subject,” as used herein generally refers to a human. In some instances, a subject can also be an animal, such as a mouse, a rat, a guinea pig, a dog, a cat, a horse, a rabbit, and various other animals. A subject can be of any age, for example, a subject can be an infant, a toddler, a child, a pre-adolescent, an adolescent, an adult, or an elderly individual.

The term “disease,” as used herein, generally refers to an abnormal physiological condition that affects part or all of a subject, such as an illness (e.g., primary ciliary dyskinesia) or another abnormality that causes defects in the action of cilia in, for example, the lining the respiratory tract (lower and upper, sinuses, Eustachian tube, middle ear), in a variety of lung cells, in the fallopian tube, or flagella of sperm cells.

The term “polynucleotide” or “nucleic acid” as used herein generally refers to a polymeric form of nucleotides of any length, either ribonucleotides or deoxyribonucleotides, that comprise purine and pyrimidine bases, purine and pyrimidine analogues, chemically or biochemically modified, natural or non-natural, or derivatized nucleotide bases. Polynucleotides include sequences of deoxyribonucleic acid (DNA), ribonucleic acid (RNA), or DNA copies of ribonucleic acid (cDNA), all of which can be recombinantly produced, artificially synthesized, or isolated and purified from natural sources. The polynucleotides and nucleic acids may exist as single-stranded or double-stranded. The backbone of the polynucleotide can comprise sugars and phosphate groups, as may typically be found in RNA or DNA, or analogues or substituted sugar or phosphate groups. A polynucleotide may comprise naturally occurring or non-naturally occurring nucleotides, such as methylated nucleotides and nucleotide analogues (or analogs).

The term “polyribonucleotide,” as used herein, generally refers to polynucleotide polymers that comprise ribonucleic acids. The term also refers to polynucleotide polymers that comprise chemically modified ribonucleotides. A polyribonucleotide can be formed of D-ribose sugars, which can be found in nature.

The term “polypeptides,” as used herein, generally refers to polymer chains comprised of amino acid residue monomers which are joined together through amide bonds (peptide bonds). A polypeptide can be a chain of at least three amino acids, a protein, a recombinant protein, an antigen, an epitope, an enzyme, a receptor, or a structure analogue or combinations thereof. As used herein, the abbreviations for the L-enantiomeric amino acids that form a polypeptide are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). X or Xaa can indicate any amino acid.

The term “engineered,” as used herein, generally refers to polynucleotides, vectors, and nucleic acid constructs that have been genetically designed and manipulated to provide a polynucleotide intracellularly. An engineered polynucleotide can be partially or fully synthesized in vitro. An engineered polynucleotide can also be cloned. An engineered polyribonucleotide can contain one or more base or sugar analogues, such as ribonucleotides not naturally-found in messenger RNAs. An engineered polyribonucleotide can contain nucleotide analogues that exist in transfer RNAs (tRNAs), ribosomal RNAs (rRNAs), guide RNAs (gRNAs), small nuclear RNA (snRNA), small nucleolar RNA (snoRNA), SmY RNA, spliced leader RNA (SL RNA), CRISPR RNA, long noncoding RNA (lncRNA), microRNA (miRNA), or another suitable RNA.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “at least one,” as used herein in connection with codon usage, generally refers one or more synonymous codon(s) (e.g., at least two, at least three, etc.) up to the entire set of synonymous codons that encode the corresponding amino acid.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present disclosure which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The term “molar percentage” or “molar %” as used herein in connection with lipid composition(s) generally refers to the molar proportion of that component lipid relative to compared to all lipids formulated or present in the lipid composition.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### Primary Ciliary Dyskinesia (PCD) & Associated Targets

The present disclosure provides, in some embodiments, compositions and methods for the treatment of conditions associated with cilia maintenance and function, with nucleic acids encoding a protein or protein fragment(s). Numerous eukaryotic cells carry appendages, which are often referred to as cilia or flagella, whose inner core comprises a cytoskeletal structure called the axoneme. The axoneme can function as the skeleton of cellular cytoskeletal structures, both giving support to the structure and, in some instances, causing it to bend. Usually, the internal structure of the axoneme is common to both cilia and flagella. Cilia are often found in the linings of the airway, the reproductive system, and other organs and tissues. Flagella are tail-like structures that, similarly to cilia, can propel cells forward, such as sperm cells.

Without properly functioning cilia in the airway, bacteria can remain in the respiratory tract and cause infection. In the respiratory tract, cilia move back and forth in a coordinated way to move mucus towards the throat. This movement of mucus helps to eliminate fluid, bacteria, and particles from the lungs. Many infants afflicted with cilia and flagella malfunction experience breathing problems at birth, which suggests that cilia play an important role in clearing fetal fluid from the lungs. Beginning in early childhood, subjects afflicted with cilia malfunction can develop frequent respiratory tract infections.

Primary ciliary dyskinesia is a condition characterized by chronic respiratory tract infections, abnormally positioned internal organs, and the inability to have children (infertility). The signs and symptoms of this condition are caused by abnormal cilia and flagella. Subjects afflicted with primary ciliary dyskinesia often have year-round nasal congestion and a chronic cough. Chronic respiratory tract infections can result in a condition called bronchiectasis, which damages the passages, called bronchi, leading from the windpipe to the lungs and can cause life-threatening breathing problems.

The methods, constructs, and compositions of this disclosure provide a method to treat primary ciliary dyskinesia (PCD), also known as immotile ciliary syndrome or Kartagener syndrome. PCD is typically considered to be a rare, ciliopathic, autosomal recessive genetic disorder that often causes defects in the action of cilia lining the respiratory tract (lower and upper, sinuses, Eustachian tube, middle ear) and fallopian tube, as well as in the flagella of sperm cells.

Some individuals with primary ciliary dyskinesia have abnormally placed organs within their chest and abdomen. These abnormalities arise early in embryonic development when the differences between the left and right sides of the body are established. About 50 percent of people with primary ciliary dyskinesia have a mirror-image reversal of their internal organs (situs inversus totalis). For example, in these individuals the heart is on the right side of the body instead of on the left. When someone afflicted with primary ciliary dyskinesia has situs inversus totalis, they are often the ones to also have Kartagener syndrome.

Approximately 12 percent of people with primary ciliary dyskinesia have a condition known as heterotaxy syndrome or situs ambiguus, which is characterized by abnormalities of the heart, liver, intestines, or spleen. These organs may be structurally abnormal or improperly positioned. In addition, affected individuals may lack a spleen (asplenia) or have multiple spleens (polysplenia). Heterotaxy syndrome results from problems establishing the left and right sides of the body during embryonic development. The severity of heterotaxy varies widely among affected individuals.

Primary ciliary dyskinesia can also lead to infertility. Vigorous movements of the flagella are can be needed to propel the sperm cells forward to the female egg cell. Because the sperm of subjects afflicted with primary ciliary dyskinesia does not move properly, males with primary ciliary dyskinesia are usually unable to father children. Infertility occurs in some affected females and it is usually associated with abnormal cilia in the fallopian tubes.

Another feature of primary ciliary dyskinesia is recurrent ear infections (otitis media), especially in young children. Otitis media can lead to permanent hearing loss if untreated. The ear infections are likely related to abnormal cilia within the inner ear.

Rarely, individuals with primary ciliary dyskinesia have an accumulation of fluid in the brain (hydrocephalus), likely due to abnormal cilia in the brain.

The polyribonucleotides of the disclosure can be used, for example, to treat a subject having or at risk of having primary ciliary dyskinesia or any other condition associated with a defect or malfunction of a gene whose function is linked to cilia maintenance and function. Non limiting examples of genes that have been associated with primary ciliary dyskinesia include: armadillo repeat containing 4 (ARMC4), chromosome 21 open reading frame 59 (C21orf59), coiled-coil domain containing 103 (CCDC103), coiled-coil domain containing 114 (CCDC114), coiled-coil domain containing 39 (CCDC39), coiled-coil domain containing 40 (CCDC40), coiled-coil domain containing 65 (CCDC65), cyclin O (CCNO), dynein (axonemal) assembly factor 1 (DNAAF1), dynein (axonemal) assembly factor 2 (DNAAF2) (e.g., DNAAF2/Ktu), dynein (axonemal) assembly factor 3 (DNAAF3), dynein (axonemal) assembly factor 4 (DNAAF4), dyslexia susceptibility 1 candidate 1 (DYX1C1), dynein (axonemal) assembly factor 5 (DNAAF5), dynein axonemal heavy chain 11 (DNAH11), dynein axonemal heavy chain 5 (DNAH5), dynein axonemal heavy chain 6 (DNAH6),dynein axonemal heavy chain 8 (DNAH8), dynein axonemal intermediate chain 1 (DNAI1), dynein axonemal intermediate chain 2 (DNAI2), dynein axonemal light chain 1 (DNAL1), dynein regulatory complex subunit 1 (DRC1), growth arrest specific 8 (GASB), axonemal central pair apparatus protein (HYDIN), leucine rich repeat containing 6 (LRRC6), leucine rich repeat containing 50 (LRRC50), NME/NM23 family member 8 (NME8), oral-facial-digital syndrome 1 (OFD1), retinitis pigmentosa GTPase regulator (RPGR), radial spoke head 1 homolog (Chlamydomonas) (RSPH1), radial spoke head 4 homolog A (Chlamydomonas) (RSPH4A), radial spoke head 9 homolog (Chlamydomonas) (RSPH9), sperm associated antigen 1(SPAG1), and zinc finger MYND-type containing 10 (ZMYND10).

In some cases, the composition comprises a nucleic acid construct encoding dynein axonemal intermediate chain 2 (DNAI2), and upon translation within the cells of a subject the construct yields a polypeptide that treats a subject having or at risk of having primary ciliary dyskinesia. The DNAI2 gene is part of the dynein complex of respiratory cilia and sperm flagella. Mutations in this gene have been associated with primary ciliary dyskinesia type 9, a disorder characterized by abnormalities of motile cilia, respiratory infections leading to chronic inflammation and bronchiectasis, and abnormalities in sperm tails.

In some cases, the composition comprises a nucleic acid construct encoding armadillo repeat containing 4 (ARMC4), and upon translation within the cells of a subject the construct yields a polypeptide that treats a subject having or at risk of having of primary ciliary dyskinesia. The protein encoded by the ARMC4 gene comprises ten Armadillo repeat motifs (ARMs) and one HEAT repeat, and has been shown to localize to the ciliary axonemes and at the ciliary base of respiratory cells. Mutations in the ARMC4 gene can cause partial outer dynein arm (ODA) defects in respiratory cilia.

In some cases, the composition comprises a nucleic acid construct encoding chromosome 21 open reading frame 59 (C21orf59), and upon translation within the cells of a subject the construct yields a polypeptide that treats a subject having or at risk of having of primary ciliary dyskinesia. The protein encoded by the C21orf59 gene can play a critical role in dynein arm assembly and motile cilia function. Mutations in this gene can result in primary ciliary dyskinesia.

In some cases, the composition comprises a nucleic acid construct encoding coiled-coil domain containing 103 (CCDC103), and upon translation within the cells of a subject the construct yields a polypeptide that treats a subject having or at risk of having of primary ciliary dyskinesia. The protein encoded by the CCDC103 gene can function as a dynein-attachment factor required for cilia motility.

In some cases, the composition comprises a nucleic acid construct encoding coiled-coil domain containing 114 (CCDC114), and upon translation within the cells of a subject the construct yields a polypeptide that treats a subject having or at risk of having of primary ciliary dyskinesia. The protein encoded by the CCDC114 gene can function as a component of the outer dynein arm docking complex in cilia cells. Mutations in this gene can cause primary ciliary dyskinesia 20.

In some cases, the composition comprises a nucleic acid construct encoding coiled-coil domain containing 39 (CCDC39), and upon translation within the cells of a subject the construct yields a polypeptide that treats a subject having or at risk of having of primary ciliary dyskinesia. The protein encoded by the CCDC39 gene can function as the assembly of dynein regulatory and inner dynein arm complexes, which regulate ciliary beat. Defects in this gene can be a cause of primary ciliary dyskinesia.

In some cases, the composition comprises a nucleic acid construct encoding coiled-coil domain containing 40 (CCDC40), and upon translation within the cells of a subject the construct yields a polypeptide that treats a subject having or at risk of having of primary ciliary dyskinesia. The protein encoded by the CCDC40 gene can function together with CCDC39 to form a molecular ruler that determines the 96 nanometer (nm) repeat length and arrangements of components in cilia and flagella (by similarity). CCDC40 may not be required for outer dynein arm complexes assembly, but it may be required for axonemal recruitment of CCDC39. In some cases, CCD40 and CCD39 can be produced from different genes administered to the subject in the same or in a separate composition. Alternatively, CCD40 and CCD39 can be produced by a single nucleic acid construct that encodes a functional component of an inner dynein arm or an outer dynein arm. Defects in the CCD40 gene can be a cause of primary ciliary dyskinesia.

In some cases, the composition comprises a nucleic acid construct encoding coiled-coil domain containing 65 (CCDC65), and upon translation within the cells of a subject the construct yields a polypeptide that treats a subject having or at risk of having of primary ciliary dyskinesia. The protein encoded by the CCDC65 gene can function as a sperm cell protein. CCDC65 has been shown to be highly expressed in adult testis, spermatocytes and spermatids. The protein plays a critical role in the assembly of the nexin-dynein regulatory complex. Mutations in this gene have been associated with primary ciliary dyskinesia type 27.

In some cases, the composition comprises a nucleic acid construct encoding cyclin O (CCNO), and upon translation within the cells of a subject the construct yields a polypeptide that treats a subject having or at risk of having of primary ciliary dyskinesia.

In some cases, the composition comprises a nucleic acid construct encoding dynein (axonemal) assembly factor 1 (DNAAF1), and upon translation within the cells of a subject the construct yields a polypeptide that treats a subject having or at risk of having of primary ciliary dyskinesia. The protein encoded by the DNAAF1 gene is thought to be cilium-specific and it can be required for the stability of the ciliary architecture. Mutations in this gene have been associated with primary ciliary dyskinesia type 13.

In some cases, the composition comprises a nucleic acid construct encoding dynein (axonemal) assembly factor 2 (DNAAF2), and upon translation within the cells of a subject the construct yields a polypeptide that treats a subject having or at risk of having of primary ciliary dyskinesia. The protein encoded by the DNAAF2 gene can be involved in the preassembly of dynein arm complexes which power cilia. Mutations in this gene have been associated with primary ciliary dyskinesia type 10 (CILD10).

In some cases, the composition comprises a nucleic acid construct encoding dynein (axonemal) assembly factor 3 (DNAAF3), and upon translation within the cells of a subject the construct yields a polypeptide that treats a subject having or at risk of having of primary ciliary dyskinesia. The protein encoded by the DNAAF3 gene can be required for the assembly of axonemal inner and outer dynein arms and it can play a role in assembling dynein complexes for transport into cilia. Mutations in this gene have been associated with primary ciliary dyskinesia type 2 (CILD2).

In some cases, the composition comprises a nucleic acid construct encoding dynein (axonemal) assembly factor 5 (DNAAF5), and upon translation within the cells of a subject the construct yields a polypeptide that treats a subject having or at risk of having of primary ciliary dyskinesia. The protein encoded by the DNAAF5 gene is thought to be required for the preassembly or stability of axonemal dynein aims, and is found only in organisms with motile cilia and flagella. Mutations in this gene have been associated with primary ciliary dyskinesia-18.

In some cases, the composition comprises a nucleic acid construct encoding dynein axonemal heavy chain 11 (DNAH11), and upon translation within the cells of a subject the construct yields a polypeptide that treats a subject having or at risk of having of primary ciliary dyskinesia. The protein encoded by the DNAH11 gene can produce a ciliary outer dynein arm protein. DNAH11 is thought to be a microtubule-dependent motor ATPase involved in the movement of respiratory cilia. Mutations in this gene have been associated with primary ciliary dyskinesia type 7 (CILD7) and heterotaxy syndrome.

In some cases, the composition comprises a nucleic acid construct encoding dynein axonemal heavy chain 5 (DNAHS), and upon translation within the cells of a subject the construct yields a polypeptide that treats a subject having or at risk of having primary ciliary dyskinesia. The DNAHS gene can provide instructions for making a protein that is part of a group (complex) of proteins called dynein. Coordinated back and forth movement of cilia can move the cell or the fluid surrounding the cell. Dynein can produce the force needed for cilia to move. More than 80 mutations of the DNAHS have been associated with primary ciliary dyskinesia. Mutations in this gene have been associated with primary ciliary dyskinesia and heterotaxy syndrome.

In some cases, the composition comprises a nucleic acid construct encoding dynein axonemal heavy chain 6 (DNAH6), and upon translation within the cells of a subject the construct yields a polypeptide that treats a subject having or at risk of having primary ciliary dyskinesia.

In some cases, the composition comprises a nucleic acid construct encoding dynein axonemal heavy chain 8 (DNAHS), and upon translation within the cells of a subject the construct yields a polypeptide that treats a subject having or at risk of having of primary ciliary dyskinesia. The protein encoded by the DNAH8 gene can function as a force generating protein of respiratory cilia. DNAH8 can produce force towards the minus ends of microtubules. Dynein has ATPase activity; the force-producing power stroke is thought to occur on release of ADP. DNAH8 can be involved in sperm motility and in sperm flagellar assembly. DNAH8 is also known as ATPase and hdhc9.

In some cases, the composition comprises a nucleic acid construct encoding dynein axonemal light chain 1 (DNAL1), and upon translation within the cells of a subject the construct yields a polypeptide that treats a subject having or at risk of having of primary ciliary dyskinesia. The protein encoded by the DNAL1 gene can function as a force generating protein of respiratory cilia. DNAL1 can function as a component of the outer dynein arms complex. This complex acts as the molecular motor that provides the force to move cilia in an ATP-dependent manner. Mutations in this gene have been associated with primary ciliary dyskinesia type 16 (CILD16).

In some cases, the composition comprises a nucleic acid construct encoding dynein regulatory complex subunit 1 (DRC1), and upon translation within the cells of a subject the construct yields a polypeptide that treats a subject having or at risk of having of primary ciliary dyskinesia. The protein encoded by the DRC1 gene can function as a force generating protein of respiratory cilia. DRC1 can encode a central component of the nexin-dynein complex (N-DRC), which regulates the assembly of ciliary dynein. Mutations in this gene have been associated with primary ciliary dyskinesia type 21 (CILD21).

In some cases, the composition comprises a nucleic acid construct encoding dynein (axonemal) assembly factor 4 (DNAAF4), and upon translation within the cells of a subject the construct yields a polypeptide that treats a subject having or at risk of having of primary ciliary dyskinesia. The protein encoded by the DNAAF4 gene can function as a force generating protein of respiratory cilia. DNAAF4 can encode a tetratricopeptide repeat domain-containing protein. The encoded protein can interact with estrogen receptors and the heat shock proteins, Hsp70 and Hsp90. Mutations in this gene are also associated with deficits in reading and spelling, and a chromosomal translocation involving this gene is associated with a susceptibility to developmental dyslexia.

In some cases, the composition comprises a nucleic acid construct encoding growth arrest specific 8 (GASB), and upon translation within the cells of a subject the construct yields a polypeptide that treats a subject having or at risk of having of primary ciliary dyskinesia.

In some cases, the composition comprises a nucleic acid construct encoding axonemal central pair apparatus protein (HYDIN), and upon translation within the cells of a subject the construct yields a polypeptide that treats a subject having or at risk of having of primary ciliary dyskinesia. The protein encoded by the HYDIN gene can function in cilia motility. Mutations in this gene have been associated with primary ciliary dyskinesia type 5 (CILDS).

In some cases, the composition comprises a nucleic acid construct encoding leucine rich repeat containing 6 (LRRC6), and upon translation within the cells of a subject the construct yields a polypeptide that treats a subject having or at risk of having of primary ciliary dyskinesia. The protein encoded by the LRRC6 gene contains several leucine-rich repeat domains and appears to be involved in the motility of cilia. Mutations in this gene have been associated with primary ciliary dyskinesia type 19 (CILD19).

In some cases, the composition comprises a nucleic acid construct encoding NME/NM23 family member 8 (NME8), and upon translation within the cells of a subject the construct yields a polypeptide that treats a subject having or at risk of having of primary ciliary dyskinesia. The protein encoded by the NME8 gene can function as a force generating protein of respiratory cilia. The NME8 protein comprises an N-terminal thioredoxin domain and three C-terminal nucleoside diphosphate kinase (NDK) domains. Mutations in this gene have been associated with primary ciliary dyskinesia type 6 (CILD6).

In some cases, the composition comprises a nucleic acid construct encoding oral-facial-digital syndrome 1 (OFD1), and upon translation within the cells of a subject the construct yields a polypeptide that treats a subject having or at risk of having of primary ciliary dyskinesia. The function of the protein produced by the OFD1 gene is not well understood, but it may play a role play a critical role in the early development of many parts of the body, including the brain, face, limbs, and kidneys. About 100 mutations in the OFD1 gene have been found in people with oral-facial-digital syndrome type I, which is the most common form of the disorder. Mutations in this gene have been associated with primary ciliary dyskinesia and Joubert syndrome.

In some cases, the composition comprises a nucleic acid construct encoding retinitis pigmentosa GTPase regulator (RPGR), and upon translation within the cells of a subject the construct yields a polypeptide that treats a subject having or at risk of having of primary ciliary dyskinesia. The protein encoded by the RPGR gene can be important for normal vision and for the function of the cilia. Mutations in this gene have been associated with primary ciliary dyskinesia, X-linked retinitis pigmentosa, progressive vision loss, chronic respiratory and sinus infections, recurrent ear infections (otitis media), and hearing loss.

In some cases, the composition comprises a nucleic acid construct encoding radial spoke head 1 homolog (RSPH1), and upon translation within the cells of a subject the construct yields a polypeptide that treats a subject having or at risk of having of primary ciliary dyskinesia. The protein encoded by the RSPH1 gene may play an important role in male meiosis and in the building of the axonemal central pair and radial spokes. Mutations in this gene have been associated with primary ciliary dyskinesia type 24 (CILD24).

In some cases, the composition comprises a nucleic acid construct encoding radial spoke head 4 homolog A (RSPH4A), and upon translation within the cells of a subject the construct yields a polypeptide that treats a subject having or at risk of having of primary ciliary dyskinesia. The protein encoded by the RSPH4A gene may be a component the radial spoke head. Mutations in this gene have been associated with primary ciliary dyskinesia type 11 (CILD11).

In some cases, the composition comprises a nucleic acid construct encoding radial spoke head 9 homolog (RSPH9), and upon translation within the cells of a subject the construct yields a polypeptide that treats a subject having or at risk of having of primary ciliary dyskinesia. The protein encoded by the RSPH9 gene may be a component the radial spoke head in motile cilia and flagella. Mutations in this gene have been associated with primary ciliary dyskinesia type 12 (CILD12).

In some cases, the composition comprises a nucleic acid construct encoding sperm associated antigen 1 (SPAG1), and upon translation within the cells of a subject the construct yields a polypeptide that treats a subject having or at risk of having of primary ciliary dyskinesia. The protein encoded by the SPAG1 gene may play a role in the cytoplasmic assembly of the ciliary dynein arms. Mutations in this gene have been associated with primary ciliary dyskinesia type 28 (CILD28).

In some cases, the composition comprises a nucleic acid construct encoding zinc finger MYND-type containing 10 (ZMYND10), and upon translation within the cells of a subject the construct yields a polypeptide that treats a subject having or at risk of having of primary ciliary dyskinesia. The protein encoded by the ZMYND10 can function in axonemal assembly of inner and outer dynein arms (IDA and ODA, respectively) for proper axoneme building for cilia motility. Mutations in this gene have been associated with primary ciliary dyskinesia type 22 (CILD22).

Compositions containing the engineered polynucleotides described herein can be administered for prophylactic and/or therapeutic treatments. In therapeutic applications, the nucleic acid constructs or vectors can be administered to a subject already suffering from a disease, such as a primary ciliary dyskinesia, in the amount sufficient to provide the amount of the encoded polypeptide that cures or at least improves the symptoms of the disease. Nucleic acid constructs, vectors, engineered polynucleotides, or compositions can also be administered to lessen a likelihood of developing, contracting, or worsening a disease. Amounts effective for this use can vary based on the severity and course of the disease or condition, the efficiency of transfection of a nucleic acid construct(s), vector(s), engineered polyribonucleotide(s), or composition(s), the affinity of an encoded polypeptide to a target molecule, previous therapy, the subject's health status, weight, response to the drugs, and the judgment of the treating physician.

In some cases, a polynucleotide of the disclosure can encode a polypeptide that is at least 5%, 10%, 15%, 20%, 25%, 30%, 35%, 40%, 45%, 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a protein associated with primary ciliary dyskinesia (or a fragment thereof), such as armadillo repeat containing 4 (ARMC4), chromosome 21 open reading frame 59 (C21orf59), coiled-coil domain containing 103 (CCDC103), coiled-coil domain containing 114 (CCDC114), coiled-coil domain containing 39 (CCDC39), coiled-coil domain containing 40 (CCDC40), coiled-coil domain containing 65 (CCDC65), cyclin O (CCNO), dynein (axonemal) assembly factor 1 (DNAAF1), dynein (axonemal) assembly factor 2 (DNAAF2), dynein (axonemal) assembly factor 3 (DNAAF3), dynein (axonemal) assembly factor 4 (DNAAF4), dynein (axonemal) assembly factor 5

(DNAAF5), dynein axonemal heavy chain 11 (DNAH11), dynein axonemal heavy chain 5 (DNAH5), dynein axonemal heavy chain 6 (DNAH6),dynein axonemal heavy chain 8 (DNAH8), dynein axonemal intermediate chain 2 (DNAI2), dynein axonemal light chain 1 (DNAL1), dynein regulatory complex subunit 1 (DRC1), growth arrest specific 8 (GAS8), axonemal central pair apparatus protein (HYDIN), leucine rich repeat containing 6 (LRRC6), NME/NM23 family member 8 (NME8), oral-facial-digital syndrome 1 (OFD1), retinitis pigmentosa GTPase regulator (RPGR), radial spoke head 1 homolog (Chlamydomonas) (RSPH1), radial spoke head 4 homolog A (Chlamydomonas) (RSPH4A), radial spoke head 9 homolog (Chlamydomonas) (RSPH9), sperm associated antigen 1(SPAG1), and zinc finger MYND-type containing 10 (ZMYND10).

TABLE 1
List of example PCD-associated protein genes

| | | | | | |
|---|---|---|---|---|---|
| DNAH5 | DNAI1 | DNAH11 | ARMC4 | ZMYND10 | CCDC114 |
| RSPH4A | LRRC6 | SPAG1 | DNAAF4 | CCDC40 | CCDC39 |
| DNAAF1 | LRRC50 | RSPH1 | DNAI2 | DAAF2 | RSPH4a |

### Codon Optimized Polynucleotides

Provided herein, in some embodiments, include a (e.g., pharmaceutical) composition that comprises a polynucleotide (e.g., comprising a particular sequence that encodes a PCD-associated gene or protein). The polynucleotide may be an mRNA. The polynucleotide may be an mRNA that encode a cytoplasmic or axonemal dynein component protein. The polynucleotide may be an mRNA of a gene of selected from the group consisting of DNAH5, ARMC4, ZMYND10, DNAAF4, CCDC40, CCDC39, DNAAF1, DNAI2, and DAAF2. The polynucleotide may be an mRNA of a gene set forth in Table 1. In some cases, the polynucleotide is not an mRNA for DNAIl. The polynucleotide may comprise a nucleic acid sequence having sequence identity to a sequence (or a fragment thereof over at least 500, 600, 700, 800, 900, 1000, 1100, 1200, 1300, 1400, 1500, 1700, 1800, 1900, or 2000 bases) listed herein. The polynucleotide may comprise a nucleic acid sequence having sequence identity to a portion of sequences listed herein. For example, the polynucleotide may comprise a nucleic acid sequence having at a least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NOs: 1-32, 61, or 62 (or a fragment over at least 500, 600, 700, 800, 900, 1000, 1100, 1200, 1300, 1400, 1500, 1700, 1800, 1900, or 2000 bases). The polynucleotide may comprise a nucleic acid sequence having at a least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence disclosed herein. In some embodiments, the nucleic acid sequence has at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a fragment over at least 500 (e.g., at least 600, 700, 800, 900, 1000, 1100, 1200, 1300, 1400, 1500, 1700, 1800, 1900, or 2000 bases) of SEQ ID NOs: 1-32, 61, or 62. In some embodiments, the nucleic acid sequence has 100% sequence identity to a sequence disclosed herein. In some embodiments, the nucleic acid has 100% sequence identity to a fragment (e.g., over at least 500, 600, 700, 800, 900, or 1,000 bases) of SEQ ID NOs: 1-32, 61, or 62. The polynucleotide may comprise a nucleic acid sequence having at a least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence disclosed herein. In some embodiments, the nucleic acid sequence has at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence over at least 1,000 bases (e.g., nucleotides 1 to 1,000) of SEQ ID NOs: 1-32, 61, or 62. In some embodiments, the nucleic acid sequence has 100% sequence identity to a sequence disclosed herein or any fragment thereof. In some embodiments, the nucleic acid has 100% sequence identity to a sequence over at least 1,000, 1100, 1200, 1300, 1400, 1500, 1700, 1800, 1900, or 2000 bases of SEQ ID NOs: 1-32, 61, or 62. Polynucleotides described herein may be DNA or RNA. The sequences disclosed throughout the specification may have a uridine (U) substituted at any location that a thymidine (T) is present. The disclosure recognizes that a sequence disclosed herein of DNA may be used to generate a corresponding RNA sequence in which instances of thymidine have been replaced with uridine. As such the sequences described herein are not limited to thymidine containing sequence, and the corresponding uridine sequences are also contemplated herein.

TABLE 2
Example nucleic acid sequences

| SEQ # | Sequence | |
|---|---|---|
| SEQ ID | ATGGAAATCGTGTACGTGTACGTCAAGAAGCGGAGCGAGTTCGGCAAGCAGTGCAACTTCAG | DNAI2 |
| NO: 1 | CGACAGACAGGCCGAGCTGAACATCGACATCATGCCCAATCCTGAGCTGGCCGAGCAGTTCG | (ORF) |
| | TGGAAAGAAACCCTGTGGACACCGGCATCCAGTGCAGCATCAGCATGTCTGAGCACGAGGCC | |
| | AACAGCGAGAGATTCGAGATGGAAACCAGAGGCGTGAACCACGTGGAAGGCGGCTGGCCCAA | |
| | AGATGTGAACCCTCTGGAACTGGAACAGACCATCCGGTTCCGCAAGAAGGTGGAAAAGGACG | |
| | AGAACTACGTGAACGCCATCATGCAGCTGGGCAGCATCATGGAACACTGCATCAAGCAGAAC | |
| | AACGCCATCGACATCTACGAGGAATACTTCAACGACGAAGAGGCCATGGAAGTGATGGAAGA | |
| | GGACCCCAGCGCCAAGACCATCAACGTGTTCAGAGATCCCCAAGAGATCAAGAGAGCCGCCA | |
| | CACACCTGAGCTGGCACCCCGACGGAAACAGAAAACTGGCCGTGGCCTACAGCTGCCTGGAC | |
| | TTCCAGAGGGCCCCTGTGGGCATGAGCAGCGACAGCTACATCTGGGACctgGAGAACCCCAA | |
| | CAAGCCCGAGCTGGCCCTGAAGCCCAGCAGCCCTCTGGTCACCCTGgagTTCAACCCCAAGG | |
| | ACAGCCACGTGCTGCTCGGCGGCTGCTACAATGGACAGATCGCCTGCTGGGACACCAGAAAG | |
| | GGCTCTCTGGTGGCCGAACTGAGCACCATCGAGAGCAGCCACAGAGATCCTGTGTACGGCAC | |
| | CATCTGGCTGCAGAGCAAGACCGGCACCGAGTGCTTCAGCGCCAGCACCGATGGCCAAGTGA | |
| | TGTGGTGGGACATCCGGAAGATGAGCGAGCCCACCGAGGTGGTCATCCTGGACATCACCAAG | |
| | AAAGAGCAGCTGGAAAACGCCCTGGGCGCCATCAGCCTGgagTTCGAGAGCACCCTGCCCAC | |
| | CAAGTTCATGGTCGGAACCGAGCAGGGCATCGTGATCAGCTGCAACAGAAAGGCCAAGACCA | |
| | GCGCCGAGAAGATCGTGTGCACCTTCCCTGGACACCACGGACCCATCTACGCCCTGCAGCGG | |
| | AATCCCTTCTACCCCAAGAACTTCCTGACCGTCGGCGACTGGACCGCCAGAATCTGGAGCGA | |
| | GGACAGCCGGGAAAGCAGCATCATGTGGACCAAGTACCACATGGCCTACCTGACCGATGCCG | |
| | CCTGGAGCCCTGTGAGACCCACCGTGTTCTTCACCACCAGAATGGACGGCACCCTGGACATC | |
| | TGGGACTTCATGTTCGAGCAGTGCGACCCCACACTGAGCCTGAAAGTGTGCGACGAGGCCCT | |
| | GTTCTGCCTGAGAGTGCAGGACAACGGCTGCCTGATCGCCTGTGGAAGCCAGCTGGGAACCA | |
| | CCACACTGCTGGAAGTGTCTCCAGGCCTGAGCACCCTGCAGAGAAACGAGAAGAACGTGGCC | |
| | AGCAGCATGTTCGAGAGAGAGACTCGGAGAGAGAAGATCCTGGAAGCCCGGCACAGAGAGAT | |
| | GCGGCTGAAAGAGAAGGGCAAAGCCGAGGGCAGAGATGAGGAACAGACAGACGAGGAACTGG | |
| | CTGTGGACCTGGAAGCACTGGTGAGCAAGGCCGAGGAAGAGTTCTTCGACATCATCTTCGCT | |
| | GAGCTGAAGAAGAAAGAGGCCGACGCCATCAAGCTGACCCCTGTGCCACAGCAGCCCAGTCC | |
| | TGAAGAGGATCAGGTGGTGGAAGAGGGCGAAGAAGCCGCTGGCGAAGAGGGCGACGAAGAGG | |
| | TGGAAGAAGATCTGGCCTGA | |
| SEQ ID | GGGAGACCCAAGCTGGCTAGCGTTTAAACTTCAGCTTGGCAATCCGGTACTGTTGGTAAAGC | DNAI2 |
| NO: 2 | CACCATGGAAATCGTGTACGTGTACGTCAAGAAGCGGAGCGAGTTCGGCAAGCAGTGCAACT | (5′ UTR, |
| | TCAGCGACAGACAGGCCGAGCTGAACATCGACATCATGCCCAATCCTGAGCTGGCCGAGCAG | ORF, and |
| | TTCGTGGAAAGAAACCCTGTGGACACCGGCATCCAGTGCAGCATCAGCATGTCTGAGCACGA | 3′ tail) |
| | GGCCAACAGCGAGAGATTCGAGATGGAAACCAGAGGCGTGAACCACGTGGAAGGCGGCTGGC | |
| | CCAAAGATGTGAACCCTCTGGAACTGGAACAGACCATCCGGTTCCGCAAGAAGGTGGAAAAG | |
| | GACGAGAACTACGTGAACGCCATCATGCAGCTGGGCAGCATCATGGAACACTGCATCAAGCA | |
| | GAACAACGCCATCGACATCTACGAGGAATACTTCAACGACGAAGAGGCCATGGAAGTGATGG | |
| | AAGAGGACCCCAGCGCCAAGACCATCAACGTGTTCAGAGATCCCCAAGAGATCAAGAGAGCC | |
| | GCCACACACCTGAGCTGGCACCCCGACGGAAACAGAAAACTGGCCGTGGCCTACAGCTGCCT | |
| | GGACTTCCAGAGGGCCCCTGTGGGCATGAGCAGCGACAGCTACATCTGGGACctgGAGAACC | |
| | CCAACAAGCCCGAGCTGGCCCTGAAGCCCAGCAGCCCTCTGGTCACCCTGgagTTCAACCCC | |
| | AAGGACAGCCACGTGCTGCTCGGCGGCTGCTACAATGGACAGATCGCCTGCTGGGACACCAG | |
| | AAAGGGCTCTCTGGTGGCCGAACTGAGCACCATCGAGAGCAGCCACAGAGATCCTGTGTACG | |
| | GCACCATCTGGCTGCAGAGCAAGACCGGCACCGAGTGCTTCAGCGCCAGCACCGATGGCCAA | |
| | GTGATGTGGTGGGACATCCGGAAGATGAGCGAGCCCACCGAGGTGGTCATCCTGGACATCAC | |
| | CAAGAAAGAGCAGCTGGAAAACGCCCTGGGCGCCATCAGCCTGgagTTCGAGAGCACCCTGC | |
| | CCACCAAGTTCATGGTCGGAACCGAGCAGGGCATCGTGATCAGCTGCAACAGAAAGGCCAAG | |
| | ACCAGCGCCGAGAAGATCGTGTGCACCTTCCCTGGACACCACGGACCCATCTACGCCCTGCA | |
| | GCGGAATCCCTTCTACCCCAAGAACTTCCTGACCGTCGGCGACTGGACCGCCAGAATCTGGA | |
| | GCGAGGACAGCCGGGAAAGCAGCATCATGTGGACCAAGTACCACATGGCCTACCTGACCGAT | |
| | GCCGCCTGGAGCCCTGTGAGACCCACCGTGTTCTTCACCACCAGAATGGACGGCACCCTGGA | |
| | CATCTGGGACTTCATGTTCGAGCAGTGCGACCCCACACTGAGCCTGAAAGTGTGCGACGAGG | |
| | CCCTGTTCTGCCTGAGAGTGCAGGACAACGGCTGCCTGATCGCCTGTGGAAGCCAGCTGGGA | |
| | ACCACCACACTGCTGGAAGTGTCTCCAGGCCTGAGCACCCTGCAGAGAAACGAGAAGAACGT | |
| | GGCCAGCAGCATGTTCGAGAGAGAGACTCGGAGAGAGAAGATCCTGGAAGCCCGGCACAGAG | |
| | AGATGCGGCTGAAAGAGAAGGGCAAAGCCGAGGGCAGAGATGAGGAACAGACAGACGAGGAA | |
| | CTGGCTGTGGACCTGGAAGCACTGGTGAGCAAGGCCGAGGAAGAGTTCTTCGACATCATCTT | |
| | CGCTGAGCTGAAGAAGAAAGAGGCCGACGCCATCAAGCTGACCCCTGTGCCACAGCAGCCCA | |
| | GTCCTGAAGAGGATCAGGTGGTGGAAGAGGGCGAAGAAGCCGCTGGCGAAGAGGGCGACGAA | |
| | GAGGTGGAAGAAGATCTGGCCTGAGAATTCTGCAGAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAATTCG | |
| SEQ ID | ATGGAAATCGTGTACGTGTACGTCAAGAAGCGGAGCGAGTTCGGCAAGCAGTGCAACTTCAG | DNAI2 |
| NO: 3 | CGACAGACAGGCCGAGCTGAACATCGACATCATGCCCAATCCTGAGCTGGCCGAGCAGTTCG | (ORF and |
| | TGGAAAGAAACCCTGTGGACACCGGCATCCAGTGCAGCATCAGCATGTCTGAGCACGAGGCC | HA tag) |
| | AACAGCGAGAGATTCGAGATGGAAACCAGAGGCGTGAACCACGTGGAAGGCGGCTGGCCCAA | |
| | AGATGTGAACCCTCTGGAACTGGAACAGACCATCCGGTTCCGCAAGAAGGTGGAAAAGGACG | |
| | AGAACTACGTGAACGCCATCATGCAGCTGGGCAGCATCATGGAACACTGCATCAAGCAGAAC | |
| | AACGCCATCGACATCTACGAGGAATACTTCAACGACGAAGAGGCCATGGAAGTGATGGAAGA | |
| | GGACCCCAGCGCCAAGACCATCAACGTGTTCAGAGATCCCCAAGAGATCAAGAGAGCCGCCA | |
| | CACACCTGAGCTGGCACCCCGACGGAAACAGAAAACTGGCCGTGGCCTACAGCTGCCTGGAC | |
| | TTCCAGAGGGCCCCTGTGGGCATGAGCAGCGACAGCTACATCTGGGACCTGGAGAACCCCAA | |
| | CAAGCCCGAGCTGGCCCTGAAGCCCAGCAGCCCTCTGGTCACCCTGGAGTTCAACCCCAAGG | |
| | ACAGCCACGTGCTGCTCGGCGGCTGCTACAATGGACAGATCGCCTGCTGGGACACCAGAAAG | |
| | GGCTCTCTGGTGGCCGAACTGAGCACCATCGAGAGCAGCCACAGAGATCCTGTGTACGGCAC | |
| | CATCTGGCTGCAGAGCAAGACCGGCACCGAGTGCTTCAGCGCCAGCACCGATGGCCAAGTGA | |
| | TGTGGTGGGACATCCGGAAGATGAGCGAGCCCACCGAGGTGGTCATCCTGGACATCACCAAG | |
| | AAAGAGCAGCTGGAAAACGCCCTGGGCGCCATCAGCCTGGAGTTCGAGAGCACCCTGCCCAC | |
| | CAAGTTCATGGTCGGAACCGAGCAGGGCATCGTGATCAGCTGCAACAGAAAGGCCAAGACCA | |
| | GCGCCGAGAAGATCGTGTGCACCTTCCCTGGACACCACGGACCCATCTACGCCCTGCAGCGG | |
| | AATCCCTTCTACCCCAAGAACTTCCTGACCGTCGGCGACTGGACCGCCAGAATCTGGAGCGA | |
| | GGACAGCCGGGAAAGCAGCATCATGTGGACCAAGTACCACATGGCCTACCTGACCGATGCCG | |
| | CCTGGAGCCCTGTGAGACCCACCGTGTTCTTCACCACCAGAATGGACGGCACCCTGGACATC | |
| | TGGGACTTCATGTTCGAGCAGTGCGACCCCACACTGAGCCTGAAAGTGTGCGACGAGGCCCT | |
| | GTTCTGCCTGAGAGTGCAGGACAACGGCTGCCTGATCGCCTGTGGAAGCCAGCTGGGAACCA | |
| | CCACACTGCTGGAAGTGTCTCCAGGCCTGAGCACCCTGCAGAGAAACGAGAAGAACGTGGCC | |
| | AGCAGCATGTTCGAGAGAGAGACTCGGAGAGAGAAGATCCTGGAAGCCCGGCACAGAGAGAT | |
| | GCGGCTGAAAGAGAAGGGCAAAGCCGAGGGCAGAGATGAGGAACAGACAGACGAGGAACTGG | |
| | CTGTGGACCTGGAAGCACTGGTGAGCAAGGCCGAGGAAGAGTTCTTCGACATCATCTTCGCT | |
| | GAGCTGAAGAAGAAAGAGGCCGACGCCATCAAGCTGACCCCTGTGCCACAGCAGCCCAGTCC | |
| | TGAAGAGGATCAGGTGGTGGAAGAGGGCGAAGAAGCCGCTGGCGAAGAGGGCGACGAAGAGG | |
| | TGGAAGAAGATCTGGCCGGCAGCGGCTACCCATACGATGTTCCTGACTATGCGTGA | |
| SEQ ID | GGGAGACCCAAGCTGGCTAGCGTTTAAACTTCAGCTTGGCAATCCGGTACTGTTGGTAAAGC | DNAI2 |
| NO: 4 | CACCATGGAAATCGTGTACGTGTACGTCAAGAAGCGGAGCGAGTTCGGCAAGCAGTGCAACT | (5′ UTR, |
| | TCAGCGACAGACAGGCCGAGCTGAACATCGACATCATGCCCAATCCTGAGCTGGCCGAGCAG | ORF, HA |
| | TTCGTGGAAAGAAACCCTGTGGACACCGGCATCCAGTGCAGCATCAGCATGTCTGAGCACGA | tag, and |
| | GGCCAACAGCGAGAGATTCGAGATGGAAACCAGAGGCGTGAACCACGTGGAAGGCGGCTGGC | 3′ tail) |
| | CCAAAGATGTGAACCCTCTGGAACTGGAACAGACCATCCGGTTCCGCAAGAAGGTGGAAAAG | |
| | GACGAGAACTACGTGAACGCCATCATGCAGCTGGGCAGCATCATGGAACACTGCATCAAGCA | |
| | GAACAACGCCATCGACATCTACGAGGAATACTTCAACGACGAAGAGGCCATGGAAGTGATGG | |
| | AAGAGGACCCCAGCGCCAAGACCATCAACGTGTTCAGAGATCCCCAAGAGATCAAGAGAGCC | |
| | GCCACACACCTGAGCTGGCACCCCGACGGAAACAGAAAACTGGCCGTGGCCTACAGCTGCCT | |
| | GGACTTCCAGAGGGCCCCTGTGGGCATGAGCAGCGACAGCTACATCTGGGACCTGGAGAACC | |
| | CCAACAAGCCCGAGCTGGCCCTGAAGCCCAGCAGCCCTCTGGTCACCCTGGAGTTCAACCCC | |
| | AAGGACAGCCACGTGCTGCTCGGCGGCTGCTACAATGGACAGATCGCCTGCTGGGACACCAG | |
| | AAAGGGCTCTCTGGTGGCCGAACTGAGCACCATCGAGAGCAGCCACAGAGATCCTGTGTACG | |
| | GCACCATCTGGCTGCAGAGCAAGACCGGCACCGAGTGCTTCAGCGCCAGCACCGATGGCCAA | |
| | GTGATGTGGTGGGACATCCGGAAGATGAGCGAGCCCACCGAGGTGGTCATCCTGGACATCAC | |
| | CAAGAAAGAGCAGCTGGAAAACGCCCTGGGCGCCATCAGCCTGGAGTTCGAGAGCACCCTGC | |
| | CCACCAAGTTCATGGTCGGAACCGAGCAGGGCATCGTGATCAGCTGCAACAGAAAGGCCAAG | |
| | ACCAGCGCCGAGAAGATCGTGTGCACCTTCCCTGGACACCACGGACCCATCTACGCCCTGCA | |
| | GCGGAATCCCTTCTACCCCAAGAACTTCCTGACCGTCGGCGACTGGACCGCCAGAATCTGGA | |
| | GCGAGGACAGCCGGGAAAGCAGCATCATGTGGACCAAGTACCACATGGCCTACCTGACCGAT | |
| | GCCGCCTGGAGCCCTGTGAGACCCACCGTGTTCTTCACCACCAGAATGGACGGCACCCTGGA | |
| | CATCTGGGACTTCATGTTCGAGCAGTGCGACCCCACACTGAGCCTGAAAGTGTGCGACGAGG | |
| | CCCTGTTCTGCCTGAGAGTGCAGGACAACGGCTGCCTGATCGCCTGTGGAAGCCAGCTGGGA | |
| | ACCACCACACTGCTGGAAGTGTCTCCAGGCCTGAGCACCCTGCAGAGAAACGAGAAGAACGT | |
| | GGCCAGCAGCATGTTCGAGAGAGAGACTCGGAGAGAGAAGATCCTGGAAGCCCGGCACAGAG | |
| | AGATGCGGCTGAAAGAGAAGGGCAAAGCCGAGGGCAGAGATGAGGAACAGACAGACGAGGAA | |
| | CTGGCTGTGGACCTGGAAGCACTGGTGAGCAAGGCCGAGGAAGAGTTCTTCGACATCATCTT | |
| | CGCTGAGCTGAAGAAGAAAGAGGCCGACGCCATCAAGCTGACCCCTGTGCCACAGCAGCCCA | |
| | GTCCTGAAGAGGATCAGGTGGTGGAAGAGGGCGAAGAAGCCGCTGGCGAAGAGGGCGACGAA | |
| | GAGGTGGAAGAAGATCTGGCCGGAAGCGGCTACCCATACGATGTTCCTGACTATGCGTGAGA | |
| | ATTCTGCAGAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAA | |
| | AAAAATTCG | |
| SEQ ID | ATGGGAGTGGCCCTGAGAAAGCTGACCCAGTGGACAGCCGCTGGACACGGAACAGGCATCCT | ARMC 4 |
| NO: 5 | GGAAATCACCCCTCTGAACGAGGCCATCCTGAAAGAAATCATCGTGTTCGTCGAGAGCTTCA | (ORF) |
| | TCTACAAGCACCCTCAAGAGGCCAAGTTCGTGTTCGTGGAACCCCTGGAATGGAACACCTCT | |
| | CTGGCCCCCAGCGCCTTCGAGAGCGGCTACGTGGTGTCTGAGACAACCGTGAAGTCCGAGGA | |
| | AGTGGACAAGAACGGCCAGCCTCTGCTGTTCCTGAGCGTGCCCCAGATCAAGATCAGgAGCT | |
| | TCGGCCAGCTGAGCCGGCTGCTGCTGATCGCCAAAACCGGCAAGCTGAAAGAGGCCCAGGCC | |
| | TGCGTGGAGGCCAACAGAGATCCCATCGTGAAGATCCTGGGCAGCGACTACAACACCATGAA | |
| | GGAAAACAGCATCGCCCTGAACATCCTGGGAAAGATCACCAGGGACGACGACCCCGAGAGCG | |
| | AGATCAAGATGAAGATCGCCATGCTGCTGAAGCAGCTGGACCTGCATCTGCTGAACCACAGC | |
| | CTGAAGCACATCAGCCTGGAAATCTCTCTGAGCCCCATGACCGTGAAGAAGGACATCGAGCT | |
| | GCTGAAACGGTTCAGCGGCAAGGGCAATCAGACCGTGCTGGAAAGCATCGAGTACACCAGCG | |
| | ACTACGAGTTCAGCAACGGCTGCAGAGCCCCACCCTGGAGACAGATCAGAGGCGAGATCTGC | |
| | TACGTGCTGGTCAAGCCCCACGATGGCGAGACACTGTGCATCACATGCTCTGCCGGCGGAGT | |
| | GTTCCTGAACGGCGGCAAGACAGATGATGAGGGCGACGTGAACTACGAGCGGAAGGGCAGCA | |
| | TCTACAAGAACCTGGTCACCTTCCTGCGGGAAAAGAGCCCCAAGTTCAGCGAGAACATGAGC | |
| | AAGCTGGGCATCAGCTTCAGCGAGGACCAGCAGAAAGAGAAGGACCAGCTGGGCAAAGCCCC | |
| | CAAGAAAGAGGAAGCCGCCGCTCTGAGAAAGGACATCAGCGGCAGCGACAAGCGGAGCCTGG | |
| | AAAAGAACCAGATCAACTTCTGGCGGAACCAGATGACCAAGAGATGGGAGCCCAGCCTGAAC | |
| | TGGAAAACCACCGTGAACTACAAAGGCAAGGGCAGCGCCAAAGAGATCCAAGAGGACAAGCA | |
| | CACAGGCAAGCTGGAAAAGCCTCGGCCCAGCGTGTCTCATGGCAGAGCACAGCTGCTGAGAA | |
| | AGAGCGCCGAGAAGATCGAGGAAACCGTGAGCGACAGCAGCAGCGAGAGCGAAGAGGACGAG | |
| | GAACCTCCTGACCACAGACAAGAAGCCAGCGCCGATCTGCCCAGCGAGTACTGGCAGATCCA | |
| | GAAGCTGGTGAAGTACCTGAAAGGCGGAAACCAGACCGCCACCGTGATCGCCCTGTGCAGCA | |
| | TGAGAGACTTCAGCCTGGCTCAAGAGACATGCCAGCTCGCCATCAGAGATGTCGGCGGACTG | |
| | GAAGTGCTGATCAACCTGCTGGAAACCGACGAAGTGAAGTGCAAGATCGGCAGCCTGAAAAT | |
| | CCTCAAAGAGATCAGCCACAATCCTCAGATCCGGCAGAACATCGTGGACCTCGGAGGCCTGC | |
| | CCATCATGGTCAACATCCTGGACAGCCCTCACAAGAGCCTGAAGTGTCTGGCCGCCGAGACA | |
| | ATCGCCAACGTGGCCAAGTTCAAGCGGGCCAGAAGAGTCGTCAGACAGCACGGCGGAATCAC | |
| | CAAACTGGTGGCCCTGCTGGACTGCGCCCACGACAGCACAAAGCCCGCTCAGAGCAGCCTGT | |
| | ACGAGGCCAGAGATGTGGAAGTGGCCAGATGTGGCGCTCTGGCCCTGTGGAGCTGCAGCAAG | |
| | AGCCACACCAACAAAGAGGCCATCAGAAAGGCTGGCGGCATCCCTCTGCTGGCCAGACTGCT | |
| | GAAAACCAGCCACGAGAACATGCTGATCCCCGTCGTGGGCACACTGCAAGAGTGTGCCAGCG | |
| | AGGAAAACTACAGAGCCGCCATCAAGGCCGAGCGGATCATCGAGAACCTCGTGAAGAATCTG | |
| | AACAGCGAGAACGAGCAGCTGCAAGAGCACTGCGCCATGGCCATCTATCAGTGCGCCGAGGA | |
| | CAAAGAAACCCGGGATCTGGTGCGGCTGCACGGCGGCCTGAAACCTCTGGCCAGCCTGCTGA | |
| | ACAACACCGACAACAAAGAACGGCTGGCCGCTGTGACAGGCGCCATCTGGAAGTGCAGCATC | |
| | AGCAAAGAAAACGTGACGAAGTTCCGCGAGTACAAGGCCATCGAGACACTCGTGGGCCTGCT | |
| | GACTGACCAACCTGAAGAGGTGCTCGTGAACGTGGTGGGAGCCCTGGGCGAGTGCTGTCAAG | |
| | AGCGGGAAAACAGAGTGATCGTGCGGAAGTGCGGAGGCATCCAGCCTCTCGTGAATCTGCTC | |
| | GTGGGCATCAATCAGGCCCTGCTGGTGAACGTGACAAAGGCCGTGGGAGCCTGTGCTGTGGA | |
| | ACCTGAGAGCATGATGATCATCGACCGGCTGGATGGCGTGCGGCTGCTGTGGAGTCTGCTGA | |
| | AGAACCCTCATCCTGACGTGAAGGCCTCTGCCGCCTGGGCTCTGTGCCCCTGCATCAAGAAT | |
| | GCCAAGGACGCCGGCGAGATGGTCaggAGCTTCGTGGGAGGCCTGGAACTGATCGTGAACCT | |
| | GCTGAAGTCTGACAACAAAGAAGTCCTGGCCAGCGTCTGCGCCGCCATCACCAACATCGCCA | |
| | AGGATCAAGAGAACCTGGCCGTGATCACCGACCATGGCGTGGTGCCACTGCTGAGCAAGCTG | |
| | GCCAACACCAACAACAACAAGCTGCGGCACCATCTGGCCGAGGCCATCAGCAGATGCTGCAT | |
| | GTGGGGCCGCAACAGAGTGGCCTTCGGAGAGCACAAAGCCGTGGCACCTCTCGTGCGGTATC | |
| | TGAAGAGCAACGACACCAATGTGCACCGGGCCACAGCCCAGGCTCTGTACCAGCTGTCTGAG | |
| | GACGCCGACAACTGCATCACCATGCACGAAAATGGCGCCGTGAAACTGCTGCTGGACATGGT | |
| | CGGAAGCCCCGACCAGGATCTGCAAGAGGCTGCTGCCGGCTGCATCAGCAACATCAGAAGGC | |
| | TGGCCCTGGCCACCGAGAAGGCCAGATACACCTGA | |
| SEQ ID | GGGAGACAAGAGAGAAAAGAAGAGCAAGAAGAAATATAAGAGCCACCATGGGAGTGGCCCTG | ARMC4 (5′ |
| NO: 6 | AGAAAGCTGACCCAGTGGACAGCCGCTGGACACGGAACAGGCATCCTGGAAATCACCCCTCT | UTR, ORF, |
| | GAACGAGGCCATCCTGAAAGAAATCATCGTGTTCGTCGAGAGCTTCATCTACAAGCACCCTC | and 3′ |
| | AAGAGGCCAAGTTCGTGTTCGTGGAACCCCTGGAATGGAACACCTCTCTGGCCCCCAGCGCC | tail) |
| | TTCGAGAGCGGCTACGTGGTGTCTGAGACAACCGTGAAGTCCGAGGAAGTGGACAAGAACGG | |
| | CCAGCCTCTGCTGTTCCTGAGCGTGCCCCAGATCAAGATCAGgAGCTTCGGCCAGCTGAGCC | |
| | GGCTGCTGCTGATCGCCAAAACCGGCAAGCTGAAAGAGGCCCAGGCCTGCGTGGAGGCCAAC | |
| | AGAGATCCCATCGTGAAGATCCTGGGCAGCGACTACAACACCATGAAGGAAAACAGCATCGC | |
| | CCTGAACATCCTGGGAAAGATCACCAGGGACGACGACCCCGAGAGCGAGATCAAGATGAAGA | |
| | TCGCCATGCTGCTGAAGCAGCTGGACCTGCATCTGCTGAACCACAGCCTGAAGCACATCAGC | |
| | CTGGAAATCTCTCTGAGCCCCATGACCGTGAAGAAGGACATCGAGCTGCTGAAACGGTTCAG | |
| | CGGCAAGGGCAATCAGACCGTGCTGGAAAGCATCGAGTACACCAGCGACTACGAGTTCAGCA | |
| | ACGGCTGCAGAGCCCCACCCTGGAGACAGATCAGAGGCGAGATCTGCTACGTGCTGGTCAAG | |
| | CCCCACGATGGCGAGACACTGTGCATCACATGCTCTGCCGGCGGAGTGTTCCTGAACGGCGG | |
| | CAAGACAGATGATGAGGGCGACGTGAACTACGAGCGGAAGGGCAGCATCTACAAGAACCTGG | |
| | TCACCTTCCTGCGGGAAAAGAGCCCCAAGTTCAGCGAGAACATGAGCAAGCTGGGCATCAGC | |
| | TTCAGCGAGGACCAGCAGAAAGAGAAGGACCAGCTGGGCAAAGCCCCCAAGAAAGAGGAAGC | |
| | CGCCGCTCTGAGAAAGGACATCAGCGGCAGCGACAAGCGGAGCCTGGAAAAGAACCAGATCA | |
| | ACTTCTGGCGGAACCAGATGACCAAGAGATGGGAGCCCAGCCTGAACTGGAAAACCACCGTG | |
| | AACTACAAAGGCAAGGGCAGCGCCAAAGAGATCCAAGAGGACAAGCACACAGGCAAGCTGGA | |
| | AAAGCCTCGGCCCAGCGTGTCTCATGGCAGAGCACAGCTGCTGAGAAAGAGCGCCGAGAAGA | |
| | TCGAGGAAACCGTGAGCGACAGCAGCAGCGAGAGCGAAGAGGACGAGGAACCTCCTGACCAC | |
| | AGACAAGAAGCCAGCGCCGATCTGCCCAGCGAGTACTGGCAGATCCAGAAGCTGGTGAAGTA | |
| | CCTGAAAGGCGGAAACCAGACCGCCACCGTGATCGCCCTGTGCAGCATGAGAGACTTCAGCC | |
| | TGGCTCAAGAGACATGCCAGCTCGCCATCAGAGATGTCGGCGGACTGGAAGTGCTGATCAAC | |
| | CTGCTGGAAACCGACGAAGTGAAGTGCAAGATCGGCAGCCTGAAAATCCTCAAAGAGATCAG | |
| | CCACAATCCTCAGATCCGGCAGAACATCGTGGACCTCGGAGGCCTGCCCATCATGGTCAACA | |
| | TCCTGGACAGCCCTCACAAGAGCCTGAAGTGTCTGGCCGCCGAGACAATCGCCAACGTGGCC | |
| | AAGTTCAAGCGGGCCAGAAGAGTCGTCAGACAGCACGGCGGAATCACCAAACTGGTGGCCCT | |
| | GCTGGACTGCGCCCACGACAGCACAAAGCCCGCTCAGAGCAGCCTGTACGAGGCCAGAGATG | |
| | TGGAAGTGGCCAGATGTGGCGCTCTGGCCCTGTGGAGCTGCAGCAAGAGCCACACCAACAAA | |
| | GAGGCCATCAGAAAGGCTGGCGGCATCCCTCTGCTGGCCAGACTGCTGAAAACCAGCCACGA | |
| | GAACATGCTGATCCCCGTCGTGGGCACACTGCAAGAGTGTGCCAGCGAGGAAAACTACAGAG | |
| | CCGCCATCAAGGCCGAGCGGATCATCGAGAACCTCGTGAAGAATCTGAACAGCGAGAACGAG | |
| | CAGCTGCAAGAGCACTGCGCCATGGCCATCTATCAGTGCGCCGAGGACAAAGAAACCCGGGA | |
| | TCTGGTGCGGCTGCACGGCGGCCTGAAACCTCTGGCCAGCCTGCTGAACAACACCGACAACA | |
| | AAGAACGGCTGGCCGCTGTGACAGGCGCCATCTGGAAGTGCAGCATCAGCAAAGAAAACGTG | |
| | ACGAAGTTCCGCGAGTACAAGGCCATCGAGACACTCGTGGGCCTGCTGACTGACCAACCTGA | |
| | AGAGGTGCTCGTGAACGTGGTGGGAGCCCTGGGCGAGTGCTGTCAAGAGCGGGAAAACAGAG | |
| | TGATCGTGCGGAAGTGCGGAGGCATCCAGCCTCTCGTGAATCTGCTCGTGGGCATCAATCAG | |
| | GCCCTGCTGGTGAACGTGACAAAGGCCGTGGGAGCCTGTGCTGTGGAACCTGAGAGCATGAT | |
| | GATCATCGACCGGCTGGATGGCGTGCGGCTGCTGTGGAGTCTGCTGAAGAACCCTCATCCTG | |
| | ACGTGAAGGCCTCTGCCGCCTGGGCTCTGTGCCCCTGCATCAAGAATGCCAAGGACGCCGGC | |
| | GAGATGGTCaggAGCTTCGTGGGAGGCCTGGAACTGATCGTGAACCTGCTGAAGTCTGACAA | |
| | CAAAGAAGTCCTGGCCAGCGTCTGCGCCGCCATCACCAACATCGCCAAGGATCAAGAGAACC | |
| | TGGCCGTGATCACCGACCATGGCGTGGTGCCACTGCTGAGCAAGCTGGCCAACACCAACAAC | |
| | AACAAGCTGCGGCACCATCTGGCCGAGGCCATCAGCAGATGCTGCATGTGGGGCCGCAACAG | |
| | AGTGGCCTTCGGAGAGCACAAAGCCGTGGCACCTCTCGTGCGGTATCTGAAGAGCAACGACA | |
| | CCAATGTGCACCGGGCCACAGCCCAGGCTCTGTACCAGCTGTCTGAGGACGCCGACAACTGC | |
| | ATCACCATGCACGAAAATGGCGCCGTGAAACTGCTGCTGGACATGGTCGGAAGCCCCGACCA | |
| | GGATCTGCAAGAGGCTGCTGCCGGCTGCATCAGCAACATCAGAAGGCTGGCCCTGGCCACCG | |
| | AGAAGGCCAGATACACCTGAGAATTCTGCAGAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAA | |
| | AAAAAAAAAAAAAAAAAAAAAAAAAAATTCG | |
| SEQ ID | ATGGGAGTGGCCCTGAGAAAGCTGACCCAGTGGACAGCCGCTGGACACGGAACAGGCATCCT | ARMC 4 |
| NO: 7 | GGAAATCACCCCTCTGAACGAGGCCATCCTGAAAGAAATCATCGTGTTCGTCGAGAGCTTCA | (ORF and |
| | TCTACAAGCACCCTCAAGAGGCCAAGTTCGTGTTCGTGGAACCCCTGGAATGGAACACCTCT | HA tag) |
| | CTGGCCCCCAGCGCCTTCGAGAGCGGCTACGTGGTGTCTGAGACAACCGTGAAGTCCGAGGA | |
| | AGTGGACAAGAACGGCCAGCCTCTGCTGTTCCTGAGCGTGCCCCAGATCAAGATCAGgAGCT | |
| | TCGGCCAGCTGAGCCGGCTGCTGCTGATCGCCAAAACCGGCAAGCTGAAAGAGGCCCAGGCC | |
| | TGCGTGGAGGCCAACAGAGATCCCATCGTGAAGATCCTGGGCAGCGACTACAACACCATGAA | |
| | GGAAAACAGCATCGCCCTGAACATCCTGGGAAAGATCACCAGGGACGACGACCCCGAGAGCG | |
| | AGATCAAGATGAAGATCGCCATGCTGCTGAAGCAGCTGGACCTGCATCTGCTGAACCACAGC | |
| | CTGAAGCACATCAGCCTGGAAATCTCTCTGAGCCCCATGACCGTGAAGAAGGACATCGAGCT | |
| | GCTGAAACGGTTCAGCGGCAAGGGCAATCAGACCGTGCTGGAAAGCATCGAGTACACCAGCG | |
| | ACTACGAGTTCAGCAACGGCTGCAGAGCCCCACCCTGGAGACAGATCAGAGGCGAGATCTGC | |
| | TACGTGCTGGTCAAGCCCCACGATGGCGAGACACTGTGCATCACATGCTCTGCCGGCGGAGT | |
| | GTTCCTGAACGGCGGCAAGACAGATGATGAGGGCGACGTGAACTACGAGCGGAAGGGCAGCA | |
| | TCTACAAGAACCTGGTCACCTTCCTGCGGGAAAAGAGCCCCAAGTTCAGCGAGAACATGAGC | |
| | AAGCTGGGCATCAGCTTCAGCGAGGACCAGCAGAAAGAGAAGGACCAGCTGGGCAAAGCCCC | |
| | CAAGAAAGAGGAAGCCGCCGCTCTGAGAAAGGACATCAGCGGCAGCGACAAGCGGAGCCTGG | |
| | AAAAGAACCAGATCAACTTCTGGCGGAACCAGATGACCAAGAGATGGGAGCCCAGCCTGAAC | |
| | TGGAAAACCACCGTGAACTACAAAGGCAAGGGCAGCGCCAAAGAGATCCAAGAGGACAAGCA | |
| | CACAGGCAAGCTGGAAAAGCCTCGGCCCAGCGTGTCTCATGGCAGAGCACAGCTGCTGAGAA | |
| | AGAGCGCCGAGAAGATCGAGGAAACCGTGAGCGACAGCAGCAGCGAGAGCGAAGAGGACGAG | |
| | GAACCTCCTGACCACAGACAAGAAGCCAGCGCCGATCTGCCCAGCGAGTACTGGCAGATCCA | |
| | GAAGCTGGTGAAGTACCTGAAAGGCGGAAACCAGACCGCCACCGTGATCGCCCTGTGCAGCA | |
| | TGAGAGACTTCAGCCTGGCTCAAGAGACATGCCAGCTCGCCATCAGAGATGTCGGCGGACTG | |
| | GAAGTGCTGATCAACCTGCTGGAAACCGACGAAGTGAAGTGCAAGATCGGCAGCCTGAAAAT | |
| | CCTCAAAGAGATCAGCCACAATCCTCAGATCCGGCAGAACATCGTGGACCTCGGAGGCCTGC | |
| | CCATCATGGTCAACATCCTGGACAGCCCTCACAAGAGCCTGAAGTGTCTGGCCGCCGAGACA | |
| | ATCGCCAACGTGGCCAAGTTCAAGCGGGCCAGAAGAGTCGTCAGACAGCACGGCGGAATCAC | |
| | CAAACTGGTGGCCCTGCTGGACTGCGCCCACGACAGCACAAAGCCCGCTCAGAGCAGCCTGT | |
| | ACGAGGCCAGAGATGTGGAAGTGGCCAGATGTGGCGCTCTGGCCCTGTGGAGCTGCAGCAAG | |
| | AGCCACACCAACAAAGAGGCCATCAGAAAGGCTGGCGGCATCCCTCTGCTGGCCAGACTGCT | |
| | GAAAACCAGCCACGAGAACATGCTGATCCCCGTCGTGGGCACACTGCAAGAGTGTGCCAGCG | |
| | AGGAAAACTACAGAGCCGCCATCAAGGCCGAGCGGATCATCGAGAACCTCGTGAAGAATCTG | |
| | AACAGCGAGAACGAGCAGCTGCAAGAGCACTGCGCCATGGCCATCTATCAGTGCGCCGAGGA | |
| | CAAAGAAACCCGGGATCTGGTGCGGCTGCACGGCGGCCTGAAACCTCTGGCCAGCCTGCTGA | |
| | ACAACACCGACAACAAAGAACGGCTGGCCGCTGTGACAGGCGCCATCTGGAAGTGCAGCATC | |
| | AGCAAAGAAAACGTGACGAAGTTCCGCGAGTACAAGGCCATCGAGACACTCGTGGGCCTGCT | |
| | GACTGACCAACCTGAAGAGGTGCTCGTGAACGTGGTGGGAGCCCTGGGCGAGTGCTGTCAAG | |
| | AGCGGGAAAACAGAGTGATCGTGCGGAAGTGCGGAGGCATCCAGCCTCTCGTGAATCTGCTC | |
| | GTGGGCATCAATCAGGCCCTGCTGGTGAACGTGACAAAGGCCGTGGGAGCCTGTGCTGTGGA | |
| | ACCTGAGAGCATGATGATCATCGACCGGCTGGATGGCGTGCGGCTGCTGTGGAGTCTGCTGA | |
| | AGAACCCTCATCCTGACGTGAAGGCCTCTGCCGCCTGGGCTCTGTGCCCCTGCATCAAGAAT | |
| | GCCAAGGACGCCGGCGAGATGGTCaggAGCTTCGTGGGAGGCCTGGAACTGATCGTGAACCT | |
| | GCTGAAGTCTGACAACAAAGAAGTCCTGGCCAGCGTCTGCGCCGCCATCACCAACATCGCCA | |
| | AGGATCAAGAGAACCTGGCCGTGATCACCGACCATGGCGTGGTGCCACTGCTGAGCAAGCTG | |
| | GCCAACACCAACAACAACAAGCTGCGGCACCATCTGGCCGAGGCCATCAGCAGATGCTGCAT | |
| | GTGGGGCCGCAACAGAGTGGCCTTCGGAGAGCACAAAGCCGTGGCACCTCTCGTGCGGTATC | |
| | TGAAGAGCAACGACACCAATGTGCACCGGGCCACAGCCCAGGCTCTGTACCAGCTGTCTGAG | |
| | GACGCCGACAACTGCATCACCATGCACGAAAATGGCGCCGTGAAACTGCTGCTGGACATGGT | |
| | CGGAAGCCCCGACCAGGATCTGCAAGAGGCTGCTGCCGGCTGCATCAGCAACATCAGAAGGC | |
| | TGGCCCTGGCCACCGAGAAGGCCAGATACACCTGAGGAAGCGGCTACCCATACGATGTTCCT | |
| | GACTATGCGTGA | |
| SEQ ID | GGGAGACAAGAGAGAAAAGAAGAGCAAGAAGAAATATAAGAGCCACCATGGGAGTGGCCCTG | ARMC4 |
| NO: 8 | AGAAAGCTGACCCAGTGGACAGCCGCTGGACACGGAACAGGCATCCTGGAAATCACCCCTCT | (,5′ UTR, |
| | GAACGAGGCCATCCTGAAAGAAATCATCGTGTTCGTCGAGAGCTTCATCTACAAGCACCCTC | ORF, HA |
| | AAGAGGCCAAGTTCGTGTTCGTGGAACCCCTGGAATGGAACACCTCTCTGGCCCCCAGCGCC | tag, and |
| | TTCGAGAGCGGCTACGTGGTGTCTGAGACAACCGTGAAGTCCGAGGAAGTGGACAAGAACGG | 3′ tail) |
| | CCAGCCTCTGCTGTTCCTGAGCGTGCCCCAGATCAAGATCAGgAGCTTCGGCCAGCTGAGCC | |
| | GGCTGCTGCTGATCGCCAAAACCGGCAAGCTGAAAGAGGCCCAGGCCTGCGTGGAGGCCAAC | |
| | AGAGATCCCATCGTGAAGATCCTGGGCAGCGACTACAACACCATGAAGGAAAACAGCATCGC | |
| | CCTGAACATCCTGGGAAAGATCACCAGGGACGACGACCCCGAGAGCGAGATCAAGATGAAGA | |
| | TCGCCATGCTGCTGAAGCAGCTGGACCTGCATCTGCTGAACCACAGCCTGAAGCACATCAGC | |
| | CTGGAAATCTCTCTGAGCCCCATGACCGTGAAGAAGGACATCGAGCTGCTGAAACGGTTCAG | |
| | CGGCAAGGGCAATCAGACCGTGCTGGAAAGCATCGAGTACACCAGCGACTACGAGTTCAGCA | |
| | ACGGCTGCAGAGCCCCACCCTGGAGACAGATCAGAGGCGAGATCTGCTACGTGCTGGTCAAG | |
| | CCCCACGATGGCGAGACACTGTGCATCACATGCTCTGCCGGCGGAGTGTTCCTGAACGGCGG | |
| | CAAGACAGATGATGAGGGCGACGTGAACTACGAGCGGAAGGGCAGCATCTACAAGAACCTGG | |
| | TCACCTTCCTGCGGGAAAAGAGCCCCAAGTTCAGCGAGAACATGAGCAAGCTGGGCATCAGC | |
| | TTCAGCGAGGACCAGCAGAAAGAGAAGGACCAGCTGGGCAAAGCCCCCAAGAAAGAGGAAGC | |
| | CGCCGCTCTGAGAAAGGACATCAGCGGCAGCGACAAGCGGAGCCTGGAAAAGAACCAGATCA | |
| | ACTTCTGGCGGAACCAGATGACCAAGAGATGGGAGCCCAGCCTGAACTGGAAAACCACCGTG | |
| | AACTACAAAGGCAAGGGCAGCGCCAAAGAGATCCAAGAGGACAAGCACACAGGCAAGCTGGA | |
| | AAAGCCTCGGCCCAGCGTGTCTCATGGCAGAGCACAGCTGCTGAGAAAGAGCGCCGAGAAGA | |
| | TCGAGGAAACCGTGAGCGACAGCAGCAGCGAGAGCGAAGAGGACGAGGAACCTCCTGACCAC | |
| | AGACAAGAAGCCAGCGCCGATCTGCCCAGCGAGTACTGGCAGATCCAGAAGCTGGTGAAGTA | |
| | CCTGAAAGGCGGAAACCAGACCGCCACCGTGATCGCCCTGTGCAGCATGAGAGACTTCAGCC | |
| | TGGCTCAAGAGACATGCCAGCTCGCCATCAGAGATGTCGGCGGACTGGAAGTGCTGATCAAC | |
| | CTGCTGGAAACCGACGAAGTGAAGTGCAAGATCGGCAGCCTGAAAATCCTCAAAGAGATCAG | |
| | CCACAATCCTCAGATCCGGCAGAACATCGTGGACCTCGGAGGCCTGCCCATCATGGTCAACA | |
| | TCCTGGACAGCCCTCACAAGAGCCTGAAGTGTCTGGCCGCCGAGACAATCGCCAACGTGGCC | |
| | AAGTTCAAGCGGGCCAGAAGAGTCGTCAGACAGCACGGCGGAATCACCAAACTGGTGGCCCT | |
| | GCTGGACTGCGCCCACGACAGCACAAAGCCCGCTCAGAGCAGCCTGTACGAGGCCAGAGATG | |
| | TGGAAGTGGCCAGATGTGGCGCTCTGGCCCTGTGGAGCTGCAGCAAGAGCCACACCAACAAA | |
| | GAGGCCATCAGAAAGGCTGGCGGCATCCCTCTGCTGGCCAGACTGCTGAAAACCAGCCACGA | |
| | GAACATGCTGATCCCCGTCGTGGGCACACTGCAAGAGTGTGCCAGCGAGGAAAACTACAGAG | |
| | CCGCCATCAAGGCCGAGCGGATCATCGAGAACCTCGTGAAGAATCTGAACAGCGAGAACGAG | |
| | CAGCTGCAAGAGCACTGCGCCATGGCCATCTATCAGTGCGCCGAGGACAAAGAAACCCGGGA | |
| | TCTGGTGCGGCTGCACGGCGGCCTGAAACCTCTGGCCAGCCTGCTGAACAACACCGACAACA | |
| | AAGAACGGCTGGCCGCTGTGACAGGCGCCATCTGGAAGTGCAGCATCAGCAAAGAAAACGTG | |
| | ACGAAGTTCCGCGAGTACAAGGCCATCGAGACACTCGTGGGCCTGCTGACTGACCAACCTGA | |
| | AGAGGTGCTCGTGAACGTGGTGGGAGCCCTGGGCGAGTGCTGTCAAGAGCGGGAAAACAGAG | |
| | TGATCGTGCGGAAGTGCGGAGGCATCCAGCCTCTCGTGAATCTGCTCGTGGGCATCAATCAG | |
| | GCCCTGCTGGTGAACGTGACAAAGGCCGTGGGAGCCTGTGCTGTGGAACCTGAGAGCATGAT | |
| | GATCATCGACCGGCTGGATGGCGTGCGGCTGCTGTGGAGTCTGCTGAAGAACCCTCATCCTG | |
| | ACGTGAAGGCCTCTGCCGCCTGGGCTCTGTGCCCCTGCATCAAGAATGCCAAGGACGCCGGC | |
| | GAGATGGTCaggAGCTTCGTGGGAGGCCTGGAACTGATCGTGAACCTGCTGAAGTCTGACAA | |
| | CAAAGAAGTCCTGGCCAGCGTCTGCGCCGCCATCACCAACATCGCCAAGGATCAAGAGAACC | |
| | TGGCCGTGATCACCGACCATGGCGTGGTGCCACTGCTGAGCAAGCTGGCCAACACCAACAAC | |
| | AACAAGCTGCGGCACCATCTGGCCGAGGCCATCAGCAGATGCTGCATGTGGGGCCGCAACAG | |
| | AGTGGCCTTCGGAGAGCACAAAGCCGTGGCACCTCTCGTGCGGTATCTGAAGAGCAACGACA | |
| | CCAATGTGCACCGGGCCACAGCCCAGGCTCTGTACCAGCTGTCTGAGGACGCCGACAACTGC | |
| | ATCACCATGCACGAAAATGGCGCCGTGAAACTGCTGCTGGACATGGTCGGAAGCCCCGACCA | |
| | GGATCTGCAAGAGGCTGCTGCCGGCTGCATCAGCAACATCAGAAGGCTGGCCCTGGCCACCG | |
| | AGAAGGCCAGATACACCGGAAGCGGCTACCCATACGATGTTCCTGACTATGCGTGAGAATTC | |
| | TGCAGAAAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAA | |
| | ATTCG | |
| SEQ ID | ATGCATCCTGAGCCATCTGAACCTGCCACAGGCGGAGCCGCCGAACTGGACTGTGCTCAAGA | DNAAF1 |
| NO: 9 | ACCTGGCGTGGAAGAGTCTGCCGGCGATCATGGATCTGCTGGAAGAGGCGGCTGCAAAGAGG | (ORF) |
| | AAATCAACGACCCCAAAGAAATCTGCGTGGGCAGCAGCGACACCAGCTACCACTCTCAGCAG | |
| | AAGCAGAGCGGCGACAACGGATCTGGCGGCCACTTCGCCCATCCAAGAGAGGACAGAGAGGA | |
| | TCGGGGCCCCAGAATGACCAAGAGCAGCCTGCAGAAGCTGTGCAAGCAGCACAAGCTGTACA | |
| | TCACCCCTGCTCTGAACGACACCCTGTACCTGCACTTCAAGGGCTTCGACCGGATCGAGAAC | |
| | CTGGAAGAGTACACCGGCCTGAGATGCCTGTGGCTGCAGAGCAATGGCATCCAGAAGATCGA | |
| | AAATCTGGAAGCCCAGACCGAGCTGCGGTGCCTGTTCCTGCAAATGAATCTGCTGCGGAAGA | |
| | TCGAGAACCTCGAGCCTCTGCAGAAACTGGACGCCCTGAACCTGAGCAACAACTACATCAAG | |
| | ACCATCGAGAATCTGAGCTGCCTGCCTGTGCTGAACACCCTGCAGATGGCCCACAACCACCT | |
| | GGAAACCGTGGAAGACATCCAGCATCTGCAAGAGTGCCTGCGGCTGTGCGTGCTGGATCTGT | |
| | CTCACAACAAGCTGAGCGACCCCGAGATCCTGAGCATCCTGGAAAGCATGCCTGACCTGCGG | |
| | GTGCTGAATCTGATGGGCAACCCCGTGATCCGGCAGATCCCCAACTACAGACGGACCGTGAC | |
| | AGTGCGGCTGAAGCACCTGACCTACCTGGACGACAGACCTGTGTTCCCCAAGGACAGAGCCT | |
| | GTGCCGAAGCCTGGGCCAGAGGCGGATATGCCGCCGAGAAAGAAGAACGGCAGCAGTGGGAG | |
| | AGCAGAGAGCGGAAGAAGATCACCGACAGCATCGAGGCCCTGGCCATGATCAAGCAGCGGGC | |
| | CGAAGAGAGAAAGCGCCAGAGAGAGTCTCAAGAGCGGGGCGAGATGACCAGCTCTGACGATG | |
| | GCGAAAATGTGCCCGCCTCTGCCGAGGGAAAAGAGGAACCTCCTGGCGACAGGGAAACCCGG | |
| | CAGAAAATGGAACTGTTCGTGAAAGAGAGCTTCGAGGCCAAGGACGAGCTGTGCCCTGAGAA | |
| | ACCCTCTGGCGAGGAACCACCTGTGGAAGCCAAGCGAGAAGATGGCGGACCTGAGCCTGAGG | |
| | GAACACTGCCTGCTGAAACTCTGCTGCTGAGCAGCCCCGTGGAAGTGAAAGGCGAGGATGGC | |
| | GACGGCGAACCTGAAGGCACACTGCCAGCTGAAGCTCCTCCACCTCCACCACCAGTCGAAGT | |
| | CAAAGGCGAAGATGGGGATCAAGAGCCCGAAGGCACTCTGCCAGCAGAGACACTGCTGCTGA | |
| | GCCCACCTGTGAAAGTGAAGGGCGAAGATGGCGACAGAGAGCCAGAGGGCACACTCCCAGCC | |
| | GAAGCACCACCACCACCTCCACTGGGAGCCGCCAGAGAAGAACCCACACCTCAGGCCGTGGC | |
| | CACCGAGGGCGTGTTCGTGACAGAACTGGATGGCACCAGAACCGAGGATCTGGAAACCATCC | |
| | GGCTGGAAACAAAAGAGACATTCTGCATCGACGATCTGCCCGACCTCGAGGACGATGACGAG | |
| | ACAGGCAAGAGCCTGGAAGATCAGAACATGTGCTTCCCCAAGATCGAAGTGATCAGCAGCCT | |
| | GAGCGACGACAGCGACCCTGAGCTGGACTACACAAGCCTGCCAGTCCTGGAAAACCTGCCCA | |
| | CCGACACACTGAGCAACATCTTCGCCGTGAGCAAGGACACCAGCAAGGCCGCCAGAGTGCCC | |
| | TTCACCGACATCTTCAAGAAAGAGGCCAAGCGCGACCTGGAAATCCGGAAGCAGGACACAAA | |
| | GAGCCCCAGACCTCTGATCCAAGAGCTGAGCGACGAGGATCCCTCTGGCCAGCTGCTGATGC | |
| | CTCCCACCTGTCAGAGAGATGCCGCTCCTCTGACCAGCAGCGGCGACAGAGACAGCGACTTC | |
| | CTGGCCGCCAGCAGCCCTGTGCCAACAGAATCTGCAGCCACCCCTCCTGAGACATGCGTGGG | |
| | AGTGGCTCAGCCCTCTCAGGCCCTGCCCACATGGGACCTGACAGCCTTCCCTGCTCCCAAGG | |
| | CCAGCTGA | |
| SEQ ID | GGGAGACAAGAGAGAAAAGAAGAGCAAGAAGAAATATAAGAGCCACCATGCATCCTGAGCCA | DNAAF1 |
| NO: 10 | TCTGAACCTGCCACAGGCGGAGCCGCCGAACTGGACTGTGCTCAAGAACCTGGCGTGGAAGA | (5′ UTR, |
| | GTCTGCCGGCGATCATGGATCTGCTGGAAGAGGCGGCTGCAAAGAGGAAATCAACGACCCCA | ORF, and |
| | AAGAAATCTGCGTGGGCAGCAGCGACACCAGCTACCACTCTCAGCAGAAGCAGAGCGGCGAC | 3′ Tail) |
| | AACGGATCTGGCGGCCACTTCGCCCATCCAAGAGAGGACAGAGAGGATCGGGGCCCCAGAAT | |
| | GACCAAGAGCAGCCTGCAGAAGCTGTGCAAGCAGCACAAGCTGTACATCACCCCTGCTCTGA | |
| | ACGACACCCTGTACCTGCACTTCAAGGGCTTCGACCGGATCGAGAACCTGGAAGAGTACACC | |
| | GGCCTGAGATGCCTGTGGCTGCAGAGCAATGGCATCCAGAAGATCGAAAATCTGGAAGCCCA | |
| | GACCGAGCTGCGGTGCCTGTTCCTGCAAATGAATCTGCTGCGGAAGATCGAGAACCTCGAGC | |
| | CTCTGCAGAAACTGGACGCCCTGAACCTGAGCAACAACTACATCAAGACCATCGAGAATCTG | |
| | AGCTGCCTGCCTGTGCTGAACACCCTGCAGATGGCCCACAACCACCTGGAAACCGTGGAAGA | |
| | CATCCAGCATCTGCAAGAGTGCCTGCGGCTGTGCGTGCTGGATCTGTCTCACAACAAGCTGA | |
| | GCGACCCCGAGATCCTGAGCATCCTGGAAAGCATGCCTGACCTGCGGGTGCTGAATCTGATG | |
| | GGCAACCCCGTGATCCGGCAGATCCCCAACTACAGACGGACCGTGACAGTGCGGCTGAAGCA | |
| | CCTGACCTACCTGGACGACAGACCTGTGTTCCCCAAGGACAGAGCCTGTGCCGAAGCCTGGG | |
| | CCAGAGGCGGATATGCCGCCGAGAAAGAAGAACGGCAGCAGTGGGAGAGCAGAGAGCGGAAG | |
| | AAGATCACCGACAGCATCGAGGCCCTGGCCATGATCAAGCAGCGGGCCGAAGAGAGAAAGCG | |
| | CCAGAGAGAGTCTCAAGAGCGGGGCGAGATGACCAGCTCTGACGATGGCGAAAATGTGCCCG | |
| | CCTCTGCCGAGGGAAAAGAGGAACCTCCTGGCGACAGGGAAACCCGGCAGAAAATGGAACTG | |
| | TTCGTGAAAGAGAGCTTCGAGGCCAAGGACGAGCTGTGCCCTGAGAAACCCTCTGGCGAGGA | |
| | ACCACCTGTGGAAGCCAAGCGAGAAGATGGCGGACCTGAGCCTGAGGGAACACTGCCTGCTG | |
| | AAACTCTGCTGCTGAGCAGCCCCGTGGAAGTGAAAGGCGAGGATGGCGACGGCGAACCTGAA | |
| | GGCACACTGCCAGCTGAAGCTCCTCCACCTCCACCACCAGTCGAAGTCAAAGGCGAAGATGG | |
| | GGATCAAGAGCCCGAAGGCACTCTGCCAGCAGAGACACTGCTGCTGAGCCCACCTGTGAAAG | |
| | TGAAGGGCGAAGATGGCGACAGAGAGCCAGAGGGCACACTCCCAGCCGAAGCACCACCACCA | |
| | CCTCCACTGGGAGCCGCCAGAGAAGAACCCACACCTCAGGCCGTGGCCACCGAGGGCGTGTT | |
| | CGTGACAGAACTGGATGGCACCAGAACCGAGGATCTGGAAACCATCCGGCTGGAAACAAAAG | |
| | AGACATTCTGCATCGACGATCTGCCCGACCTCGAGGACGATGACGAGACAGGCAAGAGCCTG | |
| | GAAGATCAGAACATGTGCTTCCCCAAGATCGAAGTGATCAGCAGCCTGAGCGACGACAGCGA | |
| | CCCTGAGCTGGACTACACAAGCCTGCCAGTCCTGGAAAACCTGCCCACCGACACACTGAGCA | |
| | ACATCTTCGCCGTGAGCAAGGACACCAGCAAGGCCGCCAGAGTGCCCTTCACCGACATCTTC | |
| | AAGAAAGAGGCCAAGCGCGACCTGGAAATCCGGAAGCAGGACACAAAGAGCCCCAGACCTCT | |
| | GATCCAAGAGCTGAGCGACGAGGATCCCTCTGGCCAGCTGCTGATGCCTCCCACCTGTCAGA | |
| | GAGATGCCGCTCCTCTGACCAGCAGCGGCGACAGAGACAGCGACTTCCTGGCCGCCAGCAGC | |
| | CCTGTGCCAACAGAATCTGCAGCCACCCCTCCTGAGACATGCGTGGGAGTGGCTCAGCCCTC | |
| | TCAGGCCCTGCCCACATGGGACCTGACAGCCTTCCCTGCTCCCAAGGCCAGCTGAGAATTCT | |
| | GCAGAAAAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAA | |
| | TTCG | |
| SEQ ID | ATGCATCCTGAGCCATCTGAACCTGCCACAGGCGGAGCCGCCGAACTGGACTGTGCTCAAGA | DNAAF1 |
| NO: 11 | ACCTGGCGTGGAAGAGTCTGCCGGCGATCATGGATCTGCTGGAAGAGGCGGCTGCAAAGAGG | (ORF and |
| | AAATCAACGACCCCAAAGAAATCTGCGTGGGCAGCAGCGACACCAGCTACCACTCTCAGCAG | HA tag) |
| | AAGCAGAGCGGCGACAACGGATCTGGCGGCCACTTCGCCCATCCAAGAGAGGACAGAGAGGA | |
| | TCGGGGCCCCAGAATGACCAAGAGCAGCCTGCAGAAGCTGTGCAAGCAGCACAAGCTGTACA | |
| | TCACCCCTGCTCTGAACGACACCCTGTACCTGCACTTCAAGGGCTTCGACCGGATCGAGAAC | |
| | CTGGAAGAGTACACCGGCCTGAGATGCCTGTGGCTGCAGAGCAATGGCATCCAGAAGATCGA | |
| | AAATCTGGAAGCCCAGACCGAGCTGCGGTGCCTGTTCCTGCAAATGAATCTGCTGCGGAAGA | |
| | TCGAGAACCTCGAGCCTCTGCAGAAACTGGACGCCCTGAACCTGAGCAACAACTACATCAAG | |
| | ACCATCGAGAATCTGAGCTGCCTGCCTGTGCTGAACACCCTGCAGATGGCCCACAACCACCT | |
| | GGAAACCGTGGAAGACATCCAGCATCTGCAAGAGTGCCTGCGGCTGTGCGTGCTGGATCTGT | |
| | CTCACAACAAGCTGAGCGACCCCGAGATCCTGAGCATCCTGGAAAGCATGCCTGACCTGCGG | |
| | GTGCTGAATCTGATGGGCAACCCCGTGATCCGGCAGATCCCCAACTACAGACGGACCGTGAC | |
| | AGTGCGGCTGAAGCACCTGACCTACCTGGACGACAGACCTGTGTTCCCCAAGGACAGAGCCT | |
| | GTGCCGAAGCCTGGGCCAGAGGCGGATATGCCGCCGAGAAAGAAGAACGGCAGCAGTGGGAG | |
| | AGCAGAGAGCGGAAGAAGATCACCGACAGCATCGAGGCCCTGGCCATGATCAAGCAGCGGGC | |
| | CGAAGAGAGAAAGCGCCAGAGAGAGTCTCAAGAGCGGGGCGAGATGACCAGCTCTGACGATG | |
| | GCGAAAATGTGCCCGCCTCTGCCGAGGGAAAAGAGGAACCTCCTGGCGACAGGGAAACCCGG | |
| | CAGAAAATGGAACTGTTCGTGAAAGAGAGCTTCGAGGCCAAGGACGAGCTGTGCCCTGAGAA | |
| | ACCCTCTGGCGAGGAACCACCTGTGGAAGCCAAGCGAGAAGATGGCGGACCTGAGCCTGAGG | |
| | GAACACTGCCTGCTGAAACTCTGCTGCTGAGCAGCCCCGTGGAAGTGAAAGGCGAGGATGGC | |
| | GACGGCGAACCTGAAGGCACACTGCCAGCTGAAGCTCCTCCACCTCCACCACCAGTCGAAGT | |
| | CAAAGGCGAAGATGGGGATCAAGAGCCCGAAGGCACTCTGCCAGCAGAGACACTGCTGCTGA | |
| | GCCCACCTGTGAAAGTGAAGGGCGAAGATGGCGACAGAGAGCCAGAGGGCACACTCCCAGCC | |
| | GAAGCACCACCACCACCTCCACTGGGAGCCGCCAGAGAAGAACCCACACCTCAGGCCGTGGC | |
| | CACCGAGGGCGTGTTCGTGACAGAACTGGATGGCACCAGAACCGAGGATCTGGAAACCATCC | |
| | GGCTGGAAACAAAAGAGACATTCTGCATCGACGATCTGCCCGACCTCGAGGACGATGACGAG | |
| | ACAGGCAAGAGCCTGGAAGATCAGAACATGTGCTTCCCCAAGATCGAAGTGATCAGCAGCCT | |
| | GAGCGACGACAGCGACCCTGAGCTGGACTACACAAGCCTGCCAGTCCTGGAAAACCTGCCCA | |
| | CCGACACACTGAGCAACATCTTCGCCGTGAGCAAGGACACCAGCAAGGCCGCCAGAGTGCCC | |
| | TTCACCGACATCTTCAAGAAAGAGGCCAAGCGCGACCTGGAAATCCGGAAGCAGGACACAAA | |
| | GAGCCCCAGACCTCTGATCCAAGAGCTGAGCGACGAGGATCCCTCTGGCCAGCTGCTGATGC | |
| | CTCCCACCTGTCAGAGAGATGCCGCTCCTCTGACCAGCAGCGGCGACAGAGACAGCGACTTC | |
| | CTGGCCGCCAGCAGCCCTGTGCCAACAGAATCTGCAGCCACCCCTCCTGAGACATGCGTGGG | |
| | AGTGGCTCAGCCCTCTCAGGCCCTGCCCACATGGGACCTGACAGCCTTCCCTGCTCCCAAGG | |
| | CCAGCGGAAGCGGCTACCCATACGATGTTCCTGACTATGCGTGA | |
| SEQ ID | GGGAGACAAGAGAGAAAAGAAGAGCAAGAAGAAATATAAGAGCCACCATGCATCCTGAGCCA | DNAAF1 |
| NO: 12 | TCTGAACCTGCCACAGGCGGAGCCGCCGAACTGGACTGTGCTCAAGAACCTGGCGTGGAAGA | (5′ UTR, |
| | GTCTGCCGGCGATCATGGATCTGCTGGAAGAGGCGGCTGCAAAGAGGAAATCAACGACCCCA | ORF, HA |
| | AAGAAATCTGCGTGGGCAGCAGCGACACCAGCTACCACTCTCAGCAGAAGCAGAGCGGCGAC | tag, and |
| | AACGGATCTGGCGGCCACTTCGCCCATCCAAGAGAGGACAGAGAGGATCGGGGCCCCAGAAT | 3′ Tail) |
| | GACCAAGAGCAGCCTGCAGAAGCTGTGCAAGCAGCACAAGCTGTACATCACCCCTGCTCTGA | |
| | ACGACACCCTGTACCTGCACTTCAAGGGCTTCGACCGGATCGAGAACCTGGAAGAGTACACC | |
| | GGCCTGAGATGCCTGTGGCTGCAGAGCAATGGCATCCAGAAGATCGAAAATCTGGAAGCCCA | |
| | GACCGAGCTGCGGTGCCTGTTCCTGCAAATGAATCTGCTGCGGAAGATCGAGAACCTCGAGC | |
| | CTCTGCAGAAACTGGACGCCCTGAACCTGAGCAACAACTACATCAAGACCATCGAGAATCTG | |
| | AGCTGCCTGCCTGTGCTGAACACCCTGCAGATGGCCCACAACCACCTGGAAACCGTGGAAGA | |
| | CATCCAGCATCTGCAAGAGTGCCTGCGGCTGTGCGTGCTGGATCTGTCTCACAACAAGCTGA | |
| | GCGACCCCGAGATCCTGAGCATCCTGGAAAGCATGCCTGACCTGCGGGTGCTGAATCTGATG | |
| | GGCAACCCCGTGATCCGGCAGATCCCCAACTACAGACGGACCGTGACAGTGCGGCTGAAGCA | |
| | CCTGACCTACCTGGACGACAGACCTGTGTTCCCCAAGGACAGAGCCTGTGCCGAAGCCTGGG | |
| | CCAGAGGCGGATATGCCGCCGAGAAAGAAGAACGGCAGCAGTGGGAGAGCAGAGAGCGGAAG | |
| | AAGATCACCGACAGCATCGAGGCCCTGGCCATGATCAAGCAGCGGGCCGAAGAGAGAAAGCG | |
| | CCAGAGAGAGTCTCAAGAGCGGGGCGAGATGACCAGCTCTGACGATGGCGAAAATGTGCCCG | |
| | CCTCTGCCGAGGGAAAAGAGGAACCTCCTGGCGACAGGGAAACCCGGCAGAAAATGGAACTG | |
| | TTCGTGAAAGAGAGCTTCGAGGCCAAGGACGAGCTGTGCCCTGAGAAACCCTCTGGCGAGGA | |
| | ACCACCTGTGGAAGCCAAGCGAGAAGATGGCGGACCTGAGCCTGAGGGAACACTGCCTGCTG | |
| | AAACTCTGCTGCTGAGCAGCCCCGTGGAAGTGAAAGGCGAGGATGGCGACGGCGAACCTGAA | |
| | GGCACACTGCCAGCTGAAGCTCCTCCACCTCCACCACCAGTCGAAGTCAAAGGCGAAGATGG | |
| | GGATCAAGAGCCCGAAGGCACTCTGCCAGCAGAGACACTGCTGCTGAGCCCACCTGTGAAAG | |
| | TGAAGGGCGAAGATGGCGACAGAGAGCCAGAGGGCACACTCCCAGCCGAAGCACCACCACCA | |
| | CCTCCACTGGGAGCCGCCAGAGAAGAACCCACACCTCAGGCCGTGGCCACCGAGGGCGTGTT | |
| | CGTGACAGAACTGGATGGCACCAGAACCGAGGATCTGGAAACCATCCGGCTGGAAACAAAAG | |
| | AGACATTCTGCATCGACGATCTGCCCGACCTCGAGGACGATGACGAGACAGGCAAGAGCCTG | |
| | GAAGATCAGAACATGTGCTTCCCCAAGATCGAAGTGATCAGCAGCCTGAGCGACGACAGCGA | |
| | CCCTGAGCTGGACTACACAAGCCTGCCAGTCCTGGAAAACCTGCCCACCGACACACTGAGCA | |
| | ACATCTTCGCCGTGAGCAAGGACACCAGCAAGGCCGCCAGAGTGCCCTTCACCGACATCTTC | |
| | AAGAAAGAGGCCAAGCGCGACCTGGAAATCCGGAAGCAGGACACAAAGAGCCCCAGACCTCT | |
| | GATCCAAGAGCTGAGCGACGAGGATCCCTCTGGCCAGCTGCTGATGCCTCCCACCTGTCAGA | |
| | GAGATGCCGCTCCTCTGACCAGCAGCGGCGACAGAGACAGCGACTTCCTGGCCGCCAGCAGC | |
| | CCTGTGCCAACAGAATCTGCAGCCACCCCTCCTGAGACATGCGTGGGAGTGGCTCAGCCCTC | |
| | TCAGGCCCTGCCCACATGGGACCTGACAGCCTTCCCTGCTCCCAAGGCCAGCGGAAGCGGCT | |
| | ACCCATACGATGTTCCTGACTATGCGTGAGAATTCTGCAGAAAAAAAAAAAAAAAAAAAAAA | |
| | AAAAAAAAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAATTCG | |
| SEQ ID | ATGGCCAAAGCCGCTGCCAGCAGCAGCCTGGAAGATCTGGATCTGAGCGGCGAGGAAGTGCA | DNAAF2 |
| NO: 13 | GAGACTGACCAGCGCCTTCCAGGACCCCGAGTTCAGACGGATGTTCAGCCAGTACGCCGAGG | (ORF) |
| | AACTGACAGACCCCGAGAACAGGCGGAGATACGAGGCCGAAATCACAGCCCTGGAAAGAGAA | |
| | CGGGGCGTCGAAGTCCGCTTCGTGCACCCTGAACCTGGCCACGTGCTGAGAACATCTCTGGA | |
| | CGGCGCCAGACGGTGCTTCGTGAACGTGTGCAGCAACGCCCTGGTGGGCGCTCCCAGCAGCA | |
| | GACCAGGATCTGGTGGCGACAGAGGCGCCGCTCCTGGATCTCACTGGAGCCTGCCCTACTCT | |
| | CTGGCCCCTGGCAGAGAGTACGCCGGCAGAAGCAGCTCTCGGTACATGGTGTACGACGTGGT | |
| | GTTCCACCCCGACGCTCTGGCTCTGGCCAGAAGGCACGAAGGCTTCAGACAGATGCTGGACG | |
| | CCACAGCTCTGGAAGCCGTGGAAAAGCAGTTCGGCGTGAAGCTGGACCGGCGGAATGCCAAG | |
| | ACACTGAAGGCCAAGTACAAGGGCACACCTGAAGCCGCCGTCCTGAGAACACCACTGCCTGG | |
| | TGTGATCCCCGCCAGACCTGATGGCGAGCCCAAAGGACCTCTGCCTGACTTCCCCTATCCCT | |
| | ACCAGTATCCAGCCGCTCCAGGACCCAGAGCACCCTCTCCACCAGAAGCTGCTCTGCAGCCA | |
| | GCTCCCACCGAGCCCAGATACAGCGTGGTGCAGAGGCACCATGTGGACCTGCAGGACTACAG | |
| | ATGCAGCAGAGACAGCGCCCCATCTCCTGTGCCTCACGAGCTGGTCATCACCATCGAACTGC | |
| | CCCTGCTGAGAAGCGCCGAACAGGCTGCACTGGAAGTGACAAGAAAGCTGCTGTGCCTGGAC | |
| | AGCAGAAAGCCCGACTACCGGCTGAGACTGAGCCTGCCATATCCTGTGGATGACGGCAGAGG | |
| | CAAGGCCCAGTTCAACAAAGCTCGGAGACAGCTGGTGGTCACCCTGCCTGTGGTGCTGCCTG | |
| | CCGCCAGAAGAGAACCTGCCGTGGCTGTGGCTGCCGCTGCTCCTGAAGAAAGCGCCGACAGA | |
| | TCTGGCACAGATGGCCAGGCCTGTGCCTCTGCCAGAGAAGGCGAAGCAGGACCCGCAAGAAG | |
| | CAGAGCCGAAGATGGCGGCCACGACACCTGTGTGGCTGGCGCAGCTGGAAGCGGCGTGACAA | |
| | CACTGGGAGATCCTGAAGTGGCCCCTCCACCAGCAGCAGCTGGCGAAGAAAGAGTGCCCAAG | |
| | CCTGGCGAGCAGGATCTGAGCAGACATGCCGGATCTCCACCTGGCAGCGTGGAAGAACCAAG | |
| | CCCTGGCGGAGAAAACTCTCCTGGCGGCGGAGGATCTCCCTGCCTGAGCAGCAGATCTCTGG | |
| | CCTGGGGAAGCAGCGCCGGAAGAGAATCTGCAAGAGGCGACAGCAGCGTGGAAACCCGGGAA | |
| | GAGTCTGAAGGCACAGGCGGACAGAGATCTGCCTGTGCCATGGGCGGACCTGGCACAAAGTC | |
| | TGGCGAACCTCTGTGCCCTCCTCTGCTGTGCAACCAGGACAAAGAGACACTGACACTGCTGA | |
| | TCCAGGTGCCACGGATCCAGCCTCAATCTCTGCAGGGCGACCTGAATCCTCTGTGGTACAAG | |
| | CTGAGATTCAGCGCCCAGGACCTGGTGTACAGCTTCTTCCTGCAATTCGCCCCAGAGAACAA | |
| | GCTGAGCACCACCGAGCCTGTGATCAGCATCAGCAGCAACAACGCCGTGATCGAGCTGGCCA | |
| | AGTCTCCTGAGTCTCACGGCCACTGGCGCGAGTGGTACTACGGCGTGAACAACGACAGCCTG | |
| | GAAGAACGGCTGTTCGTGAATGAGGAAAACGTGAACGAGTTCCTGGAAGAGGTGCTGAGCAG | |
| | CCCCTTCAAGCAGAGCATGAGCCTGACACCTCCACTGATCGAGGTGCTGCAAGTGACCGACA | |
| | ACAAGATCCAGATCAACGCCAAGCTGCAAGAGTGCAGCAACAGCGACCAGCTGCAGGGAAAA | |
| | GAGGAACGGGTCAACGAGGAAAGCCACCTGACCGAGAAAGAGTACATCGAGCACTGCAACAC | |
| | CCCCACCACCGACAGCGACAGCAGCATCGCCGTGAAGGCTCTGCAGATCGACAGCTTCGGCC | |
| | TGGTCACCTGCTTCCAGCAAGAGAGCCTGGACGTGAGCCAGATGATCCTGGGCAAGTCTCAG | |
| | CAGCCCGAGAGCAAGATGCAGAGCGAGTTCATCAAAGAGAAGAGCGCCACCTGCAGCAACGA | |
| | AGAGAAGGACAACCTGAACGAGAGCGTGATCACCGAAGAGAAAGAGACAGACGGCGACCACC | |
| | TGAGCAGCCTGCTGAACAAGACCACCGTGCACAACATCCCCGGCTTCGACAGCATCAAAGAA | |
| | ACAAACATGCAGGACGGCAGCGTGCAAGTGATCAAGGACCACGTGACCAACTGCGCCTTCAG | |
| | CTTCCAAAACAGCCTGCTGTACGACCTGGACTGA | |
| SEQ ID | GGGAGACAAGAGAGAAAAGAAGAGCAAGAAGAAATATAAGAGCCACCATGGCCAAAGCCGCT | DNAAF2 |
| NO: 14 | GCCAGCAGCAGCCTGGAAGATCTGGATCTGAGCGGCGAGGAAGTGCAGAGACTGACCAGCGC | (5′ UTR, |
| | CTTCCAGGACCCCGAGTTCAGACGGATGTTCAGCCAGTACGCCGAGGAACTGACAGACCCCG | ORF, and |
| | AGAACAGGCGGAGATACGAGGCCGAAATCACAGCCCTGGAAAGAGAACGGGGCGTCGAAGTC | 3′ Tail) |
| | CGCTTCGTGCACCCTGAACCTGGCCACGTGCTGAGAACATCTCTGGACGGCGCCAGACGGTG | |
| | CTTCGTGAACGTGTGCAGCAACGCCCTGGTGGGCGCTCCCAGCAGCAGACCAGGATCTGGTG | |
| | GCGACAGAGGCGCCGCTCCTGGATCTCACTGGAGCCTGCCCTACTCTCTGGCCCCTGGCAGA | |
| | GAGTACGCCGGCAGAAGCAGCTCTCGGTACATGGTGTACGACGTGGTGTTCCACCCCGACGC | |
| | TCTGGCTCTGGCCAGAAGGCACGAAGGCTTCAGACAGATGCTGGACGCCACAGCTCTGGAAG | |
| | CCGTGGAAAAGCAGTTCGGCGTGAAGCTGGACCGGCGGAATGCCAAGACACTGAAGGCCAAG | |
| | TACAAGGGCACACCTGAAGCCGCCGTCCTGAGAACACCACTGCCTGGTGTGATCCCCGCCAG | |
| | ACCTGATGGCGAGCCCAAAGGACCTCTGCCTGACTTCCCCTATCCCTACCAGTATCCAGCCG | |
| | CTCCAGGACCCAGAGCACCCTCTCCACCAGAAGCTGCTCTGCAGCCAGCTCCCACCGAGCCC | |
| | AGATACAGCGTGGTGCAGAGGCACCATGTGGACCTGCAGGACTACAGATGCAGCAGAGACAG | |
| | CGCCCCATCTCCTGTGCCTCACGAGCTGGTCATCACCATCGAACTGCCCCTGCTGAGAAGCG | |
| | CCGAACAGGCTGCACTGGAAGTGACAAGAAAGCTGCTGTGCCTGGACAGCAGAAAGCCCGAC | |
| | TACCGGCTGAGACTGAGCCTGCCATATCCTGTGGATGACGGCAGAGGCAAGGCCCAGTTCAA | |
| | CAAAGCTCGGAGACAGCTGGTGGTCACCCTGCCTGTGGTGCTGCCTGCCGCCAGAAGAGAAC | |
| | CTGCCGTGGCTGTGGCTGCCGCTGCTCCTGAAGAAAGCGCCGACAGATCTGGCACAGATGGC | |
| | CAGGCCTGTGCCTCTGCCAGAGAAGGCGAAGCAGGACCCGCAAGAAGCAGAGCCGAAGATGG | |
| | CGGCCACGACACCTGTGTGGCTGGCGCAGCTGGAAGCGGCGTGACAACACTGGGAGATCCTG | |
| | AAGTGGCCCCTCCACCAGCAGCAGCTGGCGAAGAAAGAGTGCCCAAGCCTGGCGAGCAGGAT | |
| | CTGAGCAGACATGCCGGATCTCCACCTGGCAGCGTGGAAGAACCAAGCCCTGGCGGAGAAAA | |
| | CTCTCCTGGCGGCGGAGGATCTCCCTGCCTGAGCAGCAGATCTCTGGCCTGGGGAAGCAGCG | |
| | CCGGAAGAGAATCTGCAAGAGGCGACAGCAGCGTGGAAACCCGGGAAGAGTCTGAAGGCACA | |
| | GGCGGACAGAGATCTGCCTGTGCCATGGGCGGACCTGGCACAAAGTCTGGCGAACCTCTGTG | |
| | CCCTCCTCTGCTGTGCAACCAGGACAAAGAGACACTGACACTGCTGATCCAGGTGCCACGGA | |
| | TCCAGCCTCAATCTCTGCAGGGCGACCTGAATCCTCTGTGGTACAAGCTGAGATTCAGCGCC | |
| | CAGGACCTGGTGTACAGCTTCTTCCTGCAATTCGCCCCAGAGAACAAGCTGAGCACCACCGA | |
| | GCCTGTGATCAGCATCAGCAGCAACAACGCCGTGATCGAGCTGGCCAAGTCTCCTGAGTCTC | |
| | ACGGCCACTGGCGCGAGTGGTACTACGGCGTGAACAACGACAGCCTGGAAGAACGGCTGTTC | |
| | GTGAATGAGGAAAACGTGAACGAGTTCCTGGAAGAGGTGCTGAGCAGCCCCTTCAAGCAGAG | |
| | CATGAGCCTGACACCTCCACTGATCGAGGTGCTGCAAGTGACCGACAACAAGATCCAGATCA | |
| | ACGCCAAGCTGCAAGAGTGCAGCAACAGCGACCAGCTGCAGGGAAAAGAGGAACGGGTCAAC | |
| | GAGGAAAGCCACCTGACCGAGAAAGAGTACATCGAGCACTGCAACACCCCCACCACCGACAG | |
| | CGACAGCAGCATCGCCGTGAAGGCTCTGCAGATCGACAGCTTCGGCCTGGTCACCTGCTTCC | |
| | AGCAAGAGAGCCTGGACGTGAGCCAGATGATCCTGGGCAAGTCTCAGCAGCCCGAGAGCAAG | |
| | ATGCAGAGCGAGTTCATCAAAGAGAAGAGCGCCACCTGCAGCAACGAAGAGAAGGACAACCT | |
| | GAACGAGAGCGTGATCACCGAAGAGAAAGAGACAGACGGCGACCACCTGAGCAGCCTGCTGA | |
| | ACAAGACCACCGTGCACAACATCCCCGGCTTCGACAGCATCAAAGAAACAAACATGCAGGAC | |
| | GGCAGCGTGCAAGTGATCAAGGACCACGTGACCAACTGCGCCTTCAGCTTCCAAAACAGCCT | |
| | GCTGTACGACCTGGACTGAGAATTCTGCAGAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAA | |
| | AAAAAAAAAAAAAAAAAAAAAAAAAATTCG | |
| SEQ ID | ATGGCCAAAGCCGCTGCCAGCAGCAGCCTGGAAGATCTGGATCTGAGCGGCGAGGAAGTGCA | DNAAF2 |
| NO: 15 | GAGACTGACCAGCGCCTTCCAGGACCCCGAGTTCAGACGGATGTTCAGCCAGTACGCCGAGG | (ORF and |
| | AACTGACAGACCCCGAGAACAGGCGGAGATACGAGGCCGAAATCACAGCCCTGGAAAGAGAA | HA Tag) |
| | CGGGGCGTCGAAGTCCGCTTCGTGCACCCTGAACCTGGCCACGTGCTGAGAACATCTCTGGA | |
| | CGGCGCCAGACGGTGCTTCGTGAACGTGTGCAGCAACGCCCTGGTGGGCGCTCCCAGCAGCA | |
| | GACCAGGATCTGGTGGCGACAGAGGCGCCGCTCCTGGATCTCACTGGAGCCTGCCCTACTCT | |
| | CTGGCCCCTGGCAGAGAGTACGCCGGCAGAAGCAGCTCTCGGTACATGGTGTACGACGTGGT | |
| | GTTCCACCCCGACGCTCTGGCTCTGGCCAGAAGGCACGAAGGCTTCAGACAGATGCTGGACG | |
| | CCACAGCTCTGGAAGCCGTGGAAAAGCAGTTCGGCGTGAAGCTGGACCGGCGGAATGCCAAG | |
| | ACACTGAAGGCCAAGTACAAGGGCACACCTGAAGCCGCCGTCCTGAGAACACCACTGCCTGG | |
| | TGTGATCCCCGCCAGACCTGATGGCGAGCCCAAAGGACCTCTGCCTGACTTCCCCTATCCCT | |
| | ACCAGTATCCAGCCGCTCCAGGACCCAGAGCACCCTCTCCACCAGAAGCTGCTCTGCAGCCA | |
| | GCTCCCACCGAGCCCAGATACAGCGTGGTGCAGAGGCACCATGTGGACCTGCAGGACTACAG | |
| | ATGCAGCAGAGACAGCGCCCCATCTCCTGTGCCTCACGAGCTGGTCATCACCATCGAACTGC | |
| | CCCTGCTGAGAAGCGCCGAACAGGCTGCACTGGAAGTGACAAGAAAGCTGCTGTGCCTGGAC | |
| | AGCAGAAAGCCCGACTACCGGCTGAGACTGAGCCTGCCATATCCTGTGGATGACGGCAGAGG | |
| | CAAGGCCCAGTTCAACAAAGCTCGGAGACAGCTGGTGGTCACCCTGCCTGTGGTGCTGCCTG | |
| | CCGCCAGAAGAGAACCTGCCGTGGCTGTGGCTGCCGCTGCTCCTGAAGAAAGCGCCGACAGA | |
| | TCTGGCACAGATGGCCAGGCCTGTGCCTCTGCCAGAGAAGGCGAAGCAGGACCCGCAAGAAG | |
| | CAGAGCCGAAGATGGCGGCCACGACACCTGTGTGGCTGGCGCAGCTGGAAGCGGCGTGACAA | |
| | CACTGGGAGATCCTGAAGTGGCCCCTCCACCAGCAGCAGCTGGCGAAGAAAGAGTGCCCAAG | |
| | CCTGGCGAGCAGGATCTGAGCAGACATGCCGGATCTCCACCTGGCAGCGTGGAAGAACCAAG | |
| | CCCTGGCGGAGAAAACTCTCCTGGCGGCGGAGGATCTCCCTGCCTGAGCAGCAGATCTCTGG | |
| | CCTGGGGAAGCAGCGCCGGAAGAGAATCTGCAAGAGGCGACAGCAGCGTGGAAACCCGGGAA | |
| | GAGTCTGAAGGCACAGGCGGACAGAGATCTGCCTGTGCCATGGGCGGACCTGGCACAAAGTC | |
| | TGGCGAACCTCTGTGCCCTCCTCTGCTGTGCAACCAGGACAAAGAGACACTGACACTGCTGA | |
| | TCCAGGTGCCACGGATCCAGCCTCAATCTCTGCAGGGCGACCTGAATCCTCTGTGGTACAAG | |
| | CTGAGATTCAGCGCCCAGGACCTGGTGTACAGCTTCTTCCTGCAATTCGCCCCAGAGAACAA | |
| | GCTGAGCACCACCGAGCCTGTGATCAGCATCAGCAGCAACAACGCCGTGATCGAGCTGGCCA | |
| | AGTCTCCTGAGTCTCACGGCCACTGGCGCGAGTGGTACTACGGCGTGAACAACGACAGCCTG | |
| | GAAGAACGGCTGTTCGTGAATGAGGAAAACGTGAACGAGTTCCTGGAAGAGGTGCTGAGCAG | |
| | CCCCTTCAAGCAGAGCATGAGCCTGACACCTCCACTGATCGAGGTGCTGCAAGTGACCGACA | |
| | ACAAGATCCAGATCAACGCCAAGCTGCAAGAGTGCAGCAACAGCGACCAGCTGCAGGGAAAA | |
| | GAGGAACGGGTCAACGAGGAAAGCCACCTGACCGAGAAAGAGTACATCGAGCACTGCAACAC | |
| | CCCCACCACCGACAGCGACAGCAGCATCGCCGTGAAGGCTCTGCAGATCGACAGCTTCGGCC | |
| | TGGTCACCTGCTTCCAGCAAGAGAGCCTGGACGTGAGCCAGATGATCCTGGGCAAGTCTCAG | |
| | CAGCCCGAGAGCAAGATGCAGAGCGAGTTCATCAAAGAGAAGAGCGCCACCTGCAGCAACGA | |
| | AGAGAAGGACAACCTGAACGAGAGCGTGATCACCGAAGAGAAAGAGACAGACGGCGACCACC | |
| | TGAGCAGCCTGCTGAACAAGACCACCGTGCACAACATCCCCGGCTTCGACAGCATCAAAGAA | |
| | ACAAACATGCAGGACGGCAGCGTGCAAGTGATCAAGGACCACGTGACCAACTGCGCCTTCAG | |
| | CTTCCAAAACAGCCTGCTGTACGACCTGGACGGAAGCGGCTACCCATACGATGTTCCTGACT | |
| | ATGCGTGA | |
| SEQ ID | GGGAgAcAAGAGAGAAAAGAAGAGCAAGAAGAAATATAAGAGCCACCATGGCCAAAGCCGCT | DNAAF2 |
| NO: 16 | GCCAGCAGCAGCCTGGAAGATCTGGATCTGAGCGGCGAGGAAGTGCAGAGACTGACCAGCGC | (5′ UTR, |
| | CTTCCAGGACCCCGAGTTCAGACGGATGTTCAGCCAGTACGCCGAGGAACTGACAGACCCCG | ORF, HA |
| | AGAACAGGCGGAGATACGAGGCCGAAATCACAGCCCTGGAAAGAGAACGGGGCGTCGAAGTC | Tag, and |
| | CGCTTCGTGCACCCTGAACCTGGCCACGTGCTGAGAACATCTCTGGACGGCGCCAGACGGTG | 3′ Tail) |
| | CTTCGTGAACGTGTGCAGCAACGCCCTGGTGGGCGCTCCCAGCAGCAGACCAGGATCTGGTG | |
| | GCGACAGAGGCGCCGCTCCTGGATCTCACTGGAGCCTGCCCTACTCTCTGGCCCCTGGCAGA | |
| | GAGTACGCCGGCAGAAGCAGCTCTCGGTACATGGTGTACGACGTGGTGTTCCACCCCGACGC | |
| | TCTGGCTCTGGCCAGAAGGCACGAAGGCTTCAGACAGATGCTGGACGCCACAGCTCTGGAAG | |
| | CCGTGGAAAAGCAGTTCGGCGTGAAGCTGGACCGGCGGAATGCCAAGACACTGAAGGCCAAG | |
| | TACAAGGGCACACCTGAAGCCGCCGTCCTGAGAACACCACTGCCTGGTGTGATCCCCGCCAG | |
| | ACCTGATGGCGAGCCCAAAGGACCTCTGCCTGACTTCCCCTATCCCTACCAGTATCCAGCCG | |
| | CTCCAGGACCCAGAGCACCCTCTCCACCAGAAGCTGCTCTGCAGCCAGCTCCCACCGAGCCC | |
| | AGATACAGCGTGGTGCAGAGGCACCATGTGGACCTGCAGGACTACAGATGCAGCAGAGACAG | |
| | CGCCCCATCTCCTGTGCCTCACGAGCTGGTCATCACCATCGAACTGCCCCTGCTGAGAAGCG | |
| | CCGAACAGGCTGCACTGGAAGTGACAAGAAAGCTGCTGTGCCTGGACAGCAGAAAGCCCGAC | |
| | TACCGGCTGAGACTGAGCCTGCCATATCCTGTGGATGACGGCAGAGGCAAGGCCCAGTTCAA | |
| | CAAAGCTCGGAGACAGCTGGTGGTCACCCTGCCTGTGGTGCTGCCTGCCGCCAGAAGAGAAC | |
| | CTGCCGTGGCTGTGGCTGCCGCTGCTCCTGAAGAAAGCGCCGACAGATCTGGCACAGATGGC | |
| | CAGGCCTGTGCCTCTGCCAGAGAAGGCGAAGCAGGACCCGCAAGAAGCAGAGCCGAAGATGG | |
| | CGGCCACGACACCTGTGTGGCTGGCGCAGCTGGAAGCGGCGTGACAACACTGGGAGATCCTG | |
| | AAGTGGCCCCTCCACCAGCAGCAGCTGGCGAAGAAAGAGTGCCCAAGCCTGGCGAGCAGGAT | |
| | CTGAGCAGACATGCCGGATCTCCACCTGGCAGCGTGGAAGAACCAAGCCCTGGCGGAGAAAA | |
| | CTCTCCTGGCGGCGGAGGATCTCCCTGCCTGAGCAGCAGATCTCTGGCCTGGGGAAGCAGCG | |
| | CCGGAAGAGAATCTGCAAGAGGCGACAGCAGCGTGGAAACCCGGGAAGAGTCTGAAGGCACA | |
| | GGCGGACAGAGATCTGCCTGTGCCATGGGCGGACCTGGCACAAAGTCTGGCGAACCTCTGTG | |
| | CCCTCCTCTGCTGTGCAACCAGGACAAAGAGACACTGACACTGCTGATCCAGGTGCCACGGA | |
| | TCCAGCCTCAATCTCTGCAGGGCGACCTGAATCCTCTGTGGTACAAGCTGAGATTCAGCGCC | |
| | CAGGACCTGGTGTACAGCTTCTTCCTGCAATTCGCCCCAGAGAACAAGCTGAGCACCACCGA | |
| | GCCTGTGATCAGCATCAGCAGCAACAACGCCGTGATCGAGCTGGCCAAGTCTCCTGAGTCTC | |
| | ACGGCCACTGGCGCGAGTGGTACTACGGCGTGAACAACGACAGCCTGGAAGAACGGCTGTTC | |
| | GTGAATGAGGAAAACGTGAACGAGTTCCTGGAAGAGGTGCTGAGCAGCCCCTTCAAGCAGAG | |
| | CATGAGCCTGACACCTCCACTGATCGAGGTGCTGCAAGTGACCGACAACAAGATCCAGATCA | |
| | ACGCCAAGCTGCAAGAGTGCAGCAACAGCGACCAGCTGCAGGGAAAAGAGGAACGGGTCAAC | |
| | GAGGAAAGCCACCTGACCGAGAAAGAGTACATCGAGCACTGCAACACCCCCACCACCGACAG | |
| | CGACAGCAGCATCGCCGTGAAGGCTCTGCAGATCGACAGCTTCGGCCTGGTCACCTGCTTCC | |
| | AGCAAGAGAGCCTGGACGTGAGCCAGATGATCCTGGGCAAGTCTCAGCAGCCCGAGAGCAAG | |
| | ATGCAGAGCGAGTTCATCAAAGAGAAGAGCGCCACCTGCAGCAACGAAGAGAAGGACAACCT | |
| | GAACGAGAGCGTGATCACCGAAGAGAAAGAGACAGACGGCGACCACCTGAGCAGCCTGCTGA | |
| | ACAAGACCACCGTGCACAACATCCCCGGCTTCGACAGCATCAAAGAAACAAACATGCAGGAC | |
| | GGCAGCGTGCAAGTGATCAAGGACCACGTGACCAACTGCGCCTTCAGCTTCCAAAACAGCCT | |
| | GCTGTACGACCTGGACGGAAGCGGCTACCCATACGATGTTCCTGACTATGCGTGAGAATTCT | |
| | GCAGAAAAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAA | |
| | TTCG | |
| SEQ ID | ATGCCTCTGCAAGTGAGCGACTACAGCTGGCAGCAGACCAAGACCGCCGTGTTCCTGAGCCT | DNAAF4 |
| NO: 17 | GCCTCTGAAGGGCGTGTGTGTGCGGGACACCGATGTGTTCTGCACCGAGAACTACCTGAAAG | (ORF) |
| | TGAACTTCCCACCCTTCCTGTTCGAGGCCTTCCTGTACGCCCCCATCGACGACGAGAGCAGC | |
| | AAGGCCAAGATCGGCAACGACACCATCGTGTTCACCCTGTACAAGAAAGAGGCCGCCATGTG | |
| | GGAGACACTGAGCGTGACAGGCGTGGACAAAGAAATGATGCAGCGGATCAGAGAGAAGAGCA | |
| | TCCTGCAGGCCCAAGAGAGAGCCAAAGAGGCCACAGAAGCCAAGGCCGCTGCCAAAAGAGAG | |
| | GACCAGAAATACGCCCTGAGCGTGATGATGAAGATCGAGGAAGAGGAACGCAAGAAAATCGA | |
| | GGACATGAAGGAAAACGAGCGGATCAAGGCCACAAAGGCCCTGGAAGCCTGGAAAGAGTACC | |
| | AGCGGAAGGCCGAGGAACAGAAGAAGATCCAGCGGGAAGAGAAGCTGTGCCAGAAAGAGAAG | |
| | CAGATCAAAGAAGAGAGAAAGAAGATCAAGTACAAGAGCCTGACACGGAACCTGGCCAGCAG | |
| | AAATCTGGCCCCCAAGGGCAGAAACAGCGAGAACATCTTCACCGAGAAGCTGAAAGAGGACA | |
| | GCATCCCCGCTCCCAGAAGCGTGGGCAGCATCAAGATCAACTTCACCCCTCGGGTGTTCCCC | |
| | ACCGCTCTGAGAGAATCTCAGGTGGCCGAAGAGGAAGAGTGGCTGCACAAACAGGCCGAGGC | |
| | TCGGAGAGCCATGAACACCGACATCGCCGAGCTGTGCGACCTGAAAGAAGAGGAAAAGAACC | |
| | CCGAGTGGCTGAAGGACAAGGGCAACAAGCTGTTCGCCACAGAGAACTACCTGGCCGCCATC | |
| | AACGCCTACAATCTGGCCATCCGGCTGAACAACAAGATGCCCCTGCTGTACCTGAACAGAGC | |
| | CGCCTGCCACCTGAAGCTGAAGAACCTGCACAAGGCCATCGAGGACAGCAGCAAGGCTCTGG | |
| | AACTGCTGATGCCTCCTGTGACCGACAACGCCAACGCCAGAATGAAGGCCCATGTGCGGAGA | |
| | GGCACCGCCTTCTGTCAGCTGGAACTGTACGTGGAAGGACTGCAGGACTACGAGGCCGCTCT | |
| | GAAGATCGACCCCAGCAACAAGATCGTGCAGATCGACGCCGAGAAGATCCGGAACGTGATCC | |
| | AGGGCACCGAGCTGAAGAGCTGA | |
| SEQ ID | GGGAGACAAGAGAGAAAAGAAGAGCAAGAAGAAATATAAGAGCCACCATGCCTCTGCAAGTG | DNAA4F |
| NO: 18 | AGCGACTACAGCTGGCAGCAGACCAAGACCGCCGTGTTCCTGAGCCTGCCTCTGAAGGGCGT | (5′ UTR, |
| | GTGTGTGCGGGACACCGATGTGTTCTGCACCGAGAACTACCTGAAAGTGAACTTCCCACCCT | ORF, and |
| | TCCTGTTCGAGGCCTTCCTGTACGCCCCCATCGACGACGAGAGCAGCAAGGCCAAGATCGGC | 3′ Tail) |
| | AACGACACCATCGTGTTCACCCTGTACAAGAAAGAGGCCGCCATGTGGGAGACACTGAGCGT | |
| | GACAGGCGTGGACAAAGAAATGATGCAGCGGATCAGAGAGAAGAGCATCCTGCAGGCCCAAG | |
| | AGAGAGCCAAAGAGGCCACAGAAGCCAAGGCCGCTGCCAAAAGAGAGGACCAGAAATACGCC | |
| | CTGAGCGTGATGATGAAGATCGAGGAAGAGGAACGCAAGAAAATCGAGGACATGAAGGAAAA | |
| | CGAGCGGATCAAGGCCACAAAGGCCCTGGAAGCCTGGAAAGAGTACCAGCGGAAGGCCGAGG | |
| | AACAGAAGAAGATCCAGCGGGAAGAGAAGCTGTGCCAGAAAGAGAAGCAGATCAAAGAAGAG | |
| | AGAAAGAAGATCAAGTACAAGAGCCTGACACGGAACCTGGCCAGCAGAAATCTGGCCCCCAA | |
| | GGGCAGAAACAGCGAGAACATCTTCACCGAGAAGCTGAAAGAGGACAGCATCCCCGCTCCCA | |
| | GAAGCGTGGGCAGCATCAAGATCAACTTCACCCCTCGGGTGTTCCCCACCGCTCTGAGAGAA | |
| | TCTCAGGTGGCCGAAGAGGAAGAGTGGCTGCACAAACAGGCCGAGGCTCGGAGAGCCATGAA | |
| | CACCGACATCGCCGAGCTGTGCGACCTGAAAGAAGAGGAAAAGAACCCCGAGTGGCTGAAGG | |
| | ACAAGGGCAACAAGCTGTTCGCCACAGAGAACTACCTGGCCGCCATCAACGCCTACAATCTG | |
| | GCCATCCGGCTGAACAACAAGATGCCCCTGCTGTACCTGAACAGAGCCGCCTGCCACCTGAA | |
| | GCTGAAGAACCTGCACAAGGCCATCGAGGACAGCAGCAAGGCTCTGGAACTGCTGATGCCTC | |
| | CTGTGACCGACAACGCCAACGCCAGAATGAAGGCCCATGTGCGGAGAGGCACCGCCTTCTGT | |
| | CAGCTGGAACTGTACGTGGAAGGACTGCAGGACTACGAGGCCGCTCTGAAGATCGACCCCAG | |
| | CAACAAGATCGTGCAGATCGACGCCGAGAAGATCCGGAACGTGATCCAGGGCACCGAGCTGA | |
| | AGAGCTGAGAATTCTGCAGAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAA | |
| | AAAAAAAAAAAAAAATTCG | |
| SEQ ID | ATGCCTCTGCAAGTGAGCGACTACAGCTGGCAGCAGACCAAGACCGCCGTGTTCCTGAGCCT | DNAAF4 |
| NO: 19 | GCCTCTGAAGGGCGTGTGTGTGCGGGACACCGATGTGTTCTGCACCGAGAACTACCTGAAAG | (ORF and |
| | TGAACTTCCCACCCTTCCTGTTCGAGGCCTTCCTGTACGCCCCCATCGACGACGAGAGCAGC | HA Tag) |
| | AAGGCCAAGATCGGCAACGACACCATCGTGTTCACCCTGTACAAGAAAGAGGCCGCCATGTG | |
| | GGAGACACTGAGCGTGACAGGCGTGGACAAAGAAATGATGCAGCGGATCAGAGAGAAGAGCA | |
| | TCCTGCAGGCCCAAGAGAGAGCCAAAGAGGCCACAGAAGCCAAGGCCGCTGCCAAAAGAGAG | |
| | GACCAGAAATACGCCCTGAGCGTGATGATGAAGATCGAGGAAGAGGAACGCAAGAAAATCGA | |
| | GGACATGAAGGAAAACGAGCGGATCAAGGCCACAAAGGCCCTGGAAGCCTGGAAAGAGTACC | |
| | AGCGGAAGGCCGAGGAACAGAAGAAGATCCAGCGGGAAGAGAAGCTGTGCCAGAAAGAGAAG | |
| | CAGATCAAAGAAGAGAGAAAGAAGATCAAGTACAAGAGCCTGACACGGAACCTGGCCAGCAG | |
| | AAATCTGGCCCCCAAGGGCAGAAACAGCGAGAACATCTTCACCGAGAAGCTGAAAGAGGACA | |
| | GCATCCCCGCTCCCAGAAGCGTGGGCAGCATCAAGATCAACTTCACCCCTCGGGTGTTCCCC | |
| | ACCGCTCTGAGAGAATCTCAGGTGGCCGAAGAGGAAGAGTGGCTGCACAAACAGGCCGAGGC | |
| | TCGGAGAGCCATGAACACCGACATCGCCGAGCTGTGCGACCTGAAAGAAGAGGAAAAGAACC | |
| | CCGAGTGGCTGAAGGACAAGGGCAACAAGCTGTTCGCCACAGAGAACTACCTGGCCGCCATC | |
| | AACGCCTACAATCTGGCCATCCGGCTGAACAACAAGATGCCCCTGCTGTACCTGAACAGAGC | |
| | CGCCTGCCACCTGAAGCTGAAGAACCTGCACAAGGCCATCGAGGACAGCAGCAAGGCTCTGG | |
| | AACTGCTGATGCCTCCTGTGACCGACAACGCCAACGCCAGAATGAAGGCCCATGTGCGGAGA | |
| | GGCACCGCCTTCTGTCAGCTGGAACTGTACGTGGAAGGACTGCAGGACTACGAGGCCGCTCT | |
| | GAAGATCGACCCCAGCAACAAGATCGTGCAGATCGACGCCGAGAAGATCCGGAACGTGATCC | |
| | AGGGCACCGAGCTGAAGAGCGGAAGCGGCTACCCATACGATGTTCCTGACTATGCGTGA | |
| SEQ ID | GGGAgAcAAGAGAGAAAAGAAGAGCAAGAAGAAATATAAGAGCCACCATGCCTCTGCAAGTG | DNAAF4 |
| NO: 20 | AGCGACTACAGCTGGCAGCAGACCAAGACCGCCGTGTTCCTGAGCCTGCCTCTGAAGGGCGT | (5′ UTR, |
| | GTGTGTGCGGGACACCGATGTGTTCTGCACCGAGAACTACCTGAAAGTGAACTTCCCACCCT | ORF, HA |
| | TCCTGTTCGAGGCCTTCCTGTACGCCCCCATCGACGACGAGAGCAGCAAGGCCAAGATCGGC | Tag, 3′ |
| | AACGACACCATCGTGTTCACCCTGTACAAGAAAGAGGCCGCCATGTGGGAGACACTGAGCGT | Tail) |
| | GACAGGCGTGGACAAAGAAATGATGCAGCGGATCAGAGAGAAGAGCATCCTGCAGGCCCAAG | |
| | AGAGAGCCAAAGAGGCCACAGAAGCCAAGGCCGCTGCCAAAAGAGAGGACCAGAAATACGCC | |
| | CTGAGCGTGATGATGAAGATCGAGGAAGAGGAACGCAAGAAAATCGAGGACATGAAGGAAAA | |
| | CGAGCGGATCAAGGCCACAAAGGCCCTGGAAGCCTGGAAAGAGTACCAGCGGAAGGCCGAGG | |
| | AACAGAAGAAGATCCAGCGGGAAGAGAAGCTGTGCCAGAAAGAGAAGCAGATCAAAGAAGAG | |
| | AGAAAGAAGATCAAGTACAAGAGCCTGACACGGAACCTGGCCAGCAGAAATCTGGCCCCCAA | |
| | GGGCAGAAACAGCGAGAACATCTTCACCGAGAAGCTGAAAGAGGACAGCATCCCCGCTCCCA | |
| | GAAGCGTGGGCAGCATCAAGATCAACTTCACCCCTCGGGTGTTCCCCACCGCTCTGAGAGAA | |
| | TCTCAGGTGGCCGAAGAGGAAGAGTGGCTGCACAAACAGGCCGAGGCTCGGAGAGCCATGAA | |
| | CACCGACATCGCCGAGCTGTGCGACCTGAAAGAAGAGGAAAAGAACCCCGAGTGGCTGAAGG | |
| | ACAAGGGCAACAAGCTGTTCGCCACAGAGAACTACCTGGCCGCCATCAACGCCTACAATCTG | |
| | GCCATCCGGCTGAACAACAAGATGCCCCTGCTGTACCTGAACAGAGCCGCCTGCCACCTGAA | |
| | GCTGAAGAACCTGCACAAGGCCATCGAGGACAGCAGCAAGGCTCTGGAACTGCTGATGCCTC | |
| | CTGTGACCGACAACGCCAACGCCAGAATGAAGGCCCATGTGCGGAGAGGCACCGCCTTCTGT | |
| | CAGCTGGAACTGTACGTGGAAGGACTGCAGGACTACGAGGCCGCTCTGAAGATCGACCCCAG | |
| | CAACAAGATCGTGCAGATCGACGCCGAGAAGATCCGGAACGTGATCCAGGGCACCGAGCTGA | |
| | AGAGCGGAAGCGGCTACCCATACGATGTTCCTGACTATGCGTGAGAATTCTGCAGAAAAAAA | |
| | AAAAAAAAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAATTCG | |
| SEQ ID | ATGGGAGATCTGGAACTGCTGCTGCCTGGCGAGGCCGAGGTGCTCGTGAGAGGCCTGAGAAG | ZMYND10 |
| NO: 21 | CTTCCCACTGAGAGAGATGGGCAGCGAAGGCTGGAATCAGCAGCACGAGAATCTGGAAAAGC | (ORF) |
| | TGAACATGCAGGCCATCCTGGACGCCACAGTGTCTCAGGGCGAGCCCATCCAAGAGCTGCTG | |
| | GTCACACACGGCAAGGTGCCAACACTGGTGGAAGAACTGATCGCCGTGGAAATGTGGAAGCA | |
| | GAAAGTGTTCCCCGTGTTCTGCCGCGTGGAAGACTTCAAGCCCCAGAACACATTCCCCATCT | |
| | ACATGGTGGTGCATCACGAGGCCAGCATCATCAACCTGCTGGAAACCGTGTTCTTCCACAAA | |
| | GAAGTGTGCGAGAGCGCCGAGGACACCGTGCTGGATCTGGTGGACTACTGCCACCGGAAGCT | |
| | GACACTGCTGGTGGCCCAATCTGGATGTGGCGGACCTCCTGAAGGCGAGGGCAGCCAGGACA | |
| | GCAACCCAATGCAAGAACTGCAGAAACAGGCCGAGCTGATGGAATTCGAGATCGCCCTGAAG | |
| | GCCCTGAGCGTGCTGAGATACATCACCGACTGCGTGGACAGCCTGAGCCTGAGCACACTGAG | |
| | CAGAATGCTGAGCACCCACAACCTGCCCTGCCTGCTGGTCGAACTGCTGGAACACAGCCCCT | |
| | GGAGCAGAAGAGAAGGCGGAAAGCTGCAGCAGTTCGAGGGCAGCAGATGGCACACAGTGGCC | |
| | CCATCTGAGCAGCAGAAGCTGAGCAAGCTGGACGGCCAAGTGTGGATCGCCCTGTACAATCT | |
| | GCTGCTGAGCCCTGAGGCACAGGCCAGATACTGCCTGACCAGCTTCGCCAAGGGCAGACTGC | |
| | TGAAGCTGCGGGCCTTCCTGACCGACACTCTGCTGGATCAGCTGCCCAATCTGGCCCATCTG | |
| | CAGAGCTTCCTGGCCCACCTGACACTGACCGAGACACAGCCTCCCAAGAAAGACCTGGTCCT | |
| | GGAACAGATCCCCGAGATCTGGGAGCGCCTGGAAAGGGAAAACAGAGGCAAGTGGCAGGCCA | |
| | TCGCCAAGCACCAGCTGCAGCACGTGTTCAGCCCCAGCGAGCAGGACCTGAGACTGCAGGCC | |
| | AGAAGATGGGCCGAGACATACAGACTGGACGTGCTGGAAGCTGTGGCCCCTGAAAGACCCAG | |
| | ATGCGCCTACTGCAGCGCCGAAGCCAGCAAGAGATGCAGCCGGTGTCAGAACGAGTGGTACT | |
| | GCTGCCGCGAGTGCCAAGTGAAGCACTGGGAGAAGCACGGCAAGACCTGTGTGCTGGCCGCA | |
| | CAGGGCGACAGAGCCAAGTGA | |
| SEQ ID | GGGAGACCCAAGCTGGCTAGCGTTTAAACTTCAGCTTGGCAATCCGGTACTGTTGGTAAAGC | ZMYND10 |
| NO: 22 | CACCATGGGAGATCTGGAACTGCTGCTGCCTGGCGAGGCCGAGGTGCTCGTGAGAGGCCTGA | (5′ UTR, |
| | GAAGCTTCCCACTGAGAGAGATGGGCAGCGAAGGCTGGAATCAGCAGCACGAGAATCTGGAA | ORF, and |
| | AAGCTGAACATGCAGGCCATCCTGGACGCCACAGTGTCTCAGGGCGAGCCCATCCAAGAGCT | 3′ Tail) |
| | GCTGGTCACACACGGCAAGGTGCCAACACTGGTGGAAGAACTGATCGCCGTGGAAATGTGGA | |
| | AGCAGAAAGTGTTCCCCGTGTTCTGCCGCGTGGAAGACTTCAAGCCCCAGAACACATTCCCC | |
| | ATCTACATGGTGGTGCATCACGAGGCCAGCATCATCAACCTGCTGGAAACCGTGTTCTTCCA | |
| | CAAAGAAGTGTGCGAGAGCGCCGAGGACACCGTGCTGGATCTGGTGGACTACTGCCACCGGA | |
| | AGCTGACACTGCTGGTGGCCCAATCTGGATGTGGCGGACCTCCTGAAGGCGAGGGCAGCCAG | |
| | GACAGCAACCCAATGCAAGAACTGCAGAAACAGGCCGAGCTGATGGAATTCGAGATCGCCCT | |
| | GAAGGCCCTGAGCGTGCTGAGATACATCACCGACTGCGTGGACAGCCTGAGCCTGAGCACAC | |
| | TGAGCAGAATGCTGAGCACCCACAACCTGCCCTGCCTGCTGGTCGAACTGCTGGAACACAGC | |
| | CCCTGGAGCAGAAGAGAAGGCGGAAAGCTGCAGCAGTTCGAGGGCAGCAGATGGCACACAGT | |
| | GGCCCCATCTGAGCAGCAGAAGCTGAGCAAGCTGGACGGCCAAGTGTGGATCGCCCTGTACA | |
| | ATCTGCTGCTGAGCCCTGAGGCACAGGCCAGATACTGCCTGACCAGCTTCGCCAAGGGCAGA | |
| | CTGCTGAAGCTGCGGGCCTTCCTGACCGACACTCTGCTGGATCAGCTGCCCAATCTGGCCCA | |
| | TCTGCAGAGCTTCCTGGCCCACCTGACACTGACCGAGACACAGCCTCCCAAGAAAGACCTGG | |
| | TCCTGGAACAGATCCCCGAGATCTGGGAGCGCCTGGAAAGGGAAAACAGAGGCAAGTGGCAG | |
| | GCCATCGCCAAGCACCAGCTGCAGCACGTGTTCAGCCCCAGCGAGCAGGACCTGAGACTGCA | |
| | GGCCAGAAGATGGGCCGAGACATACAGACTGGACGTGCTGGAAGCTGTGGCCCCTGAAAGAC | |
| | CCAGATGCGCCTACTGCAGCGCCGAAGCCAGCAAGAGATGCAGCCGGTGTCAGAACGAGTGG | |
| | TACTGCTGCCGCGAGTGCCAAGTGAAGCACTGGGAGAAGCACGGCAAGACCTGTGTGCTGGC | |
| | CGCACAGGGCGACAGAGCCAAGTGAGAATTCTGCAGAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAATTCG | |
| SEQ ID | ATGGGAGATCTGGAACTGCTGCTGCCTGGCGAGGCCGAGGTGCTCGTGAGAGGCCTGAGgAG | ZMYND10 |
| NO: 23 | CTTCCCACTGAGAGAGATGGGCAGCGAAGGCTGGAATCAGCAGCACGAGAATCTGGAAAAGC | (ORF and |
| | TGAACATGCAGGCCATCCTGGACGCCACAGTGTCTCAGGGCGAGCCCATCCAAGAGCTGCTG | HA Tag) |
| | GTCACACACGGCAAGGTGCCAACACTGGTGGAAGAACTGATCGCCGTGGAAATGTGGAAGCA | |
| | GAAAGTGTTCCCCGTGTTCTGCCGCGTGGAAGACTTCAAGCCCCAGAACACATTCCCCATCT | |
| | ACATGGTGGTGCATCACGAGGCCAGCATCATCAACCTGCTGGAAACCGTGTTCTTCCACAAA | |
| | GAAGTGTGCGAGAGCGCCGAGGACACCGTGCTGGATCTGGTGGACTACTGCCACCGGAAGCT | |
| | GACACTGCTGGTGGCCCAATCTGGATGTGGCGGACCTCCTGAAGGCGAGGGCAGCCAGGACA | |
| | GCAACCCAATGCAAGAACTGCAGAAACAGGCCGAGCTGATGGAgTTCGAGATCGCCCTGAAG | |
| | GCCCTGAGCGTGCTGAGATACATCACCGACTGCGTGGACAGCCTGAGCCTGAGCACACTGAG | |
| | CAGAATGCTGAGCACCCACAACCTGCCCTGCCTGCTGGTCGAACTGCTGGAACACAGCCCCT | |
| | GGAGCAGAAGAGAAGGCGGAAAGCTGCAGCAGTTCGAGGGCAGCAGATGGCACACAGTGGCC | |
| | CCATCTGAGCAGCAGAAGCTGAGCAAGCTGGACGGCCAAGTGTGGATCGCCCTGTACAATCT | |
| | GCTGCTGAGCCCTGAGGCACAGGCCAGATACTGCCTGACCAGCTTCGCCAAGGGCAGACTGC | |
| | TGAAGCTGCGGGCCTTCCTGACCGACACTCTGCTGGATCAGCTGCCCAATCTGGCCCATCTG | |
| | CAGAGCTTCCTGGCCCACCTGACACTGACCGAGACACAGCCTCCCAAGAAAGACCTGGTCCT | |
| | GGAACAGATCCCCGAGATCTGGGAGCGCCTGGAAAGGGAAAACAGAGGCAAGTGGCAGGCCA | |
| | TCGCCAAGCACCAGCTGCAGCACGTGTTCAGCCCCAGCGAGCAGGACCTGAGACTGCAGGCC | |
| | AGAAGATGGGCCGAGACATACAGACTGGACGTGCTGGAAGCTGTGGCCCCTGAAAGACCCAG | |
| | ATGCGCCTACTGCAGCGCCGAAGCCAGCAAGAGATGCAGCCGGTGTCAGAACGAGTGGTACT | |
| | GCTGCCGCGAGTGCCAAGTGAAGCACTGGGAGAAGCACGGCAAGACCTGTGTGCTGGCCGCA | |
| | CAGGGCGACAGAGCCAAGACCGGTGCGGCCGTTTACCCATACGATGTTCCTGACTATGCGTG | |
| | A | |
| SEQ ID | GGGAGACCCAAGCTGGCTAGCGTTTAAACTTCAGCTTGGCAATCCGGTACTGTTGGTAAAGC | ZMYND10 |
| NO: 24 | CACCATGGGAGATCTGGAACTGCTGCTGCCTGGCGAGGCCGAGGTGCTCGTGAGAGGCCTGA | (5′ UTR, |
| | GgAGCTTCCCACTGAGAGAGATGGGCAGCGAAGGCTGGAATCAGCAGCACGAGAATCTGGAA | ORF, HA |
| | AAGCTGAACATGCAGGCCATCCTGGACGCCACAGTGTCTCAGGGCGAGCCCATCCAAGAGCT | Tag, and |
| | GCTGGTCACACACGGCAAGGTGCCAACACTGGTGGAAGAACTGATCGCCGTGGAAATGTGGA | 3′ Tail) |
| | AGCAGAAAGTGTTCCCCGTGTTCTGCCGCGTGGAAGACTTCAAGCCCCAGAACACATTCCCC | |
| | ATCTACATGGTGGTGCATCACGAGGCCAGCATCATCAACCTGCTGGAAACCGTGTTCTTCCA | |
| | CAAAGAAGTGTGCGAGAGCGCCGAGGACACCGTGCTGGATCTGGTGGACTACTGCCACCGGA | |
| | AGCTGACACTGCTGGTGGCCCAATCTGGATGTGGCGGACCTCCTGAAGGCGAGGGCAGCCAG | |
| | GACAGCAACCCAATGCAAGAACTGCAGAAACAGGCCGAGCTGATGGAgTTCGAGATCGCCCT | |
| | GAAGGCCCTGAGCGTGCTGAGATACATCACCGACTGCGTGGACAGCCTGAGCCTGAGCACAC | |
| | TGAGCAGAATGCTGAGCACCCACAACCTGCCCTGCCTGCTGGTCGAACTGCTGGAACACAGC | |
| | CCCTGGAGCAGAAGAGAAGGCGGAAAGCTGCAGCAGTTCGAGGGCAGCAGATGGCACACAGT | |
| | GGCCCCATCTGAGCAGCAGAAGCTGAGCAAGCTGGACGGCCAAGTGTGGATCGCCCTGTACA | |
| | ATCTGCTGCTGAGCCCTGAGGCACAGGCCAGATACTGCCTGACCAGCTTCGCCAAGGGCAGA | |
| | CTGCTGAAGCTGCGGGCCTTCCTGACCGACACTCTGCTGGATCAGCTGCCCAATCTGGCCCA | |
| | TCTGCAGAGCTTCCTGGCCCACCTGACACTGACCGAGACACAGCCTCCCAAGAAAGACCTGG | |
| | TCCTGGAACAGATCCCCGAGATCTGGGAGCGCCTGGAAAGGGAAAACAGAGGCAAGTGGCAG | |
| | GCCATCGCCAAGCACCAGCTGCAGCACGTGTTCAGCCCCAGCGAGCAGGACCTGAGACTGCA | |
| | GGCCAGAAGATGGGCCGAGACATACAGACTGGACGTGCTGGAAGCTGTGGCCCCTGAAAGAC | |
| | CCAGATGCGCCTACTGCAGCGCCGAAGCCAGCAAGAGATGCAGCCGGTGTCAGAACGAGTGG | |
| | TACTGCTGCCGCGAGTGCCAAGTGAAGCACTGGGAGAAGCACGGCAAGACCTGTGTGCTGGC | |
| | CGCACAGGGCGACAGAGCCAAGACCGGTGCGGCCGTTTACCCATACGATGTTCCTGACTATG | |
| | CGTGAGAATTCTGCAGAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAA | |
| | AAAAAAAAAAAATTCG | |
| SEQ ID | ATGAGCAGCGAGTTCCTGGCCGAACTGCACTGGGAGGACGGCTTCGCCATCCCCGTGGCCAA | CCDC39 |
| NO: 25 | CGAGGAAAACAAGCTGCTGGAAGACCAGCTGAGCAAGCTGAAGGACGAGAGAGCCAGCCTGC | (ORF) |
| | AGGACGAGCTGAGAGAGTACGAGGAACGGATCAACAGCATGACCAGCCACTTCAAGAACGTG | |
| | AAGCAAGAGCTGAGCATCACCCAGAGCCTGTGCAAGGCCAGAGAGAGAGAAACCGAGAGCGA | |
| | GGAACACTTCAAGGCCATCGCCCAGCGCGAGCTGGGAAGAGTGAAGGACGAGATCCAGCGGC | |
| | TGGAAAACGAGATGGCCAGCATCCTGGAAAAGAAGAGCGACAAAGAGAACGGCATCTTCAAG | |
| | GCCACACAGAAGCTGGACGGCCTGAAGTGCCAGATGAACTGGGACCAGCAGGCCCTGGAAGC | |
| | CTGGCTGGAAGAGAGCGCCCACAAGGACAGCGACGCCCTGACACTGCAGAAGTACGCCCAGC | |
| | AGGACGACAACAAGATCCGGGCCCTGACCCTGCAGCTGGAAAGACTGACCCTGGAATGCAAC | |
| | CAGAAGCGGAAGATCCTGGACAACGAGCTGACCGAGACAATCAGCGCCCAGCTGGAACTGGA | |
| | CAAGGCCGCCCAGGACTTCAGAAAGATCCACAACGAGCGGCAAGAACTGATCAAGCAGTGGG | |
| | AGAACACCATCGAGCAGATGCAGAAACGCGACGGCGACATCGACAACTGCGCCCTGGAACTC | |
| | GCCCGGATCAAGCAAGAGACACGCGAGAAAGAGAACCTGGTCAAAGAGAAGATCAAGTTCCT | |
| | CGAGAGCGAGATCGGCAACAACACCGAGTTCGAGAAGCGGATCAGCGTGGCCGACAGAAAGC | |
| | TGCTGAAGTGCAGAACCGCCTACCAGGACCACGAGACAAGCCGGATCCAGCTCAAGGGCGAG | |
| | CTGGACAGCCTGAAGGCCACCGTGAACAGAACCAGCAGCGACCTGGAAGCCCTGCGGAAGAA | |
| | CATCAGCAAGATCAAGAAGGACATCCACGAGGAAACCGCCAGGCTGCAGAAAACAAAGAACC | |
| | ACAACGAGATCATCCAGACCAAGCTGAAAGAGATCACCGAAAAGACCATGAGCGTGGAAGAG | |
| | AAGGCCACAAACCTGGAAGACATGCTCAAAGAGGAAGAGAAAGACGTCAAAGAGGTGGACGT | |
| | GCAACTGAACCTGATCAAGGGCGTGCTGTTCAAGAAGGCCCAAGAGCTGCAGACCGAAACCA | |
| | TGAAGGAAAAGGCCGTCCTGAGCGAGATCGAGGGCACCAGAAGCAGCCTGAAGCACCTGAAC | |
| | CACCAGCTGCAGAAGCTCGACTTCGAGACACTGAAGCAGCAAGAGATCATGTACAGCCAGGA | |
| | CTTCCACATCCAGCAGGTCGAGCGGCGGATGAGCAGACTGAAGGGCGAGATCAACAGCGAGG | |
| | AAAAACAGGCCCTCGAGGCCAAGATCGTGGAACTGAGAAAGAGCCTCGAAGAGAAGAAGAGC | |
| | ACCTGCGGCCTGCTGGAAACCCAGATCAAGAAGCTGCACAACGACCTGTACTTCATCAAGAA | |
| | AGCCCACAGCAAGAACAGCGACGAGAAGCAGAGCCTGATGACCAAGATCAACGAGCTGAACC | |
| | TGTTCATCGACCGGAGCGAAAAAGAGCTGGACAAGGCCAAGGGCTTCAAGCAGGACCTGATG | |
| | ATCGAGGACAACCTGCTGAAGCTGGAAGTGAAGCGGACCAGAGAGATGCTGCACAGCAAGGC | |
| | CGAGGAAGTGCTGAGCCTGGAAAAGCGGAAGCAGCAGCTGTACACCGCCATGGAAGAGAGAA | |
| | CCGAAGAGATCAAGGTGCACAAGACCATGCTGGCCAGCCAGATCAGATACGTGGACCAAGAG | |
| | CGCGAGAACATCAGCACCGAGTTCAGAGAGAGACTGAGCAAGATCGAGAAGCTGAAGAACCG | |
| | CTACGAGATCCTGACCGTCGTGATGCTGCCCCCCGAGGGCGAAGAGGAAAAGACCCAGGCCT | |
| | ACTACGTGATCAAGGCAGCCCAAGAAAAAGAGGAACTCCAGAGAGAAGGCGACTGCCTGGAC | |
| | GCCAAGATCAACAAGGCCGAAAAAGAAATCTACGCCCTCGAGAACACCCTGCAGGTCCTGAA | |
| | CAGCTGCAACAACAACTACAAGCAGAGCTTCAAGAAAGTCACCCCCAGCAGCGACGAGTACG | |
| | AGCTGAAGATCCAGCTGGAAGAACAGAAAAGAGCCGTGGACGAGAAGTACAGATACAAGCAG | |
| | CGGCAGATCAGAGAGCTGCAAGAGGACATCCAGAGCATGGAAAACACCCTGGACGTGATCGA | |
| | GCACCTGGCCAACAACGTGAAAGAGAAGCTGAGCGAGAAACAGGCCTACAGCTTCCAGCTGA | |
| | GCAAAGAGACAGAGGAACAGAAGCCCAAACTGGAACGCGTGACCAAGCAGTGCGCCAAGCTG | |
| | ACAAAAGAGATCCGGCTGCTGAAAGACACCAAGGACGAAACCATGGAAGAACAAGACATCAA | |
| | GCTGCGCGAGATGAAGCAGTTCCACAAAGTGATCGACGAGATGCTGGTGGACATCATCGAAG | |
| | AGAACACAGAGATCCGCATCATCCTGCAGACCTACTTCCAGCAGAGCGGCCTGGAACTGCCC | |
| | ACCGCCAGCACAAAGGGCAGCAGACAGAGCAGCAGAAGCCCCAGCCACACAAGCCTGAGCGC | |
| | CAGAAGCAGCAGAAGCACCAGCACCAGCACCAGCCAGAGCAGCATCAAGGTGCTGGAACTCA | |
| | AGTTCCCCGCCAGCAGCAGCCTCGTGGGAAGCCCCAGCAGACCCAGCAGCGCCAGCAGCAGC | |
| | AGCAGCAACGTGAAGAGCAAGAAAAGCAGCAAGTGA | |
| SEQ ID | GGGAGACCCAAGCTGGCTAGCGTTTAAACTTCAGCTTGGCAATCCGGTACTGTTGGTAAAGC | CCDC39 |
| NO: 26 | CACCATGAGCAGCGAGTTCCTGGCCGAACTGCACTGGGAGGACGGCTTCGCCATCCCCGTGG | (5′ UTR, |
| | CCAACGAGGAAAACAAGCTGCTGGAAGACCAGCTGAGCAAGCTGAAGGACGAGAGAGCCAGC | ORF, and |
| | CTGCAGGACGAGCTGAGAGAGTACGAGGAACGGATCAACAGCATGACCAGCCACTTCAAGAA | 3′ Tail) |
| | CGTGAAGCAAGAGCTGAGCATCACCCAGAGCCTGTGCAAGGCCAGAGAGAGAGAAACCGAGA | |
| | GCGAGGAACACTTCAAGGCCATCGCCCAGCGCGAGCTGGGAAGAGTGAAGGACGAGATCCAG | |
| | CGGCTGGAAAACGAGATGGCCAGCATCCTGGAAAAGAAGAGCGACAAAGAGAACGGCATCTT | |
| | CAAGGCCACACAGAAGCTGGACGGCCTGAAGTGCCAGATGAACTGGGACCAGCAGGCCCTGG | |
| | AAGCCTGGCTGGAAGAGAGCGCCCACAAGGACAGCGACGCCCTGACACTGCAGAAGTACGCC | |
| | CAGCAGGACGACAACAAGATCCGGGCCCTGACCCTGCAGCTGGAAAGACTGACCCTGGAATG | |
| | CAACCAGAAGCGGAAGATCCTGGACAACGAGCTGACCGAGACAATCAGCGCCCAGCTGGAAC | |
| | TGGACAAGGCCGCCCAGGACTTCAGAAAGATCCACAACGAGCGGCAAGAACTGATCAAGCAG | |
| | TGGGAGAACACCATCGAGCAGATGCAGAAACGCGACGGCGACATCGACAACTGCGCCCTGGA | |
| | ACTCGCCCGGATCAAGCAAGAGACACGCGAGAAAGAGAACCTGGTCAAAGAGAAGATCAAGT | |
| | TCCTCGAGAGCGAGATCGGCAACAACACCGAGTTCGAGAAGCGGATCAGCGTGGCCGACAGA | |
| | AAGCTGCTGAAGTGCAGAACCGCCTACCAGGACCACGAGACAAGCCGGATCCAGCTCAAGGG | |
| | CGAGCTGGACAGCCTGAAGGCCACCGTGAACAGAACCAGCAGCGACCTGGAAGCCCTGCGGA | |
| | AGAACATCAGCAAGATCAAGAAGGACATCCACGAGGAAACCGCCAGGCTGCAGAAAACAAAG | |
| | AACCACAACGAGATCATCCAGACCAAGCTGAAAGAGATCACCGAAAAGACCATGAGCGTGGA | |
| | AGAGAAGGCCACAAACCTGGAAGACATGCTCAAAGAGGAAGAGAAAGACGTCAAAGAGGTGG | |
| | ACGTGCAACTGAACCTGATCAAGGGCGTGCTGTTCAAGAAGGCCCAAGAGCTGCAGACCGAA | |
| | ACCATGAAGGAAAAGGCCGTCCTGAGCGAGATCGAGGGCACCAGAAGCAGCCTGAAGCACCT | |
| | GAACCACCAGCTGCAGAAGCTCGACTTCGAGACACTGAAGCAGCAAGAGATCATGTACAGCC | |
| | AGGACTTCCACATCCAGCAGGTCGAGCGGCGGATGAGCAGACTGAAGGGCGAGATCAACAGC | |
| | GAGGAAAAACAGGCCCTCGAGGCCAAGATCGTGGAACTGAGAAAGAGCCTCGAAGAGAAGAA | |
| | GAGCACCTGCGGCCTGCTGGAAACCCAGATCAAGAAGCTGCACAACGACCTGTACTTCATCA | |
| | AGAAAGCCCACAGCAAGAACAGCGACGAGAAGCAGAGCCTGATGACCAAGATCAACGAGCTG | |
| | AACCTGTTCATCGACCGGAGCGAAAAAGAGCTGGACAAGGCCAAGGGCTTCAAGCAGGACCT | |
| | GATGATCGAGGACAACCTGCTGAAGCTGGAAGTGAAGCGGACCAGAGAGATGCTGCACAGCA | |
| | AGGCCGAGGAAGTGCTGAGCCTGGAAAAGCGGAAGCAGCAGCTGTACACCGCCATGGAAGAG | |
| | AGAACCGAAGAGATCAAGGTGCACAAGACCATGCTGGCCAGCCAGATCAGATACGTGGACCA | |
| | AGAGCGCGAGAACATCAGCACCGAGTTCAGAGAGAGACTGAGCAAGATCGAGAAGCTGAAGA | |
| | ACCGCTACGAGATCCTGACCGTCGTGATGCTGCCCCCCGAGGGCGAAGAGGAAAAGACCCAG | |
| | GCCTACTACGTGATCAAGGCAGCCCAAGAAAAAGAGGAACTCCAGAGAGAAGGCGACTGCCT | |
| | GGACGCCAAGATCAACAAGGCCGAAAAAGAAATCTACGCCCTCGAGAACACCCTGCAGGTCC | |
| | TGAACAGCTGCAACAACAACTACAAGCAGAGCTTCAAGAAAGTCACCCCCAGCAGCGACGAG | |
| | TACGAGCTGAAGATCCAGCTGGAAGAACAGAAAAGAGCCGTGGACGAGAAGTACAGATACAA | |
| | GCAGCGGCAGATCAGAGAGCTGCAAGAGGACATCCAGAGCATGGAAAACACCCTGGACGTGA | |
| | TCGAGCACCTGGCCAACAACGTGAAAGAGAAGCTGAGCGAGAAACAGGCCTACAGCTTCCAG | |
| | CTGAGCAAAGAGACAGAGGAACAGAAGCCCAAACTGGAACGCGTGACCAAGCAGTGCGCCAA | |
| | GCTGACAAAAGAGATCCGGCTGCTGAAAGACACCAAGGACGAAACCATGGAAGAACAAGACA | |
| | TCAAGCTGCGCGAGATGAAGCAGTTCCACAAAGTGATCGACGAGATGCTGGTGGACATCATC | |
| | GAAGAGAACACAGAGATCCGCATCATCCTGCAGACCTACTTCCAGCAGAGCGGCCTGGAACT | |
| | GCCCACCGCCAGCACAAAGGGCAGCAGACAGAGCAGCAGAAGCCCCAGCCACACAAGCCTGA | |
| | GCGCCAGAAGCAGCAGAAGCACCAGCACCAGCACCAGCCAGAGCAGCATCAAGGTGCTGGAA | |
| | CTCAAGTTCCCCGCCAGCAGCAGCCTCGTGGGAAGCCCCAGCAGACCCAGCAGCGCCAGCAG | |
| | CAGCAGCAGCAACGTGAAGAGCAAGAAAAGCAGCAAGTGAGAATTCtgcagAAAAAAAAAAA | |
| | AAAAAAAAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAATTCG | |
| SEQ ID | ATGAGCAGCGAGTTCCTGGCCGAACTGCACTGGGAGGACGGCTTCGCCATCCCCGTGGCCAA | CCDC39 |
| NO: 27 | CGAGGAAAACAAGCTGCTGGAAGACCAGCTGAGCAAGCTGAAGGACGAGAGAGCCAGCCTGC | (ORF and |
| | AGGACGAGCTGAGAGAGTACGAGGAACGGATCAACAGCATGACCAGCCACTTCAAGAACGTG | HA Tag) |
| | AAGCAAGAGCTGAGCATCACCCAGAGCCTGTGCAAGGCCAGAGAGAGAGAAACCGAGAGCGA | |
| | GGAACACTTCAAGGCCATCGCCCAGCGCGAGCTGGGAAGAGTGAAGGACGAGATCCAGCGGC | |
| | TGGAAAACGAGATGGCCAGCATCCTGGAAAAGAAGAGCGACAAAGAGAACGGCATCTTCAAG | |
| | GCCACACAGAAGCTGGACGGCCTGAAGTGCCAGATGAACTGGGACCAGCAGGCCCTGGAAGC | |
| | CTGGCTGGAAGAGAGCGCCCACAAGGACAGCGACGCCCTGACACTGCAGAAGTACGCCCAGC | |
| | AGGACGACAACAAGATCCGGGCCCTGACCCTGCAGCTGGAAAGACTGACCCTGGAATGCAAC | |
| | CAGAAGCGGAAGATCCTGGACAACGAGCTGACCGAGACAATCAGCGCCCAGCTGGAACTGGA | |
| | CAAGGCCGCCCAGGACTTCAGAAAGATCCACAACGAGCGGCAAGAACTGATCAAGCAGTGGG | |
| | AGAACACCATCGAGCAGATGCAGAAACGCGACGGCGACATCGACAACTGCGCCCTGGAACTC | |
| | GCCCGGATCAAGCAAGAGACACGCGAGAAAGAGAACCTGGTCAAAGAGAAGATCAAGTTCCT | |
| | CGAGAGCGAGATCGGCAACAACACCGAGTTCGAGAAGCGGATCAGCGTGGCCGACAGAAAGC | |
| | TGCTGAAGTGCAGAACCGCCTACCAGGACCACGAGACAAGCCGGATCCAGCTCAAGGGCGAG | |
| | CTGGACAGCCTGAAGGCCACCGTGAACAGAACCAGCAGCGACCTGGAAGCCCTGCGGAAGAA | |
| | CATCAGCAAGATCAAGAAGGACATCCACGAGGAAACCGCCAGGCTGCAGAAAACAAAGAACC | |
| | ACAACGAGATCATCCAGACCAAGCTGAAAGAGATCACCGAAAAGACCATGAGCGTGGAAGAG | |
| | AAGGCCACAAACCTGGAAGACATGCTCAAAGAGGAAGAGAAAGACGTCAAAGAGGTGGACGT | |
| | GCAACTGAACCTGATCAAGGGCGTGCTGTTCAAGAAGGCCCAAGAGCTGCAGACCGAAACCA | |
| | TGAAGGAAAAGGCCGTCCTGAGCGAGATCGAGGGCACCAGAAGCAGCCTGAAGCACCTGAAC | |
| | CACCAGCTGCAGAAGCTCGACTTCGAGACACTGAAGCAGCAAGAGATCATGTACAGCCAGGA | |
| | CTTCCACATCCAGCAGGTCGAGCGGCGGATGAGCAGACTGAAGGGCGAGATCAACAGCGAGG | |
| | AAAAACAGGCCCTCGAGGCCAAGATCGTGGAACTGAGAAAGAGCCTCGAAGAGAAGAAGAGC | |
| | ACCTGCGGCCTGCTGGAAACCCAGATCAAGAAGCTGCACAACGACCTGTACTTCATCAAGAA | |
| | AGCCCACAGCAAGAACAGCGACGAGAAGCAGAGCCTGATGACCAAGATCAACGAGCTGAACC | |
| | TGTTCATCGACCGGAGCGAAAAAGAGCTGGACAAGGCCAAGGGCTTCAAGCAGGACCTGATG | |
| | ATCGAGGACAACCTGCTGAAGCTGGAAGTGAAGCGGACCAGAGAGATGCTGCACAGCAAGGC | |
| | CGAGGAAGTGCTGAGCCTGGAAAAGCGGAAGCAGCAGCTGTACACCGCCATGGAAGAGAGAA | |
| | CCGAAGAGATCAAGGTGCACAAGACCATGCTGGCCAGCCAGATCAGATACGTGGACCAAGAG | |
| | CGCGAGAACATCAGCACCGAGTTCAGAGAGAGACTGAGCAAGATCGAGAAGCTGAAGAACCG | |
| | CTACGAGATCCTGACCGTCGTGATGCTGCCCCCCGAGGGCGAAGAGGAAAAGACCCAGGCCT | |
| | ACTACGTGATCAAGGCAGCCCAAGAAAAAGAGGAACTCCAGAGAGAAGGCGACTGCCTGGAC | |
| | GCCAAGATCAACAAGGCCGAAAAAGAAATCTACGCCCTCGAGAACACCCTGCAGGTCCTGAA | |
| | CAGCTGCAACAACAACTACAAGCAGAGCTTCAAGAAAGTCACCCCCAGCAGCGACGAGTACG | |
| | AGCTGAAGATCCAGCTGGAAGAACAGAAAAGAGCCGTGGACGAGAAGTACAGATACAAGCAG | |
| | CGGCAGATCAGAGAGCTGCAAGAGGACATCCAGAGCATGGAAAACACCCTGGACGTGATCGA | |
| | GCACCTGGCCAACAACGTGAAAGAGAAGCTGAGCGAGAAACAGGCCTACAGCTTCCAGCTGA | |
| | GCAAAGAGACAGAGGAACAGAAGCCCAAACTGGAACGCGTGACCAAGCAGTGCGCCAAGCTG | |
| | ACAAAAGAGATCCGGCTGCTGAAAGACACCAAGGACGAAACCATGGAAGAACAAGACATCAA | |
| | GCTGCGCGAGATGAAGCAGTTCCACAAAGTGATCGACGAGATGCTGGTGGACATCATCGAAG | |
| | AGAACACAGAGATCCGCATCATCCTGCAGACCTACTTCCAGCAGAGCGGCCTGGAACTGCCC | |
| | ACCGCCAGCACAAAGGGCAGCAGACAGAGCAGCAGAAGCCCCAGCCACACAAGCCTGAGCGC | |
| | CAGAAGCAGCAGAAGCACCAGCACCAGCACCAGCCAGAGCAGCATCAAGGTGCTGGAACTCA | |
| | AGTTCCCCGCCAGCAGCAGCCTCGTGGGAAGCCCCAGCAGACCCAGCAGCGCCAGCAGCAGC | |
| | AGCAGCAACGTGAAGAGCAAGAAAAGCAGCAAGGGAAGCGGCTACCCATACGATGTTCCTGA | |
| | CTATGCGTGA | |
| SEQ ID | GGGAGACCCAAGCTGGCTAGCGTTTAAACTTCAGCTTGGCAATCCGGTACTGTTGGTAAAGC | CCDC39 |
| NO: 28 | CACCATGAGCAGCGAGTTCCTGGCCGAACTGCACTGGGAGGACGGCTTCGCCATCCCCGTGG | (5′ UTR, |
| | CCAACGAGGAAAACAAGCTGCTGGAAGACCAGCTGAGCAAGCTGAAGGACGAGAGAGCCAGC | ORF, HA |
| | CTGCAGGACGAGCTGAGAGAGTACGAGGAACGGATCAACAGCATGACCAGCCACTTCAAGAA | Tag, and |
| | CGTGAAGCAAGAGCTGAGCATCACCCAGAGCCTGTGCAAGGCCAGAGAGAGAGAAACCGAGA | 3′ Tail) |
| | GCGAGGAACACTTCAAGGCCATCGCCCAGCGCGAGCTGGGAAGAGTGAAGGACGAGATCCAG | |
| | CGGCTGGAAAACGAGATGGCCAGCATCCTGGAAAAGAAGAGCGACAAAGAGAACGGCATCTT | |
| | CAAGGCCACACAGAAGCTGGACGGCCTGAAGTGCCAGATGAACTGGGACCAGCAGGCCCTGG | |
| | AAGCCTGGCTGGAAGAGAGCGCCCACAAGGACAGCGACGCCCTGACACTGCAGAAGTACGCC | |
| | CAGCAGGACGACAACAAGATCCGGGCCCTGACCCTGCAGCTGGAAAGACTGACCCTGGAATG | |
| | CAACCAGAAGCGGAAGATCCTGGACAACGAGCTGACCGAGACAATCAGCGCCCAGCTGGAAC | |
| | TGGACAAGGCCGCCCAGGACTTCAGAAAGATCCACAACGAGCGGCAAGAACTGATCAAGCAG | |
| | TGGGAGAACACCATCGAGCAGATGCAGAAACGCGACGGCGACATCGACAACTGCGCCCTGGA | |
| | ACTCGCCCGGATCAAGCAAGAGACACGCGAGAAAGAGAACCTGGTCAAAGAGAAGATCAAGT | |
| | TCCTCGAGAGCGAGATCGGCAACAACACCGAGTTCGAGAAGCGGATCAGCGTGGCCGACAGA | |
| | AAGCTGCTGAAGTGCAGAACCGCCTACCAGGACCACGAGACAAGCCGGATCCAGCTCAAGGG | |
| | CGAGCTGGACAGCCTGAAGGCCACCGTGAACAGAACCAGCAGCGACCTGGAAGCCCTGCGGA | |
| | AGAACATCAGCAAGATCAAGAAGGACATCCACGAGGAAACCGCCAGGCTGCAGAAAACAAAG | |
| | AACCACAACGAGATCATCCAGACCAAGCTGAAAGAGATCACCGAAAAGACCATGAGCGTGGA | |
| | AGAGAAGGCCACAAACCTGGAAGACATGCTCAAAGAGGAAGAGAAAGACGTCAAAGAGGTGG | |
| | ACGTGCAACTGAACCTGATCAAGGGCGTGCTGTTCAAGAAGGCCCAAGAGCTGCAGACCGAA | |
| | ACCATGAAGGAAAAGGCCGTCCTGAGCGAGATCGAGGGCACCAGAAGCAGCCTGAAGCACCT | |
| | GAACCACCAGCTGCAGAAGCTCGACTTCGAGACACTGAAGCAGCAAGAGATCATGTACAGCC | |
| | AGGACTTCCACATCCAGCAGGTCGAGCGGCGGATGAGCAGACTGAAGGGCGAGATCAACAGC | |
| | GAGGAAAAACAGGCCCTCGAGGCCAAGATCGTGGAACTGAGAAAGAGCCTCGAAGAGAAGAA | |
| | GAGCACCTGCGGCCTGCTGGAAACCCAGATCAAGAAGCTGCACAACGACCTGTACTTCATCA | |
| | AGAAAGCCCACAGCAAGAACAGCGACGAGAAGCAGAGCCTGATGACCAAGATCAACGAGCTG | |
| | AACCTGTTCATCGACCGGAGCGAAAAAGAGCTGGACAAGGCCAAGGGCTTCAAGCAGGACCT | |
| | GATGATCGAGGACAACCTGCTGAAGCTGGAAGTGAAGCGGACCAGAGAGATGCTGCACAGCA | |
| | AGGCCGAGGAAGTGCTGAGCCTGGAAAAGCGGAAGCAGCAGCTGTACACCGCCATGGAAGAG | |
| | AGAACCGAAGAGATCAAGGTGCACAAGACCATGCTGGCCAGCCAGATCAGATACGTGGACCA | |
| | AGAGCGCGAGAACATCAGCACCGAGTTCAGAGAGAGACTGAGCAAGATCGAGAAGCTGAAGA | |
| | ACCGCTACGAGATCCTGACCGTCGTGATGCTGCCCCCCGAGGGCGAAGAGGAAAAGACCCAG | |
| | GCCTACTACGTGATCAAGGCAGCCCAAGAAAAAGAGGAACTCCAGAGAGAAGGCGACTGCCT | |
| | GGACGCCAAGATCAACAAGGCCGAAAAAGAAATCTACGCCCTCGAGAACACCCTGCAGGTCC | |
| | TGAACAGCTGCAACAACAACTACAAGCAGAGCTTCAAGAAAGTCACCCCCAGCAGCGACGAG | |
| | TACGAGCTGAAGATCCAGCTGGAAGAACAGAAAAGAGCCGTGGACGAGAAGTACAGATACAA | |
| | GCAGCGGCAGATCAGAGAGCTGCAAGAGGACATCCAGAGCATGGAAAACACCCTGGACGTGA | |
| | TCGAGCACCTGGCCAACAACGTGAAAGAGAAGCTGAGCGAGAAACAGGCCTACAGCTTCCAG | |
| | CTGAGCAAAGAGACAGAGGAACAGAAGCCCAAACTGGAACGCGTGACCAAGCAGTGCGCCAA | |
| | GCTGACAAAAGAGATCCGGCTGCTGAAAGACACCAAGGACGAAACCATGGAAGAACAAGACA | |
| | TCAAGCTGCGCGAGATGAAGCAGTTCCACAAAGTGATCGACGAGATGCTGGTGGACATCATC | |
| | GAAGAGAACACAGAGATCCGCATCATCCTGCAGACCTACTTCCAGCAGAGCGGCCTGGAACT | |
| | GCCCACCGCCAGCACAAAGGGCAGCAGACAGAGCAGCAGAAGCCCCAGCCACACAAGCCTGA | |
| | GCGCCAGAAGCAGCAGAAGCACCAGCACCAGCACCAGCCAGAGCAGCATCAAGGTGCTGGAA | |
| | CTCAAGTTCCCCGCCAGCAGCAGCCTCGTGGGAAGCCCCAGCAGACCCAGCAGCGCCAGCAG | |
| | CAGCAGCAGCAACGTGAAGAGCAAGAAAAGCAGCAAGGGAAGCGGCTACCCATACGATGTTC | |
| | CTGACTATGCGTGAGAATTCTGCAGAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAA | |
| | AAAAAAAAAAAAAAAAAAAAATTCGGAATTCTGCAGAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAATTCG | |
| SEQ ID | ATGGCCGAACCCGGCGGAGCCGCCGGAAGAAGCCACCCCGAAGACGGCAGCGCCAGCGAGGG | CCDC40 |
| NO: 29 | CGAGAAAGAGGGCAACAACGAGAGCCACATGGTGAGCCCACCCGAGAAGGACGACGGCCAGA | (ORF) |
| | AAGGCGAAGAGGCCGTGGGAAGCACAGAGCACCCCGAGGAAGTGACCACACAGGCCGAAGCC | |
| | GCCATCGAGGAAGGCGAGGTGGAAACAGAGGGCGAAGCCGCCGTGGAGGGCGAAGAGGAAGC | |
| | CGTGAGCTACGGCGACGCCGAGAGCGAGGAAGAGTACTACTACACCGAGACAAGCAGCCCCG | |
| | AGGGCCAGATCAGCGCCGCCGACACCACCTACCCCTACTTCAGCCCCCCCCAAGAGCTGCCC | |
| | GGCGAAGAGGCCTACGACAGCGTGAGCGGCGAAGCCGGCCTGCAGGGCTTCCAGCAAGAAGC | |
| | CACAGGCCCCCCCGAGAGCCGCGAGAGAAGAGTGACAAGCCCCGAACCCAGCCACGGCGTGC | |
| | TGGGACCAAGCGAGCAGATGGGCCAAGTGACAAGCGGACCCGCCGTGGGCAGACTGACAGGC | |
| | AGCACAGAGGAACCCCAGGGACAAGTGCTGCCCATGGGAGTGCAGCACCGGTTCAGACTGAG | |
| | CCACGGCAGCGACATCGAGAGCAGCGACCTGGAAGAGTTCGTGAGCCAAGAGCCCGTGATCC | |
| | CCCCCGGCGTGCCAGACGCCCACCCCAGAGAAGGCGACCTGCCCGTGTTCCAGGACCAGATC | |
| | CAGCAGCCCAGCACCGAAGAGGGCGCCATGGCCGAGAGAGTGGAAAGCGAGGGCAGCGACGA | |
| | AGAAGCCGAGGACGAGGGAAGCCAGCTGGTGGTGCTGGACCCCGACCACCCCCTGATGGTCC | |
| | GATTCCAAGCCGCCCTGAAGAACTACCTGAACCGGCAGATCGAGAAGCTGAAGCTGGACCTC | |
| | CAAGAACTGGTGGTGGCCACAAAGCAGAGCAGAGCCCAGAGACAAGAGCTGGGCGTGAACCT | |
| | GTACGAGGTGCAGCAGCACCTGGTGCACCTGCAGAAACTGCTGGAAAAGAGCCACGACCGGC | |
| | ACGCCATGGCCAGCAGCGAGCGCAGACAGAAAGAGGAAGAACTGCAGGCCGCCCGGGCCCTG | |
| | TACACCAAAACATGCGCCGCCGCCAACGAGGAACGGAAGAAACTGGCCGCCCTGCAGACCGA | |
| | GATGGAAAACCTGGCCCTGCACCTGTTCTACATGCAGAACATCGACCAGGACATGCGGGACG | |
| | ACATCAGAGTGATGACCCAGGTGGTCAAGAAGGCCGAGACAGAGAGAATCCGGGCCGAGATC | |
| | GAGAAGAAGAAACAGGACCTGTACGTGGACCAGCTGACCACAAGAGCCCAGCAGCTGGAAGA | |
| | GGACATCGCCCTGTTCGAGGCCCAGTACCTGGCCCAGGCCGAGGACACCAGAATCCTGAGAA | |
| | AGGCCGTGAGCGAGGCCTGCACAGAGATCGACGCCATCAGCGTGGAAAAGCGGCGGATCATG | |
| | CAGCAGTGGGCCAGCAGCCTGGTGGGCATGAAGCACAGGGACGAAGCCCACAGAGCCGTGCT | |
| | GGAAGCCCTGAGAGGCTGCCAGCACCAGGCCAAGAGCACCGACGGCGAGATCGAGGCCTACA | |
| | AGAAAAGCATCATGAAGGAAGAGGAAAAGAACGAGAAGCTCGCCAGCATCCTGAACAGAACC | |
| | GAAACCGAGGCCACCCTGCTCCAGAAACTGACAACCCAGTGCCTGACCAAACAGGTGGCCCT | |
| | GCAGAGCCAGTTCAACACCTACAGACTGACCCTGCAGGACACCGAGGACGCCCTGAGCCAGG | |
| | ACCAGCTGGAACAGATGATCCTGACCGAGGAACTCCAGGCCATCAGACAGGCCATCCAGGGC | |
| | GAACTGGAACTGCGGAGAAAGACCGACGCCGCCATCAGAGAGAAGCTGCAAGAGCACATGAC | |
| | CAGCAACAAGACCACCAAGTACTTCAACCAGCTGATCCTGCGGCTGCAGAAAGAAAAGACCA | |
| | ACATGATGACACACCTGAGCAAGATCAACGGCGACATCGCCCAGACCACACTGGACATCACC | |
| | CACACCAGCAGCAGACTGGACGCCCACCAGAAAACCCTGGTGGAACTGGACCAGGACGTGAA | |
| | GAAAGTGAACGAGCTGATCACCAACAGCCAGAGCGAGATCAGCAGACGGACCATCCTGATCG | |
| | AGAGAAAGCAGGGCCTGATCAACTTCCTGAACAAGCAGCTCGAGCGGATGGTGAGCGAACTC | |
| | GGCGGAGAAGAAGTGGGCCCCCTGGAACTCGAGATCAAGCGGCTGAGCAAGCTGATCGACGA | |
| | GCACGACGGAAAGGCAGTGCAGGCCCAAGTGACCTGGCTGAGACTGCAGCAAGAGATGGTCA | |
| | AAGTGACCCAAGAGCAAGAGGAACAGCTGGCCAGCCTGGACGCCAGCAAGAAAGAACTCCAC | |
| | ATCATGGAACAGAAGAAGCTGAGGGTCGAGAGCAAGATCGAGCAAGAGAAGAAAGAGCAAAA | |
| | AGAAATCGAGCACCACATGAAGGACCTGGACAACGACCTGAAAAAGCTGAACATGCTGATGA | |
| | ACAAGAACCGCTGCAGCAGCGAAGAACTCGAGCAGAACAACAGAGTGACCGAGAACGAGTTC | |
| | GTGCGGAGCCTGAAGGCCAGCGAGAGGGAAACCATCAAGATGCAGGACAAGCTGAACCAGCT | |
| | GAGCGAGGAAAAGGCCACACTGCTGAACCAACTGGTGGAAGCCGAGCACCAGATCATGCTGT | |
| | GGGAGAAGAAAATCCAGCTGGCCAAAGAAATGCGGAGCAGCGTGGACAGCGAGATCGGCCAG | |
| | ACCGAAATCAGAGCCATGAAGGGCGAGATCCACCGGATGAAAGTGCGGCTGGGACAGCTGCT | |
| | GAAACAACAAGAGAAAATGATCCGCGCCATGGAACTGGCCGTGGCCAGACGGGAAACAGTGA | |
| | CCACCCAAGCCGAGGGACAGAGAAAGATGGACCGGAAGGCCCTGACCAGGACCGACTTCCAC | |
| | CACAAACAGCTCGAACTGCGGCGGAAGATCAGGGACGTGCGGAAGGCCACCGACGAGTGCAC | |
| | CAAGACAGTGCTGGAACTGGAAGAGACACAGCGGAACGTGAGCAGCAGCCTGCTCGAGAAGC | |
| | AAGAAAAGCTGAGCGTGATCCAGGCCGACTTCGACACCCTGGAAGCCGACCTGACAAGACTG | |
| | GGAGCCCTGAAGAGGCAGAACCTGAGCGAAATCGTGGCACTGCAGACCCGGCTGAAACACCT | |
| | GCAGGCAGTGAAAGAGGGGCGCTACGTGTTCCTGTTCCGGAGCAAGCAGAGCCTGGTGCTGG | |
| | AGAGACAGCGGCTGGACAAGCGGCTGGCCCTGATCGCCACAATCCTGGACAGAGTGCGCGAC | |
| | GAGTACCCCCAGTTCCAAGAGGCCCTGCACAAGGTGAGCCAGATGATCGCCAACAAGCTGGA | |
| | AAGCCCCGGACCCAGCTGA | |
| SEQ ID | GGGAGACCCAAGCTGGCTAGCGTTTAAACTTCAGCTTGGCAATCCGGTACTGTTGGTAAAGC | CCDC40 |
| NO: 30 | CACCATGGCCGAACCCGGCGGAGCCGCCGGAAGAAGCCACCCCGAAGACGGCAGCGCCAGCG | (5′ UTR, |
| | AGGGCGAGAAAGAGGGCAACAACGAGAGCCACATGGTGAGCCCACCCGAGAAGGACGACGGC | ORF, and |
| | CAGAAAGGCGAAGAGGCCGTGGGAAGCACAGAGCACCCCGAGGAAGTGACCACACAGGCCGA | 3′ Tail) |
| | AGCCGCCATCGAGGAAGGCGAGGTGGAAACAGAGGGCGAAGCCGCCGTGGAGGGCGAAGAGG | |
| | AAGCCGTGAGCTACGGCGACGCCGAGAGCGAGGAAGAGTACTACTACACCGAGACAAGCAGC | |
| | CCCGAGGGCCAGATCAGCGCCGCCGACACCACCTACCCCTACTTCAGCCCCCCCCAAGAGCT | |
| | GCCCGGCGAAGAGGCCTACGACAGCGTGAGCGGCGAAGCCGGCCTGCAGGGCTTCCAGCAAG | |
| | AAGCCACAGGCCCCCCCGAGAGCCGCGAGAGAAGAGTGACAAGCCCCGAACCCAGCCACGGC | |
| | GTGCTGGGACCAAGCGAGCAGATGGGCCAAGTGACAAGCGGACCCGCCGTGGGCAGACTGAC | |
| | AGGCAGCACAGAGGAACCCCAGGGACAAGTGCTGCCCATGGGAGTGCAGCACCGGTTCAGAC | |
| | TGAGCCACGGCAGCGACATCGAGAGCAGCGACCTGGAAGAGTTCGTGAGCCAAGAGCCCGTG | |
| | ATCCCCCCCGGCGTGCCAGACGCCCACCCCAGAGAAGGCGACCTGCCCGTGTTCCAGGACCA | |
| | GATCCAGCAGCCCAGCACCGAAGAGGGCGCCATGGCCGAGAGAGTGGAAAGCGAGGGCAGCG | |
| | ACGAAGAAGCCGAGGACGAGGGAAGCCAGCTGGTGGTGCTGGACCCCGACCACCCCCTGATG | |
| | GTCCGATTCCAAGCCGCCCTGAAGAACTACCTGAACCGGCAGATCGAGAAGCTGAAGCTGGA | |
| | CCTCCAAGAACTGGTGGTGGCCACAAAGCAGAGCAGAGCCCAGAGACAAGAGCTGGGCGTGA | |
| | ACCTGTACGAGGTGCAGCAGCACCTGGTGCACCTGCAGAAACTGCTGGAAAAGAGCCACGAC | |
| | CGGCACGCCATGGCCAGCAGCGAGCGCAGACAGAAAGAGGAAGAACTGCAGGCCGCCCGGGC | |
| | CCTGTACACCAAAACATGCGCCGCCGCCAACGAGGAACGGAAGAAACTGGCCGCCCTGCAGA | |
| | CCGAGATGGAAAACCTGGCCCTGCACCTGTTCTACATGCAGAACATCGACCAGGACATGCGG | |
| | GACGACATCAGAGTGATGACCCAGGTGGTCAAGAAGGCCGAGACAGAGAGAATCCGGGCCGA | |
| | GATCGAGAAGAAGAAACAGGACCTGTACGTGGACCAGCTGACCACAAGAGCCCAGCAGCTGG | |
| | AAGAGGACATCGCCCTGTTCGAGGCCCAGTACCTGGCCCAGGCCGAGGACACCAGAATCCTG | |
| | AGAAAGGCCGTGAGCGAGGCCTGCACAGAGATCGACGCCATCAGCGTGGAAAAGCGGCGGAT | |
| | CATGCAGCAGTGGGCCAGCAGCCTGGTGGGCATGAAGCACAGGGACGAAGCCCACAGAGCCG | |
| | TGCTGGAAGCCCTGAGAGGCTGCCAGCACCAGGCCAAGAGCACCGACGGCGAGATCGAGGCC | |
| | TACAAGAAAAGCATCATGAAGGAAGAGGAAAAGAACGAGAAGCTCGCCAGCATCCTGAACAG | |
| | AACCGAAACCGAGGCCACCCTGCTCCAGAAACTGACAACCCAGTGCCTGACCAAACAGGTGG | |
| | CCCTGCAGAGCCAGTTCAACACCTACAGACTGACCCTGCAGGACACCGAGGACGCCCTGAGC | |
| | CAGGACCAGCTGGAACAGATGATCCTGACCGAGGAACTCCAGGCCATCAGACAGGCCATCCA | |
| | GGGCGAACTGGAACTGCGGAGAAAGACCGACGCCGCCATCAGAGAGAAGCTGCAAGAGCACA | |
| | TGACCAGCAACAAGACCACCAAGTACTTCAACCAGCTGATCCTGCGGCTGCAGAAAGAAAAG | |
| | ACCAACATGATGACACACCTGAGCAAGATCAACGGCGACATCGCCCAGACCACACTGGACAT | |
| | CACCCACACCAGCAGCAGACTGGACGCCCACCAGAAAACCCTGGTGGAACTGGACCAGGACG | |
| | TGAAGAAAGTGAACGAGCTGATCACCAACAGCCAGAGCGAGATCAGCAGACGGACCATCCTG | |
| | ATCGAGAGAAAGCAGGGCCTGATCAACTTCCTGAACAAGCAGCTCGAGCGGATGGTGAGCGA | |
| | ACTCGGCGGAGAAGAAGTGGGCCCCCTGGAACTCGAGATCAAGCGGCTGAGCAAGCTGATCG | |
| | ACGAGCACGACGGAAAGGCAGTGCAGGCCCAAGTGACCTGGCTGAGACTGCAGCAAGAGATG | |
| | GTCAAAGTGACCCAAGAGCAAGAGGAACAGCTGGCCAGCCTGGACGCCAGCAAGAAAGAACT | |
| | CCACATCATGGAACAGAAGAAGCTGAGGGTCGAGAGCAAGATCGAGCAAGAGAAGAAAGAGC | |
| | AAAAAGAAATCGAGCACCACATGAAGGACCTGGACAACGACCTGAAAAAGCTGAACATGCTG | |
| | ATGAACAAGAACCGCTGCAGCAGCGAAGAACTCGAGCAGAACAACAGAGTGACCGAGAACGA | |
| | GTTCGTGCGGAGCCTGAAGGCCAGCGAGAGGGAAACCATCAAGATGCAGGACAAGCTGAACC | |
| | AGCTGAGCGAGGAAAAGGCCACACTGCTGAACCAACTGGTGGAAGCCGAGCACCAGATCATG | |
| | CTGTGGGAGAAGAAAATCCAGCTGGCCAAAGAAATGCGGAGCAGCGTGGACAGCGAGATCGG | |
| | CCAGACCGAAATCAGAGCCATGAAGGGCGAGATCCACCGGATGAAAGTGCGGCTGGGACAGC | |
| | TGCTGAAACAACAAGAGAAAATGATCCGCGCCATGGAACTGGCCGTGGCCAGACGGGAAACA | |
| | GTGACCACCCAAGCCGAGGGACAGAGAAAGATGGACCGGAAGGCCCTGACCAGGACCGACTT | |
| | CCACCACAAACAGCTCGAACTGCGGCGGAAGATCAGGGACGTGCGGAAGGCCACCGACGAGT | |
| | GCACCAAGACAGTGCTGGAACTGGAAGAGACACAGCGGAACGTGAGCAGCAGCCTGCTCGAG | |
| | AAGCAAGAAAAGCTGAGCGTGATCCAGGCCGACTTCGACACCCTGGAAGCCGACCTGACAAG | |
| | ACTGGGAGCCCTGAAGAGGCAGAACCTGAGCGAAATCGTGGCACTGCAGACCCGGCTGAAAC | |
| | ACCTGCAGGCAGTGAAAGAGGGGCGCTACGTGTTCCTGTTCCGGAGCAAGCAGAGCCTGGTG | |
| | CTGGAGAGACAGCGGCTGGACAAGCGGCTGGCCCTGATCGCCACAATCCTGGACAGAGTGCG | |
| | CGACGAGTACCCCCAGTTCCAAGAGGCCCTGCACAAGGTGAGCCAGATGATCGCCAACAAGC | |
| | TGGAAAGCCCCGGACCCAGCTGAGAATTCTGCAGAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAA | |
| | AAAAAAAAAAAAAAAAAAAAAAAAAAAAAATTCG | |
| SEQ ID | ATGGCTGAACCTGGCGGAGCTGCTGGAAGATCTCACCCTGAAGATGGCTCTGCCAGCGAGGG | CCDC40 |
| NO: 31 | CGAGAAAGAGGGCAACAACGAGAGCCACATGGTGAGCCCACCTGAGAAGGACGATGGCCAGA | (ORF and |
| | AAGGCGAAGAGGCCGTGGGAAGCACAGAGCACCCTGAGGAAGTGACCACACAGGCCGAAGCC | HA Tag) |
| | GCCATCGAGGAAGGCGAGGTGGAAACAGAGGGCGAAGCTGCCGTGGAGGGCGAAGAGGAAGC | |
| | TGTGAGCTACGGCGACGCCGAGAGCGAGGAAGAGTACTACTACACCGAGACAAGCAGCCCCG | |
| | AGGGCCAGATCTCTGCCGCCGACACCACCTATCCCTACTTCAGCCCTCCTCAAGAGCTGCCC | |
| | GGCGAAGAGGCCTACGACTCTGTGTCTGGCGAAGCCGGCCTGCAGGGCTTCCAGCAAGAAGC | |
| | CACAGGCCCTCCTGAGAGCCGCGAGAGAAGAGTGACAAGCCCTGAACCCTCTCACGGCGTGC | |
| | TGGGACCATCTGAGCAGATGGGCCAAGTGACATCTGGACCTGCTGTGGGCAGACTGACAGGC | |
| | AGCACAGAGGAACCTCAGGGACAAGTGCTGCCCATGGGAGTGCAGCACCGGTTCAGACTGAG | |
| | CCACGGCAGCGACATCGAGAGCAGCGACCTGGAAGAGTTCGTGAGCCAAGAGCCTGTGATCC | |
| | CTCCTGGCGTGCCAGATGCTCATCCCAGAGAAGGCGATCTGCCCGTGTTCCAGGACCAGATC | |
| | CAGCAGCCCAGCACTGAAGAGGGCGCCATGGCCGAGAGAGTGGAATCTGAGGGCTCTGACGA | |
| | AGAAGCCGAGGACGAGGGATCTCAGCTGGTGGTGCTGGATCCTGATCACCCTCTGATGGTCC | |
| | GATTCCAAGCCGCTCTGAAGAACTACCTGAACCGGCAGATCGAGAAGCTGAAGCTGGACCTC | |
| | CAAGAACTGGTGGTGGCCACAAAGCAGAGCAGAGCCCAGAGACAAGAGCTGGGCGTGAACCT | |
| | GTATGAGGTGCAGCAGCACCTGGTGCATCTGCAGAAACTGCTGGAAAAGAGCCACGACCGGC | |
| | ACGCCATGGCCAGCTCTGAGCGCAGACAGAAAGAGGAAGAACTGCAGGCTGCCCGGGCTCTG | |
| | TACACCAAAACATGTGCCGCCGCCAACGAGGAACGGAAGAAACTGGCTGCCCTGCAGACCGA | |
| | GATGGAAAACCTGGCTCTGCACCTGTTCTACATGCAGAACATCGACCAGGACATGCGGGACG | |
| | ACATCAGAGTGATGACCCAGGTGGTCAAGAAGGCCGAGACAGAGAGAATCCGGGCCGAGATC | |
| | GAGAAGAAGAAACAGGACCTGTACGTGGACCAGCTGACCACAAGAGCCCAGCAGCTGGAAGA | |
| | GGACATCGCCCTGTTCGAGGCCCAGTATCTGGCCCAGGCTGAGGACACCAGAATCCTGAGAA | |
| | AGGCCGTGAGCGAGGCCTGCACAGAGATCGATGCCATCAGCGTGGAAAAGCGGCGGATCATG | |
| | CAGCAGTGGGCCAGCTCTCTGGTGGGCATGAAGCACAGGGACGAAGCCCACAGAGCCGTGCT | |
| | GGAAGCTCTGAGAGGCTGTCAGCACCAGGCCAAGAGCACCGATGGCGAGATCGAGGCCTACA | |
| | AGAAAAGCATCATGAAGGAAGAGGAAAAGAACGAGAAGCTCGCCAGCATCCTGAACAGAACC | |
| | GAAACCGAGGCCACTCTGCTCCAGAAACTGACAACCCAGTGCCTGACCAAACAGGTGGCCCT | |
| | GCAGAGCCAGTTCAACACCTACAGACTGACCCTGCAGGACACCGAGGATGCCCTGTCTCAGG | |
| | ATCAGCTGGAACAGATGATCCTGACCGAGGAACTCCAGGCCATCAGACAGGCCATCCAGGGC | |
| | GAACTGGAACTGCGGAGAAAGACCGATGCCGCCATCAGAGAGAAGCTGCAAGAGCACATGAC | |
| | CAGCAACAAGACCACCAAGTACTTCAACCAGCTGATCCTGCGGCTGCAGAAAGAAAAGACCA | |
| | ACATGATGACACACCTGAGCAAGATCAACGGCGACATCGCCCAGACCACACTGGACATCACC | |
| | CACACCAGCAGCAGACTGGACGCCCACCAGAAAACCCTGGTGGAACTGGACCAGGACGTGAA | |
| | GAAAGTGAACGAGCTGATCACCAACAGCCAGAGCGAGATCAGCAGACGGACCATCCTGATCG | |
| | AGAGAAAGCAGGGCCTGATCAACTTCCTGAACAAGCAGCTCGAGCGGATGGTGTCTGAACTC | |
| | GGCGGAGAAGAAGTGGGCCCTCTGGAACTCGAGATCAAGCGGCTGAGCAAGCTGATCGACGA | |
| | GCACGATGGAAAGGCAGTGCAGGCCCAAGTGACCTGGCTGAGACTGCAGCAAGAGATGGTCA | |
| | AAGTGACCCAAGAGCAAGAGGAACAGCTGGCCTCTCTGGACGCCAGCAAGAAAGAACTCCAC | |
| | ATCATGGAACAGAAGAAGCTGAGGGTCGAGAGCAAGATCGAGCAAGAGAAGAAAGAGCAAAA | |
| | AGAAATCGAGCACCACATGAAGGACCTGGACAACGACCTGAAAAAGCTGAACATGCTGATGA | |
| | ACAAGAACCGCTGCAGCAGCGAAGAACTCGAGCAGAACAACAGAGTGACCGAGAACGAGTTC | |
| | GTGCGGAGCCTGAAGGCCAGCGAGAGGGAAACCATCAAGATGCAGGACAAGCTGAACCAGCT | |
| | GAGCGAGGAAAAGGCCACACTGCTGAATCAACTGGTGGAAGCCGAGCACCAGATCATGCTGT | |
| | GGGAGAAGAAAATCCAGCTGGCCAAAGAAATGCGGAGCAGCGTGGACTCTGAGATCGGCCAG | |
| | ACCGAAATCAGAGCCATGAAGGGCGAGATCCACCGGATGAAAGTGCGGCTGGGACAGCTGCT | |
| | GAAACAACAAGAGAAAATGATCCGCGCCATGGAACTGGCCGTGGCCAGACGGGAAACAGTGA | |
| | CCACTCAAGCCGAGGGACAGAGAAAGATGGACCGGAAGGCCCTGACCAGGACCGACTTCCAC | |
| | CACAAACAGCTCGAACTGCGGCGGAAGATCAGGGATGTGCGGAAGGCCACCGATGAGTGCAC | |
| | CAAGACAGTGCTGGAACTGGAAGAGACACAGCGGAACGTGAGCAGCAGCCTGCTCGAGAAGC | |
| | AAGAAAAGCTGAGCGTGATCCAGGCCGACTTCGACACCCTGGAAGCTGACCTGACAAGACTG | |
| | GGAGCCCTGAAGAGGCAGAACCTGAGCGAAATCGTGGCACTGCAGACCCGGCTGAAACATCT | |
| | GCAGGCAGTGAAAGAGGGGCGCTACGTGTTCCTGTTCCGGAGCAAGCAGAGCCTGGTGCTGG | |
| | AGAGACAGCGGCTGGACAAGCGGCTGGCCCTGATCGCCACAATCCTGGACAGAGTGCGCGAC | |
| | GAGTACCCTCAGTTCCAAGAGGCCCTGCACAAGGTGAGCCAGATGATCGCCAACAAGCTGGA | |
| | AAGCCCCGGACCCAGCGGAAGCGGCTACCCATACGATGTTCCTGACTATGCGTGA | |
| SEQ ID | GGGAGACCCAAGCTGGCTAGCGTTTAAACTTCAGCTTGGCAATCCGGTACTGTTGGTAAAGC | CCDC40 |
| NO: 32 | CACCATGGCTGAACCTGGCGGAGCTGCTGGAAGATCTCACCCTGAAGATGGCTCTGCCAGCG | (5′ UTR, |
| | AGGGCGAGAAAGAGGGCAACAACGAGAGCCACATGGTGAGCCCACCTGAGAAGGACGATGGC | ORF, HA |
| | CAGAAAGGCGAAGAGGCCGTGGGAAGCACAGAGCACCCTGAGGAAGTGACCACACAGGCCGA | Tag, and |
| | AGCCGCCATCGAGGAAGGCGAGGTGGAAACAGAGGGCGAAGCTGCCGTGGAGGGCGAAGAGG | 3′ Tail) |
| | AAGCTGTGAGCTACGGCGACGCCGAGAGCGAGGAAGAGTACTACTACACCGAGACAAGCAGC | |
| | CCCGAGGGCCAGATCTCTGCCGCCGACACCACCTATCCCTACTTCAGCCCTCCTCAAGAGCT | |
| | GCCCGGCGAAGAGGCCTACGACTCTGTGTCTGGCGAAGCCGGCCTGCAGGGCTTCCAGCAAG | |
| | AAGCCACAGGCCCTCCTGAGAGCCGCGAGAGAAGAGTGACAAGCCCTGAACCCTCTCACGGC | |
| | GTGCTGGGACCATCTGAGCAGATGGGCCAAGTGACATCTGGACCTGCTGTGGGCAGACTGAC | |
| | AGGCAGCACAGAGGAACCTCAGGGACAAGTGCTGCCCATGGGAGTGCAGCACCGGTTCAGAC | |
| | TGAGCCACGGCAGCGACATCGAGAGCAGCGACCTGGAAGAGTTCGTGAGCCAAGAGCCTGTG | |
| | ATCCCTCCTGGCGTGCCAGATGCTCATCCCAGAGAAGGCGATCTGCCCGTGTTCCAGGACCA | |
| | GATCCAGCAGCCCAGCACTGAAGAGGGCGCCATGGCCGAGAGAGTGGAATCTGAGGGCTCTG | |
| | ACGAAGAAGCCGAGGACGAGGGATCTCAGCTGGTGGTGCTGGATCCTGATCACCCTCTGATG | |
| | GTCCGATTCCAAGCCGCTCTGAAGAACTACCTGAACCGGCAGATCGAGAAGCTGAAGCTGGA | |
| | CCTCCAAGAACTGGTGGTGGCCACAAAGCAGAGCAGAGCCCAGAGACAAGAGCTGGGCGTGA | |
| | ACCTGTATGAGGTGCAGCAGCACCTGGTGCATCTGCAGAAACTGCTGGAAAAGAGCCACGAC | |
| | CGGCACGCCATGGCCAGCTCTGAGCGCAGACAGAAAGAGGAAGAACTGCAGGCTGCCCGGGC | |
| | TCTGTACACCAAAACATGTGCCGCCGCCAACGAGGAACGGAAGAAACTGGCTGCCCTGCAGA | |
| | CCGAGATGGAAAACCTGGCTCTGCACCTGTTCTACATGCAGAACATCGACCAGGACATGCGG | |
| | GACGACATCAGAGTGATGACCCAGGTGGTCAAGAAGGCCGAGACAGAGAGAATCCGGGCCGA | |
| | GATCGAGAAGAAGAAACAGGACCTGTACGTGGACCAGCTGACCACAAGAGCCCAGCAGCTGG | |
| | AAGAGGACATCGCCCTGTTCGAGGCCCAGTATCTGGCCCAGGCTGAGGACACCAGAATCCTG | |
| | AGAAAGGCCGTGAGCGAGGCCTGCACAGAGATCGATGCCATCAGCGTGGAAAAGCGGCGGAT | |
| | CATGCAGCAGTGGGCCAGCTCTCTGGTGGGCATGAAGCACAGGGACGAAGCCCACAGAGCCG | |
| | TGCTGGAAGCTCTGAGAGGCTGTCAGCACCAGGCCAAGAGCACCGATGGCGAGATCGAGGCC | |
| | TACAAGAAAAGCATCATGAAGGAAGAGGAAAAGAACGAGAAGCTCGCCAGCATCCTGAACAG | |
| | AACCGAAACCGAGGCCACTCTGCTCCAGAAACTGACAACCCAGTGCCTGACCAAACAGGTGG | |
| | CCCTGCAGAGCCAGTTCAACACCTACAGACTGACCCTGCAGGACACCGAGGATGCCCTGTCT | |
| | CAGGATCAGCTGGAACAGATGATCCTGACCGAGGAACTCCAGGCCATCAGACAGGCCATCCA | |
| | GGGCGAACTGGAACTGCGGAGAAAGACCGATGCCGCCATCAGAGAGAAGCTGCAAGAGCACA | |
| | TGACCAGCAACAAGACCACCAAGTACTTCAACCAGCTGATCCTGCGGCTGCAGAAAGAAAAG | |
| | ACCAACATGATGACACACCTGAGCAAGATCAACGGCGACATCGCCCAGACCACACTGGACAT | |
| | CACCCACACCAGCAGCAGACTGGACGCCCACCAGAAAACCCTGGTGGAACTGGACCAGGACG | |
| | TGAAGAAAGTGAACGAGCTGATCACCAACAGCCAGAGCGAGATCAGCAGACGGACCATCCTG | |
| | ATCGAGAGAAAGCAGGGCCTGATCAACTTCCTGAACAAGCAGCTCGAGCGGATGGTGTCTGA | |
| | ACTCGGCGGAGAAGAAGTGGGCCCTCTGGAACTCGAGATCAAGCGGCTGAGCAAGCTGATCG | |
| | ACGAGCACGATGGAAAGGCAGTGCAGGCCCAAGTGACCTGGCTGAGACTGCAGCAAGAGATG | |
| | GTCAAAGTGACCCAAGAGCAAGAGGAACAGCTGGCCTCTCTGGACGCCAGCAAGAAAGAACT | |
| | CCACATCATGGAACAGAAGAAGCTGAGGGTCGAGAGCAAGATCGAGCAAGAGAAGAAAGAGC | |
| | AAAAAGAAATCGAGCACCACATGAAGGACCTGGACAACGACCTGAAAAAGCTGAACATGCTG | |
| | ATGAACAAGAACCGCTGCAGCAGCGAAGAACTCGAGCAGAACAACAGAGTGACCGAGAACGA | |
| | GTTCGTGCGGAGCCTGAAGGCCAGCGAGAGGGAAACCATCAAGATGCAGGACAAGCTGAACC | |
| | AGCTGAGCGAGGAAAAGGCCACACTGCTGAATCAACTGGTGGAAGCCGAGCACCAGATCATG | |
| | CTGTGGGAGAAGAAAATCCAGCTGGCCAAAGAAATGCGGAGCAGCGTGGACTCTGAGATCGG | |
| | CCAGACCGAAATCAGAGCCATGAAGGGCGAGATCCACCGGATGAAAGTGCGGCTGGGACAGC | |
| | TGCTGAAACAACAAGAGAAAATGATCCGCGCCATGGAACTGGCCGTGGCCAGACGGGAAACA | |
| | GTGACCACTCAAGCCGAGGGACAGAGAAAGATGGACCGGAAGGCCCTGACCAGGACCGACTT | |
| | CCACCACAAACAGCTCGAACTGCGGCGGAAGATCAGGGATGTGCGGAAGGCCACCGATGAGT | |
| | GCACCAAGACAGTGCTGGAACTGGAAGAGACACAGCGGAACGTGAGCAGCAGCCTGCTCGAG | |
| | AAGCAAGAAAAGCTGAGCGTGATCCAGGCCGACTTCGACACCCTGGAAGCTGACCTGACAAG | |
| | ACTGGGAGCCCTGAAGAGGCAGAACCTGAGCGAAATCGTGGCACTGCAGACCCGGCTGAAAC | |
| | ATCTGCAGGCAGTGAAAGAGGGGCGCTACGTGTTCCTGTTCCGGAGCAAGCAGAGCCTGGTG | |
| | CTGGAGAGACAGCGGCTGGACAAGCGGCTGGCCCTGATCGCCACAATCCTGGACAGAGTGCG | |
| | CGACGAGTACCCTCAGTTCCAAGAGGCCCTGCACAAGGTGAGCCAGATGATCGCCAACAAGC | |
| | TGGAAAGCCCCGGACCCAGCGGAAGCGGCTACCCATACGATGTTCCTGACTATGCGTGAGAA | |
| | TTCtgcagAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAA | |
| | AAAATTCG | |
| SEQ ID | ATGTTCAGAATCGGCAGACGGCAGCTGTGGAAGCACAGCGTGACCAGAGTGCTGACCCAGCG | DNAH5 |
| NO: 61 | GCTGAAGGGCGAGAAAGAGGCCAAGAGAGCCCTGCTGGACGCCCGGCACAAcTACCTGTTCG | Altered |
| | CCATCGTGGCCAGCTGCCTGGACCTGAACAAGACCGAGGTGGAAGACGCCATCCTGGAAGGC | Nucleotide |
| | AACCAGATCGAGCGGATCGACCAGCTGTTCGCCGTGGGCGGACTGCGGCACCTGATGTTCTA | Usage 1 |
| | CTACCAAGACGTGGAAGAGGCCGAGACAGGCCAGCTGGGAAGCCTGGGCGGAGTGAACCTGG | |
| | TGAGCGGCAAGATCAAGAAACCCAAGGTGTTCGTGACCGAGGGCAACGACGTGGCCCTGACA | |
| | GGCGTGTGCGTGTTCTTCATCAGAACCGACCCCAGCAAGGCCATCACCCCCGACAACATCCA | |
| | CCAGGAAGTGAGCTTCAACATGCTGGACGCCGCCGACGGCGGCCTGCTGAACAGCGTGCGGA | |
| | GACTGCTGAGCGACATCTTCATCCCCGCCCTGAGAGCCACAAGCCACGGCTGGGGAGAGCTG | |
| | GAAGGACTGCAGGACGCCGCCAACATCCGGCAGGAATTCCTGAGCAGCCTGGAAGGATTCGT | |
| | GAACGTGCTGAGCGGCGCCCAGGAAAGCCTGAAAGAAAAAGTGAACCTGCGGAAGTGCGACA | |
| | TCCTGGAACTGAAAACCCTGAAAGAGCCCACCGACTACCTGACCCTGGCCAACAACCCCGAG | |
| | ACACTGGGCAAGATCGAGGACTGCATGAAAGTGTGGATCAAGCAGACCGAACAGGTGCTGGC | |
| | CGAGAACAACCAGCTGCTGAAAGAAGCCGACGACGTGGGCCCAAGAGCCGAGCTGGAACACT | |
| | GGAAGAAGCGGCTGAGCAAGTTCAACTACCTGCTGGAACAGCTGAAGAGCCCCGACGTGAAG | |
| | GCCGTGCTGGCCGTGCTGGCAGCCGCCAAGAGCAAACTGCTGAAAACCTGGCGCGAGATGGA | |
| | CATCCGGATCACCGACGCCACCAACGAGGCCAAGGACAACGTGAAGTACCTGTACACCCTGG | |
| | AAAAGTGCTGCGACCCCCTGTACAGCAGCGACCCCCTGAGCATGATGGACGCCATCCCCACC | |
| | CTGATCAACGCCATCAAGATGATCTACAGCATCAGCCACTACTACAACACCAGCGAGAAGAT | |
| | CACCAGCCTGTTCGTGAAAGTGACCAACCAGATCATCAGCGCCTGCAAGGCCTACATCACCA | |
| | ACAACGGCACCGCCAGCATCTGGAACCAGCCCCAGGACGTGGTGGAAGAGAAGATCCTGAGC | |
| | GCCATCAAGCTGAAGCAGGAATACCAGCTGTGCTTCCACAAGACCAAGCAGAAGCTGAAACA | |
| | GAACCCCAACGCCAAGCAGTTCGACTTCAGCGAGATGTACATCTTCGGCAAGTTCGAGACAT | |
| | TCCACCGGCGGCTGGCCAAGATCATCGACATCTTCACCACCCTGAAAACATACAGCGTGCTG | |
| | CAGGACAGCACCATCGAGGGCCTGGAAGACATGGCCACCAAGTACCAGGGCATCGTGGCCAC | |
| | CATCAAGAAGAAAGAGTACAACTTCCTGGACCAGCGCAAGATGGACTTCGACCAGGACTACG | |
| | AGGAATTCTGCAAGCAGACAAACGACCTGCACAACGAGCTGCGCAAGTTCATGGACGTGACC | |
| | TTCGCCAAGATCCAGAACACCAACCAGGCCCTGCGGATGCTGAAGAAGTTCGAGAGACTGAA | |
| | CATCCCCAACCTGGGCATCGACGACAAGTACCAGCTGATCCTGGAAAACTACGGCGCCGACA | |
| | TCGACATGATCAGCAAGCTGTACACAAAGCAGAAGTACGACCCCCCCCTGGCCCGGAACCAG | |
| | CCCCCCATCGCCGGCAAAATCCTGTGGGCCAGACAGCTGTTCCACCGGATCCAGCAGCCCAT | |
| | GCAGCTGTTCCAGCAGCACCCCGCCGTGCTGAGCACAGCCGAGGCCAAACCCATCATCCGGA | |
| | GCTACAACCGGATGGCCAAGGTGCTGCTGGAATTCGAGGTGCTGTTCCACCGGGCCTGGCTG | |
| | CGGCAGATCGAAGAGATCCACGTGGGACTGGAAGCCAGCCTGCTCGTGAAGGCCCCCGGAAC | |
| | CGGCGAGCTGTTCGTGAACTTCGACCCCCAGATCCTGATCCTGTTCCGGGAAACCGAGTGCA | |
| | TGGCCCAGATGGGGCTGGAAGTGAGCCCCCTGGCCACCAGCCTGTTCCAGAAGCGGGACCGG | |
| | TACAAGCGGAACTTCAGCAACATGAAGATGATGCTGGCCGAGTACCAGCGCGTGAAGAGCAA | |
| | GATCCCCGCCGCCATCGAGCAGCTGATCGTGCCCCACCTGGCCAAAGTGGACGAGGCCCTGC | |
| | AGCCAGGACTGGCCGCCCTGACATGGACCAGCCTGAACATCGAGGCCTACCTGGAAAACACA | |
| | TTCGCCAAAATCAAGGACCTGGAACTGCTGCTGGACCGCGTGAACGACCTGATCGAGTTCCG | |
| | GATCGACGCCATCCTGGAAGAGATGAGCAGCACCCCCCTGTGCCAGCTGCCCCAGGAAGAAC | |
| | CCCTGACCTGCGAAGAGTTCCTGCAGATGACCAAGGACCTGTGCGTGAACGGCGCCCAGATC | |
| | CTGCACTTCAAGAGCAGCCTGGTGGAAGAAGCCGTGAACGAGCTCGTGAACATGCTGCTGGA | |
| | CGTGGAAGTGCTGAGCGAGGAAGAGAGCGAGAAGATCAGCAACGAGAACAGCGTGAACTACA | |
| | AGAACGAGAGCAGCGCCAAGCGGGAAGAGGGCAACTTCGACACCCTGACCAGCAGCATCAAC | |
| | GCCAGAGCCAACGCCCTGCTGCTGACCACCGTGACCCGGAAGAAAAAAGAAACCGAGATGCT | |
| | GGGCGAAGAGGCCAGAGAGCTGCTGAGCCACTTCAACCACCAGAACATGGACGCCCTGCTGA | |
| | AAGTGACACGGAACACCCTGGAAGCCATCCGGAAGCGGATCCACAGCAGCCACACCATCAAC | |
| | TTCCGGGACAGCAACAGCGCCAGCAACATGAAGCAGAACAGCCTGCCCATCTTCCGGGCCAG | |
| | CGTGACACTGGCCATCCCCAACATCGTGATGGCCCCCGCCCTGGAAGACGTGCAGCAGACAC | |
| | TGAACAAGGCCGTGGAATGCATCATCAGCGTGCCCAAGGGCGTGCGGCAGTGGAGCAGCGAA | |
| | CTGCTGAGCAAGAAGAAGATCCAGGAACGGAAAATGGCCGCCCTGCAGAGCAACGAGGACAG | |
| | CGACAGCGACGTGGAAATGGGCGAGAACGAGCTGCAGGACACACTGGAAATCGCCAGCGTGA | |
| | ACCTGCCCATCCCCGTGCAGACCAAGAACTACTACAAGAACGTGAGCGAAAACAAAGAAATC | |
| | GTGAAGCTGGTGAGCGTGCTGAGCACCATCATCAACAGCACCAAGAAAGAAGTGATCACCAG | |
| | CATGGACTGCTTCAAGCGGTACAACCACATCTGGCAGAAGGGCAAAGAAGAGGCCATCAAGA | |
| | CCTTCATCACCCAGAGCCCCCTGCTGAGCGAGTTCGAGAGCCAGATCCTGTACTTCCAGAAC | |
| | CTGGAACAGGAAATCAACGCCGAGCCCGAGTACGTGTGCGTGGGCAGCATCGCCCTGTACAC | |
| | CGCCGACCTGAAGTTCGCCCTGACCGCCGAGACAAAGGCCTGGATGGTCGTGATCGGCCGGC | |
| | ACTGCAACAAAAAGTACAGAAGCGAGATGGAAAACATCTTCATGCTGATCGAGGAATTCAAC | |
| | AAGAAACTGAACCGGCCCATCAAGGACCTGGACGACATCAGAATCGCCATGGCCGCACTGAA | |
| | AGAGATCAGAGAGGAACAGATCAGCATCGACTTCCAAGTGGGCCCCATCGAGGAAAGCTACG | |
| | CCCTGCTGAACAGATACGGACTGCTGATCGCCCGGGAAGAGATCGACAAGGTGGACACCCTG | |
| | CACTACGCCTGGGAGAAGCTGCTGGCCAGAGCCGGCGAGGTGCAGAACAAACTGGTGAGCCT | |
| | GCAGCCCAGCTTCAAGAAAGAACTGATCAGCGCCGTGGAAGTGTTCCTGCAGGACTGCCACC | |
| | AGTTCTACCTGGACTACGACCTGAACGGCCCCATGGCCAGCGGCCTGAAACCCCAGGAAGCC | |
| | AGCGACCGGCTGATCATGTTCCAGAACCAGTTCGACAACATCTACCGGAAGTACATCACCTA | |
| | CACAGGCGGCGAGGAACTGTTCGGCCTGCCCGCCACACAGTACCCCCAGCTGCTGGAAATCA | |
| | AGAAGCAGCTGAACCTGCTGCAGAAGATCTACACCCTGTACAACAGCGTGATCGAGACAGTG | |
| | AACAGCTACTACGACATCCTGTGGAGCGAAGTGAACATCGAGAAGATCAACAACGAACTGCT | |
| | GGAATTCCAGAACCGGTGCCGGAAGCTGCCCAGAGCACTGAAGGACTGGCAGGCCTTCCTGG | |
| | ACCTGAAGAAAATCATCGACGACTTCAGCGAGTGCTGCCCCCTGCTGGAGTACATGGCCAGC | |
| | AAGGCCATGATGGAACGGCACTGGGAGAGAATCACCACACTGACCGGCCACAGCCTGGACGT | |
| | GGGCAACGAGAGCTTCAAGCTGCGGAACATCATGGAAGCCCCACTGCTGAAGTACAAAGAGG | |
| | AAATCGAGGACATCTGCATCAGCGCCGTGAAAGAGCGGGACATCGAGCAGAAACTGAAACAA | |
| | GTGATCAACGAGTGGGACAACAAGACCTTCACCTTCGGCAGCTTCAAGACCAGAGGCGAGCT | |
| | GCTGCTGCGGGGCGACAGCACCAGCGAGATCATCGCCAACATGGAAGACAGCCTGATGCTGC | |
| | TGGGCAGCCTGCTGAGCAACCGGTACAACATGCCCTTCAAGGCCCAGATCCAGAAATGGGTG | |
| | CAGTACCTGAGCAACAGCACCGACATCATCGAGAGCTGGATGACCGTGCAGAACCTGTGGAT | |
| | CTACCTGGAAGCCGTGTTCGTGGGCGGCGACATCGCCAAGCAGCTGCCCAAAGAGGCCAAGC | |
| | GGTTCAGCAACATCGACAAGAGCTGGGTCAAGATCATGACCAGAGCCCACGAGGTGCCCAGC | |
| | GTGGTGCAGTGCTGCGTGGGCGACGAAACACTGGGACAGCTGCTGCCCCACCTGCTGGACCA | |
| | GCTGGAAATCTGCCAGAAGAGCCTGACCGGCTACCTGGAAAAGAAACGGCTGTGCTTCCCCC | |
| | GGTTCTTCTTCGTGAGCGACCCCGCCCTGCTGGAAATCCTGGGCCAGGCCAGCGACAGCCAC | |
| | ACAATCCAGGCCCACCTGCTGAACGTGTTCGACAACATCAAGAGCGTGAAGTTCCACGAGAA | |
| | AATCTACGACCGGATCCTGAGCATCAGCAGCCAGGAAGGCGAGACAATCGAGCTGGACAAGC | |
| | CCGTGATGGCCGAGGGAAACGTGGAAGTGTGGCTGAACAGCCTGCTGGAAGAGAGCCAGAGC | |
| | AGCCTGCACCTCGTGATCAGACAGGCCGCCGCCAACATCCAGGAAACCGGCTTCCAGCTGAC | |
| | CGAGTTCCTGAGCAGCTTCCCAGCACAAGTGGGACTGCTGGGCATCCAGATGATCTGGACCA | |
| | GAGACAGCGAAGAGGCCCTGAGAAACGCCAAGTTCGACAAGAAAATCATGCAGAAAACAAAC | |
| | CAGGCATTCCTGGAACTGCTGAACACCCTGATCGACGTGACCACCCGGGACCTGAGCAGCAC | |
| | CGAGAGAGTGAAGTACGAGACACTGATCACCATCCACGTGCACCAGCGGGACATCTTCGACG | |
| | ACCTGTGCCACATGCACATCAAGAGCCCCATGGACTTCGAGTGGCTGAAGCAGTGCAGGTTC | |
| | TACTTCAACGAGGACAGCGACAAGATGATGATCCACATCACCGACGTGGCCTTCATCTACCA | |
| | GAACGAGTTCCTGGGCTGCACCGACCGCCTCGTGATCACCCCCCTGACCGACCGGTGCTACA | |
| | TCACACTGGCCCAGGCACTGGGCATGAGCATGGGAGGCGCACCAGCAGGACCCGCCGGCACA | |
| | GGCAAGACCGAAACCACCAAGGACATGGGACGCTGCCTGGGCAAATACGTGGTGGTGTTCAA | |
| | CTGCAGCGACCAGATGGACTTCCGGGGCCTGGGCCGGATCTTCAAGGGCCTGGCACAGAGCG | |
| | GAAGCTGGGGCTGCTTCGACGAGTTCAACAGAATCGACCTGCCCGTGCTGAGCGTGGCCGCA | |
| | CAGCAGATCAGCATCATCCTGACATGCAAAAAAGAGCACAAGAAGAGCTTCATCTTCACCGA | |
| | CGGCGACAACGTGACCATGAACCCCGAGTTCGGCCTGTTCCTGACAATGAACCCCGGCTACG | |
| | CCGGACGGCAGGAACTGCCCGAGAACCTGAAGATCAACTTCCGGAGCGTGGCCATGATGGTG | |
| | CCCGACCGGCAGATCATCATCAGAGTGAAACTGGCCAGCTGCGGCTTCATCGACAACGTGGT | |
| | GCTGGCCCGGAAGTTCTTCACACTGTACAAGCTGTGCGAAGAACAGCTGAGCAAACAGGTGC | |
| | ACTACGACTTCGGCCTGAGGAACATCCTGAGCGTGCTGAGAACCCTGGGAGCCGCCAAGCGG | |
| | GCCAACCCCATGGACACCGAGAGCACAATCGTGATGCGGGTGCTGCGGGACATGAACCTGAG | |
| | CAAGCTGATCGACGAGGACGAGCCCCTGTTCCTGAGCCTGATCGAGGACCTGTTCCCCAACA | |
| | TCCTGCTGGACAAGGCCGGCTACCCCGAACTGGAAGCCGCCATCAGCAGACAGGTGGAAGAG | |
| | GCCGGCCTGATCAACCACCCCCCCTGGAAACTGAAAGTGATCCAGCTGTTCGAGACACAGCG | |
| | CGTGCGGCACGGCATGATGACACTGGGACCCAGCGGAGCCGGCAAGACCACCTGCATCCACA | |
| | CACTGATGCGGGCCATGACCGACTGCGGCAAGCCCCACCGCGAGATGCGGATGAAC | |
| | CCCAAGGCCATCACCGCCCCCCAGATGTTCGGCAGACTGGACGTGGCCACCAACGACTGGAC | |
| | CGACGGCATCTTCAGCACCCTGTGGCGCAAGACCCTGCGGGCCAAGAAGGGCGAGCACATCT | |
| | GGATCATCCTGGACGGCCCCGTGGACGCCATCTGGATCGAGAACCTGAACAGCGTGCTGGAC | |
| | GACAACAAGACACTGACCCTGGCCAACGGCGACCGGATCCCCATGGCCCCCAACTGCAAGAT | |
| | CATCTTCGAGCCCCACAACATCGACAACGCCAGCCCCGCCACCGTGAGCAGAAACGGCATGG | |
| | TGTTCATGAGCAGCAGCATCCTGGACTGGAGCCCCATCCTGGAAGGCTTCCTGAAGAAGCGG | |
| | AGCCCCCAGGAAGCCGAGATCCTGAGACAGCTGTACACCGAGAGCTTCCCCGACCTGTACCG | |
| | GTTCTGCATCCAGAACCTGGAGTACAAGATGGAAGTGCTGGAAGCCTTCGTGATCACCCAGA | |
| | GCATCAACATGCTGCAGGGCCTGATCCCCCTGAAAGAACAGGGCGGAGAAGTGAGCCAGGCC | |
| | CACCTGGGCAGACTGTTCGTGTTCGCCCTGCTGTGGAGCGCCGGCGCCGCCCTGGAACTGGA | |
| | CGGAAGGCGGAGACTGGAACTGTGGCTGCGGAGCAGACCCACCGGCACCCTGGAACTGCCCC | |
| | CACCAGCCGGACCCGGCGACACCGCCTTCGACTACTACGTGGCCCCCGACGGCACCTGGACC | |
| | CACTGGAACACCCGGACCCAGGAATACCTGTACCCCAGCGACACCACCCCCGAGTACGGCAG | |
| | CATCCTGGTGCCCAACGTGGACAACGTGCGGACCGACTTCCTGATCCAGACAATCGCCAAGC | |
| | AGGGAAAGGCCGTGCTGCTGATCGGCGAGCAGGGCACAGCCAAGACCGTGATCATCAAGGGC | |
| | TTCATGAGCAAGTACGACCCCGAGTGCCACATGATCAAGAGCCTGAACTTCAGCAGCGCCAC | |
| | CACCCCACTGATGTTCCAGCGGACCATCGAGAGCTACGTGGACAAGCGGATGGGCACCACCT | |
| | ACGGCCCCCCAGCCGGCAAGAAAATGACCGTGTTCATCGACGACGTGAACATGCCCATCATC | |
| | AACGAGTGGGGCGACCAAGTGACCAACGAGATCGTGCGGCAGCTGATGGAACAGAACGGCTT | |
| | CTACAACCTGGAAAAGCCCGGCGAGTTCACCAGCATCGTGGACATCCAGTTCCTGGCCGCCA | |
| | TGATCCACCCCGGCGGCGGAAGAAACGACATCCCCCAGCGGCTGAAGCGGCAGTTCAGCATC | |
| | TTCAACTGCACCCTGCCCAGCGAGGCCAGCGTGGACAAGATCTTCGGCGTGATCGGCGTGGG | |
| | CCACTACTGCACCCAGAGAGGCTTCAGCGAGGAAGTGCGGGACAGCGTGACCAAGCTGGTGC | |
| | CCCTGACAAGACGGCTGTGGCAGATGACCAAGATCAAGATGCTGCCCACCCCCGCCAAGTTC | |
| | CACTACGTGTTCAACCTGCGGGACCTGAGCAGAGTGTGGCAGGGAATGCTGAACACCACCAG | |
| | CGAAGTGATCAAAGAGCCCAACGACCTGCTGAAGCTGTGGAAGCACGAGTGCAAGAGAGTGA | |
| | TCGCCGACCGGTTCACCGTGAGCAGCGACGTGACATGGTTCGACAAGGCCCTGGTGAGCCTG | |
| | GTGGAAGAGGAATTCGGCGAAGAGAAGAAACTGCTGGTGGACTGCGGCATCGACACCTACTT | |
| | CGTGGACTTCCTGCGCGACGCCCCCGAAGCCGCCGGCGAGACAAGCGAAGAGGCCGACGCCG | |
| | AGACACCCAAGATCTACGAGCCCATCGAGAGCTTCAGCCACCTGAAAGAAAGGCTGAACATG | |
| | TTCCTGCAGCTGTACAACGAGAGCATCCGGGGAGCCGGCATGGACATGGTGTTCTTCGCCGA | |
| | CGCCATGGTGCACCTCGTGAAGATCAGCAGAGTGATCCGGACCCCCCAGGGCAACGCCCTGC | |
| | TCGTGGGAGTGGGAGGCAGCGGCAAGCAGAGCCTGACCAGACTGGCCAGCTTCATCGCCGGC | |
| | TACGTGAGCTTCCAGATCACCCTGACCCGGAGCTACAACACCAGCAACCTGATGGAAGACCT | |
| | GAAGGTGCTGTACCGGACAGCCGGCCAGCAGGGGAAGGGCATCACCTTCATCTTCACCGACA | |
| | ACGAGATCAAGGACGAGAGCTTCCTGGAGTACATGAACAACGTGCTGAGCAGCGGCGAGGTG | |
| | AGCAACCTGTTCGCCCGGGACGAGATCGACGAGATCAACAGCGACCTGGCCAGCGTGATGAA | |
| | GAAAGAATTCCCCCGGTGCCTGCCCACAAACGAGAACCTGCACGACTACTTCATGAGCAGAG | |
| | TGCGGCAGAACCTGCACATCGTGCTGTGCTTCAGCCCCGTGGGCGAGAAGTTCAGAAACCGG | |
| | GCCCTGAAGTTCCCCGCCCTGATCAGCGGCTGCACCATCGACTGGTTCAGCCGGTGGCCCAA | |
| | GGACGCCCTGGTGGCCGTGAGCGAGCACTTCCTGACCAGCTACGACATCGACTGCAGCCTGG | |
| | AAATCAAGAAAGAGGTGGTGCAGTGCATGGGCAGCTTCCAGGACGGCGTGGCCGAGAAATGC | |
| | GTGGACTACTTCCAGCGGTTCCGGCGGAGCACCCACGTGACCCCCAAGAGCTACCTGAGCTT | |
| | CATCCAGGGCTACAAGTTCATCTACGGCGAGAAGCACGTGGAAGTGCGCACACTGGCCAACC | |
| | GGATGAACACCGGCCTGGAAAAACTGAAAGAGGCCAGCGAGAGCGTGGCCGCCCTGAGCAAA | |
| | GAACTGGAAGCCAAAGAAAAAGAACTGCAGGTGGCCAACGACAAGGCCGACATGGTGCTGAA | |
| | AGAAGTGACCATGAAGGCCCAGGCCGCCGAGAAAGTGAAAGCCGAGGTGCAGAAAGTGAAGG | |
| | ACCGGGCCCAGGCCATCGTGGACAGCATCAGCAAGGACAAGGCCATCGCCGAGGAAAAGCTG | |
| | GAAGCAGCCAAGCCCGCCCTGGAAGAGGCAGAAGCCGCCCTGCAGACCATCCGGCCCAGCGA | |
| | CATCGCCACAGTGCGGACCCTGGGAAGGCCCCCCCACCTGATCATGCGGATCATGGACTGCG | |
| | TGCTGCTGCTGTTCCAGAGAAAGGTGAGCGCCGTGAAGATCGACCTGGAAAAAAGCTGCACC | |
| | ATGCCCAGCTGGCAGGAAAGCCTGAAGCTGATGACCGCCGGCAACTTCCTGCAGAACCTGCA | |
| | GCAGTTCCCCAAGGACACCATCAACGAGGAAGTGATCGAGTTCCTGAGCCCCTACTTCGAGA | |
| | TGCCCGACTACAACATCGAAACCGCCAAACGCGTGTGCGGCAACGTGGCCGGACTGTGCAGC | |
| | TGGACCAAGGCCATGGCCAGCTTCTTCAGCATCAACAAAGAGGTGCTGCCCCTGAAGGCCAA | |
| | CCTGGTGGTGCAGGAAAACCGGCACCTGCTGGCCATGCAGGACCTGCAGAAAGCCCAGGCCG | |
| | AGCTGGACGACAAGCAGGCCGAGCTGGACGTGGTGCAGGCCGAGTACGAGCAGGCCATGACC | |
| | GAGAAGCAGACCCTGCTGGAAGACGCAGAGCGGTGCAGACACAAGATGCAGACCGCCAGCAC | |
| | CCTGATCAGCGGACTGGCCGGCGAAAAAGAGCGGTGGACCGAGCAGAGCCAGGAATTCGCCG | |
| | CCCAGACCAAGCGGCTCGTGGGAGACGTGCTGCTGGCCACCGCCTTCCTGAGCTACAGCGGC | |
| | CCCTTCAACCAGGAATTCAGGGACCTGCTGCTGAACGACTGGCGGAAAGAGATGAAGGCCAG | |
| | AAAGATCCCCTTCGGCAAGAACCTGAACCTGAGCGAGATGCTGATCGACGCCCCCACCATCA | |
| | GCGAGTGGAACCTGCAGGGACTGCCCAACGACGACCTGAGCATCCAGAACGGAATCATCGTG | |
| | ACCAAAGCCAGCAGATACCCCCTGCTGATCGACCCCCAGACACAGGGCAAGATCTGGATCAA | |
| | GAACAAAGAGAGCCGGAACGAGCTGCAGATCACCAGCCTGAACCACAAGTACTTCCGGAACC | |
| | ACCTGGAAGACAGCCTGAGCCTGGGCAGGCCACTGCTGATCGAGGACGTGGGCGAGGAACTG | |
| | GACCCAGCCCTGGACAACGTGCTGGAACGGAACTTCATCAAGACCGGCAGCACCTTCAAAGT | |
| | GAAAGTGGGCGACAAAGAAGTGGACGTGCTGGACGGCTTCCGGCTGTACATCACCACCAAGC | |
| | TGCCCAACCCCGCCTACACCCCCGAGATCAGCGCCCGGACCAGCATCATCGACTTCACCGTG | |
| | ACAATGAAGGGACTGGAAGACCAGCTGCTGGGACGCGTGATCCTGACAGAGAAGCAGGAACT | |
| | GGAAAAAGAACGGACCCACCTGATGGAAGACGTGACCGCCAACAAGCGGCGGATGAAGGAAC | |
| | TGGAAGACAACCTGCTGTACAGGCTGACCAGCACCCAGGGCAGCCTGGTGGAAGACGAGAGC | |
| | CTGATCGTGGTGCTGAGCAACACCAAGCGGACCGCAGAGGAAGTGACCCAGAAGCTGGAAAT | |
| | CAGCGCCGAGACAGAGGTGCAGATCAACAGCGCCAGAGAAGAGTACCGGCCCGTGGCCACCC | |
| | GGGGAAGCATCCTGTACTTCCTGATCACCGAGATGCGGCTCGTGAACGAGATGTACCAGACC | |
| | AGCCTGCGGCAGTTCCTGGGCCTGTTCGACCTGAGCCTGGCCAGAAGCGTGAAGAGCCCCAT | |
| | CACCAGCAAGAGAATCGCCAACATCATCGAGCACATGACCTACGAGGTGTACAAATACGCCG | |
| | CCAGAGGCCTGTACGAGGAACACAAGTTCCTGTTCACACTGCTGCTGACCCTGAAGATCGAC | |
| | ATCCAGCGGAACAGAGTGAAGCACGAAGAGTTCCTGACACTGATCAAGGGGGGAGCCAGCCT | |
| | GGACCTGAAGGCCTGCCCCCCCAAGCCCAGCAAGTGGATCCTGGACATCACCTGGCTGAACC | |
| | TGGTGGAACTGAGCAAGCTGAGACAGTTCAGCGACGTGCTGGACCAGATCAGCCGCAACGAG | |
| | AAGATGTGGAAGATCTGGTTCGACAAAGAGAACCCCGAGGAAGAACCCCTGCCCAACGCCTA | |
| | CGACAAGAGCCTGGACTGCTTCCGGCGGCTGCTGCTGATCAGAAGCTGGTGCCCCGACCGGA | |
| | CAATCGCCCAGGCCCGCAAGTACATCGTGGACAGCATGGGAGAGAAGTACGCCGAGGGCGTG | |
| | ATCCTGGACCTGGAAAAGACCTGGGAGGAAAGCGACCCCAGAACCCCCCTGATCTGCCTGCT | |
| | GAGCATGGGCAGCGACCCCACCGACAGCATCATCGCCCTGGGCAAGAGACTGAAGATCGAGA | |
| | CAAGATACGTGAGCATGGGCCAGGGCCAGGAAGTGCACGCCAGAAAGCTGCTGCAGCAGACC | |
| | ATGGCCAACGGCGGCTGGGCCCTGCTGCAGAACTGCCACCTGGGGCTGGACTTCATGGACGA | |
| | ACTGATGGACATCATCATCGAGACAGAGCTGGTGCACGACGCCTTCAGACTGTGGATGACCA | |
| | CCGAGGCCCACAAGCAGTTCCCCATCACCCTGCTGCAGATGAGCATCAAGTTCGCCAACGAC | |
| | CCCCCCCAGGGACTGAGAGCCGGCCTGAAGAGAACCTACAGCGGCGTGAGCCAGGACCTGCT | |
| | GGACGTGAGCAGCGGCAGCCAGTGGAAGCCCATGCTGTACGCCGTGGCATTCCTGCACAGCA | |
| | CCGTGCAGGAACGGCGGAAGTTCGGCGCCCTGGGATGGAACATCCCCTACGAGTTCAACCAG | |
| | GCCGACTTCAACGCCACCGTGCAGTTCATCCAGAACCACCTGGACGACATGGACGTGAAGAA | |
| | AGGGGTGAGCTGGACAACCATCCGGTACATGATCGGAGAGATCCAGTACGGCGGCAGAGTGA | |
| | CCGACGACTACGACAAGAGGCTGCTGAACACCTTCGCCAAAGTGTGGTTCAGCGAGAACATG | |
| | TTCGGCCCCGACTTCAGCTTCTACCAGGGCTACAACATCCCCAAGTGCAGCACCGTGGACAA | |
| | CTACCTGCAGTACATCCAGAGCCTGCCCGCCTACGACAGCCCCGAGGTGTTCGGACTGCACC | |
| | CCAACGCCGACATCACCTACCAGAGCAAACTGGCCAAGGACGTGCTGGACACCATCCTGGGC | |
| | ATCCAGCCCAAGGACACCAGCGGCGGAGGCGACGAAACCCGGGAAGCAGTGGTGGCCAGACT | |
| | GGCCGACGACATGCTGGAAAAGCTGCCCCCCGACTACGTGCCCTTCGAAGTGAAAGAACGCC | |
| | TGCAGAAGATGGGCCCCTTCCAGCCCATGAACATCTTCCTGAGGCAGGAAATCGACCGGATG | |
| | CAGCGGGTGCTGAGCCTCGTGCGGAGCACACTGACCGAGCTGAAACTGGCCATCGACGGCAC | |
| | CATCATCATGAGCGAGAACCTGCGGGACGCACTGGACTGCATGTTCGACGCCAGAATCCCCG | |
| | CATGGTGGAAAAAGGCCAGCTGGATCAGCAGCACCCTGGGCTTCTGGTTCACCGAACTGATC | |
| | GAGAGAAACAGCCAGTTCACCAGCTGGGTGTTCAACGGCAGACCCCACTGCTTCTGGATGAC | |
| | CGGCTTCTTCAACCCACAAGGCTTCCTGACAGCAATGCGCCAGGAAATCACCAGAGCCAACA | |
| | AGGGCTGGGCCCTGGACAACATGGTGCTGTGCAACGAAGTGACCAAGTGGATGAAGGACGAC | |
| | ATCAGCGCCCCCCCCACAGAGGGCGTGTACGTGTACGGCCTGTACCTGGAAGGCGCCGGATG | |
| | GGACAAGAGAAACATGAAGCTGATCGAGAGCAAGCCCAAGGTGCTGTTCGAGCTGATGCCCG | |
| | TGATCAGGATCTACGCCGAGAACAACACCCTGAGGGACCCCCGGTTCTACAGCTGCCCCATC | |
| | TACAAGAAACCCGTGCGCACCGACCTGAACTACATCGCCGCCGTGGACCTGAGGACAGCCCA | |
| | GACACCCGAGCACTGGGTGCTGAGAGGCGTGGCACTGCTGTGCGACGTGAAGTGA | |
| SEQ ID | ATGTTCAGAATCGGCAGACGGCAGCTGTGGAAGCACAGCGTGACCAGAGTGCTGACCCAGCG | DNAH5 |
| NO: 62 | GCTGAAGGGCGAGAAAGAGGCCAAGAGAGCCCTGCTGGACGCCCGGCACAATTACCTGTTTG | Altered |
| | CCATCGTGGCCAGCTGCCTGGACCTGAACAAGACCGAGGTGGAAGATGCCATCCTGGAAGGC | Nucleotide |
| | AACCAGATCGAGCGGATCGACCAGCTGTTTGCCGTGGGCGGACTGCGGCACCTGATGTTCTA | Usage 1 |
| | TTATCAAGACGTGGAAGAGGCCGAGACAGGCCAGCTGGGATCTCTGGGCGGAGTGAATCTGG | |
| | TGTCCGGCAAGATCAAGAAACCCAAGGTGTTCGTGACCGAGGGCAACGACGTGGCCCTGACA | |
| | GGCGTGTGCGTGTTCTTCATCAGAACCGACCCCAGCAAGGCCATCACCCCCGACAACATCCA | |
| | CCAGGAAGTGTCCTTCAACATGCTGGATGCCGCCGATGGCGGCCTGCTGAATTCTGTGCGGA | |
| | GACTGCTGAGCGACATCTTCATCCCCGCCCTGAGAGCCACATCTCACGGCTGGGGAGAGCTG | |
| | GAAGGACTGCAGGACGCCGCCAATATCCGGCAGGAATTTCTGAGCAGCCTGGAAGGATTCGT | |
| | GAACGTGCTGTCTGGCGCCCAGGAAAGCCTGAAAGAAAAAGTGAACCTGCGGAAGTGCGATA | |
| | TCCTGGAACTGAAAACCCTGAAAGAGCCCACCGACTACCTGACCCTGGCCAACAACCCTGAG | |
| | ACACTGGGCAAGATCGAGGACTGCATGAAAGTGTGGATCAAGCAGACCGAACAGGTGCTGGC | |
| | CGAGAACAACCAGCTGCTGAAAGAAGCCGACGACGTGGGCCCAAGAGCCGAGCTGGAACACT | |
| | GGAAGAAGCGGCTGAGCAAGTTCAACTACCTGCTGGAACAGCTGAAGTCCCCCGACGTGAAG | |
| | GCCGTGCTGGCTGTGCTGGCAGCCGCCAAGAGCAAACTGCTGAAAACCTGGCGCGAGATGGA | |
| | CATCCGGATCACCGACGCCACCAACGAGGCCAAGGACAACGTGAAGTACCTGTACACCCTGG | |
| | AAAAGTGCTGCGACCCCCTGTACAGCAGCGACCCTCTGAGCATGATGGACGCCATCCCTACC | |
| | CTGATCAACGCCATCAAGATGATCTACAGCATCAGCCACTACTACAACACCAGCGAGAAGAT | |
| | CACCAGCCTGTTCGTGAAAGTGACCAATCAGATCATCAGCGCCTGCAAGGCCTACATCACCA | |
| | ACAACGGCACCGCCAGCATCTGGAACCAGCCCCAGGATGTGGTGGAAGAGAAGATCCTGTCT | |
| | GCCATCAAGCTGAAGCAGGAATACCAGCTGTGTTTTCACAAGACCAAGCAGAAGCTGAAACA | |
| | GAACCCCAACGCCAAGCAGTTCGACTTCAGCGAGATGTATATCTTCGGCAAGTTCGAGACAT | |
| | TCCACCGGCGGCTGGCCAAGATCATCGACATCTTTACCACCCTGAAAACATACAGCGTGCTG | |
| | CAGGACAGCACCATCGAGGGCCTGGAAGATATGGCCACCAAGTACCAGGGCATTGTGGCCAC | |
| | CATCAAGAAGAAAGAGTACAACTTCCTGGACCAGCGCAAGATGGACTTCGACCAGGACTACG | |
| | AGGAATTCTGCAAGCAGACAAACGACCTGCACAACGAGCTGCGCAAGTTTATGGACGTGACC | |
| | TTCGCCAAGATCCAGAACACCAACCAGGCCCTGCGGATGCTGAAGAAGTTTGAGAGACTGAA | |
| | CATCCCCAACCTGGGCATCGACGATAAGTACCAGCTGATCCTGGAAAACTACGGCGCCGACA | |
| | TCGACATGATCAGCAAGCTGTACACAAAGCAGAAGTACGACCCCCCCCTGGCCCGGAATCAG | |
| | CCTCCTATCGCCGGCAAAATCCTGTGGGCTAGACAGCTGTTTCACCGGATCCAGCAGCCCAT | |
| | GCAGCTGTTCCAGCAGCACCCTGCCGTGCTGAGCACAGCCGAGGCCAAACCCATCATCCGGT | |
| | CCTACAACCGGATGGCCAAGGTGCTGCTGGAATTCGAGGTGCTGTTCCACCGGGCCTGGCTG | |
| | CGGCAGATCGAAGAGATTCACGTGGGACTGGAAGCCAGCCTGCTCGTGAAGGCTCCTGGAAC | |
| | CGGCGAGCTGTTTGTGAACTTCGACCCCCAGATCCTGATCCTGTTCCGGGAAACCGAGTGCA | |
| | TGGCCCAGATGGGGCTGGAAGTGTCTCCTCTGGCCACCTCCCTGTTCCAGAAGCGGGACCGG | |
| | TACAAGCGGAACTTCAGCAACATGAAGATGATGCTGGCTGAGTACCAGCGCGTGAAGTCCAA | |
| | GATCCCCGCTGCCATCGAGCAGCTGATCGTGCCTCACCTGGCCAAAGTGGACGAGGCCCTGC | |
| | AGCCAGGACTGGCCGCTCTGACATGGACCAGCCTGAACATCGAGGCCTATCTGGAAAACACA | |
| | TTCGCCAAAATCAAGGATCTGGAACTGCTGCTGGACCGCGTGAACGACCTGATCGAGTTCCG | |
| | GATCGACGCCATTCTGGAAGAGATGTCCAGCACCCCCCTGTGTCAGCTGCCCCAGGAAGAAC | |
| | CCCTGACCTGCGAAGAGTTCCTGCAGATGACCAAGGACCTGTGCGTGAACGGCGCCCAGATT | |
| | CTGCACTTCAAGTCCAGCCTGGTGGAAGAAGCCGTGAACGAGCTCGTGAATATGCTGCTGGA | |
| | TGTGGAAGTGCTGAGCGAGGAAGAGTCCGAGAAGATCTCCAACGAGAACAGCGTGAACTACA | |
| | AGAACGAGTCCAGCGCCAAGCGGGAAGAGGGCAACTTCGACACCCTGACCAGCTCCATCAAT | |
| | GCCAGAGCCAACGCCCTGCTGCTGACCACCGTGACCCGGAAGAAAAAAGAAACCGAGATGCT | |
| | GGGCGAAGAGGCTAGAGAGCTGCTGTCCCACTTCAACCACCAGAACATGGATGCCCTGCTGA | |
| | AAGTGACACGGAATACCCTGGAAGCCATCCGGAAGCGGATCCACAGCAGCCACACCATCAAC | |
| | TTCCGGGACAGCAACAGCGCCAGCAATATGAAGCAGAACAGCCTGCCCATCTTCCGGGCCTC | |
| | CGTGACACTGGCCATCCCCAATATCGTGATGGCCCCTGCTCTGGAAGATGTGCAGCAGACAC | |
| | TGAACAAGGCCGTGGAATGCATCATCTCCGTGCCCAAGGGCGTGCGGCAGTGGTCTAGCGAA | |
| | CTGCTGTCCAAGAAGAAGATCCAGGAACGGAAAATGGCCGCCCTGCAGTCTAACGAGGACAG | |
| | CGACTCCGACGTGGAAATGGGCGAGAATGAGCTGCAGGATACACTGGAAATCGCCTCTGTGA | |
| | ATCTGCCCATCCCCGTGCAGACCAAGAACTACTATAAGAACGTGTCCGAAAACAAAGAAATC | |
| | GTGAAGCTGGTGTCTGTGCTGTCCACCATCATCAACAGCACCAAGAAAGAAGTGATCACCTC | |
| | CATGGACTGCTTCAAGCGGTACAACCACATCTGGCAGAAGGGCAAAGAAGAGGCCATTAAGA | |
| | CCTTCATCACCCAGAGCCCCCTGCTGTCCGAGTTCGAGTCTCAGATCCTGTACTTCCAGAAC | |
| | CTGGAACAGGAAATCAACGCCGAGCCCGAGTACGTGTGTGTGGGCTCTATCGCCCTGTATAC | |
| | CGCCGACCTGAAGTTCGCCCTGACCGCCGAGACAAAGGCCTGGATGGTCGTGATCGGCCGGC | |
| | ACTGCAACAAAAAGTACAGATCCGAGATGGAAAACATCTTTATGCTGATTGAGGAATTCAAC | |
| | AAGAAACTGAACCGGCCCATTAAGGACCTGGACGACATCAGAATCGCCATGGCCGCACTGAA | |
| | AGAGATCAGAGAGGAACAGATCAGCATCGACTTCCAAGTGGGCCCCATCGAGGAAAGCTACG | |
| | CTCTGCTGAACAGATACGGACTGCTGATCGCCCGGGAAGAGATCGACAAGGTGGACACCCTG | |
| | CACTACGCCTGGGAGAAGCTGCTGGCTAGAGCCGGCGAGGTGCAGAACAAACTGGTGTCTCT | |
| | GCAGCCCAGCTTTAAGAAAGAACTGATCTCCGCCGTGGAAGTGTTTCTGCAGGACTGCCACC | |
| | AGTTCTACCTGGACTACGACCTGAACGGCCCCATGGCCTCTGGCCTGAAACCTCAGGAAGCC | |
| | TCCGACCGGCTGATTATGTTTCAGAACCAGTTCGACAATATCTACCGGAAGTACATCACCTA | |
| | CACAGGCGGCGAGGAACTGTTCGGCCTGCCTGCCACACAGTACCCCCAGCTGCTGGAAATCA | |
| | AGAAGCAGCTGAACCTGCTGCAGAAGATCTACACCCTGTACAACTCCGTGATCGAGACAGTG | |
| | AACAGCTACTACGACATCCTGTGGAGCGAAGTGAACATTGAGAAGATTAACAATGAACTGCT | |
| | GGAATTTCAGAACCGGTGCCGGAAGCTGCCCAGAGCACTGAAGGATTGGCAGGCCTTTCTGG | |
| | ATCTGAAGAAAATCATCGACGACTTCTCCGAGTGCTGCCCTCTGCTGGAGTACATGGCCTCC | |
| | AAGGCCATGATGGAACGGCACTGGGAGAGAATCACCACACTGACCGGCCACAGCCTGGACGT | |
| | GGGCAACGAGAGCTTCAAGCTGCGGAACATCATGGAAGCCCCACTGCTGAAGTACAAAGAGG | |
| | AAATCGAGGACATCTGTATCAGCGCCGTGAAAGAGCGGGATATCGAGCAGAAACTGAAACAA | |
| | GTGATCAACGAGTGGGACAACAAGACCTTTACCTTCGGCAGCTTCAAGACCAGAGGCGAGCT | |
| | GCTGCTGCGGGGCGATAGCACCTCTGAGATCATTGCCAACATGGAAGATAGCCTGATGCTGC | |
| | TGGGCTCCCTGCTGAGCAACCGGTATAACATGCCCTTCAAGGCTCAGATTCAGAAATGGGTG | |
| | CAGTACCTGAGCAACTCCACCGACATCATCGAGTCCTGGATGACCGTGCAGAACCTGTGGAT | |
| | CTACCTGGAAGCCGTGTTCGTGGGCGGCGACATTGCCAAGCAGCTGCCCAAAGAGGCTAAGC | |
| | GGTTCTCCAACATCGACAAGAGCTGGGTCAAGATCATGACCAGAGCCCACGAGGTGCCCAGC | |
| | GTGGTGCAGTGCTGTGTGGGCGACGAAACACTGGGACAGCTGCTGCCTCATCTGCTGGACCA | |
| | GCTGGAAATCTGCCAGAAGTCCCTGACCGGCTACCTGGAAAAGAAACGGCTGTGTTTCCCCC | |
| | GGTTCTTCTTCGTGTCCGACCCCGCCCTGCTGGAAATTCTGGGCCAGGCCAGCGACTCACAC | |
| | ACAATTCAGGCCCATCTGCTGAATGTGTTCGATAACATCAAGAGCGTGAAGTTCCACGAGAA | |
| | AATCTACGACCGGATCCTGAGCATCAGCTCCCAGGAAGGCGAGACAATCGAGCTGGACAAGC | |
| | CTGTGATGGCCGAGGGAAACGTGGAAGTGTGGCTGAACAGCCTGCTGGAAGAGTCCCAGAGC | |
| | AGCCTGCACCTCGTGATCAGACAGGCCGCTGCCAACATCCAGGAAACCGGCTTTCAGCTGAC | |
| | CGAGTTCCTGTCCAGCTTCCCAGCACAAGTGGGACTGCTGGGCATCCAGATGATTTGGACCA | |
| | GAGACTCCGAAGAGGCCCTGAGAAACGCCAAGTTCGATAAGAAAATTATGCAGAAAACAAAT | |
| | CAGGCATTTCTGGAACTGCTGAACACCCTGATCGACGTGACCACCCGGGACCTGAGCAGCAC | |
| | CGAGAGAGTGAAGTACGAGACACTGATCACCATCCACGTGCACCAGCGGGACATCTTCGACG | |
| | ACCTGTGCCACATGCACATCAAGTCTCCCATGGATTTCGAGTGGCTGAAGCAGTGCAGGTTC | |
| | TACTTCAACGAGGACTCCGACAAGATGATGATCCACATCACCGATGTGGCCTTTATCTATCA | |
| | GAATGAGTTCCTGGGCTGTACCGATCGCCTCGTGATTACCCCCCTGACCGACCGGTGTTACA | |
| | TCACACTGGCCCAGGCACTGGGCATGTCTATGGGAGGCGCACCAGCAGGACCTGCCGGCACA | |
| | GGCAAGACCGAAACCACCAAGGACATGGGACGCTGCCTGGGCAAATACGTGGTGGTGTTCAA | |
| | CTGCAGCGACCAGATGGATTTCCGGGGCCTGGGCCGGATCTTTAAGGGCCTGGCACAGAGCG | |
| | GAAGCTGGGGCTGCTTCGACGAGTTCAACAGAATCGACCTGCCCGTGCTGTCCGTGGCCGCA | |
| | CAGCAGATCTCCATCATCCTGACATGCAAAAAAGAGCACAAGAAGTCCTTCATCTTCACCGA | |
| | CGGCGACAATGTGACCATGAACCCCGAGTTTGGCCTGTTCCTGACAATGAACCCTGGCTACG | |
| | CCGGACGGCAGGAACTGCCCGAGAACCTGAAGATCAACTTTCGGAGTGTGGCTATGATGGTG | |
| | CCCGACCGGCAGATCATTATCAGAGTGAAACTGGCCTCCTGCGGCTTCATCGACAACGTGGT | |
| | GCTGGCTCGGAAGTTCTTCACACTGTACAAGCTGTGCGAAGAACAGCTGAGTAAACAGGTGC | |
| | ACTACGACTTCGGCCTGAGGAACATCCTGAGCGTGCTGAGAACTCTGGGAGCCGCTAAGCGG | |
| | GCCAACCCCATGGATACCGAGAGCACAATCGTGATGCGGGTGCTGCGGGACATGAACCTGTC | |
| | CAAGCTGATCGATGAGGACGAGCCCCTGTTTCTGTCTCTGATCGAGGATCTGTTTCCCAACA | |
| | TTCTGCTGGATAAGGCCGGCTACCCCGAACTGGAAGCTGCTATCAGCAGACAGGTGGAAGAG | |
| | GCTGGCCTGATCAACCACCCCCCCTGGAAACTGAAAGTGATCCAGCTGTTCGAGACACAGCG | |
| | CGTGCGGCACGGCATGATGACACTGGGACCTAGCGGAGCCGGCAAGACCACCTGTATCCACA | |
| | CACTGATGCGGGCCATGACCGATTGCGGCAAGCCCCACCGCGAGATGCGGATGAAC | |
| | CCCAAGGCCATTACCGCCCCTCAGATGTTCGGCAGACTGGACGTGGCCACCAACGACTGGAC | |
| | CGACGGCATCTTCAGCACCCTGTGGCGCAAGACCCTGCGGGCCAAGAAGGGCGAGCACATCT | |
| | GGATCATCCTGGACGGCCCCGTGGACGCCATCTGGATTGAGAACCTGAACAGCGTGCTGGAC | |
| | GACAACAAGACACTGACCCTGGCCAACGGCGACCGGATCCCCATGGCCCCCAACTGCAAGAT | |
| | CATCTTCGAGCCCCACAACATCGACAACGCCAGCCCTGCCACCGTGTCCAGAAACGGCATGG | |
| | TGTTCATGAGCAGCAGCATCCTGGATTGGAGCCCTATCCTGGAAGGCTTCCTGAAGAAGCGG | |
| | AGCCCCCAGGAAGCCGAGATCCTGAGACAGCTGTACACCGAGAGCTTCCCCGACCTGTACCG | |
| | GTTCTGCATCCAGAATCTGGAGTACAAGATGGAAGTGCTGGAAGCCTTTGTGATCACCCAGA | |
| | GCATCAACATGCTGCAGGGCCTGATCCCCCTGAAAGAACAGGGCGGAGAAGTGTCCCAGGCC | |
| | CACCTGGGCAGACTGTTCGTGTTTGCCCTGCTGTGGAGCGCTGGCGCCGCTCTGGAACTGGA | |
| | TGGAAGGCGGAGACTGGAACTGTGGCTGCGGAGCAGACCTACCGGCACCCTGGAACTGCCTC | |
| | CACCAGCTGGACCTGGCGACACCGCCTTCGATTACTACGTGGCCCCTGACGGCACCTGGACC | |
| | CACTGGAATACCCGGACCCAGGAATACCTGTACCCCAGCGACACCACCCCCGAGTACGGCTC | |
| | TATCCTGGTGCCCAACGTGGACAACGTGCGGACCGACTTCCTGATCCAGACAATCGCCAAGC | |
| | AGGGAAAGGCCGTGCTGCTGATCGGCGAGCAGGGCACAGCCAAGACCGTGATCATCAAGGGC | |
| | TTTATGTCTAAGTACGACCCCGAGTGCCACATGATCAAGAGCCTGAACTTCAGCTCCGCCAC | |
| | CACCCCACTGATGTTCCAGCGGACCATCGAGAGCTATGTGGACAAGCGGATGGGCACCACCT | |
| | ACGGCCCTCCAGCCGGCAAGAAAATGACCGTGTTCATCGACGACGTGAACATGCCCATCATC | |
| | AACGAGTGGGGCGACCAAGTGACCAACGAGATCGTGCGGCAGCTGATGGAACAGAACGGCTT | |
| | CTACAACCTGGAAAAGCCCGGCGAGTTCACCTCTATCGTGGACATCCAGTTTCTGGCCGCCA | |
| | TGATCCACCCTGGCGGCGGAAGAAACGACATCCCCCAGCGGCTGAAGCGGCAGTTCAGCATC | |
| | TTCAACTGCACCCTGCCCAGCGAGGCCAGCGTGGACAAGATCTTTGGCGTGATCGGCGTGGG | |
| | CCACTACTGCACCCAGAGAGGCTTCAGCGAGGAAGTGCGGGACAGCGTGACCAAGCTGGTGC | |
| | CTCTGACAAGACGGCTGTGGCAGATGACCAAGATCAAGATGCTGCCCACCCCCGCCAAGTTC | |
| | CACTACGTGTTCAACCTGCGGGACCTGAGCAGAGTGTGGCAGGGAATGCTGAACACCACCAG | |
| | CGAAGTGATCAAAGAGCCCAACGACCTGCTGAAGCTGTGGAAGCACGAGTGCAAGAGAGTGA | |
| | TCGCCGACCGGTTCACCGTGTCTAGCGACGTGACATGGTTCGACAAGGCCCTGGTGTCCCTG | |
| | GTGGAAGAGGAATTCGGCGAAGAGAAGAAACTGCTGGTGGACTGCGGCATCGATACCTACTT | |
| | CGTGGACTTCCTGCGCGACGCCCCTGAAGCCGCTGGCGAGACAAGTGAAGAGGCCGACGCCG | |
| | AGACACCCAAGATCTACGAGCCCATCGAGTCCTTCAGCCATCTGAAAGAAAGGCTGAATATG | |
| | TTCCTGCAGCTGTATAACGAGTCCATCCGGGGAGCCGGCATGGATATGGTGTTCTTTGCCGA | |
| | CGCCATGGTGCACCTCGTGAAGATCAGCAGAGTGATCCGGACCCCCCAGGGCAACGCTCTGC | |
| | TCGTGGGAGTGGGAGGCTCTGGCAAGCAGAGCCTGACCAGACTGGCCAGCTTTATCGCCGGC | |
| | TACGTGTCCTTCCAGATCACCCTGACCCGGTCCTACAACACCAGCAACCTGATGGAAGATCT | |
| | GAAGGTGCTGTACCGGACAGCCGGCCAGCAGGGGAAGGGCATCACCTTCATCTTCACCGACA | |
| | ATGAGATCAAGGACGAGTCTTTCCTGGAGTATATGAACAATGTGCTGAGCAGCGGCGAGGTG | |
| | TCCAACCTGTTCGCCCGGGACGAGATCGACGAGATTAACAGCGACCTGGCCTCCGTGATGAA | |
| | GAAAGAATTCCCCCGGTGCCTGCCCACAAACGAGAACCTGCACGACTACTTCATGTCCAGAG | |
| | TGCGGCAGAATCTGCACATCGTGCTGTGCTTCAGCCCCGTGGGCGAGAAGTTCAGAAACCGG | |
| | GCCCTGAAGTTCCCCGCCCTGATCAGCGGCTGCACCATCGACTGGTTCAGCCGGTGGCCTAA | |
| | GGATGCCCTGGTGGCCGTGTCCGAGCACTTTCTGACCAGCTACGACATCGACTGCAGCCTGG | |
| | AAATCAAGAAAGAGGTGGTGCAGTGCATGGGCAGCTTCCAGGACGGCGTGGCCGAGAAATGC | |
| | GTGGACTACTTCCAGCGGTTCCGGCGGAGCACCCACGTGACCCCTAAGAGCTACCTGAGCTT | |
| | CATCCAGGGCTACAAGTTCATCTACGGCGAGAAGCACGTGGAAGTGCGCACACTGGCCAACC | |
| | GGATGAACACCGGCCTGGAAAAACTGAAAGAGGCCTCCGAGAGCGTGGCCGCCCTGAGCAAA | |
| | GAACTGGAAGCCAAAGAAAAAGAACTGCAGGTGGCCAACGATAAGGCCGACATGGTGCTGAA | |
| | AGAAGTGACCATGAAGGCCCAGGCCGCCGAGAAAGTGAAAGCCGAGGTGCAGAAAGTGAAGG | |
| | ACCGGGCCCAGGCCATCGTGGACTCCATCAGCAAGGACAAGGCCATTGCCGAGGAAAAGCTG | |
| | GAAGCAGCCAAGCCCGCCCTGGAAGAGGCAGAAGCTGCTCTGCAGACCATCCGGCCCTCCGA | |
| | TATTGCCACAGTGCGGACCCTGGGAAGGCCCCCTCACCTGATCATGCGGATCATGGACTGTG | |
| | TGCTGCTGCTGTTCCAGAGAAAGGTGTCCGCCGTGAAGATCGACCTGGAAAAATCCTGCACC | |
| | ATGCCTAGCTGGCAGGAATCCCTGAAGCTGATGACCGCCGGCAACTTCCTGCAGAACCTGCA | |
| | GCAGTTCCCCAAGGACACCATCAATGAGGAAGTGATCGAGTTCCTGAGCCCCTACTTCGAGA | |
| | TGCCCGACTACAATATCGAAACCGCCAAACGCGTGTGCGGCAACGTGGCCGGACTGTGCTCT | |
| | TGGACCAAGGCTATGGCTAGCTTCTTTAGCATTAACAAAGAGGTGCTGCCTCTGAAGGCCAA | |
| | CCTGGTGGTGCAGGAAAACCGGCATCTGCTGGCCATGCAGGACCTGCAGAAAGCCCAGGCCG | |
| | AGCTGGACGATAAGCAGGCTGAGCTGGATGTGGTGCAGGCCGAGTACGAGCAGGCCATGACC | |
| | GAGAAGCAGACCCTGCTGGAAGATGCAGAGCGGTGCAGACACAAGATGCAGACCGCCAGCAC | |
| | CCTGATCTCTGGACTGGCCGGCGAAAAAGAGCGGTGGACCGAGCAGTCCCAGGAATTCGCCG | |
| | CCCAGACCAAGCGGCTCGTGGGAGATGTGCTGCTGGCCACCGCCTTTCTGAGCTACAGCGGC | |
| | CCCTTCAATCAGGAATTCAGGGACCTGCTGCTGAACGACTGGCGGAAAGAGATGAAGGCCAG | |
| | AAAGATCCCCTTCGGCAAGAATCTGAACCTGAGCGAGATGCTGATCGACGCCCCCACCATCT | |
| | CCGAGTGGAATCTGCAGGGACTGCCCAACGATGACCTGTCCATCCAGAACGGAATCATCGTG | |
| | ACCAAAGCCTCCAGATACCCCCTGCTGATTGACCCCCAGACACAGGGCAAGATTTGGATCAA | |
| | GAACAAAGAGAGCCGGAACGAGCTGCAGATCACCAGCCTGAACCACAAGTACTTCCGGAACC | |
| | ACCTGGAAGATAGCCTGAGCCTGGGCAGGCCACTGCTGATCGAGGATGTGGGCGAGGAACTG | |
| | GACCCAGCCCTGGATAACGTGCTGGAACGGAACTTCATCAAGACCGGCTCCACCTTCAAAGT | |
| | GAAAGTGGGCGACAAAGAAGTGGACGTGCTGGATGGCTTCCGGCTGTACATCACCACCAAGC | |
| | TGCCTAACCCCGCCTACACCCCTGAGATCAGCGCCCGGACCAGCATCATCGACTTCACCGTG | |
| | ACAATGAAGGGACTGGAAGATCAGCTGCTGGGACGCGTGATCCTGACAGAGAAGCAGGAACT | |
| | GGAAAAAGAACGGACCCATCTGATGGAAGATGTGACCGCCAACAAGCGGCGGATGAAGGAAC | |
| | TGGAAGATAACCTGCTGTACAGGCTGACCAGCACCCAGGGCAGTCTGGTGGAAGATGAGAGC | |
| | CTGATCGTGGTGCTGTCCAACACCAAGCGGACCGCAGAGGAAGTGACCCAGAAGCTGGAAAT | |
| | CAGCGCCGAGACAGAGGTGCAGATCAACAGCGCCAGAGAAGAGTACCGGCCTGTGGCCACCC | |
| | GGGGATCCATCCTGTACTTTCTGATCACCGAGATGCGGCTCGTGAACGAGATGTACCAGACC | |
| | AGCCTGCGGCAGTTCCTGGGCCTGTTCGATCTGTCCCTGGCCAGAAGCGTGAAGTCCCCCAT | |
| | CACCAGCAAGAGAATCGCCAACATCATCGAGCACATGACCTACGAGGTGTACAAATACGCCG | |
| | CCAGAGGCCTGTACGAGGAACACAAGTTTCTGTTCACACTGCTGCTGACCCTGAAGATCGAT | |
| | ATCCAGCGGAACAGAGTGAAGCACGAAGAGTTTCTGACACTGATCAAGGGGGGAGCCTCCCT | |
| | GGACCTGAAGGCCTGTCCTCCCAAGCCCAGCAAGTGGATCCTGGACATCACCTGGCTGAATC | |
| | TGGTGGAACTGAGCAAGCTGAGACAGTTCTCCGATGTGCTGGACCAGATCAGCCGCAACGAG | |
| | AAGATGTGGAAGATTTGGTTTGACAAAGAGAACCCCGAGGAAGAACCCCTGCCTAACGCCTA | |
| | CGATAAGAGCCTGGACTGCTTCCGGCGGCTGCTGCTGATTAGAAGCTGGTGTCCCGACCGGA | |
| | CAATCGCCCAGGCCCGCAAGTACATCGTGGATAGCATGGGAGAGAAGTACGCCGAGGGCGTG | |
| | ATCCTGGACCTGGAAAAGACCTGGGAGGAAAGCGACCCCAGAACCCCCCTGATCTGCCTGCT | |
| | GAGCATGGGCTCCGACCCCACCGACAGCATTATCGCCCTGGGCAAGAGACTGAAGATTGAGA | |
| | CAAGATACGTGTCCATGGGCCAGGGCCAGGAAGTGCACGCTAGAAAGCTGCTGCAGCAGACT | |
| | ATGGCCAATGGCGGCTGGGCCCTGCTGCAGAATTGTCACCTGGGGCTGGACTTCATGGACGA | |
| | ACTGATGGACATCATCATTGAGACAGAGCTGGTGCACGACGCCTTCAGACTGTGGATGACCA | |
| | CCGAGGCCCATAAGCAGTTTCCCATTACCCTGCTGCAGATGAGCATCAAGTTCGCCAACGAC | |
| | CCCCCTCAGGGACTGAGAGCCGGCCTGAAGAGAACCTACTCCGGCGTGTCACAGGATCTGCT | |
| | GGACGTGTCCTCTGGCAGCCAGTGGAAGCCTATGCTGTACGCCGTGGCATTCCTGCACAGCA | |
| | CCGTGCAGGAACGGCGGAAGTTTGGCGCCCTGGGATGGAACATCCCCTACGAGTTTAACCAG | |
| | GCCGACTTCAACGCCACTGTGCAGTTTATCCAGAACCATCTGGACGACATGGACGTGAAGAA | |
| | AGGGGTGTCCTGGACAACCATCCGGTACATGATCGGAGAGATCCAGTACGGCGGCAGAGTGA | |
| | CCGACGACTACGACAAGAGGCTGCTGAATACCTTCGCCAAAGTGTGGTTCTCCGAGAACATG | |
| | TTTGGCCCCGACTTCAGCTTTTACCAGGGCTATAACATCCCCAAGTGCTCCACCGTGGATAA | |
| | CTACCTGCAGTACATCCAGAGCCTGCCCGCCTACGACAGCCCTGAGGTGTTCGGACTGCACC | |
| | CCAACGCCGATATCACCTACCAGAGCAAACTGGCCAAGGATGTGCTGGATACCATCCTGGGC | |
| | ATCCAGCCCAAGGATACCAGTGGCGGAGGCGACGAAACCCGGGAAGCAGTGGTGGCTAGACT | |
| | GGCCGACGACATGCTGGAAAAGCTGCCCCCCGACTACGTGCCCTTTGAAGTGAAAGAACGCC | |
| | TGCAGAAGATGGGCCCCTTCCAGCCTATGAACATCTTCCTGAGGCAGGAAATCGACCGGATG | |
| | CAGCGGGTGCTGTCTCTCGTGCGGAGCACACTGACCGAGCTGAAACTGGCTATCGACGGCAC | |
| | CATCATCATGAGCGAGAATCTGCGGGATGCACTGGACTGCATGTTCGACGCCAGAATCCCCG | |
| | CATGGTGGAAAAAGGCCAGCTGGATCAGCTCTACCCTGGGCTTCTGGTTCACCGAACTGATC | |
| | GAGAGAAACAGCCAGTTTACCAGCTGGGTGTTCAACGGCAGACCTCACTGCTTCTGGATGAC | |
| | CGGCTTCTTCAATCCACAAGGCTTTCTGACAGCAATGCGCCAGGAAATCACCAGAGCCAACA | |
| | AGGGCTGGGCTCTGGACAATATGGTGCTGTGTAACGAAGTGACTAAGTGGATGAAGGACGAC | |
| | ATCAGCGCCCCTCCCACAGAGGGCGTGTACGTGTACGGCCTGTACCTGGAAGGCGCCGGATG | |
| | GGACAAGAGAAACATGAAGCTGATCGAGAGCAAGCCCAAGGTGCTGTTCGAGCTGATGCCCG | |
| | TGATCAGGATCTATGCCGAGAACAACACCCTGAGGGACCCCCGGTTCTACAGCTGCCCCATC | |
| | TACAAGAAACCCGTGCGCACCGACCTGAACTATATCGCCGCCGTGGACCTGAGGACAGCCCA | |
| | GACACCTGAGCATTGGGTGCTGAGAGGCGTGGCACTGCTGTGCGACGTGAAGTGA | |

The polynucleotide may comprise nucleotide analogues. In some embodiments, the nucleotide analogues replace uridines in a sequence. For example, a sequence using standard nucleotides (A, C, U, T, G) may comprises a uridine at a particular position in a sequence. A sequence may instead have a nucleotide analogue in place of the uridine. The nucleotide analogue may have structure that may still be recognized by the cellular translation machinery such that the polynucleotide comprising a nucleotide analogues may still be translated. The nucleotide analogue may be recognized as synonymous with a standard nucleotide. For example, the nucleotide analogue may be recognized as synonymous with uridine and the resulting translation product is generated as if the nucleotide analogue is a uridine. In some embodiments, at least 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of nucleotides replacing uridine within the polynucleotide are nucleotide analogues. In some embodiments, fewer than 15% of nucleotides within the polynucleotide are nucleotide analogues. In some fewer than 30% of the nucleotides are nucleotide analogues. In other cases, fewer than 27.5%, fewer than 25%, fewer than 22.5%, fewer than 20%, fewer than 17.5%, fewer than 15%, fewer than 12.5%, fewer than 10%, fewer than 7.5%, fewer than 5%, or fewer than 2.5% of the nucleotides are nucleotide analogues.

In some embodiments, the nucleotide analogue is a purine or pyrimidine analogue. In some cases, a polyribonucleotide of the disclosure comprises a modified pyrimidine, such as a modified uridine. A nucleotide analogue may be a pseudouridine (Ψ). A nucleotide analogue may be a methylpseudouridine. A nucleotide analogue may be a 1-methylpseudouridine (m¹Ψ). In some embodiments, the polynucleotide comprises a 1-methylpseudouridine. In some cases a uridine analogue is selected from pseudouridine 1-methylpseudouridine, 2-thiouridine (s²U), 5-methyluridine (m⁵U), 5-methoxyuridine (mo⁵U), 4-thiouridine (s⁴U), 5-bromouridine (Br⁵U), 2′O-methyluridine (U2′m), 2′-amino-2′-deoxyuridine (U2′NH₂), 2′-azido-2′-deoxyuridine (U2′N₃), and 2′-fluoro-2′-deoxyuridine (U2′F).

A polyribonucleotide can have the same or a mixture of different nucleotide analogues or modified nucleotides. The nucleotide analogues or modified nucleotides can have structural changes that are naturally or not naturally occurring in messenger RNA. A mixture of various analogues or modified nucleotides can be used. For example, one or more analogues within a polynucleotide can have natural modifications, while another part has modifications that are not naturally found in mRNA. Additionally, some analogues or modified ribonucleotides can have a base modification, while other modified ribonucleotides have a sugar modification. In the same way, it is possible that all modifications are base modifications or all modifications are sugar modifications or any suitable mixture thereof

A nucleotide analogue or modified nucleotide can be selected from the group comprising pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-me thylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some cases, at least about 5% of the nucleic acid construct(s), a vector(s), engineered polyribonucleotide(s), or compositions includes non-naturally occurring (e.g., modified, analogues, or engineered) uridine, adenosine, guanine, or cytosine, such as the nucleotides described herein. In some cases, 100% of the modified nucleotides in the composition are either 1-methylpseudouridine or pseudouridine. In some cases, at least about 10%, 15%, 20%, 25%, 30%, 35%, 40%, 45%, 50%, 55%, 60%, 65%, 70%, 75%, 80%, 85%, 90%, 95% of the nucleic acid construct(s), a vector(s), engineered polyribonucleotide(s), or compositions includes non-naturally occurring uracil, adenine, guanine, or cytosine. In some cases, at most about 99%, 95%, 90%, 85%, 80%, 75%, 70%, 65%, 60%, 55%, 50%, 45%, 40%, 35%, 30%, 25%, 20%, 15%, 10%, 5%, 1%, of the nucleic acid construct(s), a vector(s), engineered polyribonucleotide(s), or compositions includes non-naturally occurring uracil, adenine, guanine, or cytosine.

The polynucleotides may comprise an open reading frame (ORF) sequence. The ORF sequence may be characterized by a codon usage profile comprising: (1) a total number of codons, (2) a species number of codons (e.g. a total number of different codon types), (3) a number of each (unique) codon, and (4) a (usage) frequency of each codon among all synonymous codons (if present). The codon usage profile may be altered or compared to a corresponding wild type sequence. For example, the frequency or number of particular codons may be reduced or increased compared to a wild type sequence. The change in codon frequency of the polynucleotide may provide benefits over the wild type sequence. For example, the altered codon frequency may result in a less immunogenic polynucleotide. The polynucleotide with an altered codon frequency may result in a polynucleotide that is more quickly expressed or results in a greater amount of expression product. The polynucleotide with an altered codon frequency may have increase stability, such as increased half-life in sera, or may be less susceptible to hydrolysis or other reactions that may result in the degradation of the polynucleotide.

In some instances, the polynucleotide encodes for a polypeptide at a level that is increased by a factor of at least about 1.5 as compared to levels within cells exposed to a composition comprising a corresponding wild type sequence. In some cases, the factor is at least about 1.1, at least about 1.2, at least about 1.3, at least about 1.4, at least about 1.5, at least about 2, at least about 3, at least about 4, at least about 5, at least about 10, at least about 20, at least about 30, at least about 40, at least about 50, at least about 60, at least about 70, at least about 80, at least about 90, or at least about 100. In some cases, the factor is of about 1.1, about 1.2, about 1.3, about 1.4, about 1.5, about 2, about 3, about 4, about 5, about 10, about 20, about 30, about 40, about 50, about 60, about 70, about 80, about 90, or about 100, or a range between any two of the foregoing values.

### Codon Optimizations

In some embodiments, the polynucleotide comprises an altered nucleotide usage as compared to a corresponding wild type sequence. The altered nucleotide usage may also be referred to as a “codon optimized” sequence or be generated by way of “codon optimization”. The codon optimized polynucleotides may comprise

Altered nucleotide usage schemes aiming to reduce the number of more reactive 5′-U(U/A)-3′ dinucleotides within codons as well as across codons of modified mRNAs partially alleviate limitations imposed by the inherent chemical instability of RNA. At the same time, lowering the U-content in RNA transcripts renders them less immunogenic. The present disclosure relates to RNA transcripts comprising altered open reading frames (ORF). For example, the codon optimized or altered nucleotide usage may comprise a substantial reduction of 5′-U(U/A)-3′ dinucleotides within protein coding regions leading to stabilized therapeutic mRNAs. The codon optimized polynucleotide may comprise a codon coding for a particular amino acid to be substituted or replaced of a with a synonymous codon. The codon optimized polynucleotide may encode a same or identical polypeptide as a corresponding wild type polynucleotide, with the polynucleotide comprising a different sequence of polynucleotide than the corresponding wild type. Multiple codons may encode for a same amino acid, however the qualities of a given codon are differ between even those that code for a same amino acid. Because multiple different codons may code for a same amino acid, a particular polynucleotide may encode for a same polypeptide and have advantageous features over another polynucleotide that codes for the same polypeptide. For example, a codon optimized polynucleotides may be transcribed faster, may comprise a higher stability (in vivo or in vitro), may result in increased expression yield or full length or functional polypeptides, or may result in an increase of soluble polypeptide and a decrease in polypeptide aggregates. Without being limited to a specific mechanism, the advantageous features of a codon optimized polynucleotides may be for example, a result of improved protein folding of the expressed product based on ribosomal interactions with the polynucleotides, or may be result of decreased hydrolysis of reactive bonds in solution. For example, the codon optimization may be alter or improve characteristics relating to ribosomal binding sites, Shine-Dalgarno sequences, or ribosomal or translational pausing. The advantageous features may be a result of decreased usage of “rare codons” which may have a lower concentration of cognate tRNAs, allowing for an improved translation reaction. The advantageous features may be a result of decreased usage of “rare codons” which may have a lower concentration of cognate tRNAs, allowing for an improved translation reaction. The advantageous features may be a result of decreasing degradation via enzymatic reaction. For example, hydrolysis of oligonucleotides suggests that the reactivity of the phosphodiester bond linking two ribonucleotides in single-stranded (ss)RNA depends on the nature of those nucleotides. At pH 8.5, dinucleotide cleavage susceptibility when embedded in ssRNA dodecamers may vary by an order of magnitude. Under near physiological conditions, hydrolysis of RNA usually involves an SN2-type attack by the 2′-oxygen nucleophile on the adjacent phosphorus target center on the opposing side of the 5′-oxyanion leaving group, yielding two RNA fragments with 2′,3′-cyclic phosphate and 5′-hydroxyl termini. More reactive scissile phosphodiester bonds may include 5′-UpA-3′ (R₁=U₁, R₂=A) and 5′-CpA-3′ (R₁=C, R₂=A) because the backbone at these steps can most easily adopt the “in-line” conformation that is required for S_{N}2-type nucleophilic attack by the 2′-OH on the adjacent phosphodiester linkage. In addition, interferon-regulated dsRNA-activated antiviral pathways produce 2′-5′ oligoadenylates which bind to ankyrin repeats leading to activation of RNase L endoribonuclease. RNase L cleaves ssRNA efficiently at UA and UU dinucleotides. Lastly, U-rich sequences are potent activators of RNA sensors including Toll-like receptor 7 and 8 and RIG-I making global uridine content reduction a potentially attractive approach to reduce immunogenicity of therapeutic mRNAs.

In some embodiments, the nucleic acid sequence comprises a reduced number or frequency of at least one codon (e.g., at least 2, at least 3, at least 4, at least 5, at least 6, at least 7, at least 8, at least 9, at least 10 codons) selected from the group consisting of GCG, GCA, GCT, TGT, GAT, GAG, TTT, GGG, GGT, CAT, ATA, ATT, AAG, TTG, TTA, CTA, CTT, CTC, AAT, CCG, CCA, CCT, CAG, AGG, CGG, CGA, CGT, CGC, TCG, TCA, TCT, TCC, ACG, ACT, GTA, GTT, GTC, and TAT, as compared to a corresponding wild-type sequence, e.g. SEQ ID NO: 33-39. In some embodiments, the nucleic acid sequence comprises an increased number or frequency of at least one codon (e.g., at least 2, at least 3, at least 4, at least 5, at least 6, at least 7, at least 8, at least 9, at least 10 codons) comprising one or more codons selected from: GCC, TGC, GAC, GAA, TTC, GGA, GGC, CAC, ATC, AAA, CTG, AAC, CCT, CCC, CAA, AGA, AGC, ACA, ACC, GTG, and TAC, as compared to a corresponding wild-type sequence, e.g., SEQ ID NO: 33-39. In some embodiments, the nucleic acid sequence comprises fewer codon types encoding an amino acid as compared to a corresponding wild-type sequence, e.g., SEQ ID NO: 33-39.

TABLE 3
Example wild type sequences

| SEQ # | Sequence | |
|---|---|---|
| SEQ ID | ATGGGTGTGGCTCTGAGGAAATTGACGCAGTGGACTGCTGCCGGACATGGAACTGGAAT | Wild type |
| NO: 33 | CCTCGAAATCACCCCTCTAAATGAAGCGATATTGAAAGAAATTATTGTGTTTGTGGAGA | ARMC4 ORF |
| | GTTTTATCTATAAACATCCTCAAGAGGCAAAATTTGTTTTTGTGGAACCACTTGAATGG | |
| | AACACAAGTTTGGCGCCCTCAGCATTTGAATCAGGTTATGTTGTCAGTGAAACAACAGT | |
| | CAAATCAGAAGAAGTTGATAAAAATGGACAGCCTTTGCTATTTCTCTCTGTACCACAAA | |
| | TTAAAATTAGGAGCTTTGGGCAGCTGTCACGCTTGTTACTTATTGCCAAAACTGGGAAG | |
| | TTGAAGGAAGCCCAAGCATGTGTTGAAGCTAACAGAGACCCCATAGTAAAAATCCTGGG | |
| | CTCTGATTATAATACAATGAAAGAAAACTCAATTGCATTAAATATTCTTGGCAAAATTA | |
| | CCAGAGATGATGATCCTGAAAGTGAAATTAAGATGAAGATTGCTATGCTGCTTAAGCAA | |
| | TTGGATCTGCACCTCCTCAATCATTCTCTAAAACATATTTCATTAGAAATAAGTTTAAG | |
| | TCCCATGACGGTGAAGAAGGATATAGAACTGCTCAAACGTTTCTCAGGAAAAGGAAACC | |
| | AAACAGTCTTGGAATCTATTGAATATACCTCAGATTATGAATTTTCAAATGGATGTCGA | |
| | GCCCCACCGTGGAGACAAATTCGTGGGGAAATTTGTTATGTGCTGGTGAAACCTCACGA | |
| | TGGTGAGACTCTGTGCATTACTTGCAGTGCAGGAGGAGTATTTTTAAATGGTGGCAAAA | |
| | CAGATGATGAAGGGGACGTTAATTATGAGAGAAAAGGTTCAATTTATAAAAACCTTGTC | |
| | ACATTTTTAAGAGAAAAATCACCAAAATTTTCAGAAAATATGTCTAAATTGGGAATTAG | |
| | CTTCAGTGAAGACCAGCAAAAGGAAAAGGATCAGCTTGGCAAAGCCCCCAAGAAGGAAG | |
| | AAGCAGCTGCCCTCCGCAAAGACATTTCTGGTTCAGACAAAAGGTCACTGGAGAAGAAC | |
| | CAAATTAATTTTTGGAGGAATCAAATGACCAAGAGATGGGAACCAAGCTTAAACTGGAA | |
| | GACCACTGTTAATTACAAAGGCAAAGGCTCAGCAAAAGAAATCCAAGAGGACAAACACA | |
| | CAGGAAAACTTGAAAAACCAAGACCATCTGTTTCACACGGAAGAGCACAATTACTTCGG | |
| | AAGAGTGCTGAAAAGATTGAGGAAACTGTTAGCGATAGCTCCTCAGAAAGTGAGGAAGA | |
| | TGAAGAACCACCTGACCATCGTCAGGAAGCAAGTGCAGATTTGCCATCAGAATATTGGC | |
| | AAATTCAGAAGCTGGTGAAATATTTAAAGGGAGGAAATCAAACAGCTACAGTGATTGCG | |
| | TTGTGTTCAATGAGGGATTTCAGCTTAGCTCAAGAAACCTGCCAGTTGGCCATCAGAGA | |
| | TGTTGGAGGCCTGGAAGTGCTGATAAATTTGCTTGAAACCGATGAAGTCAAATGTAAGA | |
| | TTGGTTCATTAAAAATACTGAAGGAAATCAGTCATAATCCTCAAATCAGACAGAATATT | |
| | GTTGACCTTGGGGGCTTACCAATTATGGTGAATATACTTGATTCTCCACACAAGAGTCT | |
| | AAAATGTTTGGCAGCCGAGACTATCGCGAATGTTGCCAAGTTTAAAAGAGCACGGCGGG | |
| | TGGTGAGGCAGCACGGGGGTATCACCAAACTGGTTGCTCTACTAGACTGTGCACATGAT | |
| | TCCACAAAACCTGCCCAATCGAGTCTGTATGAGGCCAGAGACGTGGAAGTGGCTCGCTG | |
| | TGGGGCACTGGCCCTGTGGAGCTGCAGTAAGAGTCATACGAATAAAGAAGCCATCCGCA | |
| | AAGCTGGGGGCATTCCTCTGTTGGCTCGGCTGCTGAAGACTTCTCATGAAAACATGCTA | |
| | ATTCCAGTGGTGGGGACATTGCAAGAGTGTGCATCAGAGGAAAACTACCGGGCTGCAAT | |
| | CAAAGCAGAAAGGATCATTGAAAACCTTGTCAAGAACCTAAATAGTGAGAATGAGCAGC | |
| | TGCAGGAGCACTGCGCCATGGCCATTTACCAGTGTGCTGAAGATAAGGAAACCCGGGAC | |
| | CTCGTTAGGCTGCACGGAGGACTTAAGCCCTTGGCCAGTCTACTCAATAACACTGACAA | |
| | TAAAGAGCGGTTAGCTGCTGTCACAGGGGCTATATGGAAATGTTCCATCAGCAAAGAGA | |
| | ATGTTACCAAGTTTCGGGAATACAAAGCCATTGAAACCTTGGTGGGACTTCTAACAGAT | |
| | CAGCCTGAAGAAGTACTTGTGAATGTGGTTGGGGCCTTGGGAGAATGCTGCCAAGAACG | |
| | TGAAAACCGAGTCATTGTCCGGAAATGTGGTGGCATTCAACCACTTGTGAACCTCCTTG | |
| | TTGGAATAAACCAAGCTCTTCTTGTGAATGTTACAAAAGCAGTTGGTGCTTGTGCAGTA | |
| | GAACCTGAAAGTATGATGATAATTGATCGCTTAGATGGAGTTCGTTTGTTGTGGTCCCT | |
| | GCTGAAAAATCCTCACCCAGACGTGAAGGCCAGCGCAGCATGGGCACTCTGTCCATGCA | |
| | TCAAAAATGCAAAGGATGCTGGGGAAATGGTTCGTTCCTTTGTTGGTGGTTTGGAACTT | |
| | ATTGTCAATTTACTGAAATCAGATAACAAAGAAGTTCTGGCAAGTGTATGTGCTGCCAT | |
| | TACCAACATAGCAAAAGATCAAGAAAATTTAGCTGTTATCACAGATCATGGAGTTGTTC | |
| | CTTTATTGTCCAAACTGGCAAATACAAATAACAATAAATTGAGACATCATCTAGCAGAA | |
| | GCTATTTCACGTTGCTGTATGTGGGGCAGGAATAGAGTGGCCTTCGGTGAGCACAAAGC | |
| | AGTGGCTCCACTAGTGCGTTATCTGAAATCAAATGACACCAACGTGCATCGGGCGACAG | |
| | CTCAGGCCTTGTACCAACTCTCAGAAGACGCCGATAACTGCATCACCATGCATGAGAAT | |
| | GGTGCAGTAAAGCTTCTACTGGATATGGTTGGGTCCCCTGACCAGGATCTCCAGGAAGC | |
| | TGCAGCTGGTTGTATATCCAATATCCGCAGGCTGGCTCTTGCTACAGAGAAGGCAAGAT | |
| | ACACTTGA | |
| SEQ ID | ATGCACCCTGAGCCCTCGGAGCCTGCGACAGGTGGTGCAGCAGAGCTGGATTGCGCGCA | Wild type |
| NO: 34 | GGAGCCCGGCGTGGAGGAGTCTGCGGGTGACCACGGGAGCGCAGGCCGAGGGGGCTGCA | DNAAF1 |
| | AGGAAGAAATTAATGATCCTAAGGAAATATGTGTGGGTTCTTCTGACACATCCTACCAC | ORF |
| | AGCCAGCAGAAACAGAGTGGTGATAATGGGTCAGGTGGTCACTTCGCACACCCAAGAGA | |
| | AGACAGGGAAGATCGGGGCCCCAGAATGACTAAAAGTTCCCTGCAAAAACTCTGCAAGC | |
| | AGCACAAGCTTTATATTACCCCAGCATTGAATGATACGCTGTATTTACACTTTAAAGGT | |
| | TTTGATCGCATTGAGAACCTGGAAGAGTACACAGGGCTGCGCTGTCTCTGGCTGCAGAG | |
| | CAATGGAATACAGAAAATCGAAAACCTGGAGGCCCAAACTGAGTTGCGTTGCCTCTTCT | |
| | TGCAAATGAACTTGCTCCGTAAAATTGAGAACCTGGAACCTCTGCAGAAACTGGATGCT | |
| | CTTAACCTCAGCAACAATTACATCAAGACCATTGAAAACCTCTCCTGCCTCCCAGTCCT | |
| | GAACACATTGCAGATGGCCCACAATCACCTGGAGACCGTGGAGGACATTCAGCATCTAC | |
| | AAGAGTGTTTGAGGCTTTGTGTCCTTGACCTTTCGCACAACAAGCTGAGTGACCCGGAG | |
| | ATCCTGAGCATTCTGGAAAGCATGCCCGATTTGCGTGTACTGAATTTGATGGGAAACCC | |
| | GGTTATCAGACAGATTCCTAATTACAGAAGGACAGTCACTGTACGACTAAAGCACTTAA | |
| | CATACCTGGATGATAGACCAGTGTTTCCAAAGGACAGAGCTTGTGCGGAGGCCTGGGCT | |
| | AGGGGAGGGTACGCAGCTGAAAAGGAGGAGAGACAGCAGTGGGAGAGCAGGGAGCGGAA | |
| | GAAGATCACAGACAGCATTGAAGCCTTGGCCATGATCAAGCAGCGGGCAGAGGAGAGGA | |
| | AAAGACAGAGAGAGAGTCAAGAGAGAGGGGAGATGACATCTTCAGATGATGGTGAGAAT | |
| | GTGCCCGCCAGTGCGGAAGGCAAGGAGGAGCCTCCCGGGGACAGAGAAACAAGGCAGAA | |
| | GATGGAGCTATTTGTTAAGGAAAGCTTTGAGGCCAAGGACGAGCTCTGCCCGGAAAAGC | |
| | CAAGTGGAGAGGAGCCGCCTGTGGAGGCTAAAAGAGAGGATGGAGGTCCAGAGCCAGAG | |
| | GGGACCCTCCCAGCTGAGACCCTGCTACTGTCGTCACCTGTGGAGGTTAAAGGAGAGGA | |
| | CGGAGATGGAGAGCCAGAGGGGACCCTCCCAGCTGAGGCCCCACCACCCCCGCCACCTG | |
| | TGGAGGTTAAAGGAGAGGATGGAGATCAAGAGCCAGAGGGGACCCTCCCAGCTGAGACC | |
| | CTGCTACTGTCACCGCCTGTGAAGGTTAAAGGAGAGGATGGAGATCGAGAGCCAGAGGG | |
| | GACCCTCCCAGCTGAGGCCCCACCACCACCGCCCCTGGGAGCTGCCAGGGAAGAACCGA | |
| | CTCCCCAGGCTGTGGCCACTGAGGGTGTATTCGTTACAGAACTTGATGGAACGAGAACG | |
| | GAAGATTTAGAAACCATTAGACTGGAGACAAAGGAGACATTCTGCATTGATGACCTACC | |
| | TGACTTGGAAGATGATGATGAAACAGGCAAATCTCTGGAAGACCAGAATATGTGCTTTC | |
| | CGAAGATTGAGGTCATCTCGAGCTTGAGTGATGACAGTGACCCTGAACTGGACTACACG | |
| | TCACTCCCTGTGCTGGAAAACCTCCCCACAGACACTCTGTCAAATATATTTGCAGTCTC | |
| | TAAAGACACCTCAAAGGCGGCTCGGGTGCCCTTCACAGACATCTTTAAAAAAGAAGCTA | |
| | AGAGGGACTTGGAAATCCGAAAACAAGACACCAAGTCCCCAAGACCCCTGATCCAGGAG | |
| | CTCAGCGACGAGGACCCCTCTGGCCAGCTACTGATGCCCCCCACCTGCCAAAGAGATGC | |
| | TGCACCACTCACTTCCAGTGGAGACAGGGACAGCGACTTCCTTGCAGCCTCTTCTCCGG | |
| | TGCCGACTGAGAGCGCCGCCACACCCCCAGAGACGTGTGTCGGAGTTGCCCAGCCCAGC | |
| | CAAGCTCTGCCCACGTGGGACCTCACTGCATTCCCAGCACCGAAAGCATCATAG | |
| SEQ ID | ATGGCCAAAGCGGCGGCCTCCTCGTCGCTGGAGGACTTGGACCTGAGCGGAGAGGAGGT | Wild type |
| NO: 35 | CCAGCGGCTCACCTCCGCCTTCCAGGACCCGGAGTTCCGGCGAATGTTCTCCCAGTACG | DNAAF2 |
| | CCGAGGAGCTCACCGACCCGGAGAACCGGCGGCGCTACGAGGCGGAGATCACCGCGCTA | ORF |
| | GAGCGTGAGCGCGGGGTGGAAGTGCGGTTCGTGCACCCGGAGCCCGGCCATGTGCTGCG | |
| | CACCAGCCTGGACGGGGCGCGGCGCTGCTTTGTGAATGTCTGCAGCAACGCGTTGGTGG | |
| | GCGCGCCCAGCAGCCGGCCCGGCTCCGGTGGCGACCGGGGCGCAGCTCCTGGCAGCCAC | |
| | TGGTCCCTGCCCTACAGCCTGGCGCCCGGCCGCGAGTACGCGGGGCGCAGCAGCAGCCG | |
| | CTACATGGTCTACGACGTGGTCTTCCATCCAGACGCGCTTGCGCTGGCCCGGCGGCACG | |
| | AGGGCTTCCGCCAGATGCTGGACGCCACGGCCCTGGAGGCCGTCGAGAAGCAGTTCGGC | |
| | GTGAAGCTGGACCGCAGGAATGCCAAGACCCTGAAGGCCAAGTATAAGGGGACCCCAGA | |
| | GGCTGCGGTGCTGCGCACGCCCCTGCCCGGGGTCATCCCCGCAAGGCCTGACGGGGAGC | |
| | CGAAGGGTCCTCTCCCGGACTTCCCCTACCCTTACCAGTACCCGGCAGCCCCCGGGCCC | |
| | CGGGCGCCCTCCCCTCCGGAAGCGGCCTTGCAGCCCGCCCCCACCGAGCCTCGCTACAG | |
| | CGTGGTGCAGCGCCACCACGTGGACCTCCAGGATTACCGCTGCTCCAGGGACTCAGCCC | |
| | CGAGCCCCGTGCCCCATGAGCTGGTGATCACCATCGAACTGCCGCTGTTGCGCTCGGCC | |
| | GAGCAGGCGGCGCTGGAGGTAACGAGAAAGCTGCTGTGCCTCGACTCGAGGAAACCTGA | |
| | CTACCGGCTGCGGCTCTCGCTCCCGTACCCAGTGGACGATGGCCGCGGCAAGGCACAAT | |
| | TCAACAAGGCCCGGCGGCAGCTGGTGGTTACGCTGCCAGTGGTGCTGCCGGCCGCGCGC | |
| | CGGGAGCCCGCTGTCGCCGTCGCCGCCGCCGCGCCGGAAGAGTCCGCGGACCGGTCCGG | |
| | AACTGACGGCCAGGCCTGCGCTTCCGCTCGCGAGGGGGAGGCGGGACCCGCGAGGAGTC | |
| | GCGCGGAGGACGGAGGCCACGATACCTGCGTGGCTGGGGCTGCGGGCTCCGGGGTCACC | |
| | ACCCTGGGCGACCCGGAGGTGGCGCCTCCGCCGGCCGCAGCTGGAGAGGAGCGTGTCCC | |
| | CAAGCCGGGGGAGCAGGACTTGAGCAGGCACGCGGGGTCACCGCCGGGCAGCGTGGAGG | |
| | AGCCATCTCCTGGAGGAGAAAACTCACCTGGTGGCGGAGGCTCCCCTTGTTTGTCCTCC | |
| | CGGAGCCTGGCGTGGGGTTCTTCTGCGGGAAGAGAGAGTGCGCGCGGAGATAGCAGTGT | |
| | GGAAACACGCGAGGAGTCGGAGGGCACGGGCGGCCAGCGCTCAGCCTGCGCCATGGGTG | |
| | GTCCCGGGACCAAGAGCGGGGAGCCTTTGTGTCCTCCGTTACTGTGTAATCAGGACAAA | |
| | GAAACCTTGACTCTGCTCATTCAGGTGCCTCGGATCCAGCCGCAAAGTCTTCAAGGAGA | |
| | TTTGAATCCCCTCTGGTACAAATTACGCTTCTCCGCACAAGACTTAGTTTATTCCTTCT | |
| | TTTTGCAATTTGCTCCAGAGAATAAATTGAGTACCACAGAACCTGTGATTAGCATTTCT | |
| | TCAAACAATGCAGTGATAGAACTGGCAAAATCTCCAGAGAGCCATGGACATTGGAGAGA | |
| | GTGGTATTATGGTGTAAACAACGATTCTTTGGAGGAAAGGTTATTTGTCAATGAAGAAA | |
| | ATGTTAATGAGTTTCTTGAAGAGGTCCTGAGCTCTCCATTCAAACAGTCTATGTCCTTG | |
| | ACCCCACCATTAATTGAAGTTCTTCAAGTTACTGATAATAAGATTCAAATTAATGCAAA | |
| | GTTGCAAGAATGTAGTAACTCTGATCAGCTACAAGGAAAGGAGGAAAGAGTAAATGAAG | |
| | AAAGTCATCTAACTGAAAAGGAATATATAGAACATTGTAACACCCCTACAACTGATTCT | |
| | GATTCATCTATAGCAGTTAAAGCACTACAAATAGATAGCTTTGGTTTAGTTACATGCTT | |
| | TCAACAAGAGTCTCTTGATGTTTCTCAAATGATACTTGGAAAATCTCAGCAACCTGAGT | |
| | CAAAAATGCAATCTGAATTTATAAAAGAAAAAAGTGCTACTTGTTCAAATGAGGAAAAA | |
| | GATAACTTAAACGAGTCAGTAATAACTGAAGAGAAAGAAACAGATGGAGATCACCTATC | |
| | TTCATTACTGAACAAAACTACGGTTCACAATATACCTGGATTCGACAGCATAAAAGAAA | |
| | CCAATATGCAGGATGGTAGTGTGCAGGTCATTAAAGATCATGTGACCAATTGTGCATTC | |
| | AGTTTTCAGAATTCTTTGCTATATGATTTGGATTAA | |
| SEQ ID | ATGCCTCTTCAGGTTAGCGATTACAGCTGGCAGCAGACGAAGACTGCGGTCTTTCTGTC | Wild type |
| NO: 36 | TCTGCCCCTCAAAGGCGTGTGCGTCAGAGACACGGACGTGTTCTGCACGGAAAACTATC | DNAAF4 |
| | TGAAGGTCAACTTTCCTCCATTTTTATTTGAGGCATTTCTTTATGCTCCCATAGACGAT | ORF |
| | GAGAGCAGCAAAGCAAAGATTGGGAATGACACCATTGTCTTCACCTTGTATAAAAAAGA | |
| | AGCGGCCATGTGGGAGACCCTTTCTGTGACGGGTGTTGACAAAGAGATGATGCAAAGAA | |
| | TTAGAGAAAAATCTATTTTACAAGCACAAGAGAGAGCAAAAGAAGCTACAGAAGCAAAA | |
| | GCTGCAGCAAAGCGGGAAGATCAAAAATACGCACTAAGTGTCATGATGAAGATTGAAGA | |
| | AGAAGAGAGGAAAAAAATAGAAGATATGAAAGAAAATGAACGGATAAAAGCCACTAAAG | |
| | CATTGGAAGCCTGGAAAGAATATCAAAGAAAAGCTGAGGAGCAAAAAAAAATTCAGAGA | |
| | GAAGAGAAATTATGTCAAAAAGAAAAGCAAATTAAAGAAGAAAGAAAAAAAATAAAATA | |
| | TAAGAGTCTTACTAGAAATTTGGCATCTAGAAATCTTGCTCCAAAAGGGAGAAATTCAG | |
| | AAAATATATTTACTGAGAAGTTAAAGGAAGACAGTATTCCTGCTCCTCGCTCTGTTGGC | |
| | AGTATTAAAATCAACTTTACCCCTCGAGTATTCCCAACAGCTCTTCGTGAATCACAAGT | |
| | AGCAGAAGAGGAGGAGTGGCTACACAAACAAGCTGAGGCACGAAGAGCAATGAATACTG | |
| | ACATAGCTGAACTTTGCGATTTAAAAGAAGAAGAAAAGAACCCAGAATGGTTGAAGGAT | |
| | AAAGGAAACAAATTGTTTGCAACGGAAAACTATTTGGCAGCTATCAATGCATATAATTT | |
| | AGCCATAAGACTAAATAATAAGATGCCACTATTGTATTTGAACCGGGCTGCTTGCCACC | |
| | TAAAACTAAAAAACTTACACAAGGCTATTGAAGATTCTTCTAAGGCACTGGAATTATTG | |
| | ATGCCACCTGTTACAGACAATGCTAATGCAAGAATGAAGGCACATGTACGACGTGGAAC | |
| | AGCATTCTGTCAACTAGAATTGTATGTAGAAGGCCTACAGGATTATGAAGCGGCACTTA | |
| | AGATTGATCCATCCAACAAAATTGTACAAATTGATGCTGAGAAGATTCGGAATGTAATT | |
| | CAAGGAACAGAACTAAAATCTTAA | |
| SEQ ID | ATGGGAGACCTGGAACTGCTGCTGCCCGGGGAAGCTGAAGTGCTGGTGCGGGGTCTGCG | Wild type |
| NO: 37 | CAGCTTCCCGCTACGCGAGATGGGCTCCGAAGGGTGGAACCAGCAGCATGAGAACCTGG | ZMYND10 |
| | AGAAGCTGAACATGCAAGCCATCCTCGATGCCACAGTCAGCCAGGGCGAGCCCATTCAG | ORF |
| | GAGCTGCTGGTCACCCATGGGAAGGTCCCAACACTGGTGGAGGAGCTGATCGCAGTGGA | |
| | GATGTGGAAGCAGAAGGTGTTCCCTGTGTTCTGCAGGGTGGAGGACTTCAAGCCCCAGA | |
| | ACACCTTCCCCATCTACATGGTGGTGCACCACGAGGCCTCCATCATCAACCTCTTGGAG | |
| | ACAGTGTTCTTCCACAAGGAGGTGTGTGAGTCAGCAGAAGACACTGTCTTGGACTTGGT | |
| | AGACTATTGCCACCGCAAACTGACCCTGCTGGTGGCCCAGAGTGGCTGTGGTGGCCCCC | |
| | CTGAGGGGGAGGGATCCCAGGACAGCAACCCCATGCAGGAGCTGCAGAAGCAGGCAGAG | |
| | CTGATGGAATTTGAGATTGCACTGAAGGCCCTCTCAGTACTACGCTACATCACAGACTG | |
| | TGTGGACAGCCTCTCTCTCAGCACCTTGAGCCGTATGCTTAGCACACACAACCTGCCCT | |
| | GCCTCCTGGTGGAACTGCTGGAGCATAGTCCCTGGAGCCGGCGGGAAGGAGGCAAGCTG | |
| | CAGCAGTTCGAGGGCAGCCGTTGGCATACTGTGGCCCCCTCAGAGCAGCAAAAGCTGAG | |
| | CAAGTTGGACGGGCAAGTGTGGATCGCCCTGTACAACCTGCTGCTAAGCCCTGAGGCTC | |
| | AGGCGCGCTACTGCCTCACAAGTTTTGCCAAGGGACGGCTACTCAAGCTTCGGGCCTTC | |
| | CTCACAGACACACTGCTGGACCAGCTGCCCAACCTGGCCCACTTGCAGAGTTTCCTGGC | |
| | CCATCTGACCCTAACTGAAACCCAGCCTCCTAAGAAGGACCTGGTGTTGGAACAGATCC | |
| | CAGAAATCTGGGAGCGGCTGGAGCGAGAAAACAGAGGCAAGTGGCAGGCAATTGCCAAG | |
| | CACCAGCTCCAGCATGTGTTCAGCCCCTCAGAGCAGGACCTGCGGCTGCAGGCGCGAAG | |
| | GTGGGCTGAGACCTACAGGCTGGATGTGCTAGAGGCAGTGGCTCCAGAGCGGCCCCGCT | |
| | GTGCTTACTGCAGTGCAGAGGCTTCTAAGCGCTGCTCACGATGCCAGAATGAGTGGTAT | |
| | TGCTGCAGGGAGTGCCAAGTCAAGCACTGGGAAAAGCATGGAAAGACTTGTGTCCTGGC | |
| | AGCCCAGGGTGACAGAGCCAAATGA | |
| SEQ ID | ATGAGTAGCGAATTCCTGGCTGAGCTGCACTGGGAGGATGGGTTCGCCATCCCGGTGGC | Wild type |
| NO: 38 | GAACGAGGAGAACAAGCTACTGGAAGATCAGTTGTCAAAGCTGAAGGATGAAAGAGCAA | CCDC39 |
| | GCTTGCAAGATGAGTTACGTGAGTATGAAGAGCGAATTAATTCTATGACTTCTCACTTC | ORF |
| | AAAAATGTTAAGCAAGAGCTCTCAATTACACAGTCTCTTTGCAAAGCAAGGGAGCGTGA | |
| | GACTGAAAGTGAAGAACATTTTAAGGCCATTGCTCAAAGAGAATTGGGACGAGTGAAAG | |
| | ATGAAATTCAACGGCTGGAAAATGAGATGGCTTCAATACTGGAAAAGAAAAGTGATAAA | |
| | GAAAATGGCATATTTAAAGCCACTCAAAAATTGGATGGTTTGAAATGTCAAATGAACTG | |
| | GGACCAGCAAGCATTGGAGGCCTGGTTAGAAGAATCAGCTCATAAAGATAGTGATGCTC | |
| | TCACTCTCCAGAAGTATGCACAACAAGATGATAATAAAATCAGGGCACTGACTCTGCAA | |
| | TTAGAAAGACTAACTTTGGAATGTAATCAGAAAAGAAAGATACTTGACAACGAACTTAC | |
| | AGAGACTATAAGCGCACAGTTAGAATTGGATAAAGCAGCACAAGATTTTCGTAAGATTC | |
| | ATAATGAAAGACAAGAACTCATTAAACAATGGGAGAACACAATAGAACAGATGCAGAAG | |
| | AGGGATGGAGACATAGATAACTGTGCTTTGGAATTAGCAAGGATAAAGCAGGAAACGAG | |
| | AGAAAAAGAAAATTTGGTTAAAGAAAAGATCAAGTTTTTGGAAAGTGAGATTGGGAATA | |
| | ACACAGAGTTTGAGAAAAGAATTTCTGTGGCTGATCGTAAACTTTTAAAATGTAGAACG | |
| | GCATATCAGGACCATGAAACTAGTAGAATTCAGCTGAAGGGTGAGCTGGATTCTTTAAA | |
| | AGCCACTGTGAATAGAACTTCCAGTGATTTAGAAGCTCTGAGGAAAAATATTTCCAAGA | |
| | TAAAGAAGGACATTCATGAAGAAACAGCAAGGTTACAAAAAACTAAAAATCATAATGAG | |
| | ATAATACAAACAAAATTAAAGGAGATAACTGAGAAAACCATGTCTGTAGAAGAGAAAGC | |
| | TACTAATTTGGAAGATATGCTAAAGGAGGAGGAAAAAGATGTGAAGGAAGTAGATGTTC | |
| | AACTGAACCTCATAAAAGGTGTGCTGTTTAAGAAAGCTCAGGAGTTACAGACTGAGACA | |
| | ATGAAAGAAAAAGCTGTTTTATCAGAAATTGAAGGAACTCGTTCCTCTCTGAAACATCT | |
| | CAACCATCAGTTACAAAAACTGGATTTTGAAACCTTGAAGCAGCAAGAAATTATGTACA | |
| | GCCAGGATTTTCACATTCAACAAGTGGAACGGAGAATGTCACGGTTAAAGGGAGAAATT | |
| | AATTCAGAAGAAAAACAAGCGCTTGAAGCAAAAATTGTTGAACTTAGGAAGTCTTTGGA | |
| | AGAGAAAAAATCTACATGTGGCCTTTTGGAAACACAGATCAAGAAGCTTCATAATGATC | |
| | TTTATTTTATCAAGAAGGCACATAGTAAAAACAGTGATGAAAAACAGTCCCTTATGACC | |
| | AAAATAAATGAACTAAACCTTTTCATCGACAGATCAGAGAAAGAACTTGATAAAGCCAA | |
| | AGGTTTTAAGCAGGATTTGATGATAGAGGACAATCTTTTAAAACTTGAAGTTAAGCGTA | |
| | CTCGAGAAATGCTTCACAGTAAGGCAGAAGAAGTTCTTTCCCTAGAAAAAAGAAAACAG | |
| | CAATTATACACAGCAATGGAAGAGCGAACTGAAGAAATCAAGGTTCATAAAACAATGCT | |
| | TGCGTCACAAATAAGATATGTTGATCAAGAACGGGAAAACATAAGCACTGAGTTTCGCG | |
| | AGCGGCTAAGTAAAATTGAGAAGCTGAAGAATAGATATGAAATTCTGACTGTTGTTATG | |
| | CTGCCTCCTGAAGGAGAAGAGGAGAAAACACAGGCCTATTATGTAATAAAGGCTGCTCA | |
| | AGAAAAAGAAGAACTTCAAAGGGAAGGTGACTGTTTGGATGCCAAGATCAACAAAGCTG | |
| | AAAAAGAAATCTACGCTCTAGAAAATACCCTTCAAGTGCTGAACAGCTGTAACAACAAT | |
| | TATAAGCAATCTTTTAAAAAAGTGACTCCATCTAGTGATGAGTATGAGCTAAAAATTCA | |
| | ACTAGAAGAACAAAAAAGAGCTGTTGATGAAAAATACAGATACAAACAAAGACAAATCA | |
| | GAGAACTTCAAGAAGACATCCAGAGCATGGAAAATACATTAGATGTTATAGAACATTTG | |
| | GCAAATAATGTTAAAGAAAAGTTATCAGAGAAGCAGGCTTATTCATTTCAACTAAGTAA | |
| | AGAAACGGAGGAGCAGAAGCCAAAATTAGAAAGAGTGACCAAACAGTGTGCAAAACTCA | |
| | CAAAGGAAATCCGTCTTTTGAAAGACACAAAAGATGAAACAATGGAAGAACAAGACATC | |
| | AAACTTCGTGAAATGAAACAGTTTCACAAAGTTATTGATGAAATGTTAGTTGATATCAT | |
| | AGAAGAAAATACTGAGATCCGTATTATCCTTCAAACATACTTTCAACAGAGTGGGTTAG | |
| | AACTACCTACAGCTAGCACAAAAGGCAGTCGTCAGAGCTCTAGATCTCCTTCACATACT | |
| | TCACTATCAGCAAGGTCATCTAGGAGTACAAGTACATCTACTTCTCAGTCTTCAATTAA | |
| | AGTACTGGAGCTTAAATTCCCGGCCTCCTCTTCACTAGTAGGCAGCCCTTCTAGGCCAT | |
| | CTAGTGCTAGTAGTAGCTCTAGTAATGTTAAGAGCAAAAAGAGCAGCAAATAA | |
| SEQ ID | ATGGCGGAACCGGGCGGCGCGGCGGGCCGGTCCCATCCGGAAGATGGATCGGCTTCTGA | Wild type |
| NO: 39 | GGGAGAGAAGGAAGGGAATAATGAAAGCCACATGGTGTCACCACCAGAGAAGGATGATG | CCDC40 |
| | GCCAGAAAGGTGAAGAAGCTGTCGGTAGCACAGAGCATCCTGAGGAAGTCACAACCCAA | ORF |
| | GCGGAAGCTGCAATTGAAGAGGGGGAGGTGGAGACAGAAGGGGAAGCAGCAGTGGAAGG | |
| | GGAAGAGGAGGCTGTGTCCTATGGAGATGCTGAAAGCGAAGAGGAATATTACTATACAG | |
| | AAACTTCATCCCCGGAAGGGCAAATCAGTGCTGCAGATACGACTTACCCGTATTTCAGT | |
| | CCTCCTCAGGAACTGCCTGGAGAGGAGGCATACGATAGTGTTAGCGGGGAGGCTGGTCT | |
| | CCAAGGCTTCCAGCAAGAGGCCACCGGTCCACCAGAATCCAGAGAAAGGAGGGTCACCT | |
| | CCCCAGAGCCATCCCACGGAGTCTTAGGCCCGTCGGAGCAAATGGGCCAGGTCACCTCT | |
| | GGGCCAGCAGTGGGCAGATTGACAGGATCCACAGAGGAGCCCCAGGGGCAGGTGCTCCC | |
| | AATGGGCGTCCAGCACCGCTTCCGGCTGAGCCACGGGAGCGACATCGAGTCCTCAGACC | |
| | TGGAGGAGTTCGTCTCGCAGGAGCCAGTGATCCCCCCAGGGGTGCCCGATGCCCACCCC | |
| | AGGGAAGGAGACCTGCCAGTGTTCCAGGACCAGATCCAGCAGCCCAGCACCGAGGAGGG | |
| | GGCCATGGCAGAGAGAGTGGAGTCCGAGGGGAGTGACGAGGAAGCAGAAGACGAAGGGT | |
| | CCCAGCTGGTGGTTTTGGACCCAGACCACCCCCTGATGGTAAGATTCCAGGCTGCCCTG | |
| | AAGAACTACCTGAACCGACAGATCGAAAAGTTGAAGCTGGACCTCCAAGAGCTGGTTGT | |
| | GGCTACCAAGCAGAGCCGAGCCCAGCGGCAGGAGCTGGGGGTGAATCTCTATGAGGTGC | |
| | AGCAGCACCTGGTACACCTGCAGAAGCTGCTGGAGAAGAGTCACGACCGCCACGCAATG | |
| | GCCTCGAGCGAGCGCAGGCAGAAGGAGGAGGAGCTGCAGGCCGCCCGCGCTCTCTACAC | |
| | CAAGACCTGCGCAGCCGCCAACGAGGAGCGCAAAAAGTTGGCGGCTCTGCAGACTGAGA | |
| | TGGAGAACTTGGCCCTGCATCTCTTCTACATGCAGAACATCGACCAGGACATGCGTGAC | |
| | GACATCCGCGTGATGACACAAGTGGTAAAGAAGGCCGAGACGGAGAGGATCCGGGCAGA | |
| | AATCGAGAAGAAAAAGCAGGACCTGTATGTGGACCAGCTCACCACTCGAGCCCAGCAAC | |
| | TGGAAGAAGACATTGCCCTGTTTGAGGCTCAGTACTTGGCCCAAGCTGAGGACACCCGG | |
| | ATTTTAAGGAAAGCAGTGAGTGAGGCCTGCACCGAGATCGACGCCATCAGCGTGGAGAA | |
| | GAGGCGCATCATGCAGCAATGGGCCAGCAGCCTGGTGGGCATGAAGCACCGCGACGAGG | |
| | CGCACAGGGCGGTGCTGGAGGCGCTCAGAGGATGCCAGCATCAAGCCAAATCCACCGAC | |
| | GGCGAGATTGAGGCCTATAAGAAATCCATCATGAAGGAGGAAGAAAAGAACGAGAAGCT | |
| | GGCGAGCATCCTGAACCGGACAGAGACGGAAGCCACACTGCTGCAGAAGCTCACCACCC | |
| | AGTGCCTGACCAAGCAGGTGGCCCTGCAGAGCCAGTTCAATACCTACAGGCTCACCCTG | |
| | CAGGACACAGAGGATGCCCTCAGCCAGGACCAGCTGGAACAAATGATACTCACGGAGGA | |
| | GTTGCAGGCCATCCGCCAAGCCATCCAGGGCGAGCTGGAGCTCAGGAGGAAGACGGATG | |
| | CTGCCATCCGGGAGAAGCTGCAGGAGCACATGACCTCCAACAAGACCACCAAATACTTC | |
| | AACCAGCTCATCCTGAGGCTGCAGAAGGAGAAGACCAACATGATGACACATCTTTCCAA | |
| | AATCAACGGTGACATTGCCCAGACCACCCTGGACATCACACACACCAGCAGCAGGCTGG | |
| | ACGCACACCAGAAGACCCTGGTGGAGCTGGACCAGGACGTGAAGAAAGTCAACGAGCTC | |
| | ATCACCAACAGCCAGAGCGAGATCTCCCGGCGCACGATCCTGATCGAGAGGAAGCAAGG | |
| | GCTCATCAACTTCCTCAACAAGCAGCTGGAGCGGATGGTCTCCGAGCTGGGGGGGGAAG | |
| | AAGTGGGGCCCCTGGAGCTTGAAATCAAAAGGCTGAGCAAGCTGATCGACGAGCACGAT | |
| | GGCAAGGCGGTCCAGGCCCAGGTGACCTGGCTGCGCCTGCAGCAGGAGATGGTCAAGGT | |
| | GACACAGGAGCAGGAGGAGCAGCTGGCCTCCCTGGACGCATCCAAGAAGGAGCTCCACA | |
| | TCATGGAGCAGAAGAAACTACGAGTAGAAAGCAAGATTGAGCAGGAGAAGAAGGAGCAG | |
| | AAGGAGATCGAGCACCACATGAAGGACCTGGACAACGACCTGAAGAAGCTCAACATGTT | |
| | GATGAATAAAAACCGGTGCAGCTCGGAGGAGCTGGAGCAGAACAACCGGGTGACAGAGA | |
| | ATGAGTTCGTGCGCTCGCTGAAGGCCTCTGAGAGGGAGACCATCAAGATGCAGGACAAG | |
| | CTGAACCAGCTCAGCGAGGAGAAGGCGACCCTCCTGAATCAACTGGTGGAAGCAGAACA | |
| | CCAGATTATGCTTTGGGAGAAAAAAATCCAACTGGCAAAAGAGATGCGTTCCTCAGTGG | |
| | ATTCCGAGATCGGCCAGACGGAGATCCGGGCCATGAAGGGCGAGATCCACAGGATGAAG | |
| | GTCAGGCTCGGGCAGCTGCTGAAGCAGCAGGAGAAGATGATCCGTGCCATGGAGTTGGC | |
| | GGTTGCCCGCAGAGAGACCGTCACCACCCAGGCCGAGGGGCAGCGCAAGATGGACAGGA | |
| | AGGCGCTCACCCGCACCGACTTCCACCACAAGCAGCTTGAGCTGCGCCGGAAAATCAGG | |
| | GACGTTCGCAAGGCCACCGATGAGTGCACCAAAACCGTCCTGGAACTGGAAGAAACACA | |
| | AAGAAATGTGAGCAGCTCCCTCCTAGAGAAGCAGGAAAAGCTGTCGGTGATTCAGGCAG | |
| | ACTTCGACACACTCGAGGCCGACCTCACCCGGCTTGGGGCCCTCAAACGACAGAACCTT | |
| | TCAGAGATCGTGGCCCTGCAGACACGCCTTAAGCACCTGCAGGCTGTGAAGGAGGGGCG | |
| | CTACGTGTTCCTGTTCCGCTCCAAGCAGTCCCTAGTGCTGGAGCGCCAGCGCCTGGACA | |
| | AGCGACTGGCTCTCATCGCCACCATCCTGGACCGCGTGCGGGACGAGTACCCCCAGTTC | |
| | CAGGAGGCCCTGCACAAGGTCAGCCAGATGATCGCCAACAAGCTCGAGTCACCAGGGCC | |
| | CTCCTAG | |

In some cases, a codon coding for a particular amino acid in the polypeptide may be substituted or replaced with a synonymous codon. For example, a codon coding for leucine may be substituted for another codon coding for leucine. In this way, the resulting translation products may be identical with the polynucleotide differing in sequence. At least one type of an isoleucine-encoding codons in the corresponding wild-type sequence may be substituted with a synonymous codon type in the nucleic acid sequence. At least one type of a valine-encoding codons in the corresponding wild-type sequence may be substituted with a synonymous codon type in the nucleic acid sequence. At least one type of an alanine-encoding codons in the corresponding wild-type sequence may be substituted with a synonymous codon type in the nucleic acid sequence. At least one type of a glycine-encoding codons in the corresponding wild-type sequence may be substituted with a synonymous codon type in the nucleic acid sequence. At least one type of a proline-encoding codons in the corresponding wild-type sequence may be substituted with a synonymous codon type in the nucleic acid sequence. At least one type of a threonine-encoding codons in the corresponding wild-type sequence may be substituted with a synonymous codon type in the nucleic acid sequence. At least one type of a leucine-encoding codons in the corresponding wild-type sequence may be substituted with a synonymous codon type in the nucleic acid sequence. At least one type of an arginine-encoding codons in the corresponding wild-type sequence may be substituted with a synonymous codon type in the nucleic acid sequence. At least one type of a serine-encoding codons in the corresponding wild-type sequence may be substituted with a synonymous codon type in the nucleic acid sequence.

In some aspects described herein, a particular codon of a particular amino acid comprises a percentage or amount of the total number of codons for that particular amino acid the polynucleotide. This may be referred to a “codon frequency”. For example, at least 50% of the total codons encoding a particular amino acid in the polynucleotide may be encoded by a first codon sequence. For example, at least 55% of the total codons encoding a particular amino acid in the polynucleotide may be encoded by a first codon sequence. At least 5%, 10%, 20%, 30%, 40%, 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or more of the total codons encoding a particular amino in the polynucleotide may be encoded by a first codon sequence. In some cases, no more than 5%, 10%, 20%, 30%, 40%, 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or less of the total codons encoding a particular amino in the polynucleotide are encoded by a first codon sequence. At least about 50%, 55%, 60%, 65%, 70%, 75%, 80%, 85%, 90%, or 95% phenylalanine-encoding codons of the synthetic polynucleotide may be TTC (as opposed to TTT). At least about 50%, 55%, 60%, 65%, 70%, 75%, 80%, 85%, 90%, or 95% cysteine-encoding codons of the synthetic polynucleotide may be TGC (as opposed to TGT). At least about 50%, 55%, 60%, 65%, 70%, 75%, 80%, 85%, 90%, or 95% aspartic acid-encoding codons of the synthetic polynucleotide may be GAC (as opposed to GAT). At least about 50%, 55%, 60%, 65%, 70%, 75%, 80%, 85%, 90%, or 95% glutamic acid-encoding codons of the synthetic polynucleotide may be GAG (as opposed to GAA). At least about 50%, 55%, 60%, 65%, 70%, 75%, 80%, 85%, 90%, or 95% histidine-encoding codons of the synthetic polynucleotide may be CAC (as opposed to CAT). At least about 50%, 55%, 60%, 65%, 70%, 75%, 80%, 85%, 90%, or 95% lysine-encoding codons of the synthetic polynucleotide may be AAG (as opposed to AAA). At least about 50%, 55%, 60%, 65%, 70%, 75%, 80%, 85%, 90%, or 95% asparagine-encoding codons of the synthetic polynucleotide may be AAC (as opposed to AAT). At least about 50%, 55%, 60%, 65%, 70%, 75%, 80%, 85%, 90%, or 95% glutamine-encoding codons of the synthetic polynucleotide may be CAG (as opposed to CAA). At least about 50%, 55%, 60%, 65%, 70%, 75%, 80%, 85%, 90%, or 95% tyrosine-encoding codons of the synthetic polynucleotide may be TAC (as opposed to TAT). At least about 50%, 55%, 60%, 65%, 70%, 75%, 80%, 85%, 90%, or 95% isoleucine-encoding codons of the synthetic polynucleotide may be ATC. At least about 90% phenylalanine-encoding codons of the synthetic polynucleotide may be TTC (as opposed to TTT). At least about 60% cysteine-encoding codons of the synthetic polynucleotide may be TGC (as opposed to TGT). At least about 70% aspartic acid-encoding codons of the synthetic polynucleotide may be GAC (as opposed to GAT). At least about 50% glutamic acid-encoding codons of the synthetic polynucleotide may be GAG (as opposed to GAA). At least about 60% histidine-encoding codons of the synthetic polynucleotide may be CAC (as opposed to CAT). At least about 60% lysine-encoding codons of the synthetic polynucleotide may be AAG (as opposed to AAA). At least about 60% asparagine-encoding codons of the synthetic polynucleotide may be AAC (as opposed to AAT). At least about 70% glutamine-encoding codons of the synthetic polynucleotide may be CAG (as opposed to CAA). At least about 80% tyrosine-encoding codons of the synthetic polynucleotide may be TAC (as opposed to TAT). At least about 90% isoleucine-encoding codons of the synthetic polynucleotide may be ATC.

In some embodiments, a particular amino acid the polynucleotide may be encoded by a number of different codon sequences. For example, a particular amino acid in the polynucleotide may be encoded by no more than 2 different codon sequences. In some cases, the polynucleotide comprises no more than 2 types of isoleucine-encoding codons.

In some embodiments, a particular amino acid in the polynucleotide may be encoded by no more than 3 different codon sequences. The polynucleotide may comprise no more than 3 types of alanine (Ala)-encoding codons. The polynucleotide may comprise no more than 3 types of glycine (Gly)-encoding codons. The polynucleotide may comprise no more than 3 types of proline (Pro)-encoding codons. The polynucleotide may comprise no more than 3 types of threonine (Thr)-encoding codons.

In some embodiments, a particular amino acid in the polynucleotide may be encoded by no more than 4 different codon sequences. The polynucleotide may comprise no more than 4 types of arginine (Arg)-encoding codons. The polynucleotide may comprise no more than 4 types of serine (Ser)-encoding codons. In some embodiments, a particular amino acid in the polynucleotide may be encoded by no more than 5 different codon sequences. The polynucleotide may comprise no more than 5 types of arginine (Arg)-encoding codons. The polynucleotide may comprise no more than 5 types of serine (Ser)-encoding codons. In some embodiments, a particular amino acid in the polynucleotide may be encoded by no more than 6 different codon sequences. In some embodiments, a particular amino acid in the polynucleotide may be encoded by 1 or more different codon sequences. In some embodiments, a particular amino acid in the polynucleotide may be encoded by 2 or more different codon sequences. In some embodiments, a particular amino acid in the polynucleotide may be encoded by 3 or more different codon sequences. In some embodiments, a particular amino acid in the polynucleotide may be encoded by 4 or more different codon sequences. In some embodiments, a particular amino acid in the polynucleotide may be encoded by 5 or more different codon sequences. In some embodiments, a particular amino acid in the polynucleotide may be encoded by 6 or more different codon sequences.

In some cases, a frequency of a first codon sequence of a is higher, lower or the same as a frequency of a second codon sequence encoding for a particular amino acid in the polynucleotide. For example, a frequency of a first codon may be higher than a frequency of second codon for a particular amino acid in the polynucleotide. The frequency of GCC codon may be higher than a frequency of GCT codon. The frequency of GCT codon may be lower than a frequency of GCA codon. The frequency of GCT codon may be higher than a frequency of GCA codon.

In some embodiments, the codon usage for alanine-encoding codons in the polynucleotide may have a particular parameter. For example, a frequency of GCG codon may be no more than about 10% or 5%. A frequency of GCA codon may be no more than about 20%. A frequency of GCT codon may be at least about 1%, 5%, 10%, 15%, 20%, or 25%. A frequency of GCT codon may be no more than about 30%, 25%, 20%, 15%, 10%, or 5%. A frequency of GCC codon may be at least about 60%, 70%, 80%, or 90%. A frequency of GCC codon may be no more than about 95%, 90%, 85%, 80%, or 75%. The frequency of GCC codon may be higher than a frequency of GCT codon. The frequency of GCT codon may be lower than a frequency of GCA codon. The frequency of GCT codon may be higher than a frequency of GCA codon.

In some embodiments, the codon usage for glycine-encoding codons the polynucleotide may have a particular parameter. For example, a frequency of GGC codon may be lower than a frequency of GGA codon. For example, a frequency of GGC codon may be higher than a frequency of GGA codon. A frequency of GGG codon may be no more than about 10% or 5%. A frequency of GGG codon may be least about 1%. A frequency of GGA codon may be no more than about 30% or 20%. A frequency of GGA codon may be at least about 10% or 20%. A frequency of GGT codon may be more than about 10% or 5%. A frequency of GGC codon may be no more than about 90%, 80%, or 70%. A frequency of GGC codon may be least about 60%, 70%, or 80%.

In some embodiments, the codon usage for proline-encoding codons the polynucleotide may have a particular parameter. For example, a frequency of CCC codon may be lower than a frequency of CCT codon. A frequency of CCC codon may be higher than a frequency of CCT codon. A frequency of CCC codon may be lower than a frequency of CCA codon. A frequency of CCC codon may be higher than a frequency of CCA codon. A frequency of CCT codon may be lower than a frequency of CCA codon. A frequency of CCT codon may be higher than a frequency of CCA codon. A frequency of CCG codon may be no more than about 10% or 5%. frequency of CCA codon may be no more than about 30%, 20%, or 10%. A frequency of CCA codon may be at least about 5%, 10%, 15%, 20%, or 25%. A frequency of CCT codon may be no more than about 60%, 50%, 40%, or 30%. A frequency of CCT codon may be at least about 20%, 30%, 40%, or 50%. A frequency of CCC codon may be no more than about 60%, 50%, or 40%. A frequency of CCC codon may be at least about 30%, 40%, 50%, 60%, or 70%.

In some embodiments, the codon usage for threonine-encoding codons the polynucleotide may have a particular parameter. For example, a frequency of ACA codon may be higher than a frequency of ACT codon. A frequency of ACC codon may be higher than a frequency of ACT codon. A frequency of ACC codon may be lower than a frequency of ACA codon. A frequency of ACC codon may be higher than a frequency of ACA codon. A frequency of ACG codon may be no more than about 10% or 5%. A frequency of ACA codon may be no more than about 60%, 50%, 40%, or 30%. A frequency of ACA codon may be at least about 10%, 20%, 30%, 40%, or 50%. A frequency of ACT codon may be no more than about 10% or 5%. A frequency of ACC codon may be no more than about 90%, 80%, 70%, 60%, or 50%. A frequency of ACC codon may be at least about 40%, 50%, 60%, 70%, or 80%.

In some embodiments, the codon usage for arginine-encoding codons the polynucleotide may have a particular parameter. For example, a frequency of AGA codon may be lower than a frequency of AGG codon. A frequency of AGA codon may be higher than a frequency of AGG codon. A frequency of AGA codon may be lower than a frequency of CGG codon. A frequency of AGA codon may be higher than a frequency of CGG codon. A frequency of CGG codon may be higher than a frequency of CGA codon. A frequency of CGG codon may be higher than a frequency of CGC codon. A frequency of AGG codon may be no more than about 10%. A frequency of AGG codon may be less than about 10%. A frequency of AGA codon may be no more than about 70%, 60%, or 50%. A frequency of AGA codon may be at least about 40%, 50%, 60%, or 70%. A frequency of CGG codon may be no more than about 50%, 40%, or 30%. A frequency of CGG codon may be at least about 20%, 30%, or 40%. A frequency of CGA codon may be at least about 1%. A frequency of CGA codon may be no more than about 10% or 5%. A frequency of CGT codon may be no more about 10% or 5%. A frequency of CGC codon may be no more than about 20%, 10%, or 5%. A frequency of CGC codon may be at least about 1%, 2%, 3%, 4%, or 5%.

In some embodiments, the codon usage for serine-encoding codons the polynucleotide may have a particular parameter. For example, a frequency of AGC codon may be higher than a frequency of TCT codon. A frequency of TCT codon may be higher than a frequency of TCG codon. A frequency of TCT codon may be higher than a frequency of TCA codon. A frequency of TCT codon may be higher than a frequency of TCC codon. A frequency of AGT codon may be no more than about 10%. A frequency of AGT codon may be at least about 1%. A frequency of AGC codon may be no more about 95%, 90%, 85%, or 80%. A frequency of AGC codon may be at least about 70%, 80%, or 90%. A frequency of TCG codon may be no more than about 10% or 5%. A frequency of TCA codon may be no more than about 10% or 5%. A frequency of TCT codon may be no more than about 30%, 20%, or 10%. A frequency of TCT codon may be at least about 10%, or 20%. A frequency of TCC codon may be no more than about 10% or 5%.

A polypeptide sequence can be engineered to have a desired altered codon usage, such as the altered codon usage of SEQ ID NOs: 1-32, 61, or 62. Computer software can be used, for example, to generate the codon usages of sequences, such as polynucleotide sequences disclosed herein. A polypeptide sequence can share a % homology to an amino acid sequence of an endogenous polypeptide. A polypeptide sequence can share at most 10% homology, at most 20% homology, at most 30% homology, at most 40% homology, at most 50% homology, at most 60% homology, at most 70% homology, at most 80% homology, at most 90% homology, or at most 99% homology with an amino acid sequence of an endogenous polypeptide. Various methods and software programs can be used to determine the homology between two or more peptides, such as NCBI BLAST, Clustal W, MAFFT, Clustal Omega, AlignMe, Praline, or another suitable method or algorithm.

In some instances the polynucleotide comprises sequence encoding a for a tag. The tag may be a polypeptide sequence that may be used for purifying polypeptide. For example the tag may be a poly(His) tag, a MBP (maltose binding protein) tag, Strep-tag a GST (Glutathione-S-transferase) tag. The tag may be a polypeptide sequence that may be used to monitor expression. For example, the tag may be a HA-tag, a FLAG tag, a Myc-tag, a MycFLAG -tag, an ALFA-tag, a V5-tag, or a Spot-tag. In some embodiments, the polynucleotide of the present disclosure comprises SEQ ID NO 40.

TABLE 4
Example Tag sequences

| SEQ ID NO: | GGAAGCGGCTACCCATACGATGTTCCTGACT | HA-Tag |
|---|---|---|
| 40 | ATGCG | |

### Untranslated Regions

In some instances, the polynucleotide, nucleic acid construct, vector, or composition also comprises the genetic code of 5′ untranslated region(s) (UTR(s)) and 3′ UTR(s) such as one or more set forth in SEQ ID NOs 41-55 (as shown below), or any subset thereof. The untranslated regions may be also known as a “noncoding region” or “non-coding region”. In some embodiments, the nucleic acid sequence of the present disclosure comprises one or more sequences (e.g., one or two) set forth in SEQ ID NOs 41-55, or any subset thereof. In some embodiments, the nucleic acid sequence of the present disclosure comprises a sequence set forth in SEQ ID NOs 41-48 or 54. In some embodiments, the nucleic acid sequence of the present disclosure comprises a sequence set forth in SEQ ID NOs 49-53 or 55. In some embodiments, the nucleic acid sequence of the present disclosure comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to one set forth in SEQ ID NOs 41-55. In some embodiments, the nucleic acid sequence of the present disclosure comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to one set forth in SEQ ID NOs 41-48 and 54. In some embodiments, the nucleic acid sequence of the present disclosure comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO 54. In some embodiments, the nucleic acid sequence of the present disclosure comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to one set forth in SEQ ID NOs 49-53 and 55. In some embodiments, the nucleic acid sequence of the present disclosure comprises a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO 55.

TABLE 5
Example UTR sequences

| | | SEQ ID |
|---|---|---|
| UTR | DNA sequence (from 5′ to 3′) | NO. |
| α-globin 5′ | GGGAGACATAAACCCTGGCGCGCTCGCGGCCCGGCACTCTTCTGGTCCCCACAGACTC | 41 |
| UTR (HBA1) | AGAGAGAAGCCACC | |
| α-globin 5′ | GGGAGACATAAACCCTGGCGCGCTCGCGGGCCGGCACTCTTCTGGTCCCCACAGACTC | 42 |
| UTR | AGAGAGAAGCCACC | |
| (HBA2) | | |
| α-globin 5′ | GGGAGACTCTTCTGGTCCCCACAGACTCAGAGAGAACGCCACC | 43 |
| UTR | | |
| IRES of | GTTATTTTCCACCATATTGCCGTCTTTTGGCAATGTGAGGGCCCGGAAACCTGGCCCT | 44 |
| EMCV 5′- | GTCTTCTTGACGAGCATTCCTAGGGGTCTTTCCCCTCTCGCCAAAGGAATGCAAGGTC | |
| UTR | TGTTGAATGTCGTGAAGGAAGCAGTTCCTCTGGAAGCTTCTTGAAGACAAACAACGTC | |
| | TGTAGCGACCCTTTGCAGGCAGCGGAACCCCCCACCTGGCGACAGGTGCCTCTGCGGC | |
| | CAAAAGCCACGTGTATAAGATACACCTGCAAAGGCGGCACAACCCCAGTGCCACGTTG | |
| | TGAGTTGGATAGTTGTGGAAAGAGTCAAATGGCTCTCCTCAAGCGTATTCAACAAGGG | |
| | GCTGAAGGATGCCCAGAAGGTACCCCATTGTATGGGATCTGATCTGGGGCCTCGGTGC | |
| | ACATGCTTTACGTGTGTTTAGTCGAGGTTAAAAAACGTCTAGGCCCCCCGAACCACGG | |
| | GGACGTGGTTTTCCTTTGAAAAACACGATGATAATATGGCCACAACC | |
| IRES of TEV | AAATAACAAATCTCAACACAACATATACAAAACAAACGAATCTCAAGCAATCAAGCAT | 45 |
| 5′-UTR | TCTACTTCTATTGCAGCAATTTAAATCATTTCTTTTAAAGCAAAAGCAATTTTCTGAA | |
| | AATTTTCACCATTTACGAACGATAGCA | |
| ssRNA1 | GGGAGACAAGAGAGAAAAGAAGAGCAAGAAGAAATATAAGAGCCACC | 46 |
| 5′UTR | | |
| ssRNA2 | GGGAGACCCAAGCTGGCTAGCGTTTAAACTTAAGCTTGGCAATCCGGTACTGTTGGTA | 47 |
| 5′UTR | AAGCCACC | |
| ssRNA 3 + | GGGAGACCCAAGCTGGCTAGCGTTTAAACTTAAGCTTTCCTTTCCGGGCCGGCTGGGC | 48 |
| native 5′ UTR | GCGCCGAAGCGCCTGCGCCTTGGCTGCTGGTCGGTTGCTGGGTAACCGCGTCAGGGAG | |
| | TTGGATTCTATCCTGCAAGGGCACGGGGACCCACAACGACGGCTGTCCCTAAAGAACC | |
| | GTTGCGACTGGTAACTGAAGTGGAAGAGAGTCCAGATTTCTTGTGTGTGGTCAAGGAG | |
| | ACGGACAAACTTTTTGTCTTCAGACGAGGGAGCGTTTTGTAGGCTCTCCAGGGGTTGA | |
| | G | |
| TMV 3′-UTR | GGATTGTGTCCGTAATCACACGTGGTGCGTACGATAACGCATAGTGTTTTTCCCTCCA | 49 |
| | CTTAAATCGAAGGGTTGTGTCTTGGATCGCGCGGGTCAAATGTATATGGTTCATATAC | |
| | ATCCGCAGGCACGTAATAAAGCGAGGGGTTCGAATCCCCCCGTTACCCCCGGTAGGGG | |
| | CCCATTGTCTTC | |
| MALAT1 3′- | TCAGTAGGGTCATGAAGGTTTTTCTTTTCCTGAGAAAACAACACGTATTGTTTTCTCA | 50 |
| UTR | GGTTTTGCTTTTTGGCCTTTTTCTAGCTTAAAAAAAAAAAAAGCAAAATTGTCTTC | |
| NEAT2 3′- | TCAGTAGGGTTGTAAAGGTTTTTCTTTTCCTGAGAAAACAACCTTTTGTTTTCTCAGG | 51 |
| UTR | TTTTGCTTTTTGGCCTTTCCCTAGCTTTAAAAAAAAAAAAGCAAAATTGTCTTC | |
| histone cluster | GAAGTGGCGGTTCGGCCGGAGGTTCCATCGTATCCAAAAGGCTCTTTTCAGAGCCACC | 52 |
| 2, H3c 3′- | CATTGTCTTC | |
| UTR | | |
| Native 3′ | GGGGCTGGCCTCAGTCTCTGTCCCATCGCTTGAATACAGTACTCCTAGGGCTTGACCC | 53 |
| UTR | TGGTACCCAGCCCAGCCTTAGCACCCAGCATGTGACCCCACTCCTGATCAGGTCCCAG | |
| | CATCTTCCCTTCTTGTTCTGTTCCTTAAGGTCCCAGCACCTTACCCCAGGACTTGGTC | |
| | TTCAACCACCATTACCCCTCTAACTTTGCACAAATAAACCTGTGTAGAAACCCACCCC | |
| | AAAAAAA | |
| ssRNA2 | GGGAGACCCAAGCTGGCTAGCGTTTAAACTTCAGCTTGGCAATCCGGTACTGTTGGTA | 54 |
| 5′UTR | AAGCCACC | |
| (A32C) | | |
| 3′ UTR | GAATTCTGCAGAAAAAAAAAAAAAAAAAAAAAAAAAAAAAAAAAAAAAAAAAAAAAAA | 55 |
| poly(A) | AAAAAAAAAAAAAAAAAAAAAAAAAAAAAAAAAAAAAAAAAAAAAAAAAAAAAAAAAA | |
| | AAAAAAAAAAAAAAATTCG | |

A nucleic acid construct(s), a vector(s), or an engineered polyribonucleotide(s) of the disclosure can comprise one or more untranslated regions. An untranslated region can comprise any number of modified or unmodified nucleotides. Untranslated regions (UTRs) of a gene are transcribed but not translated into a polypeptide. In some cases, an untranslated sequence can increase the stability of the nucleic acid molecule and the efficiency of translation. The regulatory features of a UTR can be incorporated into the modified mRNA molecules of the present disclosure, for instance, to increase the stability of the molecule. The specific features can also be incorporated to ensure controlled down-regulation of the transcript in case they are misdirected to undesired organs sites. Some 5′ UTRs play roles in translation initiation. A 5′ UTR can comprise a Kozak sequence which is involved in the process by which the ribosome initiates translation of many genes. Kozak sequences can have the consensus GCC(R)CCAUGG, where R is a purine (adenine or guanine) that is located three bases upstream of the start codon (AUG). 5′ UTRs may form secondary structures which are involved in binding of translation elongation factor. In some cases, one can increase the stability and protein production of the engineered polynucleotide molecules of the disclosure, by engineering the features typically found in abundantly expressed genes of specific target organs. For example, introduction of 5′UTR of liver-expressed mRNA, such as albumin, serum amyloid A, Apolipoprotein AB/E, transferrin, alpha fetoprotein, erythropoietin, or Factor VIII, can be used to increase expression of an engineered polynucleotide in a liver. Likewise, use of 5′ UTR from muscle proteins (MyoD, Myosin, Myoglobin, Myogenin, Herculin), for endothelial cells (Tie-1, CD36), for myeloid cells (C/EBP, AML1, G-CSF, GM-CSF, CD1b, MSR, Fr-1, i-NOS), for leukocytes (CD45, CD18), for adipose tissue (CD36, GLUT4, ACRP30, adiponectin) and for lung epithelial cells (SP-A/B/C/D) can be used to increase expression of an engineered polynucleotide in a desired cell or tissue.

Other non-UTR sequences can be incorporated into the 5′ (or 3′ UTR) UTRs of the polyribonucleotides of the present disclosure. The 5′ and/or 3′ UTRs can provide stability and/or translation efficiency of polyribonucleotides. For example, introns or portions of intron sequences can be incorporated into the flanking regions of an engineered polyribonucleotide. Incorporation of intronic sequences can also increase the rate of translation of the polyribonucleotide.

3′ UTRs may have stretches of Adenosines and Uridines embedded therein. These AU rich signatures are particularly prevalent in genes with high rates of turnover. Based on their sequence features and functional properties, the AU rich elements (AREs) can be separated into classes: Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif c-Jun and Myogenin are two well-studied examples of this class. Proteins binding to the AREs may destabilize the messenger, whereas members of the ELAV family, such as HuR, may increase the stability of mRNA. HuR may bind to AREs of all the three classes. Engineering the HuR specific binding sites into the 3′ UTR of nucleic acid molecules can lead to HuR binding and thus, stabilization of the message in vivo.

Engineering of 3′ UTR AU rich elements (AREs) can be used to modulate the stability of an engineered polyribonucleotide. One or more copies of an ARE can be engineered into a polyribonucleotide to modulate the stability of a polyribonucleotide. AREs can be identified, removed or mutated to increase the intracellular stability and thus increase translation and production of the resultant protein. Transfection experiments can be conducted in relevant cell lines, using engineered polyribonucleotides and protein production can be assayed at various time points post-transfection. For example, cells can be transfected with different ARE-engineering molecules and by using an ELISA kit to the relevant protein and assaying protein produced at 6 hours, 12 hours, 24 hours, 48 hours, and 7 days post-transfection.

An untranslated region can comprise any number of nucleotides. An untranslated region can comprise a length of about 1 to about 10 bases or base pairs, about 10 to about 20 bases or base pairs, about 20 to about 50 bases or base pairs, about 50 to about 100 bases or base pairs, about 100 to about 500 bases or base pairs, about 500 to about 1000 bases or base pairs, about 1000 to about 2000 bases or base pairs, about 2000 to about 3000 bases or base pairs, about 3000 to about 4000 bases or base pairs, about 4000 to about 5000 bases or base pairs, about 5000 to about 6000 bases or base pairs, about 6000 to about 7000 bases or base pairs, about 7000 to about 8000 bases or base pairs, about 8000 to about 9000 bases or base pairs, or about 9000 to about 10000 bases or base pairs in length. An untranslated region can comprise a length of for example, at least 1 base or base pair, 2 bases or base pairs, 3 bases or base pairs, 4 bases or base pairs, 5 bases or base pairs, 6 bases or base pairs, 7 bases or base pairs, 8 bases or base pairs, 9 bases or base pairs, 10 bases or base pairs, 20 bases or base pairs, 30 bases or base pairs, 40 bases or base pairs, 50 bases or base pairs, 60 bases or base pairs, 70 bases or base pairs, 80 bases or base pairs, 90 bases or base pairs, 100 bases or base pairs, 200 bases or base pairs, 300 bases or base pairs, 400 bases or base pairs, 500 bases or base pairs, 600 bases or base pairs, 700 bases or base pairs, 800 bases or base pairs, 900 bases or base pairs, 1000 bases or base pairs, 2000 bases or base pairs, 3000 bases or base pairs, 4000 bases or base pairs, 5000 bases or base pairs, 6000 bases or base pairs, 7000 bases or base pairs, 8000 bases or base pairs, 9000 bases or base pairs, or 10000 bases or base pairs in length.

An engineered polyribonucleotide of the disclosure can comprise one or more introns. An intron can comprise any number of modified or unmodified nucleotides. An intron can comprise, for example, at least 1 base or base pair, 50 bases or base pairs, 100 bases or base pairs, 150 bases or base pairs, 200 bases or base pairs, 300 bases or base pairs, 400 bases or base pairs, 500 bases or base pairs, 600 bases or base pairs, 700 bases or base pairs, 800 bases or base pairs, 900 bases or base pairs, 1000 bases or base pairs, 2000 bases or base pairs, 3000 bases or base pairs, 4000 bases or base pairs, or 5000 bases or base pairs. In some cases, an intron can comprise, for example, at most 10000 bases or base pairs, 5000 bases or base pairs, 4000 bases or base pairs, 3000 bases or base pairs, 2000 bases or base pairs, 1000 bases or base pairs, 900 bases or base pairs, 800 bases or base pairs, 700 bases or base pairs, 600 bases or base pairs, 500 bases or base pairs, 400 bases or base pairs, 300 bases or base pairs, 200 bases or base pairs, or 100 bases or base pairs.

In some cases, a percentage of the nucleotides in an intron are modified. For instance, in some cases, fewer than 99%, 95%, 90%, 85%, 80%, 75%, 70%, 65%, 60%, 55%, 50%, 45%, 40%, 35%, 30%, 25%, 20%, 15%, 10%, 5% or 1% of the nucleotides in an intron are modified. In some cases, all of the nucleotides in an intron are modified.

A nucleic acid construct(s), a vector(s), or an engineered polyribonucleotide(s) of the disclosure can comprise one or more promoter sequences and any associated regulatory sequences. A promoter sequence and/or an associated regulatory sequence can comprise any number of modified or unmodified nucleotides, and any number of nucleic acid analogues. Promoter sequences and/or any associated regulatory sequences can comprise, for example, at least 2 bases or base pairs, 3 bases or base pairs, 4 bases or base pairs, 5 bases or base pairs, 6 bases or base pairs, 7 bases or base pairs, 8 bases or base pairs, 9 bases or base pairs, 10 bases or base pairs, 11 bases or base pairs, 12 bases or base pairs, 13 bases or base pairs, 14 bases or base pairs, 15 bases or base pairs, 16 bases or base pairs, 17 bases or base pairs, 18 bases or base pairs, 19 bases or base pairs, 20 bases or base pairs, 21 bases or base pairs, 22 bases or base pairs, 23 bases or base pairs, 24 bases or base pairs, 25 bases or base pairs, 26 bases or base pairs, 27 bases or base pairs, 28 bases or base pairs, 29 bases or base pairs, 30 bases or base pairs, 35 bases or base pairs, 40 bases or base pairs, 50 bases or base pairs, 75 bases or base pairs, 100 bases or base pairs, 150 bases or base pairs, 200 bases or base pairs, 300 bases or base pairs, 400 bases or base pairs, 500 bases or base pairs, 600 bases or base pairs, 700 bases or base pairs, 800 bases or base pairs, 900 bases or base pairs, 1000 bases or base pairs, 2000 bases or base pairs, 3000 bases or base pairs, 4000 bases or base pairs, 5000 bases or base pairs, at least 10000 bases or base pairs or more. A promoter sequence and/or an associated regulatory sequence can comprise any number of modified or unmodified nucleotides, for example, at most 10000 bases or base pairs, 5000 bases or base pairs, 4000 bases or base pairs, 3000 bases or base pairs, 2000 bases or base pairs, 1000 bases or base pairs, 900 bases or base pairs, 800 bases or base pairs, 700 bases or base pairs, 600 bases or base pairs, 500 bases or base pairs, 400 bases or base pairs, 300 bases or base pairs, 200 bases or base pairs, 100 bases or base pairs, 75 bases or base pairs, 50 bases or base pairs, 40 bases or base pairs, 35 bases or base pairs, 30 bases or base pairs, 29 bases or base pairs, 28 bases or base pairs, 27 bases or base pairs, 26 bases or base pairs, 25 bases or base pairs, 24 bases or base pairs, 23 bases or base pairs, 22 bases or base pairs, 21 bases or base pairs, 20 bases or base pairs, 19 bases or base pairs, 18 bases or base pairs, 17 bases or base pairs, 16 bases or base pairs, 15 bases or base pairs, 14 bases or base pairs, 13 bases or base pairs, 12 bases or base pairs, 11 bases or base pairs, 10 bases or base pairs, 9 bases or base pairs, 8 bases or base pairs, 7 bases or base pairs, 6 bases or base pairs, 5 bases or base pairs, 4 bases or base pairs, 3 bases or base pairs or 2 bases or base pairs.

In some cases, less than all of the nucleotides in the promoter sequence or associated regulatory region are nucleotide analogues or modified nucleotides. For instance, in some cases, less than or equal to 99%, 95%, 90%, 85%, 80%, 75%, 70%, 65%, 60%, 55%, 50%, 45%, 40%, 35%, 30%, 25%, 20%, 15%, 10%, or 5% of the nucleotides in a promoter or associated regulatory region. In some cases, all of the nucleotides in a promoter or associated regulatory region are nucleic acid analogues or modified nucleotides.

A nucleic acid construct(s), a vector(s), an engineered polyribonucleotide(s), or compositions of the disclosure can comprise an engineered 5′ cap structure, or a 5′-cap can be added to a polyribonucleotide intracellularly. The 5′cap structure of an mRNA can be involved in binding to the mRNA Cap Binding Protein (CBP), which is responsible for mRNA stability in the cell and translation competency through the association of CBP with poly(A) binding protein to form the mature pseudo-circular mRNA species. The 5′cap structure can also be involved in nuclear export, increases in mRNA stability, and in assisting the removal of 5′ proximal introns during mRNA splicing.

A 5′-cap structure may improve a pharmacokinetic characteristic of the polynucleotide in a subject or in a solution. For example, the 5′-cap structure may allow a polynucleotide to have a longer half-life than a corresponding polynucleotide without a 5′ cap structure. Without being limited to specific mechanism, the 5′ cap structure may reduce degradation of coding sequences in a polynucleotide. The 5′-cap structure may affect or promote the translation process of the polynucleotide.

A nucleic acid construct(s), a vector(s), or an engineered polyribonucleotide(s) can be 5′-end capped generating a 5′-GpppN-3′-triphosphate linkage between a terminal guanosine cap residue and the 5′-terminal transcribed sense nucleotide of the mRNA molecule. The cap-structure can comprise a modified or unmodified 7-methylguanosine linked to the first nucleotide via a 5′-5′ triphosphate bridge. This 5′-guanylate cap can then be methylated to generate an N7-methyl-guanylate residue (Cap-0 structure). The ribose sugars of the terminal and/or anteterminal transcribed nucleotides of the 5′end of the mRNA may optionally also be 2′-O-methylated (Cap-1 structure). 5′-decapping through hydrolysis and cleavage of the guanylate cap structure may target a nucleic acid molecule, such as an mRNA molecule, for degradation.

In some cases, a cap can comprise further modifications, including the methylation of the 2′ hydroxy-groups of the first 2 ribose sugars of the 5′ end of the mRNA. For instance, an eukaryotic cap-1 has a methylated 2′-hydroxy group on the first ribose sugar, while a cap-2 has methylated 2′-hydroxy groups on the first two ribose sugars. The 5′ cap can be chemically similar to the 3′ end of an RNA molecule (the 5′ carbon of the cap ribose is bonded, and the free 3′-hydroxyls on both 5′- and 3′- ends of the capped transcripts. Such double modification can provide significant resistance to 5′ exonucleases. Non-limiting examples of 5′ cap structures that can be used with an engineered polyribonucleotide include, but are not limited to, m⁷G(5′)ppp(5′)N (Cap-0), m⁷G(5′)ppp(5′)N1mpNp (Cap-1), and m⁷G(5′)-ppp(5′)N1mpN2mp (Cap-2).

Modifications to the modified mRNA of the present disclosure may generate a non-hydrolyzable cap structure preventing decapping and thus increasing mRNA half-life while facilitating efficient translation. Because cap structure hydrolysis requires cleavage of 5′-ppp-5′triphosphate linkages, modified nucleotides may be used during the capping reaction. For example, a Vaccinia Capping Enzyme from New England Biolabs (Ipswich, MA) may be used with guanosine α-thiophosphate nucleotides according to the manufacturer's instructions to create a phosphorothioate linkage in the 5′-ppp-5′ cap. Additional modified guanosine nucleotides may be used such as α-methyl-phosphonate and seleno-phosphate nucleotides. Additional modifications include, but are not limited to, 2′-O-methylation of the ribose sugars of 5′-terminal and/or 5′-anteterminal nucleotides of the mRNA on the 2′-hydroxyl group of the sugar ring. Multiple distinct 5′-cap structures can be used to generate the 5′-cap of a polyribonucleotide.

The modified mRNA may be capped post-transcriptionally. According to the present disclosure, 5′ terminal caps may include endogenous caps or cap analogues. According to the present disclosure, a 5′ terminal cap may comprise a guanine analogue. Useful guanine analogues include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

Further, a nucleic acid construct(s), a vector(s), or an engineered polyribonucleotide(s) can contain one or more internal ribosome entry site(s) (IRES). IRES sequences can initiate protein synthesis in absence of the 5′ cap structure. An IRES sequence can also be the sole ribosome binding site, or it can serve as one of multiple ribosome binding sites of an mRNA. Engineered polyribonucleotides containing more than one functional ribosome binding site can encode several peptides or polypeptides that are translated by the ribosomes (“polycistronic or multicistronic polynucleotides”). An engineered polynucleotide described here can comprise at least 1 IRES sequence, two IRES sequences, three IRES sequences, four IRES sequences, five IRES sequences, six IRES sequences, seven IRES sequences, eight IRES sequences, nine IRES sequences, ten IRES sequences, or another suitable number are present in an engineered polyribonucleotide. Examples of IRES sequences that can be used according to the present disclosure include without limitation, those from tobacco etch virus (TEV), picornaviruses (e.g., FMDV), pest viruses (CFFV), polio viruses (PV), encephalomyocarditis viruses (EMCV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV). An IRES sequence can be derived, for example, from commercially available vectors such as the IRES sequences available from Clontech™, GeneCopoeia™, or Sigma-Aldrich™. IRES sequences can be, for example, at least 150 bases or base pairs, 200 bases or base pairs, 300 bases or base pairs, 400 bases or base pairs, 500 bases or base pairs, 600 bases or base pairs, 700 bases or base pairs, 800 bases or base pairs, 900 bases or base pairs, 1000 bases or base pairs, 2000 bases or base pairs, 3000 bases or base pairs, 4000 bases or base pairs, 5000 bases or base pairs, or 10000 bases or base pairs. IRES sequences can at most 10000 bases or base pairs, 5000 bases or base pairs, 4000 bases or base pairs, 3000 bases or base pairs, 2000 bases or base pairs, 1000 bases or base pairs, 900 bases or base pairs, 800 bases or base pairs, 700 bases or base pairs, 600 bases or base pairs, 500 bases or base pairs, 400 bases or base pairs, 300 bases or base pairs, 200 bases or base pairs, 100 bases or base pairs, 50 bases or base pairs, or 10 bases or base pairs.

An engineered polyribonucleotide of the disclosure can comprise a polyA (poly-adenosine) sequence or polyA tail. A polyA sequence (e.g., polyA tail) can comprise any number of nucleotides. A polyA sequence can comprise a length of about 1 to about 10 bases or base pairs, about 10 to about 20 bases or base pairs, about 20 to about 50 bases or base pairs, about 50 to about 100 bases or base pairs, about 100 to about 500 bases or base pairs, about 500 to about 1000 bases or base pairs, about 1000 to about 2000 bases or base pairs, about 2000 to about 3000 bases or base pairs, about 3000 to about 4000 bases or base pairs, about 4000 to about 5000 bases or base pairs, about 5000 to about 6000 bases or base pairs, about 6000 to about 7000 bases or base pairs, about 7000 to about 8000 bases or base pairs, about 8000 to about 9000 bases or base pairs, or about 9000 to about 10000 bases or base pairs in length. In some examples, a polyA sequence is at least about 100, 110, 120, 130, 140, 150, 160, 170, 180, 190, or 200 nucleotides in length. A polyA sequence can comprise a length of for example, at least 1 base or base pair, 2 bases or base pairs, 3 bases or base pairs, 4 bases or base pairs, 5 bases or base pairs, 6 bases or base pairs, 7 bases or base pairs, 8 bases or base pairs, 9 bases or base pairs, 10 bases or base pairs, 20 bases or base pairs, 30 bases or base pairs, 40 bases or base pairs, 50 bases or base pairs, 60 bases or base pairs, 70 bases or base pairs, 80 bases or base pairs, 90 bases or base pairs, 100 bases or base pairs, 200 bases or base pairs, 300 bases or base pairs, 400 bases or base pairs, 500 bases or base pairs, 600 bases or base pairs, 700 bases or base pairs, 800 bases or base pairs, 900 bases or base pairs, 1000 bases or base pairs, 2000 bases or base pairs, 3000 bases or base pairs, 4000 bases or base pairs, 5000 bases or base pairs, 6000 bases or base pairs, 7000 bases or base pairs, 8000 bases or base pairs, 9000 bases or base pairs, or 10000 bases or base pairs in length. A polyA sequence can comprise a length of at most 500 bases or base pairs, 400 bases or base pairs, 300 bases or base pairs, 200 bases or base pairs 100 bases or base pairs, 90 bases or base pairs, 80 bases or base pairs, 70 bases or base pairs, 60 bases or base pairs, 50 bases or base pairs, 40 bases or base pairs, 30 bases or base pairs, 20 bases or base pairs, 10 bases or base pairs, or 5 bases or base pairs.

A polyA tail may improve a pharmacokinetic characteristic of the polynucleotide in a subject or in a solution. For example, the polyA tail may allow a polynucleotide to have a longer half-life than a corresponding polynucleotide without a polyA tail. Without being limited to specific mechanism, the polyA tail may reduce degradation of coding sequences in a polynucleotide. The terminal end of a polyA tail may be hydrolyzed or otherwise degraded and prevent the hydrolysis of terminal end of a coding sequence. The length of polyA tail may influence the pharmacokinetic characteristics of the polynucleotide. For example, a polynucleotide with a longer polyA tail may have a longer half-life than a corresponding polynucleotide with a shorter polyA tail.

In some cases, a percentage of the nucleotides in a poly-A sequence are modified. For instance, in some cases, fewer than 99%, 95%, 90%, 85%, 80%, 75%, 70%, 65%, 60%, 55%, 50%, 45%, 40%, 35%, 30%, 25%, 20%, 15%, 10%, 5% or 1% of the nucleotides in a poly-A sequence are modified. In some cases, all of the nucleotides in a poly-A are modified.

A linker sequence can comprise any number of nucleotides. A linker can be attached to the modified nucleobase at an N-3 or C-5 position. The linker attached to the nucleobase can be diethylene glycol, dipropylene glycol, triethylene glycol, tripropylene glycol, tetraethylene glycol, tetraethylene glycol, divalent alkyl, alkenyl, alkynyl moiety, ester, amide, or an ether moiety. A linker sequence can comprise a length of about 1 to about 10 bases or base pairs, about 10 to about 20 bases or base pairs, about 20 to about 50 bases or base pairs, about 50 to about 100 bases or base pairs, about 100 to about 500 bases or base pairs, about 500 to about 1000 bases or base pairs, about 1000 to about 2000 bases or base pairs, about 2000 to about 3000 bases or base pairs, about 3000 to about 4000 bases or base pairs, about 4000 to about 5000 bases or base pairs, about 5000 to about 6000 bases or base pairs, about 6000 to about 7000 bases or base pairs, about 7000 to about 8000 bases or base pairs, about 8000 to about 9000 bases or base pairs, or about 9000 to about 10000 bases or base pairs in length. A linker sequence can comprise a length of for example, at least 1 base or base pair, 2 bases or base pairs, 3 bases or base pairs, 4 bases or base pairs, 5 bases or base pairs, 6 bases or base pairs, 7 bases or base pairs, 8 bases or base pairs, 9 bases or base pairs, 10 bases or base pairs, 20 bases or base pairs, 30 bases or base pairs, 40 bases or base pairs, 50 bases or base pairs, 60 bases or base pairs, 70 bases or base pairs, 80 bases or base pairs, 90 bases or base pairs, 100 bases or base pairs, 200 bases or base pairs, 300 bases or base pairs, 400 bases or base pairs, 500 bases or base pairs, 600 bases or base pairs, 700 bases or base pairs, 800 bases or base pairs, 900 bases or base pairs, 1000 bases or base pairs, 2000 bases or base pairs, 3000 bases or base pairs, 4000 bases or base pairs, 5000 bases or base pairs, 6000 bases or base pairs, 7000 bases or base pairs, 8000 bases or base pairs, 9000 bases or base pairs, or at least 10000 bases or base pairs in length. A linker at most 10000 bases or base pairs, 5000 bases or base pairs, 4000 bases or base pairs, 3000 bases or base pairs, 2000 bases or base pairs, 1000 bases or base pairs, 900 bases or base pairs, 800 bases or base pairs, 700 bases or base pairs, 600 bases or base pairs, 500 bases or base pairs, 400 bases or base pairs, 300 bases or base pairs, 200 bases or base pairs, or 100 bases or base pairs in length.

In some cases, a percentage of the nucleotides in a linker sequence are modified. For instance, in some cases, fewer than 99%, 95%, 90%, 85%, 80%, 75%, 70%, 65%, 60%, 55%, 50%, 45%, 40%, 35%, 30%, 25%, 20%, 15%, 10%, 5% or 1% of the nucleotides in a linker sequence are modified. In some cases, all of the nucleotides in a linker sequence are modified.

In some cases, a nucleic acid construct(s), a vector(s), or an engineered polyribonucleotide(s) can include at least one stop codon before the 3′untranslated region (UTR). In some cases, a nucleic acid construct(s), a vector(s), or an engineered polyribonucleotide(s) includes multiple stop codons. The stop codon can be selected from TGA, TAA and TAG. The stop codon may be modified or unmodified. In some cases, the nucleic acid construct(s), vector(s), or engineered polyribonucleotide(s) includes the stop codon TGA and one additional stop codon. In some cases, the nucleic acid construct(s), vector(s), or engineered polyribonucleotide(s) includes the addition of the TAA stop codon.

### Encoded Polypeptides

In some cases, the disclosure a polynucleotide that encodes for armadillo repeat containing 4 (ARMC4), chromosome 21 open reading frame 59 (C21orf59), coiled-coil domain containing 103 (CCDC103), coiled-coil domain containing 114 (CCDC114), coiled-coil domain containing 39 (CCDC39), coiled-coil domain containing 40 (CCDC40), coiled-coil domain containing 65 (CCDC65), dynein (axonemal) assembly factor 1 (DNAAF1), dynein (axonemal) assembly factor 2 (DNAAF2), dynein (axonemal) assembly factor 3 (DNAAF3), dynein (axonemal) assembly factor 4 (DNAAF4), dynein (axonemal) assembly factor 5 (DNAAFS), dynein axonemal heavy chain 11 (DNAH11), dynein axonemal heavy chain 5 (DNAHS), dynein axonemal heavy chain 8 (DNAH8), dynein axonemal intermediate chain 2 (DNAI2), dynein axonemal light chain 1 (DNAL1), dynein regulatory complex subunit 1 (DRC1), axonemal central pair apparatus protein (HYDIN), leucine rich repeat containing 6 (LRRC6), NME/NM23 family member 8 (NME8), oral-facial-digital syndrome 1 (OFD1), retinitis pigmentosa GTPase regulator (RPGR), radial spoke head 1 homolog (Chlamydomonas) (RSPH1), radial spoke head 4 homolog A (Chlamydomonas) (RSPH4A), radial spoke head 9 homolog (Chlamydomonas) (RSPH9), sperm associated antigen 1(SPAG1), and zinc finger MYND-type containing 10 (ZMYND10) , or a variant of any of the aforementioned.

The encoded polypeptides are polymer chains comprised of amino acid residue monomers which are joined together through amide bonds (peptide bonds). The amino acids may be of the L-optical isomer, the D-optical isomer or a combination thereof. A polypeptide can be a chain of at least three amino acids, peptide-mimetics, a protein, a recombinant protein, an antibody (monoclonal or polyclonal), an antigen, an epitope, an enzyme, a receptor, a vitamin, or a structure analogue or combinations thereof. A polyribonucleotide that is translated within a subject's body can generate an ample supply of specific peptides or proteins within a cell, a tissue, or across many cells and tissues of a subject. In some cases, a polyribonucleotide can be translated in vivo within the cytosol of a specific target cell(s) type or target tissue.

In some cases, a polyribonucleotide can be translated in vivo to provide a protein whose gene has been associated with primary ciliary dyskinesia, a functional fragment thereof. In some case, polyribonucleotide can be translated in vivo to provide a protein whose gene is selected from the group consisting of pDNAH5, ARMC4, ZMYND10, DNAAF4, CCDC40, CCDC39, DNAAF1, DNAI2, or DAAF2.

In some cases, a polyribonucleotide can be translated in vivo in various non-target cell types or target tissue(s). Non-limiting examples of cells that be target or non-target cells include: a) skin cells, e.g.: keratinocytes, melanocytes, urothelial cells; b) neural cells, e.g.: neurons, Schwann cells, oligodentrocytes, astrocytes; c) liver cells, e.g.: hepatocytes; d) intestinal cells, e.g.: goblet cell, enterocytes; e) blood cells; e.g.: lymphoid or myeloid cells; and f) germ cells; e.g.: sperm and eggs. Non-limiting examples of tissues include connective tissue, muscle tissue, nervous tissue, or epithelial tissue. In some cases, a target cell or a target tissue is a cancerous cell, tissue, or organ.

A polynucleotide sequence can be derived from one or more species. For example, a polynucleotide sequence can be derived from a human (Homo sapiens), a mouse (e.g., Mus musculus), a rat (e.g., Rattus norvegicus or Rattus rattus), a microorganism (e.g., Chlamydomonas genus), or any other suitable creature. A polynucleotide sequence can be a chimeric combination of the sequence of one or more species.

In some cases, the endogenous translational machinery can add a post-translational modification to the encoded peptide. A post-translational modification can involve the addition of hydrophobic groups that can target the polypeptide for membrane localization, the addition of cofactors for increased enzymatic activity, or the addition of smaller chemical groups. The encoded polypeptide can also be post-translationally modified to receive the addition of other peptides or protein moieties. For instance, ubiquitination can lead to the covalent linkage of ubiquitin to the encoded polypeptide, SUMOylation can lead to the covalent linkage of SUMO (Small Ubiquitin-related MOdifier) to the encoded polypeptide, ISGylation can lead to the covalent linkage of ISG15 (Interferon-Stimulate Gene 15).

In some cases, the encoded polypeptide can be post-translationally modified to undergo other types of structural changes. For instance, the encoded polypeptide can be proteolytically cleaved, and one or more proteolytic fragments can modulate the activity of an intracellular pathway. The encoded polypeptide can be folded intracellularly. In some cases, the encoded polypeptide is folded in the presence of co-factors and molecular chaperones. A folded polypeptide can have a secondary structure and a tertiary structure. A folded polypeptide can associate with other folded peptides to form a quaternary structure. A folded-peptide can form a functional multi-subunit complex, such as an antibody molecule, which has a tetrameric quaternary structure. Various polypeptides that form classes or isotypes of antibodies can be expressed from a polyribonucleotide.

The encoded polypeptide can be post-translationally modified to change the chemical nature of the encoded amino acids. For instance, the encoded polypeptide can undergo post-translational citrullination or deimination, the conversion of arginine to citrulline. The encoded polypeptide can undergo post-translation deamidation; the conversion of glutamine to glutamic acid or asparagine to aspartic acid. The encoded polypeptide can undergo elimination, the conversion of an alkene by beta-elimination of phosphothreonine and phosphoserine, or dehydration of threonine and serine, as well as by decarboxylation of cysteine. The encoded peptide can also undergo carbamylation, the conversion of lysine to homocitrulline. An encoded peptide can also undergo racemization, for example, racemization of proline by prolyl isomerase or racemization of serine by protein-serine epimerase. In some cases, an encoded peptide can undergo serine, threonine, and tyrosine phosphorylation.

The activity of a plurality of biomolecules can be modulated by a molecule encoded by a polyribonucleotide. Non-limiting examples of molecules whose activities can be modulated by an encoded polynucleotide include: amino acids, peptides, peptide-mimetics, proteins, recombinant proteins antibodies (monoclonal or polyclonal), antibody fragments, antigens, epitopes, carbohydrates, lipids, fatty acids, enzymes, natural products, nucleic acids (including DNA, RNA, nucleosides, nucleotides, structure analogues or combinations thereof), nutrients, receptors, and vitamins.

### Lipid Formulations

The compositions may comprise engineered polyribonucleotides, vectors, or nucleic acid constructs. “Naked” polynucleotide compositions can be successfully administered to a subject, and uptaken by a subject's cell, without the aid of carriers, stabilizers, diluents, dispersing agents, suspending agents, thickening agents, and/or excipients (Wolff et al. 1990, Science, 247, 1465-1468). However, in many instances, encapsulation of polynucleotides with formulations that can increase the endocytotic uptake can increase the effectiveness of a composition of the disclosure. To overcome this challenge, in some cases, the composition comprises a nucleic acid construct, a vector, or an isolated nucleic acid encoding dynein axonemal intermediate chain 1, wherein the nucleic acid construct comprises a complementary deoxyribonucleic acid encoding dynein axonemal intermediate chain 1, which composition is formulated for administration to a subject.

Another technical challenge underlying the delivery of polyribonucleotides to multicellular organisms is to identify a composition that provides a high efficiency delivery of polyribonucleotides that are translated within a cell or a tissue of a subject. It has been recognized that administration of naked nucleic acids may be highly inefficient and may not provide a suitable approach for administration of a polynucleotide to a multicellular organism.

To solve this challenge, a composition comprising an engineered polyribonucleotide can be encapsulated or formulated with a pharmaceutical carrier. The formulation may be, but is not limited to, nanoparticles, nanocapsules poly(lactic-co-glycolic acid) (PLGA) microspheres, lipidoids, lipoplex, liposome, polymers, carbohydrates (including simple sugars), cationic lipids, fibrin gel, fibrin hydrogel, fibrin glue, fibrin sealant, fibrinogen, thrombin, rapidly eliminated lipid nanoparticles (reLNPs) and combinations thereof. A composition comprising an engineered polyribonucleotide disclosed herein can comprise from about 1% to about 99% weight by volume of a carrier system. The amount of carrier present in a carrier system is based upon several different factors or choices made by the formulator, for example, the final concentration of the polyribonucleotide and the amount of solubilizing agent. Various carriers have been shown useful in delivery of different classes of therapeutic agents. Among these carriers, biodegradable nanoparticles formulated from biocompatible polymers poly(D,L-lactide-co-glycolide) (PLGA) and polylactide (PLA) have shown the potential for sustained intracellular delivery of different therapeutic agents.

Provided herein include (e.g., pharmaceutical) compositions comprising a polynucleotide as described herein. The pharmaceutical composition may comprise a polynucleotide combined with a lipid composition. A pharmaceutical composition may comprise a polynucleotide combined with a lipid composition, which polynucleotide (1) encodes a primary ciliary dyskinesia (PCD)-associated protein and (2) comprises a nucleic acid sequence having at least about 70% sequence identity over at least 100 bases to a sequence selected from SEQ ID NOs: 1-32, 61, or 62. The pharmaceutical compositions may comprise a cationic lipid or cationic polymer.

The pharmaceutical composition may further comprise a phospholipid or other zwitterionic lipids. In various embodiments described herein, the phospholipid may contain one or two long chain (e.g., C₆-C₂₄) alkyl or alkenyl groups, a glycerol or a sphingosine, one or two phosphate groups, and, optionally, a small organic molecule. The small organic molecule may be an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge.

The pharmaceutical composition may further comprise a polymer-conjugated lipid (e.g., poly(ethylene glycol) (PEG)-conjugated lipid). In various embodiments described herein in the “lipid formulations” section, the PEG lipid is a diglyceride which also comprises a PEG chain attached to the glycerol group. In other embodiments, the PEG lipid is a compound which contains one or more C₆-C₂₄ long chain alkyl or alkenyl group or a C₆-C₂₄ fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present disclosure are taught by U.S. Pat. No. 5,820,873, WO 2010/141069, or U.S. Pat. No. 8,450,298, which is incorporated herein by reference.

The pharmaceutical composition may further comprise a steroid or steroid derivative. In various embodiments described herein, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula:

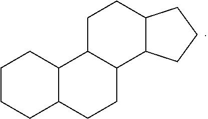 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

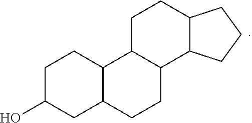 [SMILES: OC1CCC2C(CCC3C4CCCC4CCC23)C1]

In some embodiments of the present disclosure, the steroid or steroid derivative is a cholestane or cholestane derivative. In a cholestane, the ring structure is further defined by the formula:

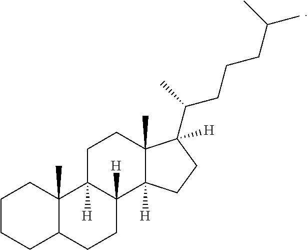

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestere and a sterol or a derivative thereof.

The pharmaceutical formulation may be formulated in a nanoparticle or a nanocapsule. The pharmaceutical formulation may be formulated for local or systemic administration. For example, formulations may be nanoparticle based formulations of nucleic acid constructs, engineered polyribonucleotides, or vectors that are able to translocate following administration to a subject. In some instances, the administration is pulmonary and the engineered polyribonucleotides can move intact either actively or passively from the site of administration to the systemic blood supply and subsequently to be deposited in different cells or tissues, such as, e.g., the breast. This translocation of the nanoparticle comprising an engineered polyribonucleotide encoding a therapeutic protein, such as, e.g., dynein axonemal intermediate chain 1 (DNAI1), armadillo repeat containing 4 (ARMC4), chromosome 21 open reading frame 59 (C21orf59), coiled-coil domain containing 103 (CCDC103), coiled-coil domain containing 114 (CCDC114), coiled-coil domain containing 39 (CCDC39), coiled-coil domain containing 40 (CCDC40), coiled-coil domain containing 65 (CCDC65), cyclin O (CCNO), dynein (axonemal) assembly factor 1 (DNAAF1), dynein (axonemal) assembly factor 2 (DNAAF2), dynein (axonemal) assembly factor 3 (DNAAF3), dynein (axonemal) assembly factor 4 (DNAAF4), dynein (axonemal) assembly factor 5 (DNAAF5), dynein axonemal heavy chain 11 (DNAH11), dynein axonemal heavy chain 5 (DNAHS), dynein axonemal heavy chain 6 (DNAH6),dynein axonemal heavy chain 8 (DNAH8), dynein axonemal intermediate chain 2 (DNAI2), dynein axonemal light chain 1 (DNAL1), dynein regulatory complex subunit 1 (DRC1), growth arrest specific 8 (GAS8), axonemal central pair apparatus protein (HYDIN), leucine rich repeat containing 6 (LRRC6), NME/NM23 family member 8 (NME8), oral-facial-digital syndrome 1 (OFD1), retinitis pigmentosa GTPase regulator (RPGR), radial spoke head 1 homolog (Chlamydomonas) (RSPH1), radial spoke head 4 homolog A (Chlamydomonas) (RSPH4A), radial spoke head 9 homolog (Chlamydomonas) (RSPH9), sperm associated antigen 1(SPAG1), and zinc finger MYND-type containing 10 (ZMYND10) or a functional fragment thereof, constitutes non-invasive systemic delivery of an active pharmaceutical ingredient beyond the lung to result in the production of a functional protein to systemically accessible non-lung cells or tissues.

A nanoparticle can be a particle of particle size from about 10 nanometers (nm) to 5000 nm, 10 nm to 1000 nm, or 60 nm to 500 nm, or 70 nm to 300 nm. In some examples, a nanoparticle has a particle size from about 60 nm to 225 nm. The nanoparticle can include an encapsulating agent (e.g., coating) that encapsulates one or more polyribonucleotides, which may be engineered polyribonucleotides. The nanoparticle can include engineered and/or naturally occurring polyribonucleotides. The encapsulating agent can be a polymeric material, such as PEI or PEG.

A lipidoid or lipid nanoparticle which may be used as a delivery agent may include a lipid which may be selected from the group consisting of C12-200, MD1, 98N12-5, DLin-DMA, DLin-K-DMA, DLin-KC2-DMA, DLin-MC3-DMA, PLGA, PEG, PEG-DMG, PEGylated lipids and analogues thereof. A suitable nanoparticle can comprise one or more lipids in various ratios. For example, a composition of the disclosure can comprise a 40:30:25:5 ratio of C12-200:DOPE:Cholesterol:DMG-PEG2000 or a 40:20:35:5 ratio of HGT5001:DOPE:Cholesterol: DMG-PEG2000. A nanoparticle can include at least 1, 2, 3, 4, 5, 6, 7, 8, 9, 10 lipids or another suitable number of lipids. A nanoparticle can be formed of any suitable ratio of lipids selected from the group consisting of C12-200, MD1, 98N12-5, DLin-DMA, DLin-K-DMA, DLin-KC2-DMA, DLin-MC3-DMA, PLGA, PEG, PEG-DMG.

The mean size of the nanoparticle formulation may comprise the modified mRNA between 60 nanometers (nm) and 225 nm. The polydispersity index PDI of the nanoparticle formulation comprising the modified mRNA can be between 0.03 and 0.15. The zeta potential of the nanoparticle formulation may be from −10 to +10 at a pH of 7.4. The formulations of modified mRNA may comprise a fusogenic lipid, cholesterol and a PEG lipid. The formulation may have a molar ratio 50:10:38.5:1.5-3.0 (cationic lipid:fusogenic lipid: cholesterol: polyethylene glycol (PEG) lipid). The PEG lipid may be selected from, but is not limited to PEG-c-DOMG, PEG-DMG. The fusogenic lipid may be DSPC. A lipid nanoparticle of the present disclosure can be formulated in a sealant such as, but not limited to, a fibrin sealant.

In some embodiments, the polynucleotide is present in the (e.g., pharmaceutical) composition at a concentration of no more than 1 mg/mL. In some embodiments, the polynucleotide is present in the (e.g., pharmaceutical) composition at a concentration of no more than 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, or less. In some embodiments, the polynucleotide is present in the (e.g., pharmaceutical) composition at a concentration of at least 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, or more. In some embodiments, the polynucleotide is present in the (e.g., pharmaceutical) composition at a concentration of any one of the following values or within a range of any two of the following values: 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, or a range between any two of the foregoing values.

In some embodiments, the pharmaceutical formulation may be a dry powder formulation. The dry powder formulation may comprise a polynucleotides and nanocapsules or nanoparticles as described elsewhere herein. The dry powder formulation may be administered to a subject in the dry powder form. The dry powder formulation may be generated by spray drying the components of the formulation. The dry powder formulation may allow the structure or function of the polynucleotides to be maintained (e.g., after storage). The dry powder formulation may allow the structure or function of the nanocapsules of nanoparticles to be maintained (e.g., after storage). For example, the dry powder formulation may maintain an encapsulation or interaction of the polynucleotides with the nanoparticles.

### Kits

Provided herein, in some embodiments, include a kit comprising a (e.g., pharmaceutical) composition described herein, a container, and a label or package insert on or associated with the container.

### Methods of Treatment

Provided herein include methods for treating a subject (e.g., a patient with a disease and/or a lab animal with a condition). In some cases, the condition is primary ciliary dyskinesia (PCD) or Kartagener syndrome. In some cases, the condition is broadly associated with defects in one or more proteins that function within cell structures known as cilia. In some cases, the subject is a human. Treatment may be provided to the subject before clinical onset of disease. Treatment may be provided to the subject after clinical onset of disease. Treatment may be provided to the subject on or after 1 minute, 5 minutes, 10 minutes, 30 minutes, 1 hour, 2 hours, 3 hours, 4 hours, 5 hours, 6 hours, 12 hours, 1 day, 1 week, 6 months, 12 months, or 2 years after clinical onset of the disease. Treatment may be provided to the subject for a time period that is greater than or equal to 1 minute, 10 minutes, 30 minutes, 1 hour, 2 hours, 3 hours, 4 hours, 5 hours, 6 hours, 12 hours, 1 day, 1 week, 1 month, 6 months, 12 months, 2 years or more after clinical onset of the disease. Treatment may be provided to the subject for a time period that is less than or equal to 2 years, 12 months, 6 months, 1 month, 1 week, 1 day, 12 hours, 6 hours, 5 hours, 4 hours, 3 hours, 2 hours, 1 hour, 30 minutes, 10 minutes, or 1 minute after clinical onset of the disease. Treatment may also include treating a human in a clinical trial.

Provided here include methods of treating a subject having or suspected of having primary ciliary dyskinesia (PCD), comprising administering to the subject a (e.g., pharmaceutical) composition as provided hereinabove or elsewhere herein, thereby resulting in a heterologous expression of the PCD-associated protein within cells of the subject. The (e.g., pharmaceutical) compositions as described hereinabove or elsewhere herein may be effective at treating a subject having PCD. The (e.g., pharmaceutical) compositions as described hereinabove or elsewhere herein may be effective at treating a subject suspected of having PCD. The (e.g., pharmaceutical) compositions may alleviate or eliminate symptoms of PCD in the subject (e.g., regardless whether the subject has been determined to have PCD).

The present disclosure provides a method for treating a subject having or suspected of having primary ciliary dyskinesia (PCD), comprising administering to the subject in need thereof a (e.g. pharmaceutical) composition comprising a polynucleotide combined with a lipid composition, which polynucleotide (1) encodes a primary ciliary dyskinesia (PCD)-associated protein and (2) comprises a nucleic acid sequence having at least about 70% sequence identity over at least 100 bases to a sequence selected from SEQ ID NOs: 1-32, 61, or 62, thereby resulting in a heterologous expression of the PCD-associated protein within cells of the subject.

The method for treating a subject having or suspected of having primary ciliary dyskinesia (PCD), may comprise administering to the subject in need thereof a pharmaceutical composition comprising a polynucleotide coupled to a lipid composition, which polynucleotide (1) encodes a primary ciliary dyskinesia (PCD)-associated protein and (2) comprises a nucleic acid sequence having at least about 70% sequence identity over at least 100 bases to a sequence selected from SEQ ID NOs: 1-32, 61, or 62, thereby resulting in a heterologous expression of the PCD-associated protein within cells of the subject.

The methods as described herein may comprises treating or administering a composition to the subject. In some cases, the subject may be determined to have PCD. The subject may be observed or determined to have a genetic or expression profile that is aberrant from a health individual. An aberrant genetic profile or expression profile may be indicative of a particular disease or disorder. The subject may be determined to exhibit aberrant expression or activity of a PCD-associated gene or protein. The aberrant expression or activity may be an excess or increased activity of a protein or gene that results in a disease state. The aberrant expression or activity may be a decrease or loss of activity of a protein or gene that results in a disease state. The aberrant expression may be a loss of activity such that a particular function of a protein is lost. The aberrant expression may be alleviated by the introduction of a composition that increases the expression of a protein and allows a regain of protein function in a cell or organ.

The cells comprising aberrant expression and/or the cells wherein the composition are administered to may be a particular type of cell or located in a particular area of the body of the subject. The cells may be lung cells. The cells may be located in the lung of the subject. The cells may be undifferentiated or differentiated. In some embodiments, the cells are ciliated cells. In some embodiments, the ciliated cells are ciliated epithelial cells. For example, the ciliated cells may be ciliated airway epithelial cells. In some embodiments, the epithelial cells are undifferentiated. In some embodiments, the epithelial cells are differentiated.

### List of Embodiments

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A synthetic polynucleotide encoding a primary ciliary dyskinesia (PCD)-associated protein, wherein said synthetic polynucleotide comprises a nucleic acid sequence (e.g., an open reading frame (ORF) sequence) having at least about 70% sequence identity over at least 500 or 1000 bases to a sequence selected from SEQ ID NOs: 1-32, 61, or 62.

Embodiment 2. The synthetic polynucleotide of Embodiment 1, wherein said nucleic acid sequence has at least about 75% (e.g., at least about 80%, 81%, 82%, 83%, 84%, 85%, 86%, 87%, 88%, 89%, 90%, 91%, 92%, 93%, 94%, 95%, 96%, 97%, 98%, or 99%) sequence identity over at least 500 or 1000 bases to a sequence selected from SEQ ID NOs: 1-32, 61, or 62.

Embodiment 3. The synthetic polynucleotide of Embodiment 1 or 2, wherein said nucleic acid sequence has 100% sequence identity over at least 500 or 1000 bases to a sequence selected from SEQ ID NOs: 1-32, 61, or 62.

Embodiment 4. The synthetic polynucleotide of Embodiment 1, wherein said nucleic acid sequence has at least about 70% sequence identity to a sequence selected from SEQ ID NOs: 1-32, 61, or 62.

Embodiment 5. The synthetic polynucleotide of Embodiment 4, wherein said nucleic acid sequence has at least about 75% (e.g., at least about 80%, 81%, 82%, 83%, 84%, 85%, 86%, 87%, 88%, 89%, 90%, 91%, 92%, 93%, 94%, 95%, 96%, 97%, 98%, or 99%) sequence identity to a sequence selected from SEQ ID NOs: 1-32, 61, or 62.

Embodiment 6. The synthetic polynucleotide of any one of Embodiments 1-5, wherein said nucleic acid sequence comprises a reduced number or frequency of at least one codon selected from the group consisting of GCG, GCA, GCT, TGT, GAT, GAG, TTT, GGG, GGT, CAT, ATA, ATT, AAG, TTG, TTA, CTA, CTT, CTC, AAT, CCG, CCA, CCT, CAG, AGG, CGG, CGA, CGT, CGC, TCG, TCA, TCT, TCC, ACG, ACT, GTA, GTT, GTC, and TAT, as compared to a corresponding wild-type sequence selected from SEQ ID NOs: 33-39.

Embodiment 7. The synthetic polynucleotide of any one of Embodiments 1-5, wherein said nucleic acid sequence comprises an increased number or frequency of at least one codon comprising one or more codons selected from: GCC, TGC, GAC, GAA, TTC, GGA, GGC, CAC, ATC, AAA, CTG, AAC, CCT, CCC, CAA, AGA, AGC, ACA, ACC, GTG, and TAC, as compared to a corresponding wild-type sequence selected from SEQ ID NOs: 33-39.

Embodiment 8. The synthetic polynucleotide of any one of Embodiments 1-7, wherein said nucleic acid sequence comprises fewer codon types encoding an amino acid as compared to a corresponding wild-type sequence selected from SEQ ID NOs: 33-39.

Embodiment 9. The synthetic polynucleotide of any one of Embodiments 1-8, wherein at least one type of an isoleucine-encoding codon in said corresponding wild-type sequence is substituted with a synonymous codon type in said nucleic acid sequence.

Embodiment 10. The synthetic polynucleotide of any one of Embodiments 1-9, wherein at least one type of a valine-encoding codon in said corresponding wild-type sequence is substituted with a synonymous codon type in said nucleic acid sequence.

Embodiment 11. The synthetic polynucleotide of any one of Embodiments 1-10, wherein at least one type of an alanine-encoding codon in said corresponding wild-type sequence is substituted with a synonymous codon type in said nucleic acid sequence.

Embodiment 12. The synthetic polynucleotide of any one of Embodiments 1-11, wherein at least one type of a glycine-encoding codon in said corresponding wild-type sequence is substituted with a synonymous codon type in said nucleic acid sequence.

Embodiment 13. The synthetic polynucleotide of any one of Embodiments 1-12, wherein at least one type of a proline-encoding codon in said corresponding wild-type sequence is substituted with a synonymous codon type in said nucleic acid sequence.

Embodiment 14. The synthetic polynucleotide of any one of Embodiments 1-13, wherein at least one type of a threonine-encoding codon in said corresponding wild-type sequence is substituted with a synonymous codon type in said nucleic acid sequence.

Embodiment 15. The synthetic polynucleotide of any one of Embodiments 1-14, wherein at least one type of a leucine-encoding codon in said corresponding wild-type sequence is substituted with a synonymous codon type in said nucleic acid sequence.

Embodiment 16. The synthetic polynucleotide of any one of Embodiments 1-15, wherein at least one type of an arginine-encoding codon in said corresponding wild-type sequence is substituted with a synonymous codon type in said nucleic acid sequence.

Embodiment 17. The synthetic polynucleotide of any one of Embodiments 1-16, wherein at least one type of a serine-encoding codon in said corresponding wild-type sequence is substituted with a synonymous codon type in said nucleic acid sequence.

Embodiment 18. The synthetic polynucleotide of any one of Embodiments 1-17, wherein at least about 90% phenylalanine-encoding codons of said synthetic polynucleotide are TTC (as opposed to TTT).

Embodiment 19. The synthetic polynucleotide of any one of Embodiments 1-18, wherein at least about 60% cysteine-encoding codons of said synthetic polynucleotide are TGC (as opposed to TGT).

Embodiment 20. The synthetic polynucleotide of any one of Embodiments 1-19, wherein at least about 70% aspartic acid-encoding codons of said synthetic polynucleotide are GAC (as opposed to GAT).

Embodiment 21. The synthetic polynucleotide of any one of Embodiments 1-20, wherein at least about 50% glutamic acid-encoding codons of said synthetic polynucleotide are GAG (as opposed to GAA).

Embodiment 22. The synthetic polynucleotide of any one of Embodiments 1-21, wherein at least about 60% histidine-encoding codons of said synthetic polynucleotide are CAC (as opposed to CAT).

Embodiment 23. The synthetic polynucleotide of any one of Embodiments 1-22, wherein at least about 60% lysine-encoding codons of said synthetic polynucleotide are AAG (as opposed to AAA).

Embodiment 24. The synthetic polynucleotide of any one of Embodiments 1-23, wherein at least about 60% asparagine-encoding codons of said synthetic polynucleotide are AAC (as opposed to AAT).

Embodiment 25. The synthetic polynucleotide of any one of Embodiments 1-24, wherein at least about 70% glutamine-encoding codons of said synthetic polynucleotide are CAG (as opposed to CAA).

Embodiment 26. The synthetic polynucleotide of any one of Embodiments 1-25, wherein at least about 80% tyrosine-encoding codons of said synthetic polynucleotide are TAC (as opposed to TAT).

Embodiment 27. The synthetic polynucleotide of any one of Embodiments 1-26, wherein at least about 90% isoleucine-encoding codons of said synthetic polynucleotide are ATC.

Embodiment 28. The synthetic polynucleotide of any one of Embodiments 1-26, wherein said synthetic polynucleotide comprises no more than 2 types of isoleucine-encoding codons.

Embodiment 29. The synthetic polynucleotide of any one of Embodiments 1-28, wherein said synthetic polynucleotide comprises no more than 3 types of alanine (Ala)-encoding codons.

Embodiment 30. The synthetic polynucleotide of any one of Embodiments 1-29, wherein said synthetic polynucleotide comprises no more than 3 types of glycine (Gly)-encoding codons.

Embodiment 31. The synthetic polynucleotide of any one of Embodiments 1-30, wherein said synthetic polynucleotide comprises no more than 3 types of proline (Pro)-encoding codons.

Embodiment 32. The synthetic polynucleotide of any one of Embodiments 1-31, wherein said synthetic polynucleotide comprises no more than 3 types of threonine (Thr)-encoding codons.

Embodiment 33. The synthetic polynucleotide of any one of Embodiments 1-32, wherein said synthetic polynucleotide comprises no more than 5 or 4 type(s) of arginine (Arg)-encoding codons.

Embodiment 34. The synthetic polynucleotide of any one of Embodiments 1-33, wherein said synthetic polynucleotide comprises no more than 5 or 4 type(s) of serine (Ser)-encoding codons.

Embodiment 35. The synthetic polynucleotide of any one of Embodiments 1-34, a frequency of GCC codon is higher than a frequency of GCA codon.

Embodiment 36. The synthetic polynucleotide of any one of Embodiments 1-35, a frequency of GCC codon is higher than a frequency of GCT codon.

Embodiment 37. The synthetic polynucleotide of any one of Embodiments 1-36, a frequency of GCT codon is lower than a frequency of GCA codon.

Embodiment 38. The synthetic polynucleotide of any one of Embodiments 1-37, a frequency of GCT codon is higher than a frequency of GCA codon.

Embodiment 39. The synthetic polynucleotide of any one of Embodiments 1-38, a frequency of GCG codon is no more than about 10% or 5%.

Embodiment 40. The synthetic polynucleotide of any one of Embodiments 1-39, a frequency of GCA codon is no more than about 20%.

Embodiment 41. The synthetic polynucleotide of any one of Embodiments 1-40, a frequency of GCT codon is at least about 1%, 5%, 10%, 15%, 20%, or 25%.

Embodiment 42. The synthetic polynucleotide of any one of Embodiments 1-41, a frequency of GCT codon is no more than about 30%, 25%, 20%, 15%, 10%, or 5%.

Embodiment 43. The synthetic polynucleotide of any one of Embodiments 1-42, a frequency of GCC codon is at least about 60%, 70%, 80%, or 90%.

Embodiment 44. The synthetic polynucleotide of any one of Embodiments 1-43, a frequency of GCC codon is no more than about 95%, 90%, 85%, 80%, or 75%.

Embodiment 45. The synthetic polynucleotide of any one of Embodiments 1-44, a frequency of GGC codon is lower than a frequency of GGA codon.

Embodiment 46. The synthetic polynucleotide of any one of Embodiments 1-45, a frequency of GGC codon is higher than a frequency of GGA codon.

Embodiment 47. The synthetic polynucleotide of any one of Embodiments 1-46, a frequency of GGG codon is no more than about 10% or 5%.

Embodiment 48. The synthetic polynucleotide of any one of Embodiments 1-47, a frequency of GGG codon is at least about 1%.

Embodiment 49. The synthetic polynucleotide of any one of Embodiments 1-48, a frequency of GGA codon is no more than about 30% or 20%.

Embodiment 50. The synthetic polynucleotide of any one of Embodiments 1-49, a frequency of GGA codon is at least about 10% or 20%.

Embodiment 51. The synthetic polynucleotide of any one of Embodiments 1-50, a frequency of GGT codon is no more than about 10% or 5%.

Embodiment 52. The synthetic polynucleotide of any one of Embodiments 1-51, a frequency of GGC codon is no more than about 90%, 80%, or 70%.

Embodiment 53. The synthetic polynucleotide of any one of Embodiments 1-52, a frequency of GGC codon is at least about 60%, 70%, or 80%.

Embodiment 54. The synthetic polynucleotide of any one of Embodiments 1-53, a frequency of CCC codon is lower than a frequency of CCT codon.

Embodiment 55. The synthetic polynucleotide of any one of Embodiments 1-54, a frequency of CCC codon is higher than a frequency of CCT codon.

Embodiment 56. The synthetic polynucleotide of any one of Embodiments 1-55, a frequency of CCC codon is lower than a frequency of CCA codon.

Embodiment 57. The synthetic polynucleotide of any one of Embodiments 1-56, a frequency of CCC codon is higher than a frequency of CCA codon.

Embodiment 58. The synthetic polynucleotide of any one of Embodiments 1-57, a frequency of CCT codon is lower than a frequency of CCA codon.

Embodiment 59. The synthetic polynucleotide of any one of Embodiments 1-58, a frequency of CCT codon is higher than a frequency of CCA codon.

Embodiment 60. The synthetic polynucleotide of any one of Embodiments 1-59, a frequency of CCG codon is no more than about 10% or 5%.

Embodiment 61. The synthetic polynucleotide of any one of Embodiments 1-60, a frequency of CCA codon is no more than about 30%, 20%, or 10%.

Embodiment 62. The synthetic polynucleotide of any one of Embodiments 1-61, a frequency of CCA codon is at least about 5%, 10%, 15%, 20%, or 25%.

Embodiment 63. The synthetic polynucleotide of any one of Embodiments 1-62, a frequency of CCT codon is no more than about 60%, 50%, 40%, or 30%.

Embodiment 64. The synthetic polynucleotide of any one of Embodiments 1-63, a frequency of CCT codon is at least about 20%, 30%, 40%, or 50%.

Embodiment 65. The synthetic polynucleotide of any one of Embodiments 1-63, a frequency of CCC codon is no more than about 60%, 50%, or 40%.

Embodiment 66. The synthetic polynucleotide of any one of Embodiments 1-65, a frequency of CCC codon is at least about 30%, 40%, 50%, 60%, or 70%.

Embodiment 67. The synthetic polynucleotide of any one of Embodiments 1-66, a frequency of ACA codon is higher than a frequency of ACT codon.

Embodiment 68. The synthetic polynucleotide of any one of Embodiments 1-66, a frequency of ACC codon is higher than a frequency of ACT codon.

Embodiment 69. The synthetic polynucleotide of any one of Embodiments 1-68, a frequency of ACC codon is lower than a frequency of ACA codon.

Embodiment 70. The synthetic polynucleotide of any one of Embodiments 1-69, a frequency of ACC codon is higher than a frequency of ACA codon.

Embodiment 71. The synthetic polynucleotide of any one of Embodiments 1-70, a frequency of ACG codon is no more than about 10% or 5%.

Embodiment 72. The synthetic polynucleotide of any one of Embodiments 1-71, a frequency of ACA codon is no more than about 60%, 50%, 40%, or 30%.

Embodiment 73. The synthetic polynucleotide of any one of Embodiments 1-72, a frequency of ACA codon is at least about 10%, 20%, 30%, 40%, or 50%.

Embodiment 74. The synthetic polynucleotide of any one of Embodiments 1-73, a frequency of ACT codon is no more than about 10% or 5%.

Embodiment 75. The synthetic polynucleotide of any one of Embodiments 1-74, a frequency of ACC codon is no more than about 90%, 80%, 70%, 60%, or 50%.

Embodiment 76. The synthetic polynucleotide of any one of Embodiments 1-75, a frequency of ACC codon is at least about 40%, 50%, 60%, 70%, or 80%.

Embodiment 77. The synthetic polynucleotide of any one of Embodiments 1-76, a frequency of AGA codon is lower than a frequency of AGG codon.

Embodiment 78. The synthetic polynucleotide of any one of Embodiments 1-77, a frequency of AGA codon is higher than a frequency of AGG codon.

Embodiment 79. The synthetic polynucleotide of any one of Embodiments 1-78, a frequency of AGA codon is lower than a frequency of CGG codon.

Embodiment 80. The synthetic polynucleotide of any one of Embodiments 1-79, a frequency of AGA codon is higher than a frequency of CGG codon.

Embodiment 81. The synthetic polynucleotide of any one of Embodiments 1-80, a frequency of CGG codon is higher than a frequency of CGA codon.

Embodiment 82. The synthetic polynucleotide of any one of Embodiments 1-81, a frequency of CGG codon is higher than a frequency of CGC codon.

Embodiment 83. The synthetic polynucleotide of any one of Embodiments 1-82, a frequency of AGG codon is no more than about 10%.

Embodiment 84. The synthetic polynucleotide of any one of Embodiments 1-83, a frequency of AGG codon is less than about 10%.

Embodiment 85. The synthetic polynucleotide of any one of Embodiments 1-84, a frequency of AGA codon is no more than about 70%, 60%, or 50%.

Embodiment 86. The synthetic polynucleotide of any one of Embodiments 1-85, a frequency of AGA codon is at least about 40%, 50%, 60%, or 70%.

Embodiment 87. The synthetic polynucleotide of any one of Embodiments 1-86, a frequency of CGG codon is no more than about 50%, 40%, or 30%.

Embodiment 88. The synthetic polynucleotide of any one of Embodiments 1-87, a frequency of CGG codon is at least about 20%, 30%, or 40%.

Embodiment 89. The synthetic polynucleotide of any one of Embodiments 1-88, a frequency of CGA codon is at least about 1%.

Embodiment 90. The synthetic polynucleotide of any one of Embodiments 1-89, a frequency of CGA codon is no more than about 10% or 5%.

Embodiment 91. The synthetic polynucleotide of any one of Embodiments 1-90, a frequency of CGT codon is no more about 10% or 5%.

Embodiment 92. The synthetic polynucleotide of any one of Embodiments 1-91, a frequency of CGC codon is no more than about 20%, 10%, or 5%.

Embodiment 93. The synthetic polynucleotide of any one of Embodiments 1-92, a frequency of CGC codon is at least about 1%, 2%, 3%, 4%, or 5%.

Embodiment 94. The synthetic polynucleotide of any one of Embodiments 1-93, a frequency of AGC codon is higher than a frequency of TCT codon.

Embodiment 95. The synthetic polynucleotide of any one of Embodiments 1-94, a frequency of TCT codon is higher than a frequency of TCG codon.

Embodiment 96. The synthetic polynucleotide of any one of Embodiments 1-95, a frequency of TCT codon is higher than a frequency of TCA codon.

Embodiment 97. The synthetic polynucleotide of any one of Embodiments 1-96, a frequency of TCT codon is higher than a frequency of TCC codon.

Embodiment 98. The synthetic polynucleotide of any one of Embodiments 1-97, a frequency of AGT codon is no more than about 10%.

Embodiment 99. The synthetic polynucleotide of any one of Embodiments 1-98, a frequency of AGT codon is at least about 1%.

Embodiment 100. The synthetic polynucleotide of any one of Embodiments 1-99, a frequency of AGC codon is no more about 95%, 90%, 85%, or 80%.

Embodiment 101. The synthetic polynucleotide of any one of Embodiments 1-100, a frequency of AGC codon is at least about 70%, 80%, or 90%.

Embodiment 102. The synthetic polynucleotide of any one of Embodiments 1-101, a frequency of TCG codon is no more than about 10% or 5%.

Embodiment 103. The synthetic polynucleotide of any one of Embodiments 1-102, a frequency of TCA codon is no more than about 10% or 5%.

Embodiment 104. The synthetic polynucleotide of any one of Embodiments 1-103, a frequency of TCT codon is no more than about 30%, 20%, or 10%.

Embodiment 105. The synthetic polynucleotide of any one of Embodiments 1-104, a frequency of TCT codon is at least about 10%, or 20%.

Embodiment 106. The synthetic polynucleotide of any one of Embodiments 1-105, a frequency of TCC codon is no more than about 10% or 5%.

Embodiment 107. The synthetic polynucleotide of any one of Embodiments 1-106, wherein said synthetic polynucleotide further comprises a 3′ or 5′ noncoding region.

Embodiment 108. The synthetic polynucleotide of Embodiment 108, wherein said 3′ or 5′ noncoding region enhances an expression of said PCD-associated polypeptide encoded by said synthetic polynucleotide within cells.

Embodiment 109. The synthetic polynucleotide of any one of Embodiments 1-108, wherein said synthetic polynucleotide further comprises a 5′ cap structure.

Embodiment 110. The synthetic polynucleotide of Embodiment 109, wherein said 5′ cap structure improves a pharmacokinetic characteristic (e.g., a prolonged half-life) of said polynucleotide in a subject.

Embodiment 111. The synthetic polynucleotide of Embodiments 109 or 110, wherein said 5′cap structure is a Cap-1 structure.

Embodiment 112. The synthetic polynucleotide of any one of Embodiments 107-110, wherein said 3′ noncoding region comprises a poly adenosine tail.

Embodiment 113. The synthetic polynucleotide of Embodiment 112, wherein said poly adenosine tail comprises at most 200 adenosines.

Embodiment 114. The synthetic polynucleotide of Embodiment 112 or 113, wherein said poly adenosine tail improves a pharmacokinetic characteristic (e.g., a prolonged half-life) of said polynucleotide in a subject.

Embodiment 115. The synthetic polynucleotide of any one of Embodiments 1-114, wherein said synthetic polynucleotide encodes a cytoplasmic dynein assembly factor.

Embodiment 116. The synthetic polynucleotide of any one of Embodiments 1-115, wherein said synthetic polynucleotide encodes a cytoplasmic or axonemal dynein component protein.

Embodiment 117. The synthetic polynucleotide of any one of Embodiments 1-116, wherein said synthetic polynucleotide is a messenger ribonucleotide (mRNA) of a gene set forth in Table 1.

Embodiment 118. The synthetic polynucleotide of Embodiment 117, wherein said synthetic polynucleotide is an mRNA of a gene selected from the group consisting of DNAHS, ARMC4, ZMYND10, DNAAF4, CCDC40, CCDC39, DNAAF1, DNAI2, and DAAF2.

Embodiment 119. The synthetic polynucleotide of any one of Embodiments 1-118, wherein said synthetic polynucleotide is not a messenger ribonucleotide (mRNA) of DNAIl.

Embodiment 120. The synthetic polynucleotide of any one of Embodiments 1-119, wherein said synthetic polynucleotide comprises one or more nucleoside analogue(s) (e.g., one or more uridine analogue(s), such as 1-methylpseudouridine).

Embodiment 121. The synthetic polynucleotide of any one of Embodiments 1-120, wherein no more than 50% of nucleosides within said synthetic polynucleotide are nucleoside analogue(s) (e.g., uridine analogue(s), such as 1-methylpseudouridine).

Embodiment 122. The synthetic polynucleotide of any one of Embodiments 1-120, wherein no more than 20% of nucleosides within said synthetic polynucleotide are nucleoside analogue(s).

Embodiment 123. The synthetic polynucleotide of any one of Embodiments 1-121, wherein substantially all (e.g., at least about 80%, 90%, 95%, 97%, or 99%) nucleosides replacing uridine within said synthetic polynucleotide are nucleoside analogues.

Embodiment 124. A pharmaceutical composition comprising a synthetic polynucleotide of any one of Embodiments 1-123 combined with a lipid composition.

Embodiment 125. A pharmaceutical composition comprising a polynucleotide combined with a lipid composition, which polynucleotide (1) encodes a primary ciliary dyskinesia (PCD)-associated protein and (2) comprises a nucleic acid sequence having at least about 70% sequence identity over at least 500 or 1000 bases to a sequence selected from SEQ ID NOs: 1-32, 61, or 62.

Embodiment 126. The pharmaceutical composition of Embodiment 124 or 125, wherein said pharmaceutical composition comprises a cationic lipid or a cationic polymer.

Embodiment 127. The pharmaceutical composition of any one of Embodiments 124-126, wherein said pharmaceutical composition further comprises a phospholipid.

Embodiment 128. The pharmaceutical composition of any one of Embodiments 124-127, wherein said pharmaceutical composition further comprises a polymer-conjugated lipid (e.g., poly(ethylene glycol) (PEG)-conjugated lipid).

Embodiment 129. The pharmaceutical composition of any one of Embodiments 124-128, wherein said pharmaceutical composition further comprises a steroid or steroid derivative.

Embodiment 130. The pharmaceutical composition of any one of Embodiments 128-129, wherein said pharmaceutical formulation is formulated in a nanoparticle or a nanocapsule.

Embodiment 131. The pharmaceutical composition of any one of Embodiments 124-130, wherein said pharmaceutical formulation is formulated for local or systemic administration.

Embodiment 132. A method for treating a subject having or suspected of having primary ciliary dyskinesia (PCD), comprising administering to said subject in need thereof a composition comprising a synthetic polynucleotide of any one of Embodiments 1-123, thereby resulting in a heterologous expression of said PCD-associated protein within cells of said subject.

Embodiment 133. A method for treating a subject having or suspected of having primary ciliary dyskinesia (PCD), comprising administering to said subject in need thereof a composition comprising a synthetic polynucleotide that encodes a PCD-associated protein, which synthetic polynucleotide comprises a nucleic acid sequence having at least about 70% sequence identity over at least 500 or 1,000 bases to a sequence selected from SEQ ID NOs: 1-32, 61, or 62, thereby resulting in a heterologous expression of said PCD-associated protein within cells of said subject.

Embodiment 134. A method for treating a subject having or suspected of having primary ciliary dyskinesia (PCD), comprising administering to said subject in need thereof a pharmaceutical composition of any one of Embodiments 121-128, thereby resulting in a heterologous expression of said PCD-associated protein within cells of said subject.

Embodiment 135. A method for treating a subject having or suspected of having primary ciliary dyskinesia (PCD), comprising administering to said subject in need thereof a pharmaceutical composition comprising a polynucleotide combined with a lipid composition, which polynucleotide (1) encodes a primary ciliary dyskinesia (PCD)-associated protein and (2) comprises a nucleic acid sequence having at least about 70% sequence identity over at least 500 or 1,000 bases to a sequence selected from SEQ ID NOs: 1-32, 61, or 62, thereby resulting in a heterologous expression of said PCD-associated protein within cells of said subject.

Embodiment 136. The method of any one of Embodiments 132-135, wherein said subject is a human.

Embodiment 137. The method of any one of Embodiments 132-136, wherein said subject is determined to have an aberrant expression or activity of a PCD-associated gene or protein.

Embodiment 138. The method of any one of Embodiments 132-137, wherein said cells are ciliated cells.

Embodiment 139. The method of any one of Embodiments 132-137, wherein said cells are differentiated cells.

Embodiment 140. The method of any one of Embodiments 132-137, wherein said cells are undifferentiated cells.

Embodiment 141. The method of Embodiment 138 wherein said ciliated cells are ciliated epithelial cells (e.g., ciliated airway epithelial cells).

Embodiment 142. The method of Embodiment 141, wherein said ciliated epithelial cells are undifferentiated.

Embodiment 143. The method of Embodiment 141, wherein said ciliated epithelial cells are differentiated.

Embodiment 144. The method of any one of Embodiments 132-143, wherein said (e.g., ciliated) cells are in a lung of said subject.

### EXAMPLES

### Example 1: Production of mRNA That a PCD-Associated Protein

DNA corresponding to the genes of DNAHS, DNAI1, DNAI2, DNAAF1, DNAAF2, DNAAF3, DNAAF4, AMRC4, and ZMYND10 were synthesized at GenScript and each gene was provided as a separate pUC57 plasmid. In vitro transcription procedure was used for RNA production utilizing unmodified nucleotides, or modified nucleotides (e.g., 1-methylpseudouridine (m1Ψ)). Capping reaction was carried out using Vaccinia Virus capping system and cap 2′-O-methyl transferase.

### Example 2. Expression of PCD-Associated Proteins in Mammalian Cells

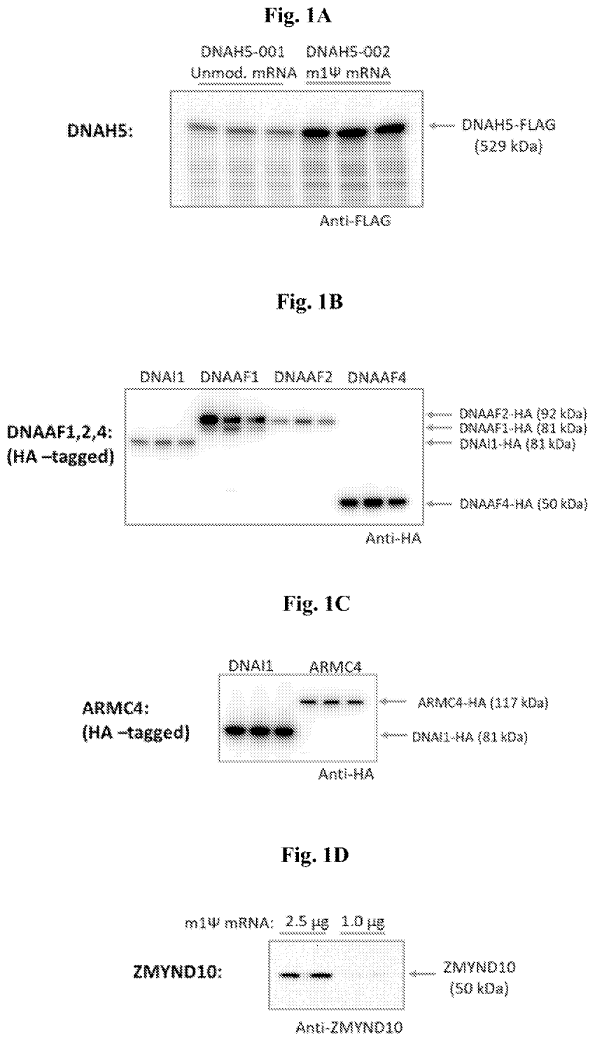

This experiment demonstrates the expression (translation) of PCD associated proteins in A549 cells. FIG. 1A is a western blot illustrating the translations of DNAH5 using mRNA with modified and unmodified nucleotides at 6 hours post-transfection. For this experiment, 1.25×10⁶ A549 cells/well in a 6 well plate were transfected with 2.5 μg of DNAH5 RNA using 3.75 μl messenger max transfection reagent. 6 hours post transfection, cells were trypsinized, pelleted, and the pellet was lysed in RIPA buffer. The blot was probed with anti-FLAG antibody. DNAH5 is observed to express using both the unmodified and modified nucleotides with increase expression using the mRNA with 1-methylpseudouridine. FIGS. 1B and 1C show a western blot illustrating the translations of HA-tagged DNAAF1, 2, 4 and ARMC4 using mRNA with unmodified nucleotides. A similar protocol as described for DNAH5 was performed. The blot was probed with anti-HA antibody. An HA-tagged DNAI1 was used as positive control. DNAAF1, 2, 4 and ARMC4 were observed to express. FIG. 1D shows a western blot illustrating the translations of ZMYND10 using mRNA with modified nucleotides (1-methylpseudouridine). A similar protocol as described for DNAH5 was performed, with a transfection of 2.5 ug or 1.0 ug mRNA in a well. The blot was probed with anti-ZMYND10 antibody. ZMYND10 was observed to express in the cells.

### Example 3. Expression of DNAI1/DNAI2 and Co-Immunoprecipitation

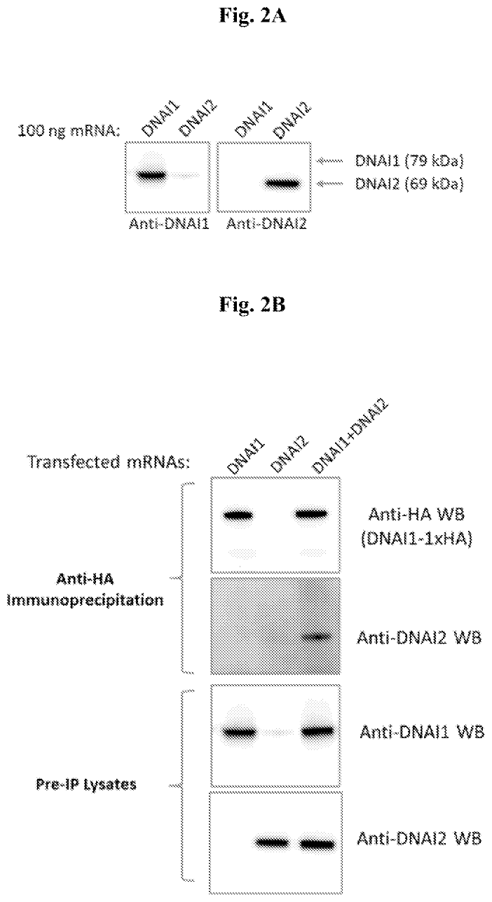

A549 cells were transfected with modified (e.g., 1-methylpseudouridine) mRNA of DNAIl/DNAI2 using MessengerMax. 3 sets of cells were generated, one with a transfection of DNAI1-1xHA, one with DNAI2-FLAG, another with a co-transfection of DNAI1-1xHA and DNAI2-FLAG. The cells were harvested after 6 hours and then lysed. FIG. 2A shows the detection of DNAIl/DNAI2. In cells transfected with DNAIl, a strong band for DNAI1 is observed while no DNAI2 is detected. However, when DNAI2 is transfected, a strong band for DNAI2 is observed and a weak, but detectable DNAI1 is also observed. FIG. 2B shows the western blots of the sets of proteins captured via immunoprecipitation with the anti-HA antibody. Cells were transfected with mRNA encoding DNAI1-HA and/or DNAI2-FLAG. Extracts were precipitated with anti-HA. For cells transfected with DNAIl-HA, after immunoprecipitation with anti-HA, a strong band for DNAI1 was detected. When cells were transfected with mRNA encoding DNAI2-FLAG, no band could be observed for DNAI2 after precipitation with anti-HA tag, but presence of DNAI2 was confirmed with anti-DNAI2 in the pre-IP lysates. In cells transfected with mRNAs encoding both DNAIl-HA and DNAI2-FLAG, DNAI2 could be observed co-immunoprecipitating with DNAI1 As a control the pre-IP lysates were also blotted with anti-DNA1 and anti-DNAI2 and show expression of their respective protein.

### Example 4. Detection of PCD-Associated Proteins mRNA Delivery to a Subject

A subject having or suspected of having primary ciliary dyskinesia (PCD) is given a treatment by administering a composition as described herein. The subject is monitored at regular intervals for expression of PCD-associated proteins in the lungs. A sample of lung tissue from the subject is taken comprising ciliated cells of the lung. The cells are harvested and prepared for RNA isolation. cDNA is produced from the RNA using a first strand synthesis kit and random hexamer. qPCR reactions are run using a set of forward and reverse primers and a fluorescent probe, specific to each of a PCD-associated protein and another set specific to a control or housekeeping gene for expression normalization. Expression of PCD-associated proteins is detected using a fluorescent readout corresponding the probes for PCD-associated proteins.

### Example 5. Treatment of CCDC39 Negative Cells (PCD Cells)

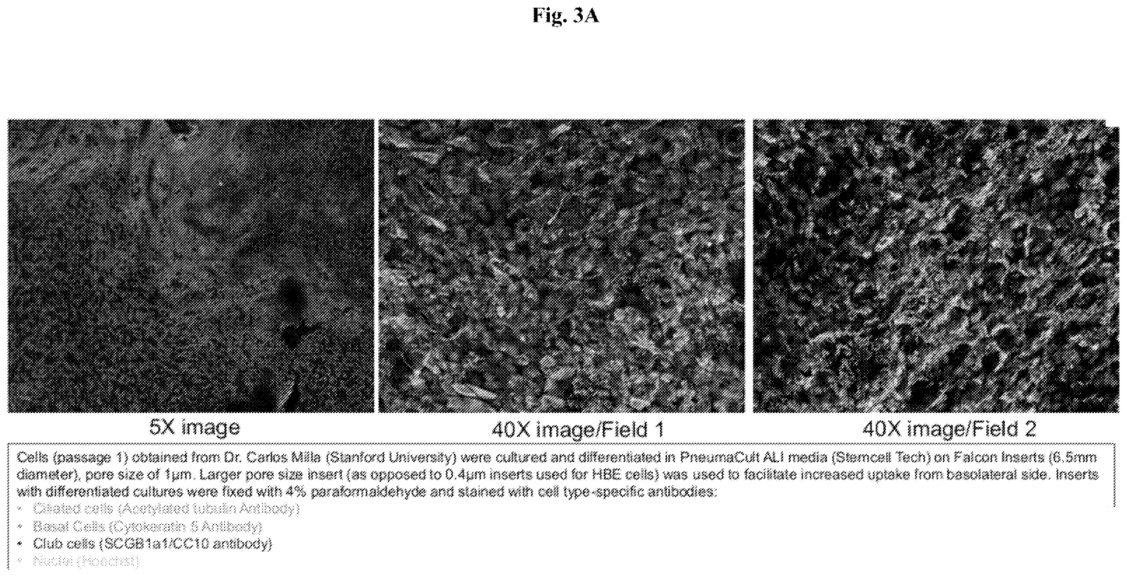

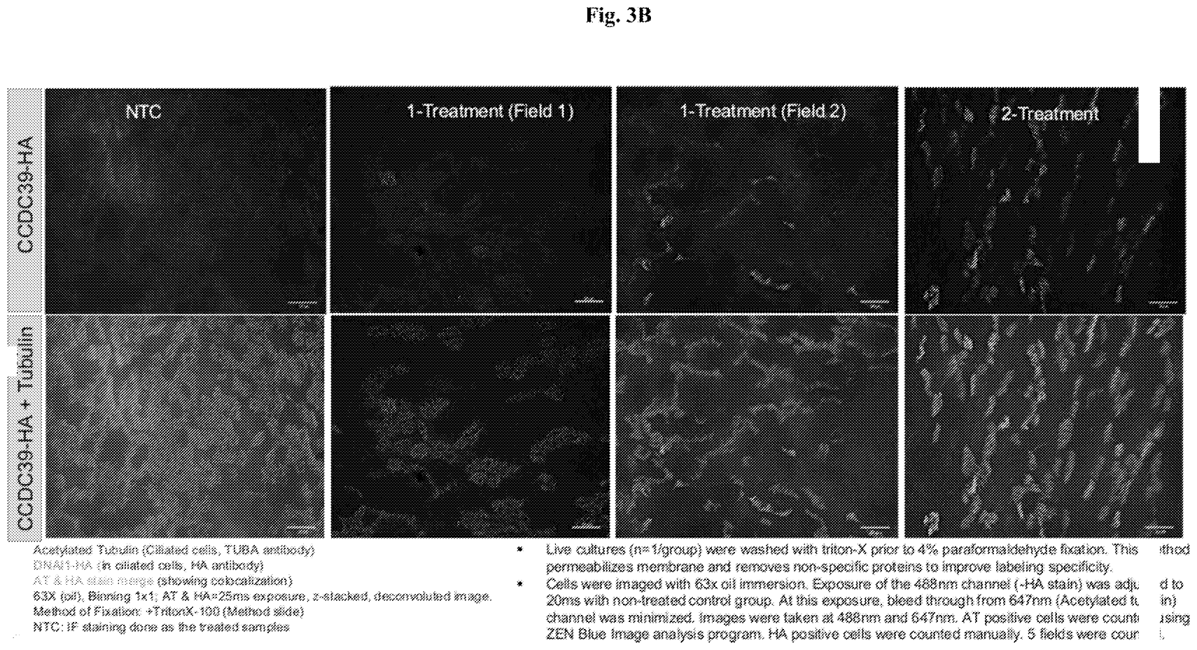

Human nasal epithelial cells were obtained from PCD patients and cultured to obtain human nasal epithelial culture (HNEC, negative for CCDC39 expression). The cells were differentiated on inserts (6.5 mm in diameter), pore size of 1 μm. Larger pore size insert (as opposed to, for example, 0.4 μm inserts) was used to facilitate increased uptake from basolateral side. Inserts with differentiated cultures were fixed with 4% paraformaldehyde. FIG. 3A illustrates immunofluorescent staining of the fixed cells with cell type-specific antibodies: ciliated cell (acetylated tubulin antibody); basal cell (cytokeratin 5 antibody); club cells (SCGB1a1/CC10 antibody), and nuclei (Hoechst). Cilia activity was not detected in differentiated PCD cultures. When cultured in similar conditions, normal HNEC cilia activity could be read by Sisson-Ammons Video Analysis system (SAVA) 14 days after the cells contacted with differentiation media. Nasal cultures generated increased mucus than human bronchial epithelial (HBE) cultures.

The HNEC cells were treated with CCDC39 mRNA (e.g., mRNA comprising modified nucleotides such as 1-methylpseudouridine; CCDC39 mRNA also contained an HA tag for expression detection). The CCDC39 mRNA was encapsulated or formulated with a nanoparticle described herein. FIG. 3B illustrates axoneme incorporation (72 hours after treatment with CCDC39 mRNA) of CCDC39-HA in the CCDC39 negative PCD cell (HNEC) after single dose or two doe treatment. Live cell cultures (n=1/group) were washed with triton-X prior to 4% paraformaldehyde fixation for permeabilizing membrane and removing non-specific proteins to improve labeling specificity. Cells were imaged with 63× oil immersion. Exposure of the 488 nm channel (-HA stain) was adjusted to 20 ms with non-treated control group. At this exposure, bleed through from 647 nm (acetylated tubulin) channel was minimized. Images were taken at 488 nm and 647 nm. AT positive cells were counted using ZEN Blue Image analysis program. Antibody used: acetylated tubulin (AT, ciliated cells, TUBA antibody); and CCDC39-HA (in ciliated cells, HA antibody) with AT and HA stain merge (showing colocalization).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A synthetic polynucleotide encoding a primary ciliary dyskinesia (PCD)-associated protein, wherein said synthetic polynucleotide comprises a nucleic acid sequence (e.g., an open reading frame (ORF) sequence) having at least about 70% sequence identity over at least 500 or 1000 bases to a sequence selected from SEQ ID NOs: 1-32, 61, or 62.

2. The synthetic polynucleotide of claim 1, wherein said nucleic acid sequence has at least about 75% (e.g., at least about 80%, 81%, 82%, 83%, 84%, 85%, 86%, 87%, 88%, 89%, 90%, 91%, 92%, 93%, 94%, 95%, 96%, 97%, 98%, or 99%) sequence identity over at least 500 or 1000 bases to a sequence selected from SEQ ID NOs: 1-32, 61, or 62.

3. The synthetic polynucleotide of claim 1, wherein said nucleic acid sequence has 100% sequence identity over at least 500 or 1000 bases to a sequence selected from SEQ ID NOs: 1-32, 61, or 62.

4. The synthetic polynucleotide of claim 1, wherein said nucleic acid sequence has at least about 70% sequence identity to a sequence selected from SEQ ID NOs: 1-32, 61, or 62.

5. The synthetic polynucleotide of claim 4, wherein said nucleic acid sequence has at least about 75% (e.g., at least about 80%, 81%, 82%, 83%, 84%, 85%, 86%, 87%, 88%, 89%, 90%, 91%, 92%, 93%, 94%, 95%, 96%, 97%, 98%, or 99%) sequence identity to a sequence selected from SEQ ID NOs: 1-32, 61, or 62.

6. The synthetic polynucleotide of claim 1, wherein said nucleic acid sequence comprises a reduced number or frequency of at least one codon selected from the group consisting of GCG, GCA, GCT, TGT, GAT, GAG, TTT, GGG, GGT, CAT, ATA, ATT, AAG, TTG, TTA, CTA, CTT, CTC, AAT, CCG, CCA, CCT, CAG, AGG, CGG, CGA, CGT, CGC, TCG, TCA, TCT, TCC, ACG, ACT, GTA, GTT, GTC, and TAT, as compared to a corresponding wild-type sequence selected from SEQ ID NOs: 33-39.

7. The synthetic polynucleotide of claim 1, wherein said nucleic acid sequence comprises an increased number or frequency of at least one codon comprising one or more codons selected from: GCC, TGC, GAC, GAA, TTC, GGA, GGC, CAC, ATC, AAA, CTG, AAC, CCT, CCC, CAA, AGA, AGC, ACA, ACC, GTG, and TAC, as compared to a corresponding wild-type sequence selected from SEQ ID NOs: 33-39.

8. The synthetic polynucleotide of claim 1, wherein said nucleic acid sequence comprises fewer codon types encoding an amino acid as compared to a corresponding wild-type sequence selected from SEQ ID NOs: 33-39.

9. The synthetic polynucleotide of claim 8, wherein at least one type of an isoleucine-encoding codon in said corresponding wild-type sequence is substituted with a synonymous codon type in said nucleic acid sequence.

10. The synthetic polynucleotide of claim 8, wherein at least one type of a valine-encoding codon in said corresponding wild-type sequence is substituted with a synonymous codon type in said nucleic acid sequence.

11. The synthetic polynucleotide of claim 8, wherein at least one type of an alanine-encoding codon in said corresponding wild-type sequence is substituted with a synonymous codon type in said nucleic acid sequence.

12. The synthetic polynucleotide of claim 8, wherein at least one type of a glycine-encoding codon in said corresponding wild-type sequence is substituted with a synonymous codon type in said nucleic acid sequence.

13. The synthetic polynucleotide of claim 8, wherein at least one type of a proline-encoding codon in said corresponding wild-type sequence is substituted with a synonymous codon type in said nucleic acid sequence.

14. The synthetic polynucleotide of claim 8, wherein at least one type of a threonine-encoding codon in said corresponding wild-type sequence is substituted with a synonymous codon type in said nucleic acid sequence.

15. The synthetic polynucleotide of claim 8, wherein at least one type of a leucine-encoding codon in said corresponding wild-type sequence is substituted with a synonymous codon type in said nucleic acid sequence.

16. The synthetic polynucleotide of claim 8, wherein at least one type of an arginine-encoding codon in said corresponding wild-type sequence is substituted with a synonymous codon type in said nucleic acid sequence.

17. The synthetic polynucleotide of claim 8, wherein at least one type of a serine-encoding codon in said corresponding wild-type sequence is substituted with a synonymous codon type in said nucleic acid sequence.

18. The synthetic polynucleotide of claim 1, wherein at least about 90% phenylalanine-encoding codons of said synthetic polynucleotide are TTC (as opposed to TTT).

19. The synthetic polynucleotide of claim 1, wherein at least about 60% cysteine-encoding codons of said synthetic polynucleotide are TGC (as opposed to TGT).

20. The synthetic polynucleotide of claim 1, wherein at least about 70% aspartic acid-encoding codons of said synthetic polynucleotide are GAC (as opposed to GAT).

21. The synthetic polynucleotide of claim 1, wherein at least about 50% glutamic acid-encoding codons of said synthetic polynucleotide are GAG (as opposed to GAA).

22. The synthetic polynucleotide of claim 1, wherein at least about 60% histidine-encoding codons of said synthetic polynucleotide are CAC (as opposed to CAT).

23. The synthetic polynucleotide of claim 1, wherein at least about 60% lysine-encoding codons of said synthetic polynucleotide are AAG (as opposed to AAA).

24. The synthetic polynucleotide of claim 1, wherein at least about 60% asparagine-encoding codons of said synthetic polynucleotide are AAC (as opposed to AAT).

25. The synthetic polynucleotide of claim 1, wherein at least about 70% glutamine-encoding codons of said synthetic polynucleotide are CAG (as opposed to CAA).

26. The synthetic polynucleotide of claim 1, wherein at least about 80% tyrosine-encoding codons of said synthetic polynucleotide are TAC (as opposed to TAT).

27. The synthetic polynucleotide of claim 1, wherein at least about 90% isoleucine-encoding codons of said synthetic polynucleotide are ATC.

28. The synthetic polynucleotide of claim 1, wherein said synthetic polynucleotide comprises no more than 2 types of isoleucine-encoding codons.

29. The synthetic polynucleotide of claim 1, wherein said synthetic polynucleotide comprises no more than 3 types of alanine (Ala)-encoding codons.

30. The synthetic polynucleotide of claim 1, wherein said synthetic polynucleotide comprises no more than 3 types of glycine (Gly)-encoding codons.

31. The synthetic polynucleotide of claim 1, wherein said synthetic polynucleotide comprises no more than 3 types of proline (Pro)-encoding codons.

32. The synthetic polynucleotide of claim 1, wherein said synthetic polynucleotide comprises no more than 3 types of threonine (Thr)-encoding codons.

33. The synthetic polynucleotide of claim 1, wherein said synthetic polynucleotide comprises no more than 5 or 4 type(s) of arginine (Arg)-encoding codons.

34. The synthetic polynucleotide of claim 1, wherein said synthetic polynucleotide comprises no more than 5 or 4 type(s) of serine (Ser)-encoding codons.

35. The synthetic polynucleotide of claim 1, a frequency of GCC codon is higher than a frequency of GCA codon.

36. The synthetic polynucleotide of claim 1, a frequency of GCC codon is higher than a frequency of GCT codon.

37. The synthetic polynucleotide of claim 1, a frequency of GCT codon is lower than a frequency of GCA codon.

38. The synthetic polynucleotide of claim 1, a frequency of GCT codon is higher than a frequency of GCA codon.

39. The synthetic polynucleotide of claim 1, a frequency of GCG codon is no more than about 10% or 5%.

40. The synthetic polynucleotide of claim 1, a frequency of GCA codon is no more than about 20%.

41. The synthetic polynucleotide of claim 1, a frequency of GCT codon is at least about 1%, 5%, 10%, 15%, 20%, or 25%.

42. The synthetic polynucleotide of claim 1, a frequency of GCT codon is no more than about 30%, 25%, 20%, 15%, 10%, or 5%.

43. The synthetic polynucleotide of claim 1, a frequency of GCC codon is at least about 60%, 70%, 80%, or 90%.

44. The synthetic polynucleotide of claim 1, a frequency of GCC codon is no more than about 95%, 90%, 85%, 80%, or 75%.

45. The synthetic polynucleotide of claim 1, a frequency of GGC codon is lower than a frequency of GGA codon.

46. The synthetic polynucleotide of claim 1, a frequency of GGC codon is higher than a frequency of GGA codon.

47. The synthetic polynucleotide of claim 1, a frequency of GGG codon is no more than about 10% or 5%.

48. The synthetic polynucleotide of claim 1, a frequency of GGG codon is at least about 1%.

49. The synthetic polynucleotide of claim 1, a frequency of GGA codon is no more than about 30% or 20%.

50. The synthetic polynucleotide of claim 1, a frequency of GGA codon is at least about 10% or 20%.

51. The synthetic polynucleotide of claim 1, a frequency of GGT codon is no more than about 10% or 5%.

52. The synthetic polynucleotide of claim 1, a frequency of GGC codon is no more than about 90%, 80%, or 70%.

53. The synthetic polynucleotide of claim 1, a frequency of GGC codon is at least about 60%, 70%, or 80%.

54. The synthetic polynucleotide of claim 1, a frequency of CCC codon is lower than a frequency of CCT codon.

55. The synthetic polynucleotide of claim 1, a frequency of CCC codon is higher than a frequency of CCT codon.

56. The synthetic polynucleotide of claim 1, a frequency of CCC codon is lower than a frequency of CCA codon.

57. The synthetic polynucleotide of claim 1, a frequency of CCC codon is higher than a frequency of CCA codon.

58. The synthetic polynucleotide of claim 1, a frequency of CCT codon is lower than a frequency of CCA codon.

59. The synthetic polynucleotide of claim 1, a frequency of CCT codon is higher than a frequency of CCA codon.

60. The synthetic polynucleotide of claim 1, a frequency of CCG codon is no more than about 10% or 5%

61. The synthetic polynucleotide of claim 1, a frequency of CCA codon is no more than about 30%, 20%, or 10%.

62. The synthetic polynucleotide of claim 1, a frequency of CCA codon is at least about 5%, 10%, 15%, 20%, or 25%.

63. The synthetic polynucleotide of claim 1, a frequency of CCT codon is no more than about 60%, 50%, 40%, or 30%.

64. The synthetic polynucleotide of claim 1, a frequency of CCT codon is at least about 20%, 30%, 40%, or 50%.

65. The synthetic polynucleotide of claim 1, a frequency of CCC codon is no more than about 60%, 50%, or 40%.

66. The synthetic polynucleotide of claim 1, a frequency of CCC codon is at least about 30%, 40%, 50%, 60%, or 70%.

67. The synthetic polynucleotide of claim 1, a frequency of ACA codon is higher than a frequency of ACT codon.

68. The synthetic polynucleotide of claim 1, a frequency of ACC codon is higher than a frequency of ACT codon.

69. The synthetic polynucleotide of claim 1, a frequency of ACC codon is lower than a frequency of ACA codon.

70. The synthetic polynucleotide of claim 1, a frequency of ACC codon is higher than a frequency of ACA codon.

71. The synthetic polynucleotide of claim 1, a frequency of ACG codon is no more than about 10% or 5%.

72. The synthetic polynucleotide of claim 1, a frequency of ACA codon is no more than about 60%, 50%, 40%, or 30%.

73. The synthetic polynucleotide of claim 1, a frequency of ACA codon is at least about 10%, 20%, 30%, 40%, or 50%.

74. The synthetic polynucleotide of claim 1, a frequency of ACT codon is no more than about 10% or 5%.

75. The synthetic polynucleotide of claim 1, a frequency of ACC codon is no more than about 90%, 80%, 70%, 60%, or 50%.

76. The synthetic polynucleotide of claim 1, a frequency of ACC codon is at least about 40%, 50%, 60%, 70%, or 80%.

77. The synthetic polynucleotide of claim 1, a frequency of AGA codon is lower than a frequency of AGG codon.

78. The synthetic polynucleotide of claim 1, a frequency of AGA codon is higher than a frequency of AGG codon.

79. The synthetic polynucleotide of claim 1, a frequency of AGA codon is lower than a frequency of CGG codon.

80. The synthetic polynucleotide of claim 1, a frequency of AGA codon is higher than a frequency of CGG codon.

81. The synthetic polynucleotide of claim 1, a frequency of CGG codon is higher than a frequency of CGA codon.

82. The synthetic polynucleotide of claim 1, a frequency of CGG codon is higher than a frequency of CGC codon.

83. The synthetic polynucleotide of claim 1, a frequency of AGG codon is no more than about 10%.

84. The synthetic polynucleotide of claim 1, a frequency of AGG codon is less than about 10%.

85. The synthetic polynucleotide of claim 1, a frequency of AGA codon is no more than about 70%, 60%, or 50%.

86. The synthetic polynucleotide of claim 1, a frequency of AGA codon is at least about 40%, 50%, 60%, or 70%.

87. The synthetic polynucleotide of claim 1, a frequency of CGG codon is no more than about 50%, 40%, or 30%.

88. The synthetic polynucleotide of claim 1, a frequency of CGG codon is at least about 20%, 30%, or 40%.

89. The synthetic polynucleotide of claim 1, a frequency of CGA codon is at least about 1%.

90. The synthetic polynucleotide of claim 1, a frequency of CGA codon is no more than about 10% or 5%.

91. The synthetic polynucleotide of claim 1, a frequency of CGT codon is no more about 10% or 5%.

92. The synthetic polynucleotide of claim 1, a frequency of CGC codon is no more than about 20%, 10%, or 5%.

93. The synthetic polynucleotide of claim 1, a frequency of CGC codon is at least about 1%, 2%, 3%, 4%, or 5%.

94. The synthetic polynucleotide of claim 1, a frequency of AGC codon is higher than a frequency of TCT codon.

95. The synthetic polynucleotide of claim 1, a frequency of TCT codon is higher than a frequency of TCG codon.

96. The synthetic polynucleotide of claim 1, a frequency of TCT codon is higher than a frequency of TCA codon.

97. The synthetic polynucleotide of claim 1, a frequency of TCT codon is higher than a frequency of TCC codon.

98. The synthetic polynucleotide of claim 1, a frequency of AGT codon is no more than about 10%.

99. The synthetic polynucleotide of claim 1, a frequency of AGT codon is at least about 1%.

100. The synthetic polynucleotide of claim 1, a frequency of AGC codon is no more about 95%, 90%, 85%, or 80%.

101. The synthetic polynucleotide of claim 1, a frequency of AGC codon is at least about 70%, 80%, or 90%.

102. The synthetic polynucleotide of claim 1, a frequency of TCG codon is no more than about 10% or 5%.

103. The synthetic polynucleotide of claim 1, a frequency of TCA codon is no more than about 10% or 5%.

104. The synthetic polynucleotide of claim 1, a frequency of TCT codon is no more than about 30%, 20%, or 10%.

105. The synthetic polynucleotide of claim 1, a frequency of TCT codon is at least about 10%, or 20%.

106. The synthetic polynucleotide of claim 1, a frequency of TCC codon is no more than about 10% or 5%.

107. The synthetic polynucleotide of claim 1, wherein said synthetic polynucleotide further comprises a 3′ or 5′ noncoding region.

108. The synthetic polynucleotide of claim 107, wherein said 3′ or 5′ noncoding region enhances an expression of said PCD-associated polypeptide encoded by said synthetic polynucleotide within cells.

109. The synthetic polynucleotide of claim 1, wherein said synthetic polynucleotide further comprises a 5′ cap structure.

110. The synthetic polynucleotide of claim 109, wherein said 5′ cap structure improves a pharmacokinetic characteristic (e.g., a prolonged half-life) of said polynucleotide in a subject.

111. The synthetic polynucleotide of claim 109, wherein said 5′cap structure is a Cap-1 structure.

112. The synthetic polynucleotide of claim 107, wherein said 3′ noncoding region comprises a poly adenosine tail.

113. The synthetic polynucleotide of claim 112, wherein said poly adenosine tail comprises at most 200 adenosines.

114. The synthetic polynucleotide of claim 112, wherein said poly adenosine tail improves a pharmacokinetic characteristic (e.g., a prolonged half-life) of said polynucleotide in a subject.

115. The synthetic polynucleotide of claim 1, wherein said synthetic polynucleotide encodes a cytoplasmic dynein assembly factor.

116. The synthetic polynucleotide of claim 1, wherein said synthetic polynucleotide encodes a cytoplasmic or axonemal dynein component protein.

117. The synthetic polynucleotide of claim 1, wherein said synthetic polynucleotide is a messenger ribonucleotide (mRNA) of a gene set forth in Table 1.

118. The synthetic polynucleotide of claim 117, wherein said synthetic polynucleotide is an mRNA of a gene selected from the group consisting of DNAHS, ARMC4, ZMYND10, DNAAF4, CCDC40, CCDC39, DNAAF1, DNAI2, and DAAF2.

119. The synthetic polynucleotide of claim 1, wherein said synthetic polynucleotide is not a messenger ribonucleotide (mRNA) of DNAI1.

120. The synthetic polynucleotide of claim 1, wherein said synthetic polynucleotide comprises one or more nucleoside analogue(s) (e.g., one or more uridine analogue(s), such as 1-methylpseudouridine).

121. The synthetic polynucleotide of claim 1, wherein no more than 50% of nucleosides within said synthetic polynucleotide are nucleoside analogue(s) (e.g., uridine analogue(s), such as 1-methylpseudouridine).

122. The synthetic polynucleotide of claim 1, wherein no more than 20% of nucleosides within said synthetic polynucleotide are nucleoside analogue(s).

123. The synthetic polynucleotide of claim 1, wherein substantially all (e.g., at least about 80%, 90%, 95%, 97%, or 99%) nucleosides replacing uridine within said synthetic polynucleotide are nucleoside analogues.

124. A pharmaceutical composition comprising a synthetic polynucleotide of any one of claims 1-123 combined with a lipid composition.

125. A pharmaceutical composition comprising a polynucleotide combined with a lipid composition, which polynucleotide (1) encodes a primary ciliary dyskinesia (PCD)-associated protein and (2) comprises a nucleic acid sequence having at least about 70% sequence identity over at least 500 or 1000 bases to a sequence selected from SEQ ID NOs: 1-32, 61, or 62.

126. The pharmaceutical composition of claim 124, wherein said pharmaceutical composition comprises a cationic lipid or a cationic polymer.

127. The pharmaceutical composition of claim 124, wherein said pharmaceutical composition further comprises a phospholipid.

128. The pharmaceutical composition of claim 124, wherein said pharmaceutical composition further comprises a polymer-conjugated lipid (e.g., poly(ethylene glycol) (PEG)-conjugated lipid).

129. The pharmaceutical composition of claim 124, wherein said pharmaceutical composition further comprises a steroid or steroid derivative.

130. The pharmaceutical composition of claim 124, wherein said pharmaceutical formulation is formulated in a nanoparticle or a nanocapsule.

131. The pharmaceutical composition of claim 124, wherein said pharmaceutical formulation is formulated for local or systemic administration.

132. A method for treating a subject having or suspected of having primary ciliary dyskinesia (PCD), comprising administering to said subject in need thereof a composition comprising a synthetic polynucleotide of any one of claims 1-123, thereby resulting in a heterologous expression of said PCD-associated protein within cells of said subject.

133. A method for treating a subject having or suspected of having primary ciliary dyskinesia (PCD), comprising administering to said subject in need thereof a composition comprising a synthetic polynucleotide that encodes a PCD-associated protein, which synthetic polynucleotide comprises a nucleic acid sequence having at least about 70% sequence identity over at least 500 or 1,000 bases to a sequence selected from SEQ SEQ ID NOs: 1-32, 61, or 62, thereby resulting in a heterologous expression of said PCD-associated protein within cells of said subject.

134. A method for treating a subject having or suspected of having primary ciliary dyskinesia (PCD), comprising administering to said subject in need thereof a pharmaceutical composition of any one of claims 121-128, thereby resulting in a heterologous expression of said PCD-associated protein within cells of said subject.

135. A method for treating a subject having or suspected of having primary ciliary dyskinesia (PCD), comprising administering to said subject in need thereof a pharmaceutical composition comprising a polynucleotide combined with a lipid composition, which polynucleotide (1) encodes a primary ciliary dyskinesia (PCD)-associated protein and (2) comprises a nucleic acid sequence having at least about 70% sequence identity over at least 500 or 1,000 bases to a sequence selected from SEQ ID NOs: 1-32, 61, or 62, thereby resulting in a heterologous expression of said PCD-associated protein within cells of said subject.

136. The method of claim 132, wherein said subject is a human.

137. The method of claim 132, wherein said subject is determined to have an aberrant expression or activity of a PCD-associated gene or protein.

138. The method of claim 132, wherein said cells are ciliated cells.

139. The method of claim 132, wherein said cells are differentiated cells.

140. The method of claim 132, wherein said cells are undifferentiated cells.

141. The method of claim 138, wherein said ciliated cells are ciliated epithelial cells (e.g., ciliated airway epithelial cells).

142. The method of claim 141, wherein said ciliated epithelial cells are undifferentiated.

143. The method of claim 141, wherein said ciliated epithelial cells are differentiated.

144. The method of claim 132, wherein said (e.g., ciliated) cells are in a lung of said subject.
